(19) United States
(12) Patent Application Publication
(10) Pub. No.: US 2025/0002473 A1
(43) Pub. Date: Jan. 2, 2025

(54) QUINOXALINE DERIVED SULFONAMIDES WITH EGFR DEGRADATION ACTIVITIES AND THEIR USES THEREOF

(71) Applicant: Accutar Biotechnology Inc., Cranbury, NJ (US)

(72) Inventors: Ji LIU, Shanghai (CN); Pin HUANG, Shanghai (CN); Ke LIU, Shanghai (CN); Wei HE, Zionsville, IN (US); Yimin QIAN, Plainsboro, NJ (US); Jie FAN, New York, NY (US); Robert Luo, New York, NY (US)

(73) Assignee: Accutar Biotechnology Inc., Cranbury, NJ (US)

(21) Appl. No.: 18/744,381

(22) Filed: Jun. 14, 2024

Related U.S. Application Data
(60) Provisional application No. 63/508,605, filed on Jun. 16, 2023.

(51) Int. Cl. C07D 401/14; A61K 31/506; C07D 413/14; C07D 471/10; C07D 487/10

(52) U.S. Cl. CPC C07D 401/14; A61K 31/506; C07D 413/14; C07D 471/10; C07D 487/10

(57) ABSTRACT

The present disclosure relates to at least one entity chosen from a compound of Formula (I), tautomers, stereoisomers or a mixture of stereoisomers, hydrates, and deuterated derivatives thereof, and pharmaceutically acceptable salts of any of the foregoing, and their use as epidermal growth factor receptor (EGFR) degraders for the prevention and treatment of diseases and conditions, e.g., cancer. The present disclosure also relates to pharmaceutical compositions containing such entities, and their use in treating or preventing a disease or disorder associated with EGFR.

This application claims benefit of priority to U.S. Provisional Patent Application No. 63/508,605, filed Jun. 16, 2023, which is hereby incorporated by reference in its entirety.

The present disclosure relates to novel quinoxaline derived sulfonamides, pharmaceutical compositions containing such compounds, and their use as epidermal growth factor receptor (EGFR) degraders for the prevention and treatment of diseases and conditions, such as cancer.

Protein kinases are a group of enzymes that regulate diverse, important biological processes including, for example, cell growth, proliferation, survival, invasion and differentiation, organ formation, tissue repair and regeneration. Protein kinases exert their physiological functions through catalyzing the phosphorylation of protein and thereby modulating cellular activities. Because protein kinases have profound effects on cells, their activities are highly regulated. Kinases are turned on or off by phosphorylation (sometimes by autophosphorylation), by binding of activator proteins or inhibitor proteins, or small molecules, or by controlling their location in the cell relative to their substrates.

EGFR is a transmembrane protein tyrosine kinase member of the erbB receptor family. Upon binding of a growth factor ligand such as epidermal growth factor (EGF), the receptor can homo-dimerize with another EGFR molecule or hetero-dimerize with another family member such as erbB2 (HER2), erbB3 (HER3), or erbB4 (HER4).

Homo- and/or hetero-dimerization of erbB receptors results in the phosphorylation of key tyrosine residues in the intracellular domain and leads to the stimulation of numerous intracellular signal transduction pathways involved in cell proliferation and survival. Deregulation of erbB family signaling promotes proliferation, invasion, metastasis, angiogenesis, and tumor cell survival and has been described in many human cancers, including those of the lung, head and neck and breast.

The erbB family therefore represents a rational target for anticancer drug development and a number of small molecule agents targeting EGFR or erbB2 are now clinically available, including gefitinib (Iressa®), erlotinib (TARCEVA®) and lapatinib (TYKERB®), dacomitinib (Vizimpro®), neratinib (Nerlynx®), tucatinib (Tukysa®), Osimertinib (Tagrisso®), afatinib (Gilotrif®), and mobocertinib (Exkivity®)

In 2004 it was reported (Science Vol. 304, 1497-500 and New England. Journal of Medicine Vol. 350, 2129-39) that activating mutations in EGFR correlated with response to gefitinib therapy in non-small-cell lung cancer (NSCLC). The most common EGFR activating mutations, L858R and delE746_A750, result in an increase in affinity for small molecule tyrosine kinase inhibitors such as gefitinib and erlotinib and a decrease in affinity for adenosine triphosphate (ATP) relative to wild type (WT) EGFR. Ultimately, acquired resistance to therapy with gefitinib or erlotinib arises, for example by mutation of the gatekeeper residue T790M, which is reportedly detected in 50% of clinically resistant patients. This mutation is not believed to hinder the binding of gefitinib or erlotinib to EGFR sterically, merely to alter the affinity to ATP to levels comparable to WT EGFR.

In view of the importance of this mutation in resistance to existing therapies targeting EGFR, it is believed that agents that can inhibit EGFR harboring the gatekeeper mutation may be especially useful in the treatment of cancer.

Osimertinib was developed to conquer the gatekeeper mutation and granted accelerated approval in the USA for the treatment of patients with metastatic EGFR T790M mutation-positive NSCLC who have progressed on or after EGFR TKI therapy. However, acquired resistance to Osimertinib therapy ultimately arises too. The most common ternary EGFR mutation is EGFR C797S, which accounts for 10-26% of cases of resistance to second-line osimertinib treatment and 7% of cases of resistance to first line osimertinib treatment. The EGFR C797S mutation, in which cysteine at codon 797 within the ATP-binding site is substituted for by serine, results in the loss of the covalent bond between osimertinib and the mutant EGFR. In view of the importance of EGFR C797S or other rare C797X mutations in resistance to existing therapies targeting EGFR T790M mutation, it is believed that agents that can inhibit EGFR harboring C797 mutations may be especially useful in the treatment of cancer.

Exon20 insertion mutations represents the third most common erbB family activating mutations in NSCLC. EGFR exon 20 insertion mutations collectively represent approximately 4% to 10% of all EGFR mutations. Most of EGFR exon 20 insertion mutations occur near the end of aC-helix after residue Met766, with EGFR D770_N771insSVD and V769_D770insASV accounting for about 40% of them. As EGFR exon20 insertion mutations, erbB2 exon20 insertion mutations occur in a similar prevalence in NSCLC and also in a similar position after residue Met774, with erbB2A775_G776insYVMA accounting for about 80% of them. See, Jang, J. et al. Angew. Chem. Int. Ed. (2018) Vol. 57 (36), 11629-11633.

HER2 mutations are reportedly present in about 2-4% of NSCLC (See, Stephens et al. Nature (2004) Vol. 431, 525-526). The most common mutation is an in-frame insertion within exon 20. In 83% of patients having HER2 associated NSCLC, a four amino acid YVMA insertion mutation occurs at codon 775 in exon 20 of HER2 (see, e.g., Arcila et al. Clin Cancer Res (2012) Vol. 18, 4910-4918). The exon 20 insertion results in increased HER2 kinase activity and enhanced signaling through downstream pathways, resulting in increased survival, invasiveness, and tumorigenicity (see, e.g., Wang et al. Cancer Cell (2006) Vol. 10, 25-38). Tumors harboring the HER2 YVMA mutation are largely resistant to known EGFR inhibitors.

Exon 20 insertion mutations are not restricted to lung cancer. Recent analysis of sinonasal squamous cell carcinoma (SNSCC), a rare form of head and neck cancer, demonstrated a remarkably high frequency of EGFR mutations (77% of SNSCC tumors), the majority of which were exon 20 insertions (88% of all EGFR mutations) (see, e.g., Udager, A. M. et al. Cancer Res. (2015) Vol. 75, 2600-2606).

Exon 20 insertion mutations rarely respond to treatment with currently approved EGFR and HER2 Tyrosine Kinase inhibitors (TKIs), such as gefitinib, erlotinib or afatinib, or chemotherapies.

There remains a need for compounds that may exhibit favorable potency profiles against WT EGFR versus activating mutant forms of EGFR/erbB2 (e.g., L858R EGFR mutant, or the delE746_A750 mutant or the Exon19 deletion EGFR mutant, or EGFR/erbB2 exon20 insertion mutations) and/or resistant mutant forms of EGFR (e.g., T790M EGFR mutant), and/or selectivity over other enzyme receptors which may make the compounds especially promising for development as therapeutic agents. In this regard, there remains a need for compounds that show a higher inhibition of certain activating or resistant mutant forms of EGFR and HER2 while at the same time showing relatively low inhibition of WT EGFR. Such compounds may be expected to be more suitable as therapeutic agents, particularly for the treatment of diseases, such as cancer, due to reduction of toxicology associated with WT EGFR inhibition. Such toxicologies are known to manifest themselves in humans as skin rashes and/or diarrhea.

The present disclosure provides, in some embodiments, quinoxaline derived sulfonamide compounds that have high potency against one or more mutant forms of EGFR and/or HER2 while at the same showing relatively low inhibition of WT EGFR.

In some embodiments, the present disclosure provides compounds, compositions, and methods for degrading epidermal growth factor receptors.

The present disclosure provides a compound represented by Formula (I), or a tautomer, a stereoisomer or a mixture of stereoisomers, a pharmaceutically acceptable salt, a hydrate, or a deuterated derivative thereof:

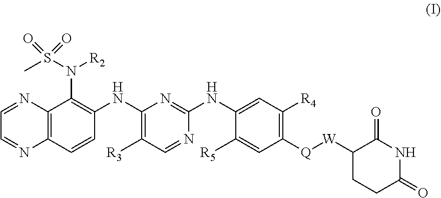

wherein:
R₁ is selected from hydroxy, amino, halogen, C₁-C₆ alkyl, C₃-C₆ cycloalkyl, C₁-C₆ haloalkyl, and C₁-C₆ alkoxy;
R₂ is selected from hydrogen, C₁-C₆ alkyl, C₃-C₈ cycloalkyl, and C₁-C₆ haloalkyl;
R₃ is selected from hydrogen, halogen, C₁-C₆ alkyl, C₃-C₆ cycloalkyl, C₁-C₆ haloalkyl, and C₁-C₆ alkoxy;
R₄ is selected from hydrogen, halogen, C₁-C₆ alkyl, C₃-C₆ cycloalkyl, C₁-C₄ alkenyl, C₁-C₅ alkoxy, C₃-C₆ cycloalkenyl, C₃-C₈ heterocyclyl, 5- to 10-membered heteroaryl, and 5- to 10-membered aryl; wherein each of the C₃-C₆ cycloalkyl, C₃-C₈ heterocyclyl, 5- to 10-membered heteroaryl, and 5- to 10-membered aryl is optionally substituted with 1-3 groups independently selected from halogen, hydroxy, amino, oxo, C₁-C₆ alkyl, C₃-C₆ cycloalkyl, and C₁-C₆ haloalkyl;
R₅ is selected from C₁-C₆ alkyl, deuterated C₁-C₆ alkyl, C₁-C₆ haloalkyl, C₃-C₆ cycloalkyl, deuterated C₁-C₆ alkoxy, C₁-C₆ alkoxy, and C₁-C₆ haloalkoxy;
Q is

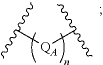

wherein each Q_{A} is independently selected from —C(H)(R_{Q})—, —O—, —N(R_{Q})—, —S(O)₂—, —C(O)—, —NH—, —C(O)N(R_{Q})—, C₃-C₆ cycloalkyl, 3- to 12-membered heterocycle, 6- to 12-membered bridged heterocycle, 6- to 12-membered spiro heterocycle, and 6- to 12-membered spiro cycloalkyl; wherein each of the C₃-C₆ cycloalkyl, 3- to 12-membered heterocycle, 6- to 12-membered bridged heterocycle, 6- to 12-membered spiro heterocycle, and 6- to 12-membered spiro cycloalkyl is optionally substituted with 1-3 R_{Q}, and wherein each R_{Q} is independently selected from hydrogen, hydroxyl, C₁-C₆ alkyl, and C₃-C₆ cycloalkyl;
n is 1, 2, 3, 4, 5, 6, 7, 8, 9, or 10; and
W is

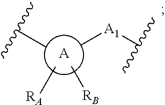

wherein Ring A is selected from 6- to 12-membered aryl and 5- to 12-membered heteroaryl; wherein A₁ is selected from a bond, —C(O) NH—, —NH—, —C(O)N(C₁-C₆ alkyl)-, and —C(O)O—; wherein R_{A} and R_{B}, which may be the same or different, are each independently selected from hydrogen, halogen, C₁-C₆ alkyl, C₃-C₆ cycloalkyl, and oxo; or R_{A} and R_{B} together with the carbon atom(s) to which they are attached form a 3- to 6-membered cycloalkyl or a 3- to 6-membered heterocycle.

In some embodiments, the present disclosure provides a compound represented by Formula (I), or a tautomer, a stereoisomer or a mixture of stereoisomers, a pharmaceutically acceptable salt, a hydrate, or a deuterated derivative thereof:

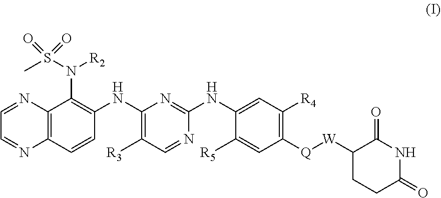

wherein:
R₁ is selected from hydroxy, amino, halogen, C₁-C₆ alkyl, C₃-C₆ cycloalkyl, C₁-C₆ haloalkyl, and C₁-C₆ alkoxy;
R₂ is selected from hydrogen, C₁-C₆ alkyl, C₃-C₆ cycloalkyl, and C₁-C₆ haloalkyl;
R₃ is selected from hydrogen, halogen, C₁-C₆ alkyl, C₃-C₆ cycloalkyl, C₁-C₆ haloalkyl, and C₁-C₆ alkoxy;
R₄ is selected from hydrogen, halogen, C₁-C₆ alkyl, C₃-C₆ cycloalkyl, C₁-C₄ alkenyl, C₁-C₆ alkoxy, C₃-C₆ cycloalkenyl, C₃-C₆ heterocyclyl, 5- to 10-membered heteroaryl, and 5- to 10-membered aryl; wherein each of the C₃-C₈ heterocyclyl, 5- to 10-membered heteroaryl, and 5- to 10-membered aryl is optionally substituted with 1-3 groups independently selected from halogen, hydroxy, amino, oxo, C₁-C₈ alkyl, C₃-C₆ cycloalkyl, and C₁-C₆ haloalkyl;
R₅ is selected from C₁-C₆ alkyl, deuterated C₁-C₆ alkyl, C₁-C₆ haloalkyl, C₃-C₆ cycloalkyl, deuterated C₁-C₆ alkoxy, C₁-C₆ alkoxy, and C₁-C₆ haloalkoxy;
Q is

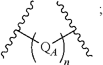

wherein each Q_{A} is independently selected from —C(H)(R_{Q})—, —O—, —N(R_{Q})—, —S(O)₂—, —C(O)—, —C(O)N(R_{Q})—, C₃-C₆ cycloalkyl, 3- to 12-membered heterocycle, 6- to 12-membered bridged heterocycle, 6- to 12-membered spiro heterocycle, and 6- to 12-membered spiro cycloalkyl; wherein each R_{Q} is independently selected from hydrogen, C₁-C₆ alkyl, and C₃-C₆ cycloalkyl;
n is 1, 2, 3, 4, 5, 6, 7, 8, 9, or 10; and
W is

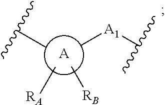

wherein Ring A is selected from 6- to 12-membered aryl and 5- to 12-membered heteroaryl; wherein A₁ is selected from a bond, —C(O) NH—, —C(O)N(C₁-C₆ alkyl)-, and —C(O)O—; wherein R_{A} and Re, which may be the same or different, are each independently selected from hydrogen, halogen, C₁-C₆ alkyl, C₃-C₅ cycloalkyl, and oxo; or R_{A} and R_{B} together with the carbon atom(s) to which they are attached form a 3- to 6-membered cycloalkyl or a 3- to 6-membered heterocycle.

Also disclosed herein is a method of treating a disease or disorder, in a subject in need thereof, comprising administering to said subject at least one entity selected from the compounds of Formula (I), tautomers thereof, stereoisomers thereof, pharmaceutically acceptable salts thereof, hydrates thereof, and deuterated derivatives thereof, or a pharmaceutical composition comprising at least one entity selected from the compounds of Formula (I), tautomers thereof, stereoisomers thereof, pharmaceutically acceptable salts thereof, hydrates thereof, and deuterated derivatives thereof, and at least one pharmaceutically acceptable carrier or excipient. In some embodiments, the pharmaceutical composition of the present disclosure may be for use in (or in the manufacture of medicaments for) the treatment of the disease or disorder in the subject in need thereof.

In some embodiments, a therapeutically effective amount of a pharmaceutical composition of the present disclosure may be administered to a subject diagnosed with a disease or disorder. In some embodiments, the disease or disorder is cancer. In some embodiments, the cancer is selected from pancreatic cancer, breast cancer, glioblastoma multiforme, head and neck cancer, and lung cancer. In some embodiments, the lung cancer is non-small cell lung cancer (NSCLC). In some embodiments, the NSCLC is selected from adenocarcinoma, squamous cell carcinoma, and large cell carcinoma.

### BRIEF DESCRIPTION OF THE FIGURES

FIG. 1 is a diagram illustrating the synthesis of intermediate-1.

FIG. 2 is a diagram illustrating the synthesis of intermediate-2.

FIG. 3 is a diagram illustrating the synthesis of intermediate-3.

FIG. 4 is a diagram illustrating the synthesis of intermediate-4.

FIG. 5 is a diagram illustrating the synthesis of intermediate-5.

FIG. 6 is a diagram illustrating the synthesis of intermediate-6.

FIG. 7 is a diagram illustrating the synthesis of Compound 1.

FIG. 8 is a diagram illustrating the synthesis of Compound 2.

FIG. 9 is a diagram illustrating the synthesis of Compound 3.

FIG. 10 is a diagram illustrating the synthesis of Compound 4.

FIG. 11 is a diagram illustrating the synthesis of Compound 5.

FIG. 12 is a diagram illustrating the synthesis of Compound 6.

FIG. 13 is a diagram illustrating the synthesis of Compound 7.

FIG. 14 is a diagram illustrating the synthesis of Compound 8.

FIG. 15 is a diagram illustrating the synthesis of Compound 9.

FIG. 16 is a diagram illustrating the synthesis of Compound 10.

FIG. 17 is a diagram illustrating the synthesis of Compound 11.

FIG. 18 is a diagram illustrating the synthesis of Compound 77.

FIG. 19 is a diagram illustrating the synthesis of Compound 35.

FIG. 20 is a diagram illustrating the synthesis of Compound 36.

FIG. 21 is a diagram illustrating the synthesis of Compound 37.

FIG. 22 is a diagram illustrating the synthesis of Compound 38.

FIG. 23 is a diagram illustrating the synthesis of Compound 39.

FIG. 24 is a diagram illustrating the synthesis of Compound 40.

FIG. 25 is a diagram illustrating the synthesis of Compound 41.

FIG. 26 is a diagram illustrating the synthesis of Compound 47.

FIG. 27 is a diagram illustrating the synthesis of Compound 93.

FIG. 28 is a diagram illustrating the synthesis of Compound 90.

FIG. 29 is a diagram illustrating the synthesis of Compound 54.

FIG. 30 is a diagram illustrating the synthesis of Compound 28.

FIG. 31 is a diagram illustrating the synthesis of Compound 32.

FIG. 32 is a diagram illustrating the synthesis of Compound 70.

FIG. 33 is a diagram illustrating the synthesis of Compound 78.

FIG. 34 is a diagram illustrating the synthesis of Compound 79.

FIG. 35 is a diagram illustrating the synthesis of Compound 80.

FIG. 36 is a diagram illustrating the synthesis of Compound 81.

FIG. 37 is a diagram illustrating the synthesis of Compound 82.

FIG. 38 is a diagram illustrating the synthesis of Compound 83.

FIG. 39 is a diagram illustrating the synthesis of Compound 84.

FIG. 40 is a diagram illustrating the synthesis of Compound 85.

FIG. 41 is a diagram illustrating the synthesis of Compound 86.

FIG. 42 is a diagram illustrating the synthesis of Compound 87.

FIG. 43 is a diagram illustrating the synthesis of Compound 88.

FIG. 44 is a diagram illustrating the synthesis of Compound 89.

FIG. 45 illustrates the EGFR degradative activity of exemplary compound 11 of the present disclosure and a comparator Compound A in engineered BAF3_EGFR Del19/T790M/C797S cells 24 hours after administration.

FIG. 46 illustrates the EGFR degradative activity of exemplary compound 11 of the present disclosure and a comparator Compound A in engineered BAF3_EGFR ASV cells 24 hours after administration.

FIG. 47 illustrates the EGFR degradative activity of exemplary compound 11 of the present disclosure and a comparator Compound A in engineered BAF3_EGFR SVD cells 24 hours after administration.

FIG. 48 illustrates the EGFR degradative activity of exemplary compound 11 of the present disclosure and a comparator Compound A in engineered BAF3_HER2YVMA cells 24 hours after administration.

FIG. 49 illustrates the EGFR degradative activity of exemplary compound 11 of the present disclosure and a comparator Compound A in NCl-H2073 cells 24 hours after administration.

FIG. 50 illustrates the cell growth inhibitory activity of exemplary compound of the present disclosure and a comparator Compound A in engineered BA/F3 cells and NCl-H2073 cells three days after administration.

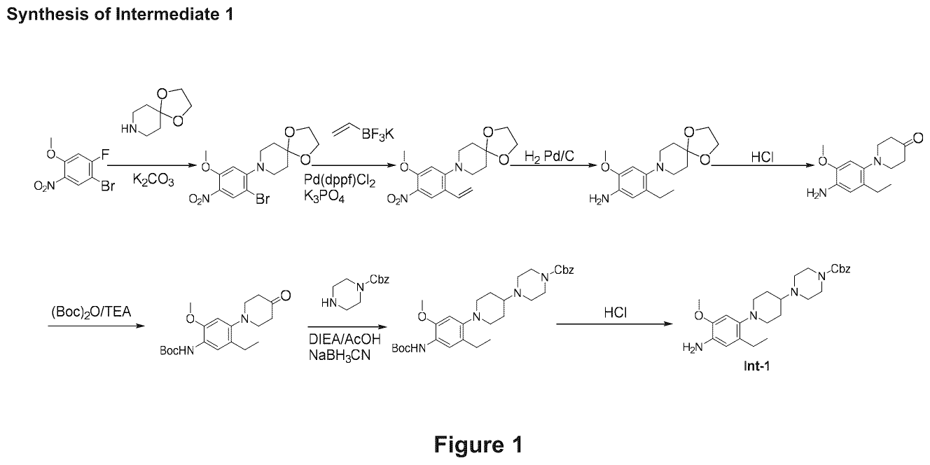

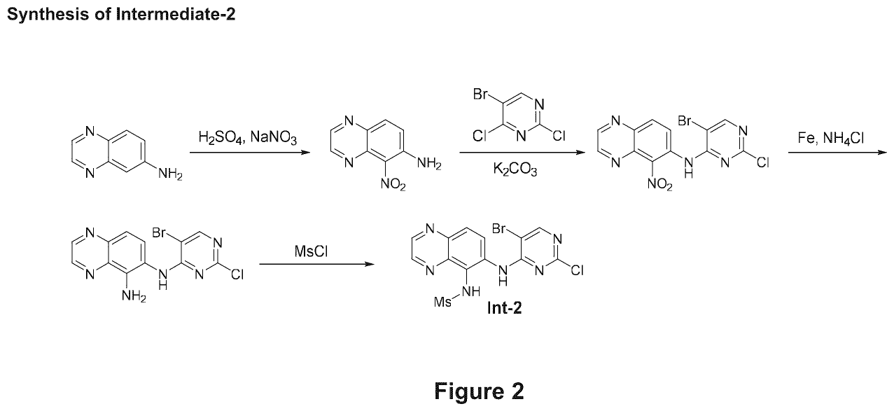

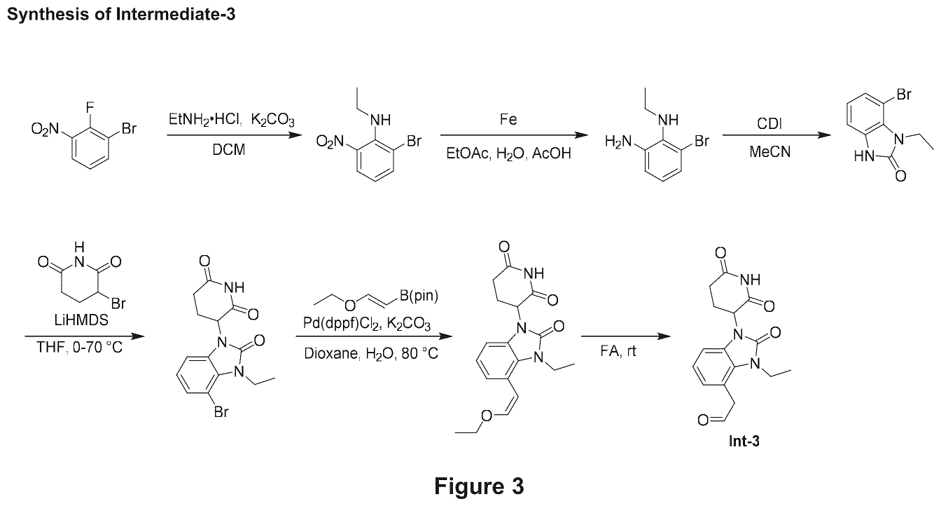

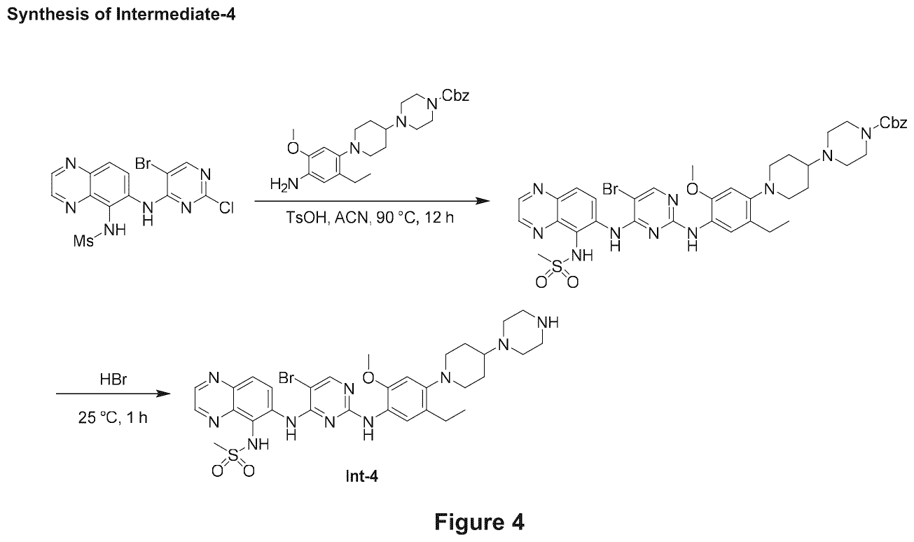

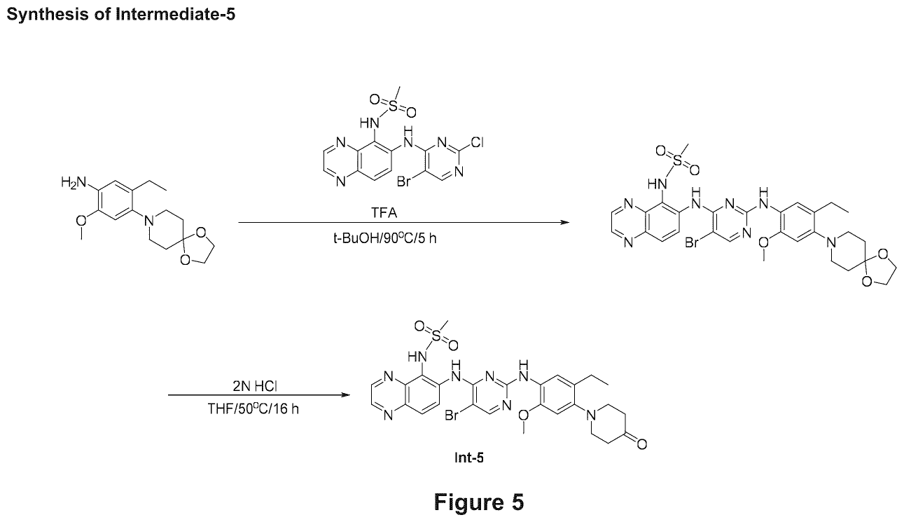

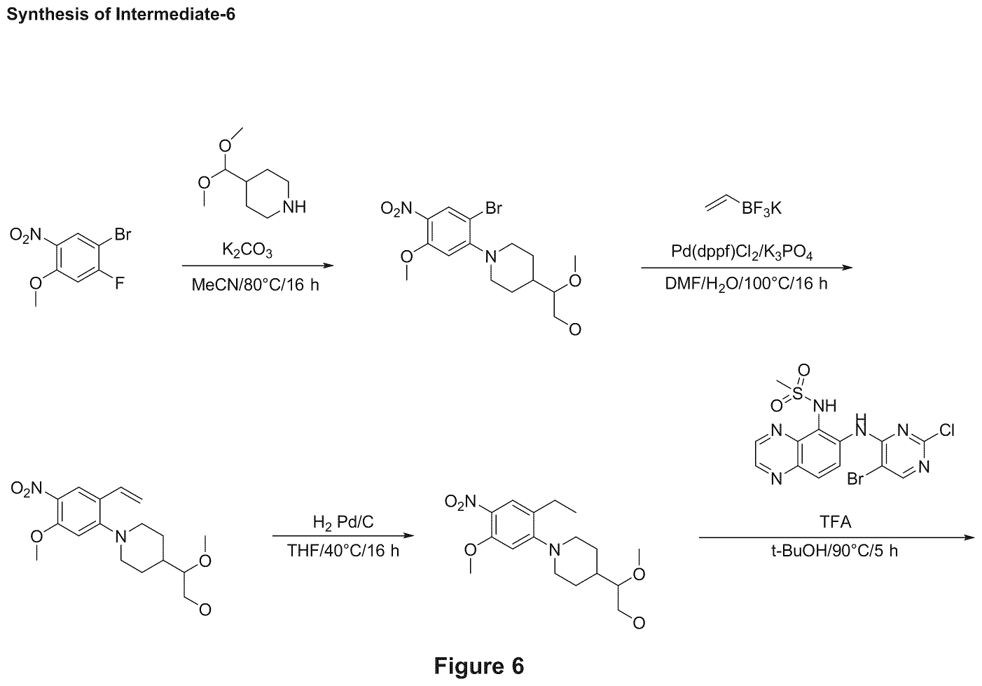

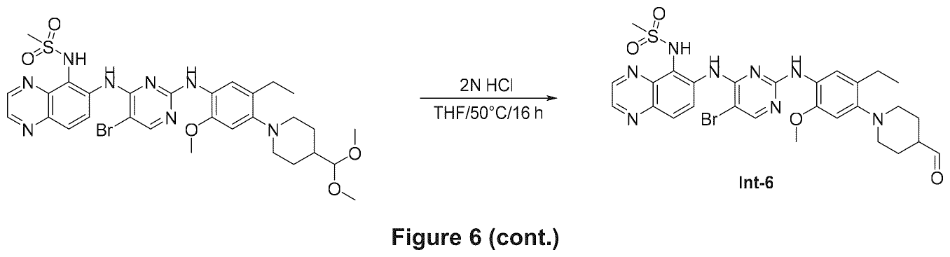

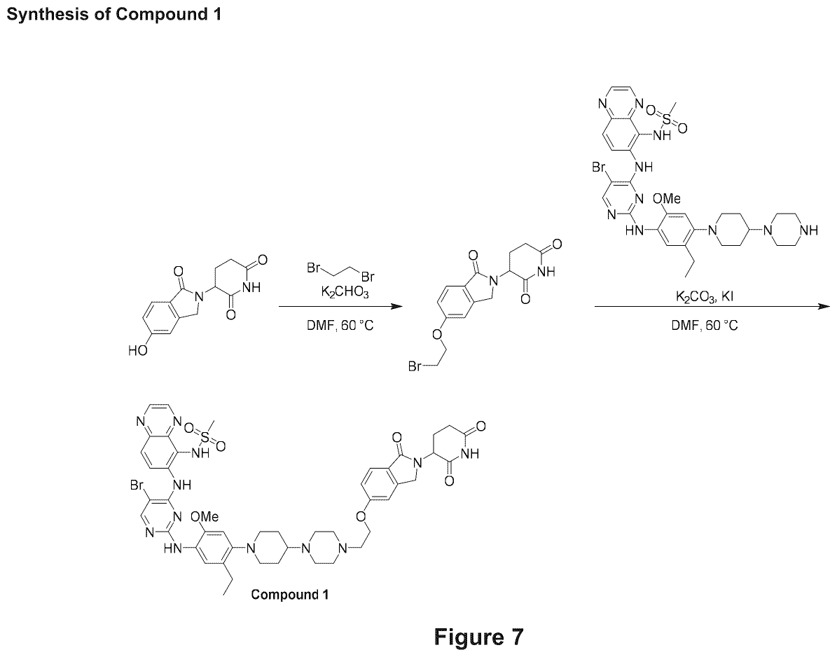

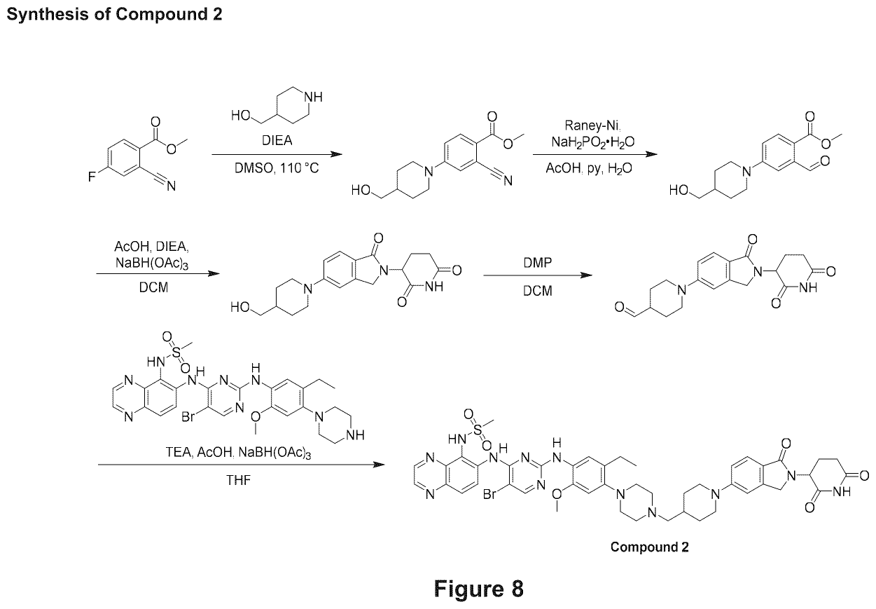

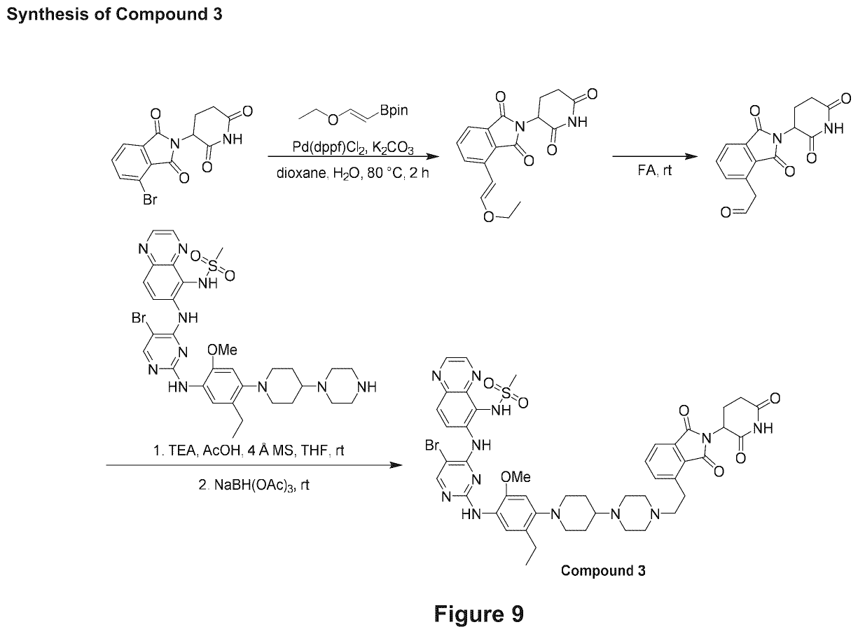

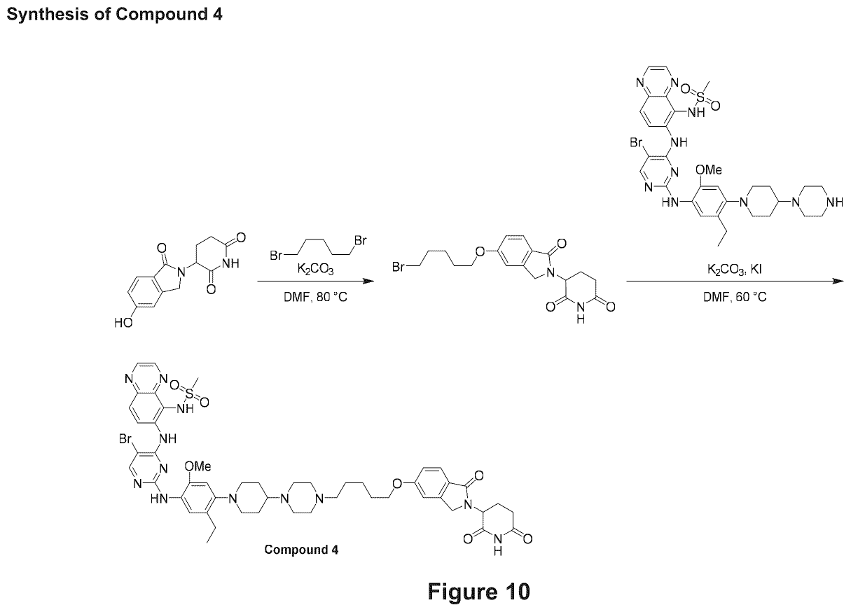

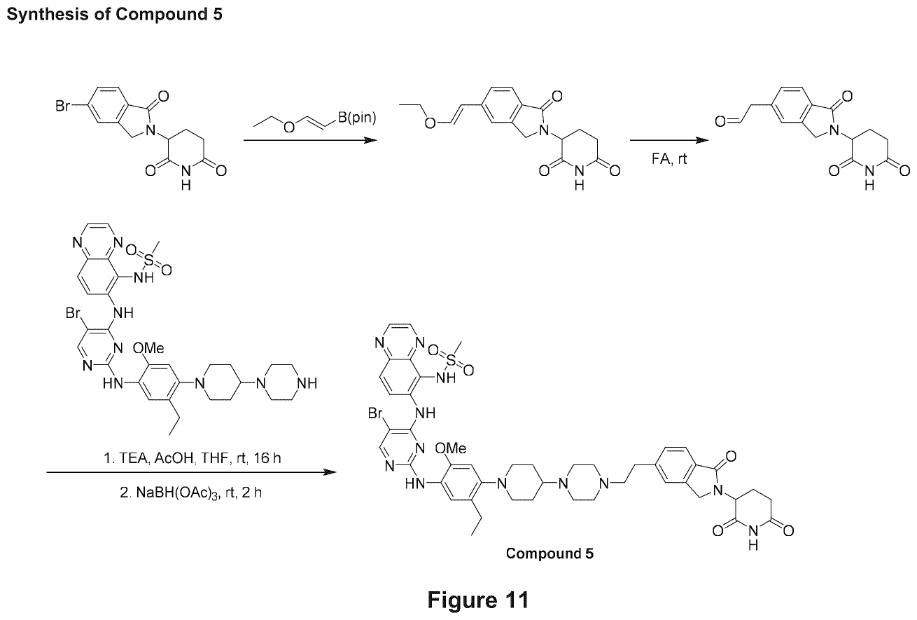

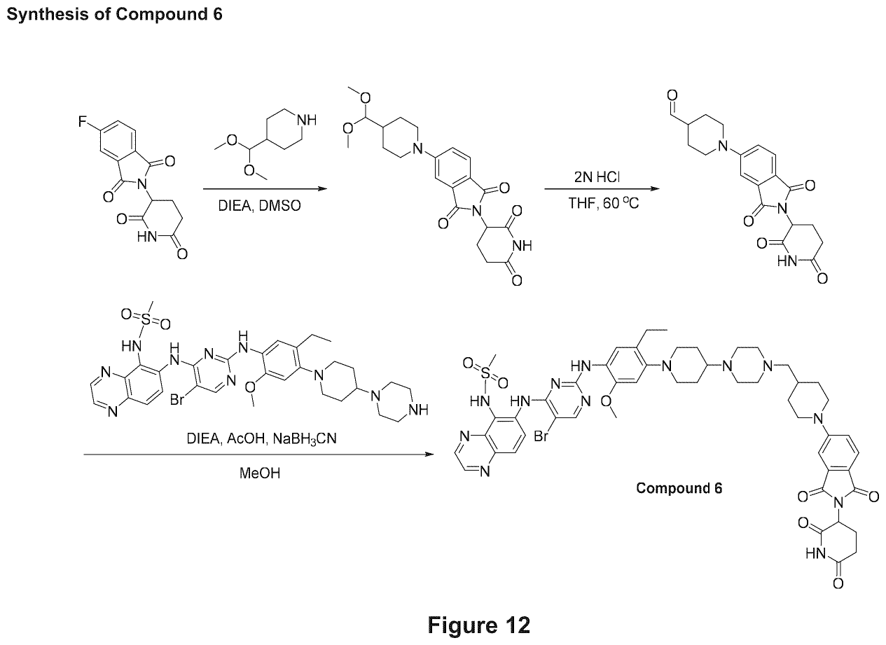

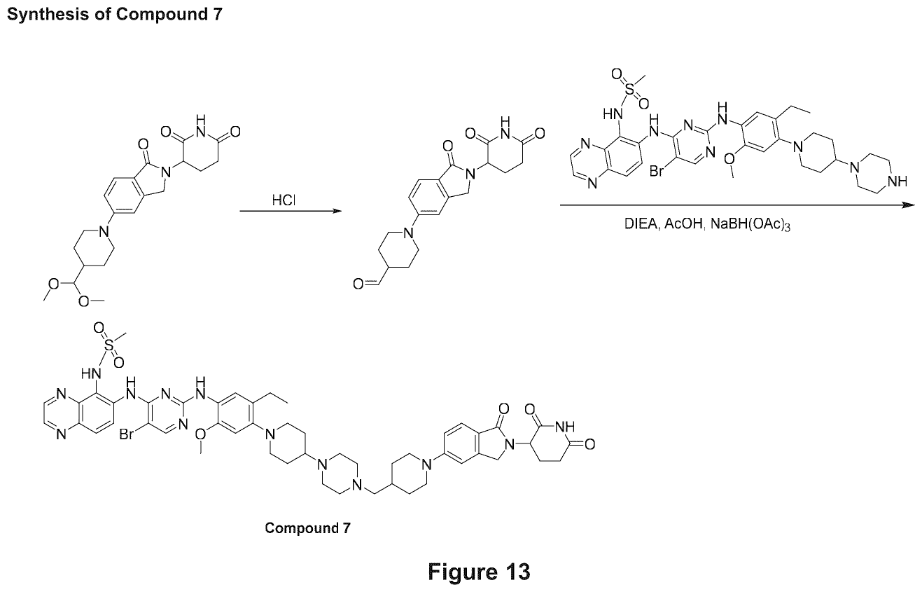

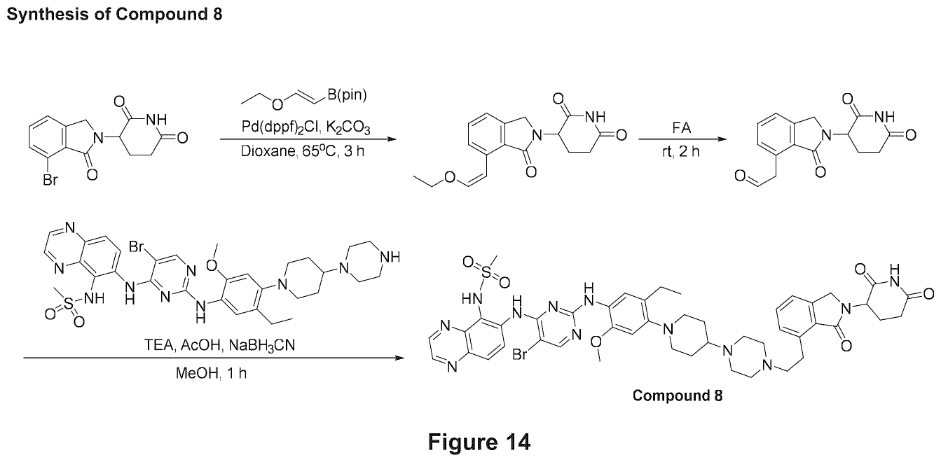

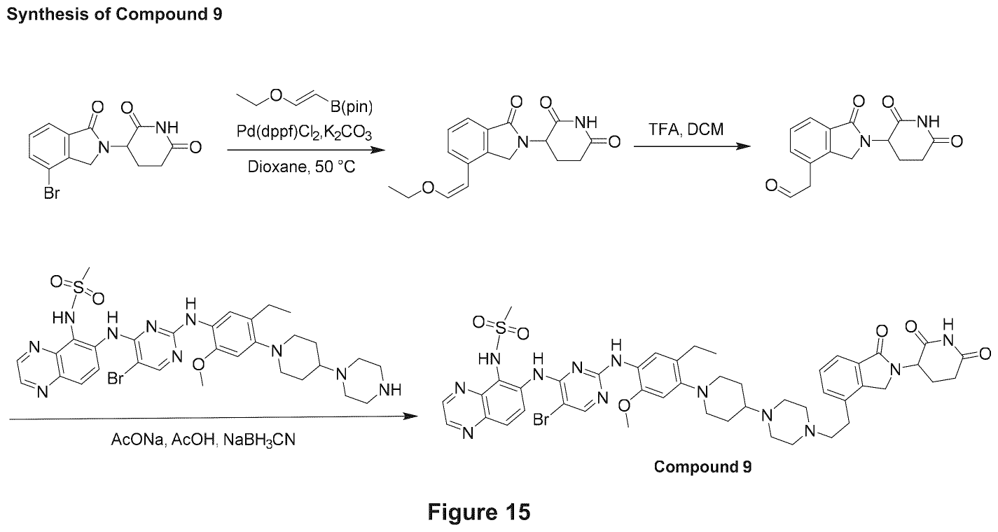

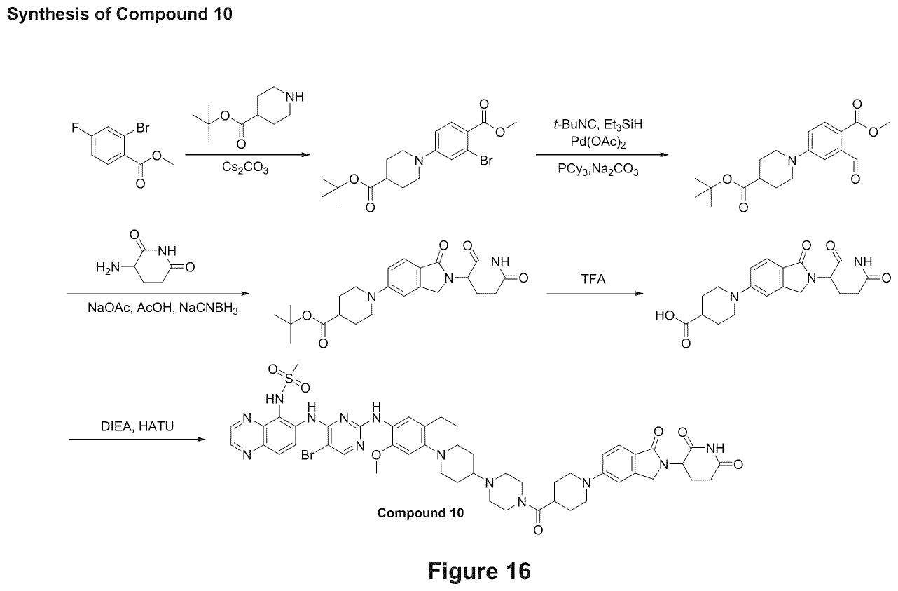

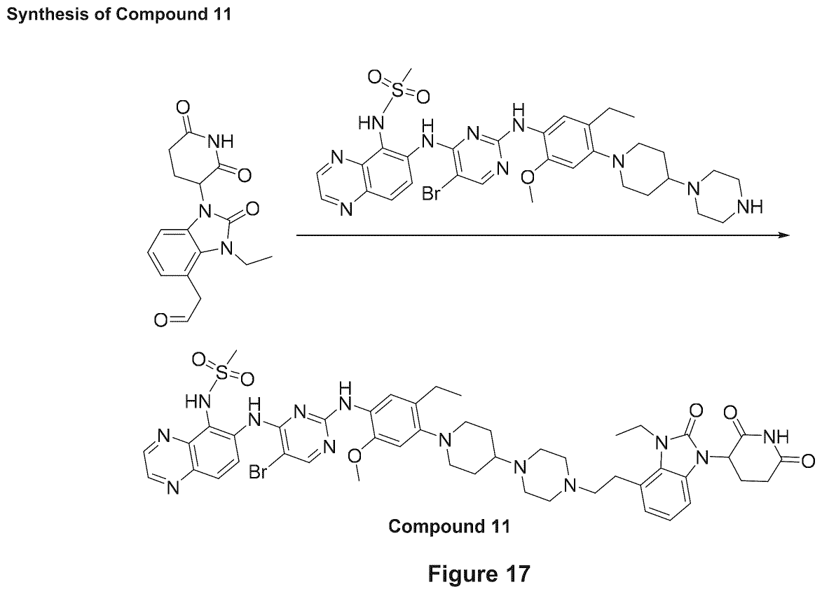

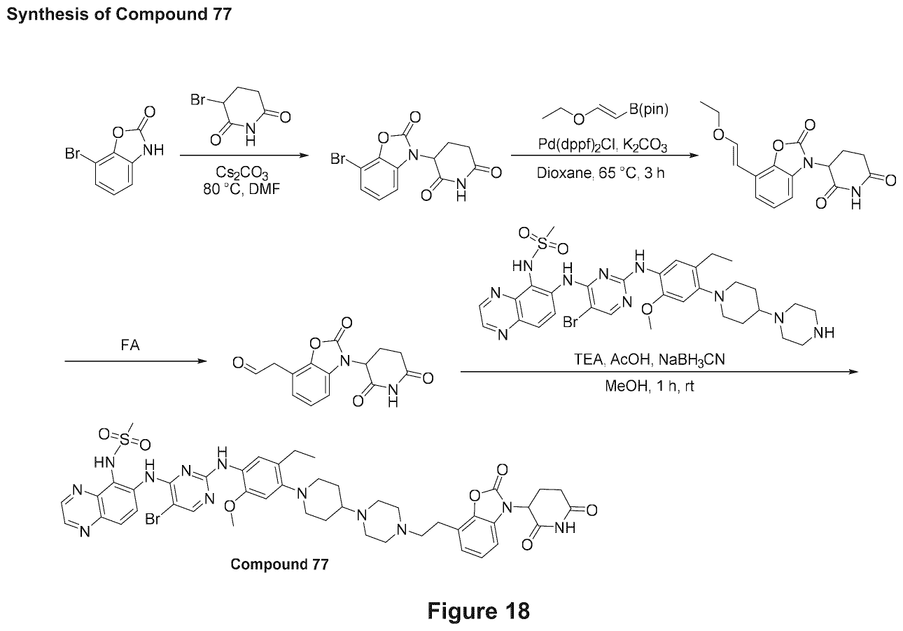

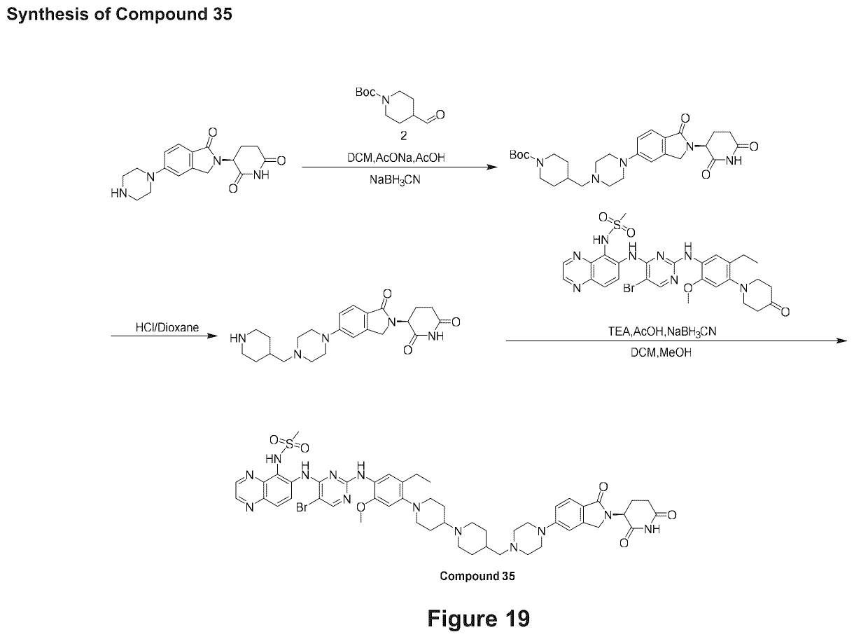

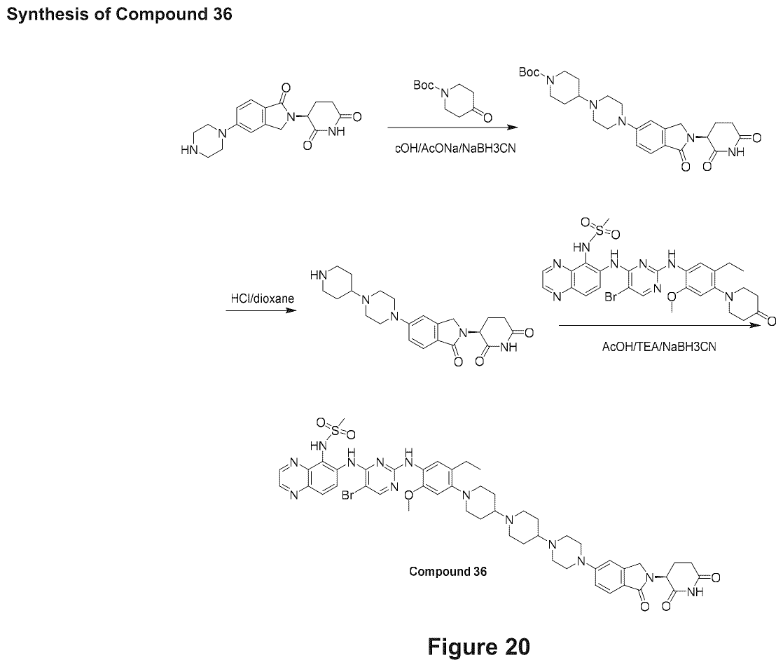

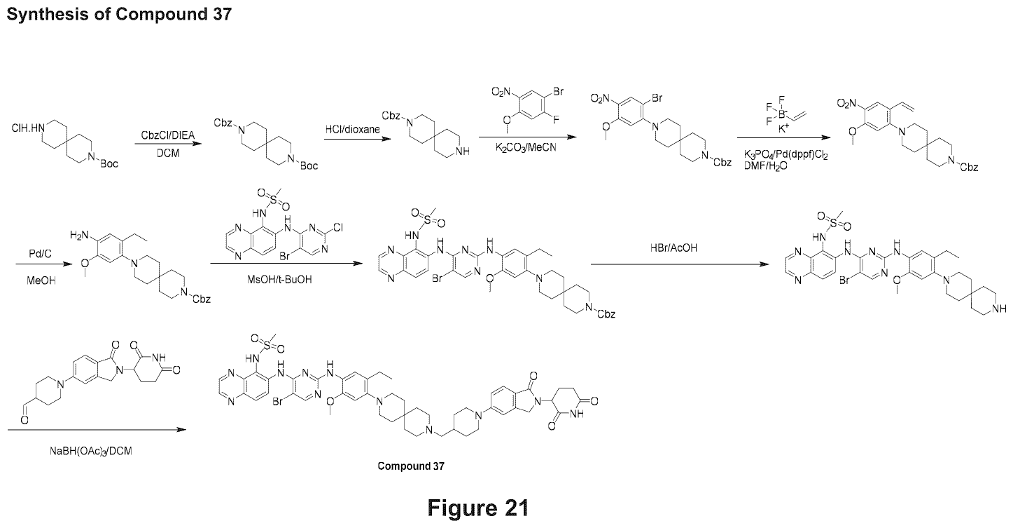

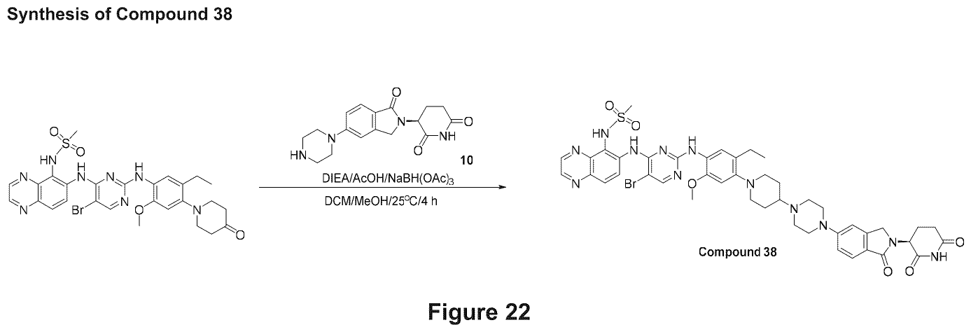

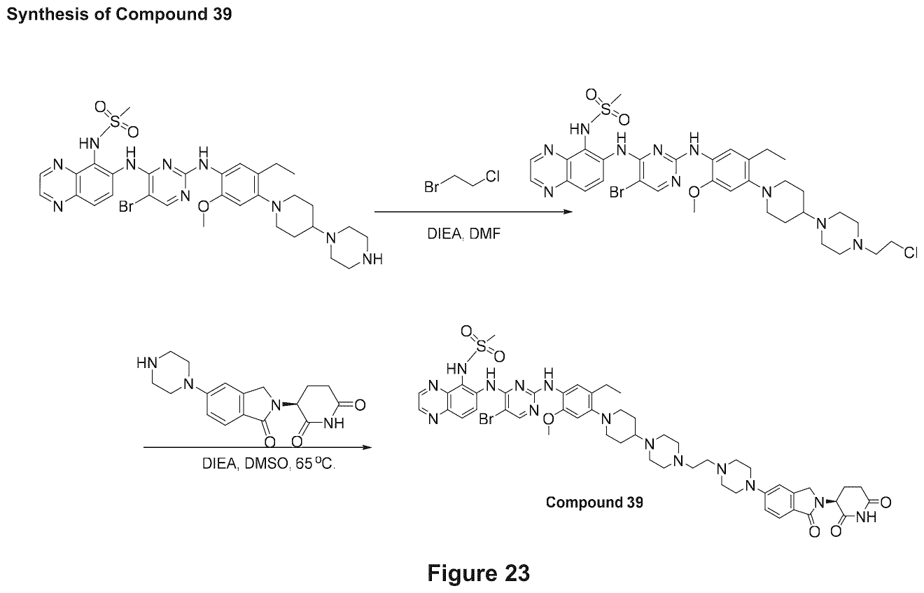

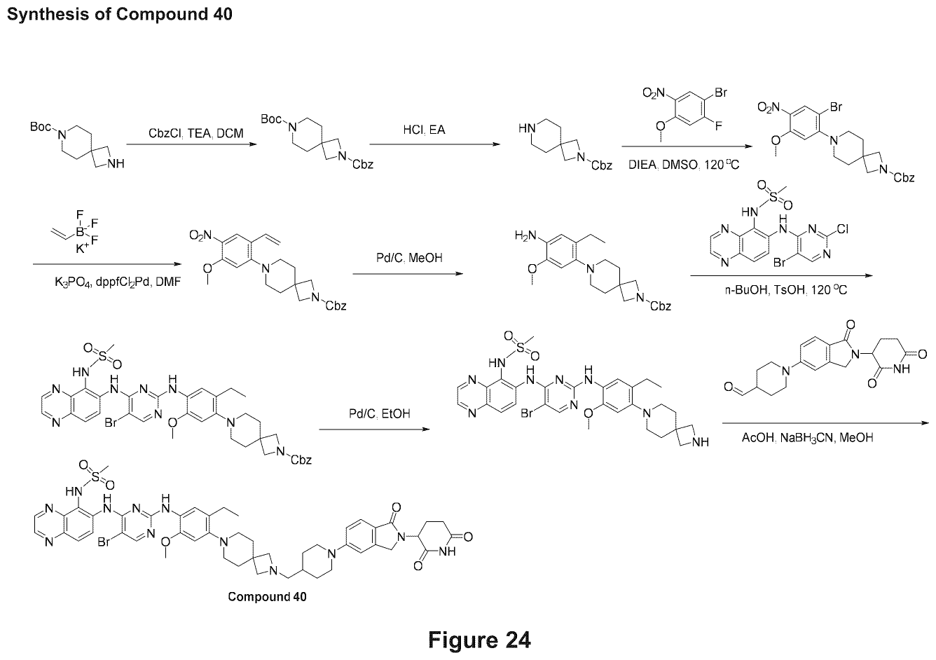

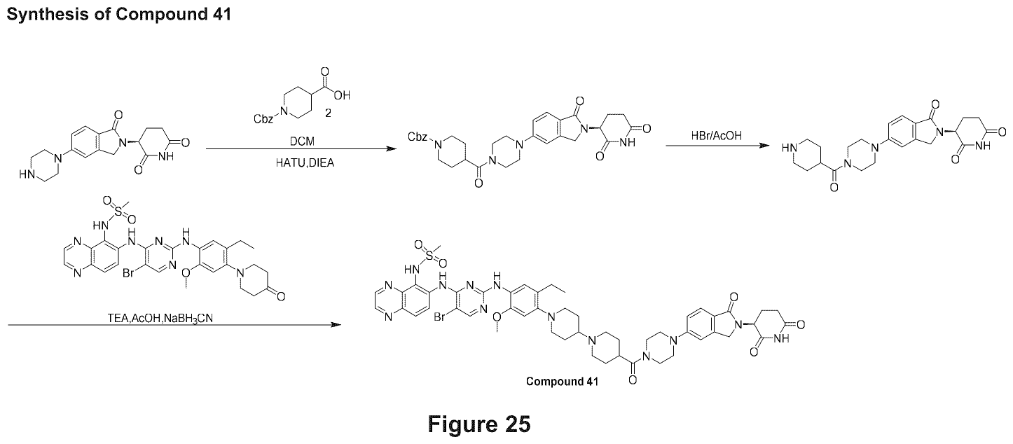

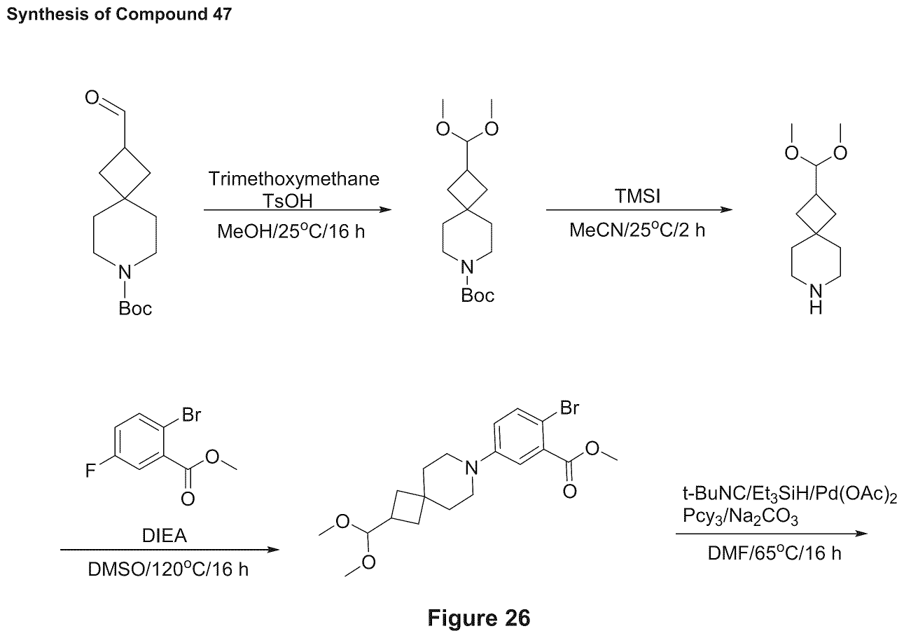

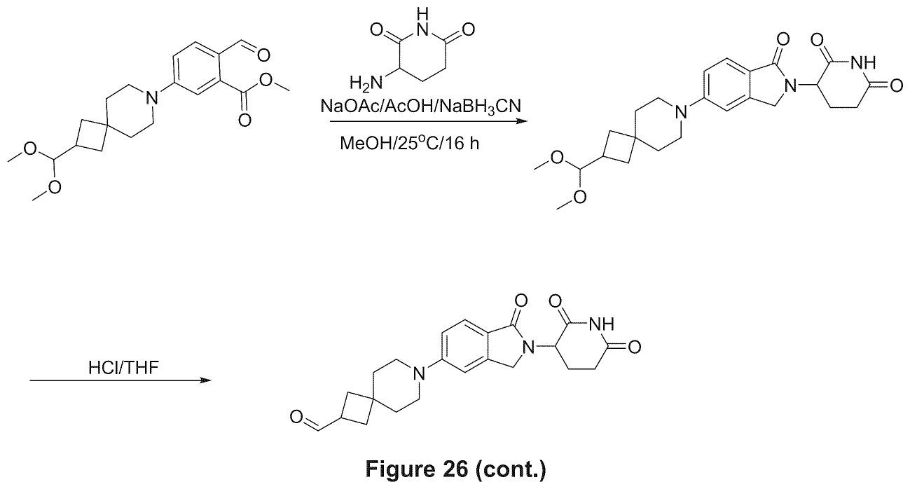

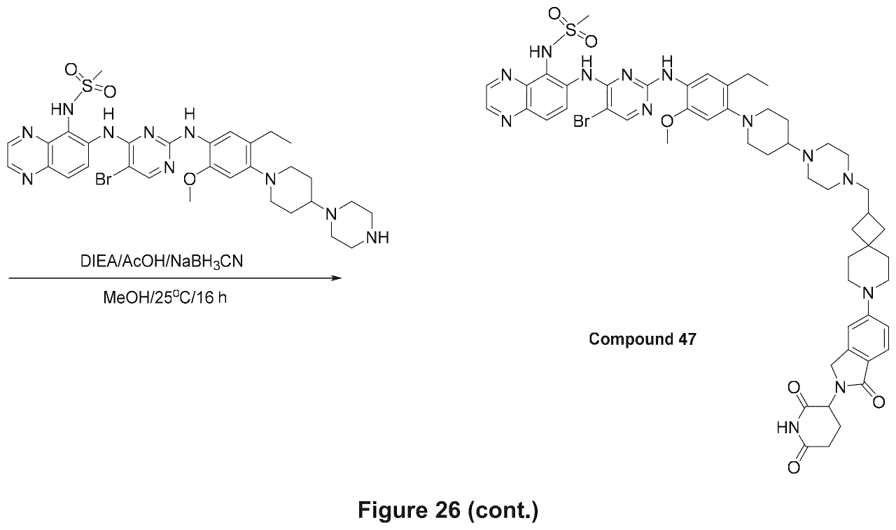

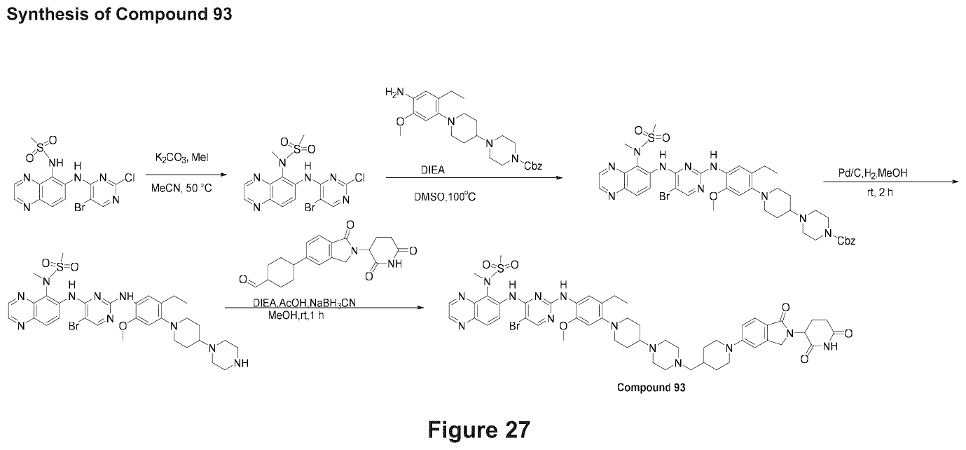

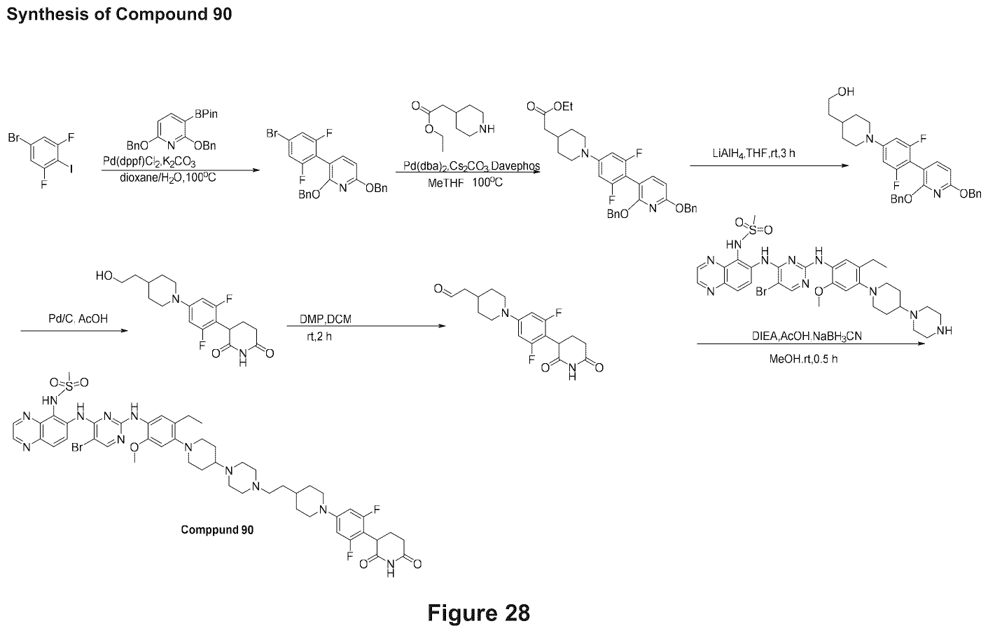

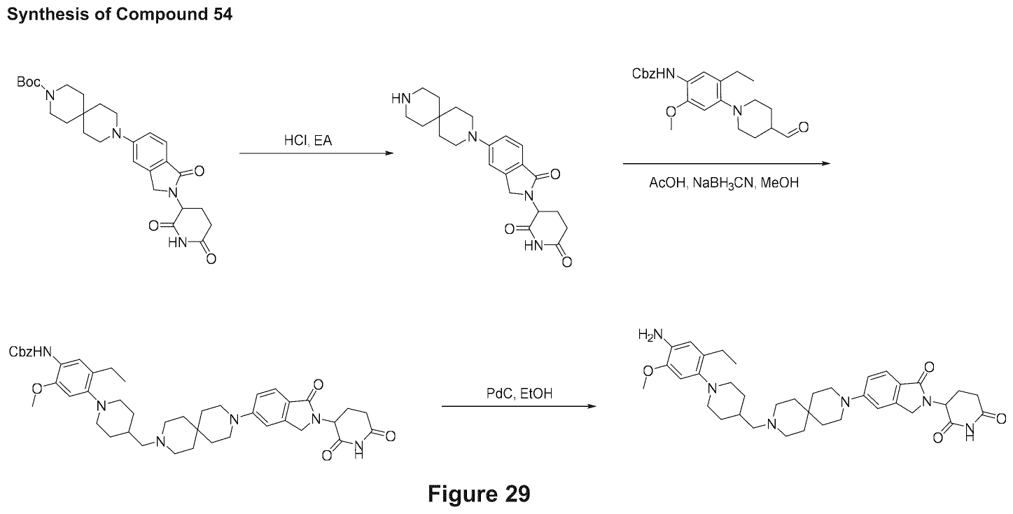

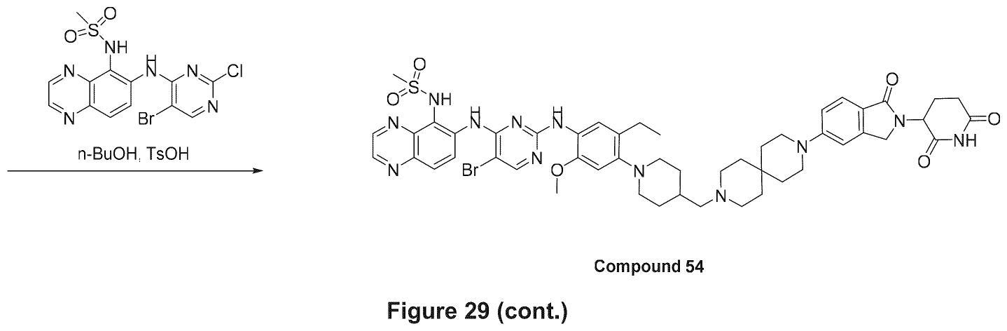

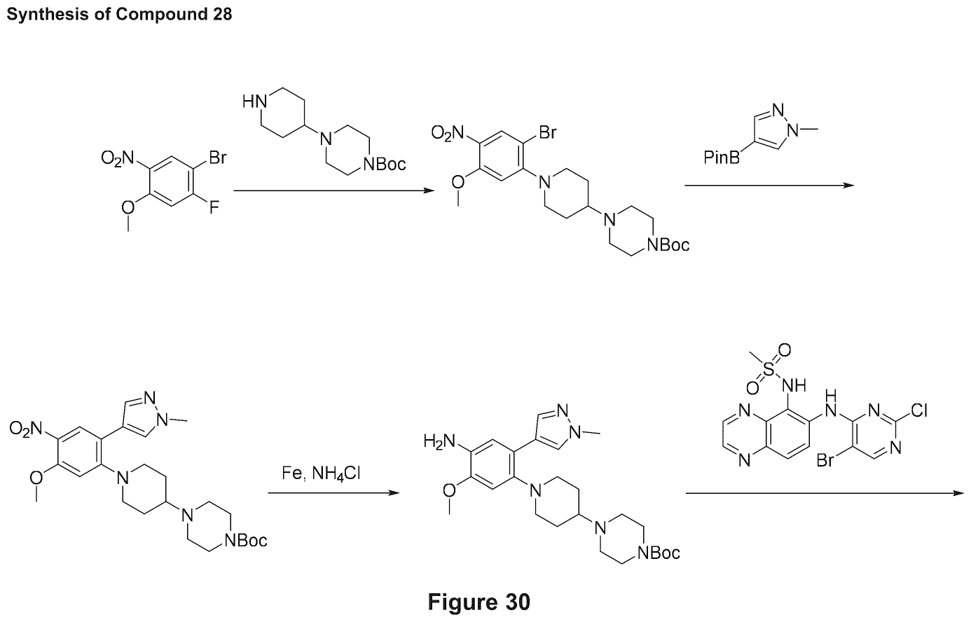

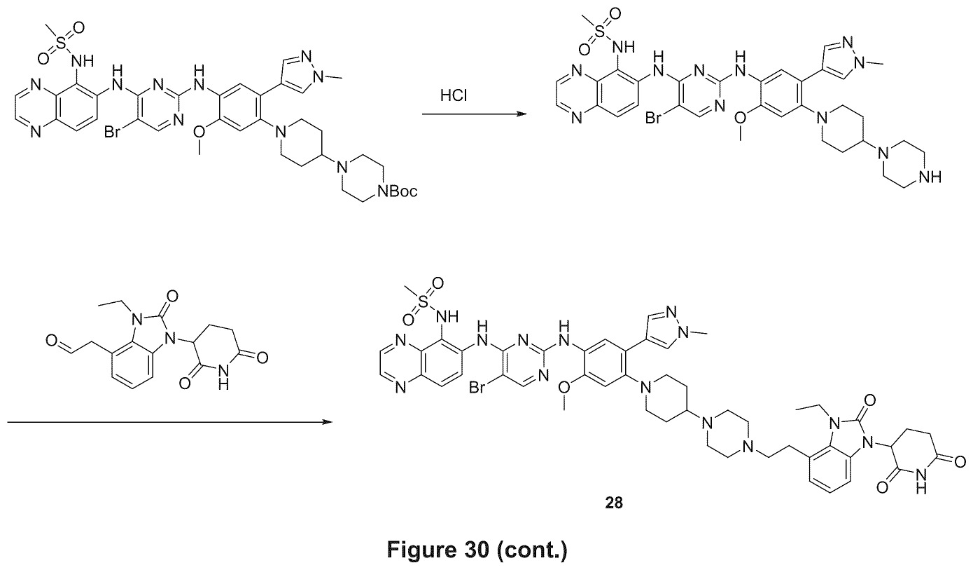

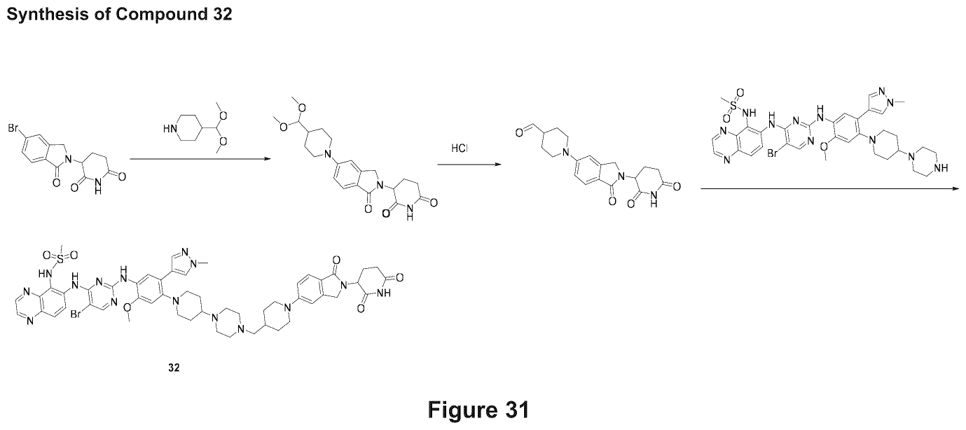

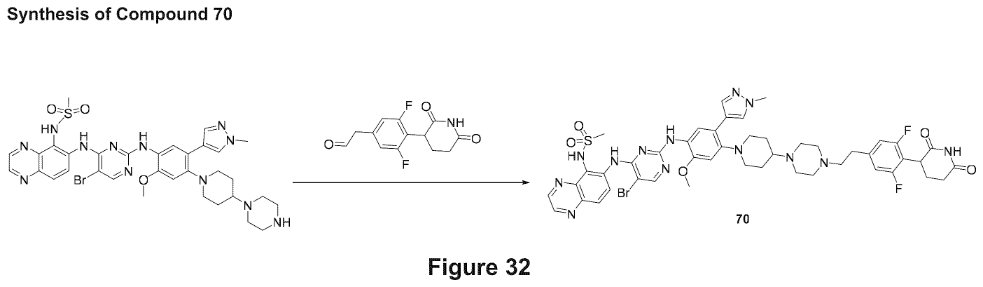

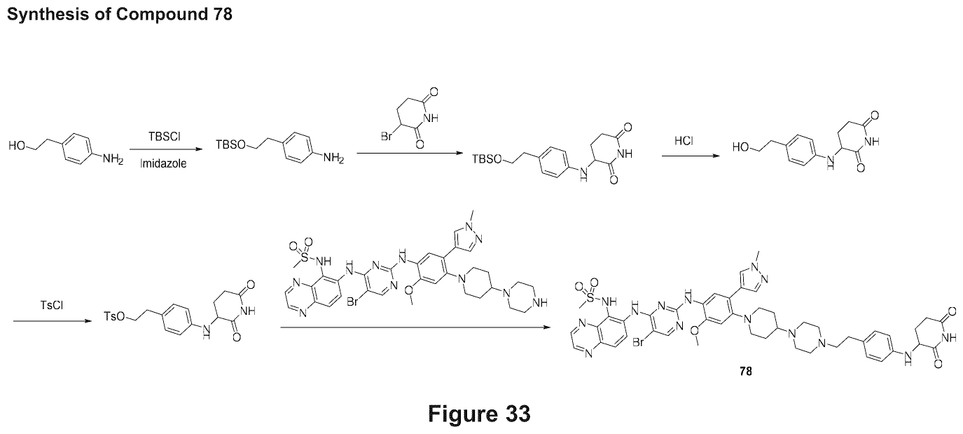

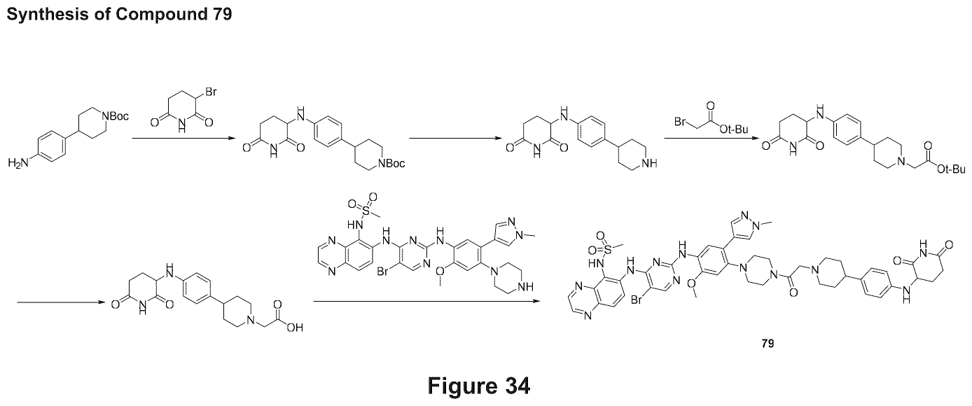

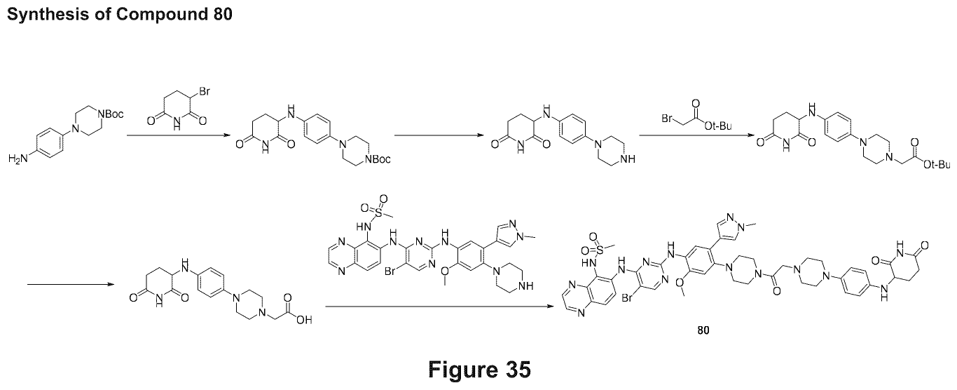

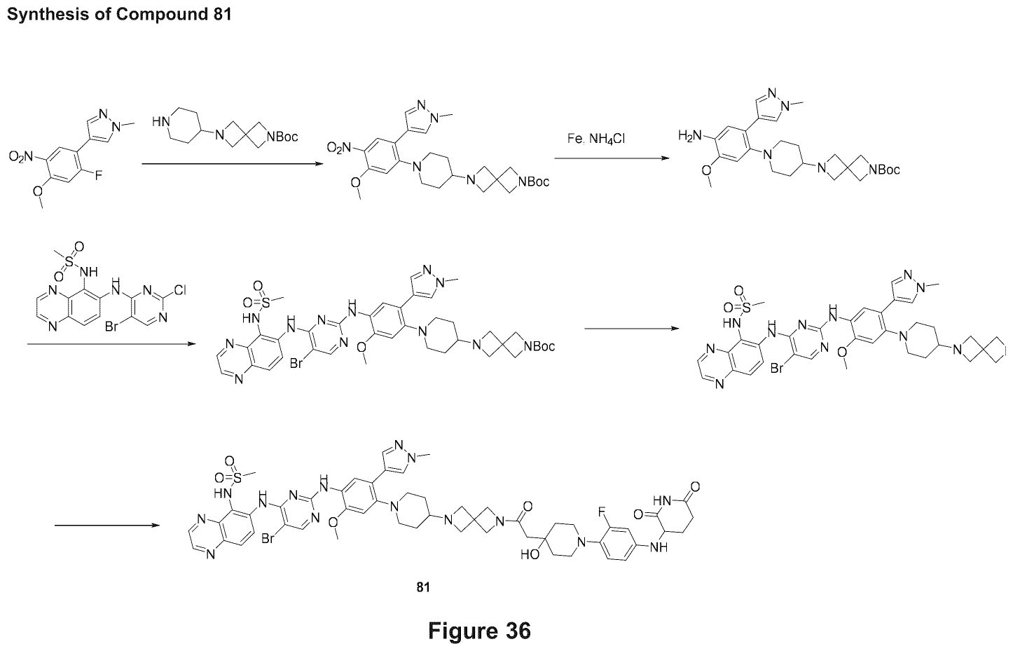

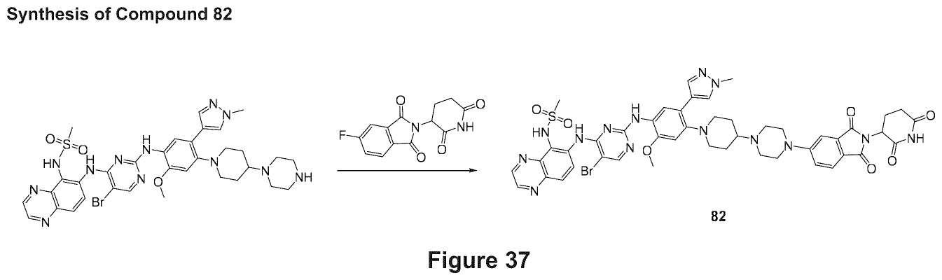

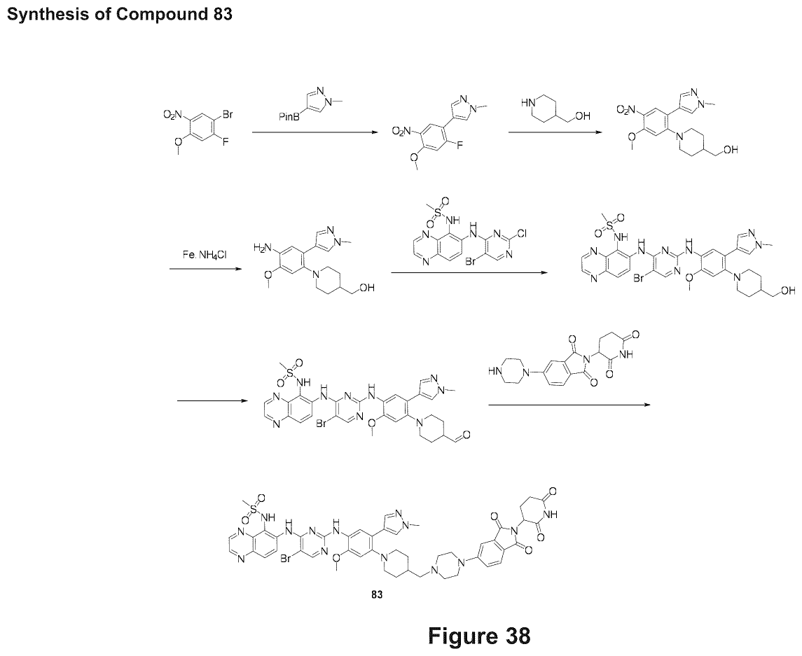

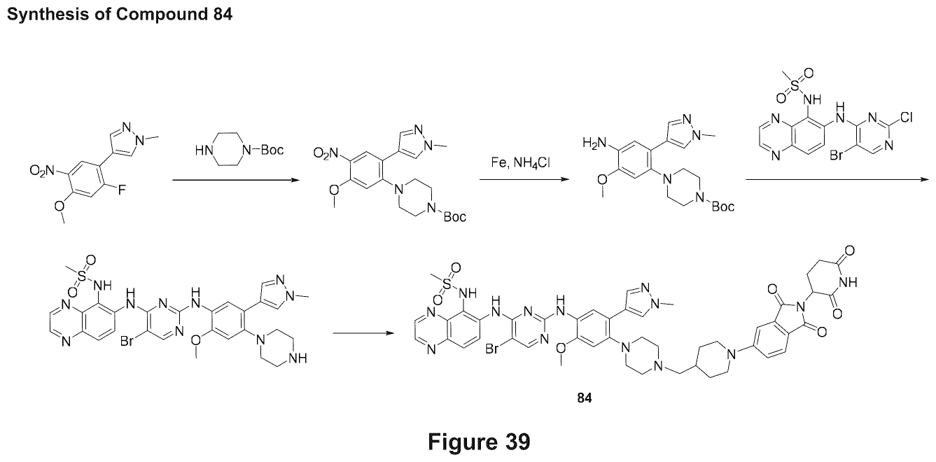

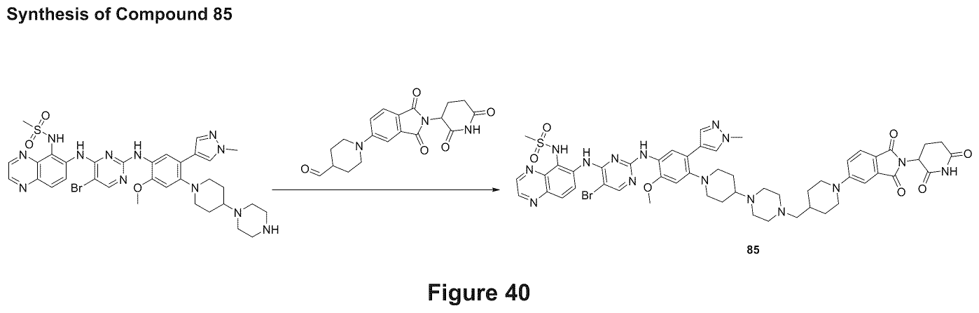

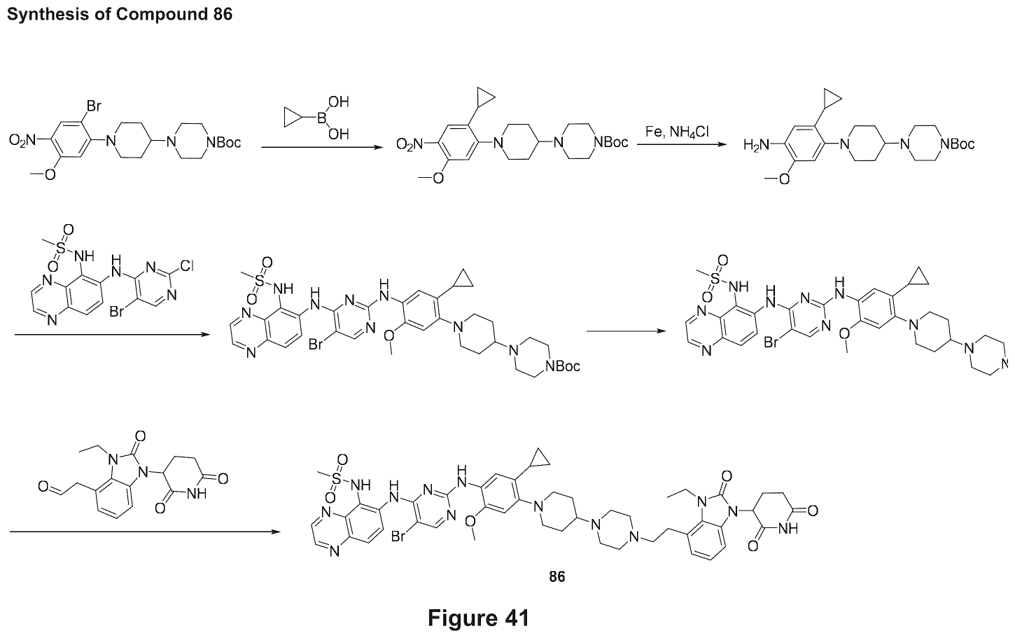

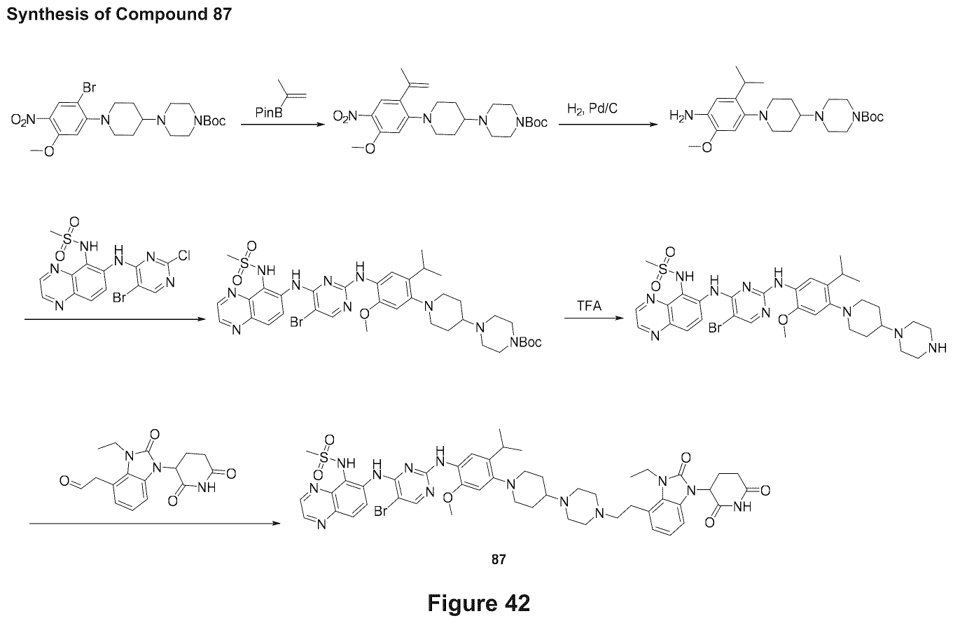

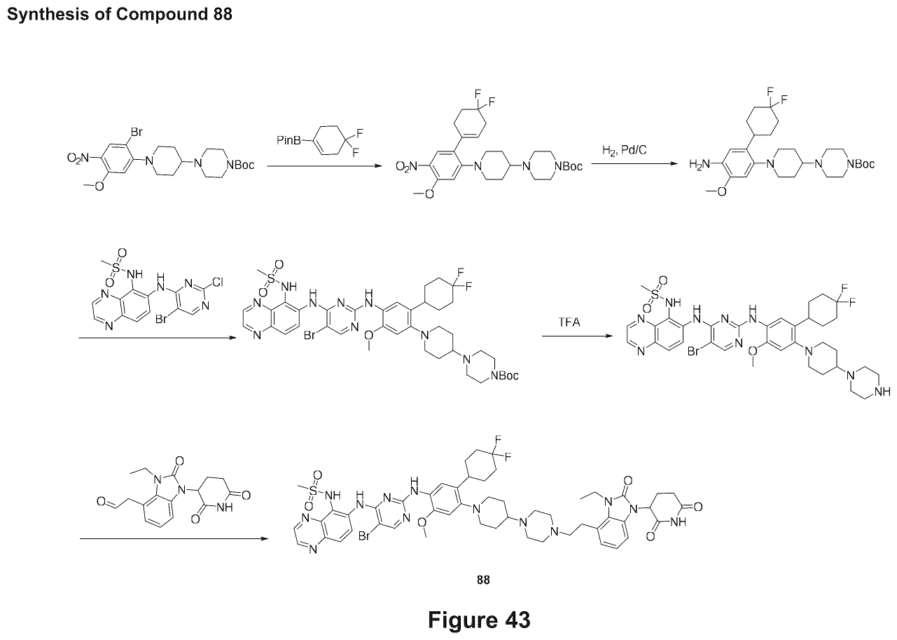

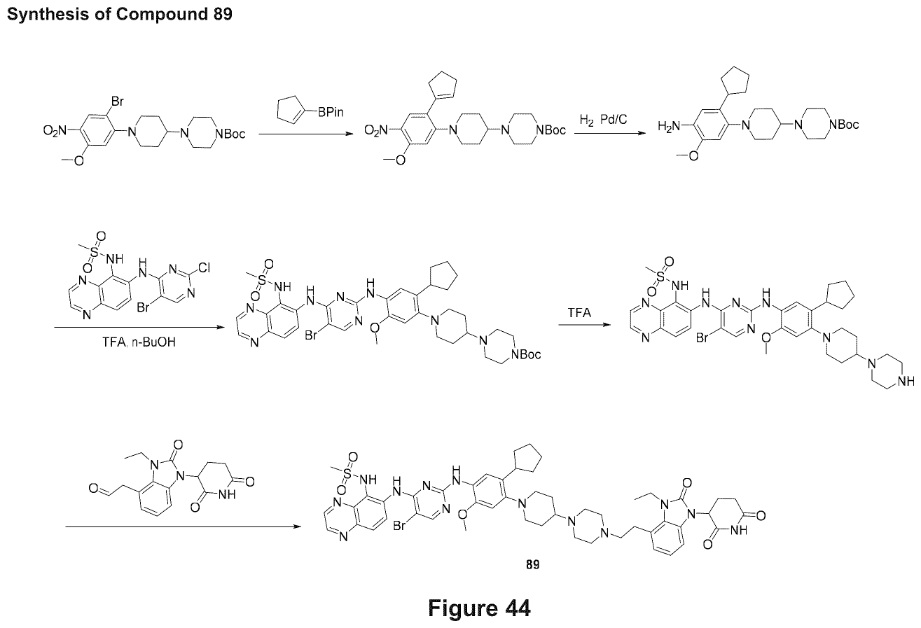

### DETAILED DESCRIPTION

### Definitions

As used herein, “cancer” refers to diseases, disorders, and conditions that involve abnormal cell growth with the potential to invade or spread to other parts of the body. Exemplary cancers include, but are not limited to, breast cancer, ovarian cancer, uterine cancer, endometrial cancer, cervical cancer, bladder cancer, prostate cancer, lung cancer, stomach cancer, esophageal cancer, colorectal cancer, small bowel cancer, pancreatic cancer, liver cancer, kidney cancer, head and neck cancer, skin cancer, bone cancer, thyroid cancer, peritoneal cancer, and brain cancer.

“Subject” refers to an animal, such as a mammal, that has been or will be the object of treatment, observation, or experiment. The methods described herein may be useful for both human therapy and veterinary applications. In one embodiment, the subject is a human.

As used herein, “treatment” or “treating” refers to an amelioration of a disease or disorder, or at least one discernible symptom thereof. In another embodiment, “treatment” or “treating” refers to an amelioration of at least one measurable physical parameter, not necessarily discernible by the patient. In yet another embodiment, “treatment” or “treating” refers to inhibiting the progression of a disease or disorder, either physically, e.g., stabilization of a discernible symptom, physiologically, e.g., stabilization of a physical parameter, or both. In yet another embodiment, “treatment” or “treating” refers to delaying the onset of a disease or disorder.

A dash (“−”) that is not between two letters or symbols is used to indicate a point of attachment for a substituent. For example, —CN is attached through the carbon atom.

By “optional” or “optionally”, it is meant that the subsequently described event or circumstance may or may not occur, and that the description includes instances where the event or circumstance occurs and instances in which is does not. It will be understood by those skilled in the art, with respect to any group containing one or more substituents, that such groups are not intended to introduce any substitution or substitution patterns that are sterically impractical, synthetically non-feasible and/or inherently unstable.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkenyl” as used herein refers to an unsaturated alkyl group having a carbon-carbon double bond.

The term “alkoxy” as used herein refers to an alkyl or cycloalkyl covalently bonded to an oxygen atom.

The term “alkyl” as used herein refers to a saturated straight or branched hydrocarbon, such as a straight or branched group of 1-8 carbon atoms, referred to herein as “C₁-C₈ alkyl”. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, and octyl. In some embodiments, “alkyl” is a straight-chain hydrocarbon. In some embodiments, “alkyl” is a branched hydrocarbon.

The term “alkynyl” as used herein refers to an unsaturated alkyl group having a carbon-carbon triple bond.

The term “aryl” as used herein refers to a mono-, bi-, or other multi-carbocyclic, aromatic ring system with 5 to 14 ring atoms. The aryl group can optionally be a fused ring wherein the carbocyclic, aromatic ring system is fused to one or more rings selected from aryls, cycloalkyls, heteroaryls, and heterocyclyls. The aryl groups of this present disclosure can be substituted with groups selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone. Exemplary aryl groups include, but are not limited to, phenyl, anthracenyl, fluorenyl, indenyl, azulenyl, and naphthyl, as well as benzo-fused carbocyclic moieties such as 5,6,7,8-tetrahydronaphthyl. Exemplary aryl groups also include but are not limited to a monocyclic aromatic ring system, wherein the ring comprises 6 carbon atoms, referred to herein as “C₆-aryl.”

The term “cycloalkyl” as used herein refers to a saturated or unsaturated cyclic, bicyclic, or bridged bicyclic hydrocarbon group of 3-16 carbons, or 3-8 carbons, referred to herein as “C₃-C₈ cycloalkyl,” derived from a cycloalkane. Exemplary cycloalkyl groups include, but are not limited to, cyclohexanes, cyclohexenes, cyclopentanes, and cyclopentenes. Cycloalkyl groups may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Cycloalkyl groups can be fused to other cycloalkyl (saturated or partially unsaturated), aryl, or heterocyclyl groups, to form a bicycle, tetracycle, etc. The term “cycloalkyl” also includes bridged and spiro-fused cyclic structures which may or may not contain heteroatoms.

The terms “halo” or “halogen” as used herein refer to —F, —Cl, —Br, and/or —I.

The term “haloalkyl group” as used herein refers to an alkyl group substituted with one or more halogen atoms.

The term “heteroaryl” as used herein refers to a mono-, bi-, or multi-cyclic, aromatic ring system containing one or more heteroatoms, for example 1-4 heteroatoms, such as nitrogen, oxygen, and sulfur. Heteroaryls can be substituted with one or more substituents including alkoxy, aryloxy, alkyl, alkenyl, alkynyl, amide, amino, aryl, arylalkyl, carbamate, carboxyl, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heteroaryls can also optionally be a fused ring wherein the aromatic ring system containing one or more heteroatoms is fused to a non-aromatic ring. Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3)- and (1,2,4)-triazolyl, pyrazinyl, pyrimidinyl, tetrazolyl, furyl, thienyl, isoxazolyl, thiazolyl, furyl, phenyl, isoxazolyl, and oxazolyl. Exemplary heteroaryl groups include, but are not limited to, a monocyclic aromatic ring, wherein the ring comprises 2-5 carbon atoms and 1-3 heteroatoms, referred to herein as “(C₂-C₅) heteroaryl.” In some embodiments, a heteroaryl contains 5 to 10 ring atoms, 1 to 4 of which are heteroatoms selected from N, O, and S. In some embodiments, a heteroaryl contains 5 to 8 ring atoms, 1 to 4 of which are heteroatoms selected from N, O, and S.

The terms “heterocycle,” “heterocyclyl,” or “heterocyclic” as used herein each refer to a saturated or unsaturated 3- to 18-membered ring containing one, two, three, or four heteroatoms independently selected from nitrogen, oxygen, phosphorus, and sulfur. Heterocycles can be aromatic (heteroaryls) or non-aromatic. Heterocycles can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heterocycles also include bicyclic, tricyclic, and tetracyclic groups in which any of the above heterocyclic rings is fused to one or two rings independently selected from aryls, cycloalkyls, and heterocycles. Heterocycles also include bridged and spiro-fused cyclic structures which may or may not contain heteroatoms. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl. In some embodiments, a heterocycle contains 5 to 10 ring atoms, 1 to 4 of which are heteroatoms selected from N, O, and S. In some embodiments, a heterocycle contains 5 to 8 ring atoms, 1 to 4 of which are heteroatoms selected from N, O, and S.

The terms “hydroxy” and “hydroxyl” as used herein refer to —OH.

The term “oxo” as used herein refers to a double bond to an oxygen atom (i.e., ═O). For example, when two geminal groups on a carbon atom are “taken together to form an oxo”, then a carbonyl (i.e., C═O) is formed.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

As used herein, the term “pharmaceutically acceptable salt” refers to a salt form of a compound of this disclosure wherein the salt is nontoxic. Pharmaceutically acceptable salts of the compounds of this disclosure include those derived from suitable inorganic and organic acids and bases. A “free base” form of a compound, for example, does not contain an ionically bonded salt.

The phrase “deuterated derivatives”, in reference to one or more compounds or formulae of the present disclosure, is intended to encompass deuterated analogs of any one of the referenced compounds and pharmaceutically acceptable salts of those deuterated analogs.

One of ordinary skill in the art would recognize that, when an amount of “a compound or a pharmaceutically acceptable salt thereof” is disclosed, the amount of the pharmaceutically acceptable salt form of the compound is the amount equivalent to the concentration of the free base of the compound. It is noted that the disclosed amounts of the compounds or their pharmaceutically acceptable salts thereof herein are based upon their free base form.

Suitable pharmaceutically acceptable salts are, for example, those disclosed in S. M. Berge, et al. J. Pharmaceutical Sciences, 1977, 66, 1-19. For example, Table 1 of that article provides the following pharmaceutically acceptable salts:

TABLE 1
Pharmaceutically Acceptable Salts

| | | |
|---|---|---|
| Acetate | Aluminum | Benzathine |
| Benzenesulfonate | Benzoate | Bicarbonate |
| Bitartrate | Bromide | Calcium |
| Calcium edetate | Camsylate | Carbonate |
| Chloride | Chloroprocaine | Choline |
| Citrate | Diethanolamine | Dihydrochloride |
| Edetate | Edisylate | Estolate |
| Esylate | Ethylenediamine | Fumarate |
| Gluceptate | Gluconate | Glutamate |
| Glycollylarsanilate | Hexylresorcinate | Hydrabamine |
| Hydrobromide | Hydrochloride | Hydroxynaphthoate |
| Iodide | Isethionate | Lactate |
| Lactobionate | Lithium | Magnesium |
| Malate | Maleate | Mandelate |
| Meglumine | Mesylate | Methylbromide |
| Methylnitrate | Methylsulfate | Mucate |
| Napsylate | Nitrate | Pamoate (Embonate) |
| Pantothenate | Phosphate/diphosphate | Polygalacturonate |
| Potassium | Procaine | Salicylate |
| Sodium | Stearate | Subacetate |
| Succinate | Sulfate | Tannate |
| Tartrate | Teociate | Triethiodide |
| Zinc | | |

Non-limiting examples of pharmaceutically acceptable acid addition salts include: salts formed with inorganic acids, such as hydrochloric acid, hydrobromic acid, phosphoric acid, sulfuric acid, or perchloric acid; salts formed with organic acids, such as acetic acid, oxalic acid, maleic acid, tartaric acid, citric acid, succinic acid or malonic acid; and salts formed by using other methods used in the art, such as ion exchange. Non-limiting examples of pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Pharmaceutically acceptable salts derived from appropriate bases include alkali metal, alkaline earth metal, ammonium, and N⁺ (C₁₋₄alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

As used herein, nomenclature for compounds including organic compounds, can be given using common names, IUPAC, IUBMB, or CAS recommendations for nomenclature. One of skill in the art can readily ascertain the structure of a compound if given a name, either by systemic reduction of compound structure using naming conventions, or by commercially available software, such as CHEMDRAW™ (Cambridgesoft Corporation, U.S.A.).

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present disclosure encompasses various stereoisomers of these compounds and mixtures thereof. Stereoisomers include enantiomers and diastereomers. Mixtures of enantiomers or diastereomers may be designated “(+)” in nomenclature, but the skilled artisan will recognize that a structure may denote a chiral center implicitly. In some embodiments, an enantiomer or stereoisomer may be provided substantially free of the corresponding enantiomer.

In some embodiments, the compound is a racemic mixture of(S)- and (R)-isomers. In other embodiments, provided herein is a mixture of compounds wherein individual compounds of the mixture exist predominately in an(S)- or (R)-isomeric configuration. For example, the compound mixture has an(S)-enantiomeric excess of greater than about 55%, about 60%, about 65%, about 70%, about 75%, about 80%, about 85%, about 90%, about 95%, about 96%, about 97%, about 98%, about 99%, about 99.5%, or more. In other embodiments, the compound mixture has an(S)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more. In other embodiments, the compound mixture has an (R)-enantiomeric purity of greater than about 55%, about 60%, about 65%, about 70%, about 75%, about 80%, about 85%, about 90%, about 95%, about 96%, about 97%, about 98%, about 99%, about 99.5% or more. In some other embodiments, the compound mixture has an (R)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5% or more.

Individual stereoisomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by: (1) attachment of a mixture of enantiomers to a chiral auxiliary, separation of the resulting mixture of diastereomers by recrystallization or chromatography and liberation of the optically pure product from the auxiliary; (2) salt formation employing an optically active resolving agent; or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known stereoselective synthesis methods.

Geometric isomers can also exist in the compounds of the present disclosure. The present disclosure encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the E and Z isomers.

Substituents around a carbon-carbon double bond alternatively can be referred to as “cis” or “trans,” where “cis” represents substituents on the same side of the double bond and “trans” represents substituents on opposite sides of the double bond. The arrangements of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The compounds disclosed herein may exist as tautomers and both tautomeric forms are intended to be encompassed by the scope of the present disclosure, even if only one tautomeric structure is depicted.

Additionally, unless otherwise stated, structures described herein are also meant to include compounds that differ only in the presence of one or more isotopically enriched atoms. For example, compounds having the present structures except for the replacement of hydrogen by deuterium (²H) or tritium (³H), or the replacement of a carbon by a ¹³C- or ¹⁴C-carbon atom are within the scope of this disclosure. Such compounds may be useful as, for example, analytical tools, probes in biological assays, or therapeutic agents.

### Compounds

The present disclosure is directed to a compound of Formula (I), or a tautomer, a stereoisomer or a mixture of stereoisomers, a pharmaceutically acceptable salt, a hydrate, or a deuterated derivative thereof:

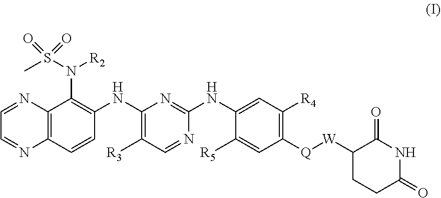

wherein:
R₁ is selected from hydroxy, amino, halogen, C₁-C₆ alkyl, C₃-C₆ cycloalkyl, C₁-C₆ haloalkyl, and C₁-C₆ alkoxy;
R₂ is selected from hydrogen, C₁-C₆ alkyl, C₃-C₈ cycloalkyl, and C₁-C₆ haloalkyl;
R₃ is selected from hydrogen, halogen, C₁-C₆ alkyl, C₃-C₆ cycloalkyl, C₁-C₆ haloalkyl, and C₁-C₆ alkoxy;
R₄ is selected from hydrogen, halogen, C₁-C₆ alkyl, C₃-C₆ cycloalkyl, C₁-C₄ alkenyl, C₁-C₅ alkoxy, C₃-C₆ cycloalkenyl, C₃-C₆ heterocyclyl, 5- to 10-membered heteroaryl, and 5- to 10-membered aryl; wherein each of the C₃-C₆ cycloalkyl, C₃-C₈ heterocyclyl, 5- to 10-membered heteroaryl, and 5- to 10-membered aryl is optionally substituted with 1-3 groups independently selected from halogen, hydroxy, amino, oxo, C₁-C₆ alkyl, C₅-C₆ cycloalkyl, and C₁-C₆ haloalkyl;
R₅ is selected from C₁-C₆ alkyl, deuterated C₁-C₆ alkyl, C₁-C₆ haloalkyl, C₃-C₆ cycloalkyl, deuterated C₁-C₆ alkoxy, C₁-C₆ alkoxy, and C₁-C₆ haloalkoxy;
Q is

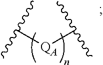

wherein each Q_{A} is independently selected from —C(H)(R_{Q})—, —O—, —N(R_{Q})—, —S(O)₂—, —C(O)—, —NH—, —C(O)N(R_{Q})—, C₅-C₆ cycloalkyl, 3- to 12-membered heterocycle, 6- to 12-membered bridged heterocycle, 6- to 12-membered spiro heterocycle, and 6- to 12-membered spiro cycloalkyl; wherein each of the C₃-C₆ cycloalkyl, 3- to 12-membered heterocycle, 6- to 12-membered bridged heterocycle, 6- to 12-membered spiro heterocycle, and 6- to 12-membered spiro cycloalkyl is optionally substituted with 1-3 R_{Q}, and wherein each R_{Q} is independently selected from hydrogen, hydroxyl, C₁-C₆ alkyl, and C₃-C₆ cycloalkyl;
n is 1, 2, 3, 4, 5, 6, 7, 8, 9, or 10; and
W is

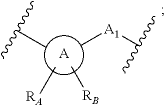

wherein Ring A is selected from 6- to 12-membered aryl and 5- to 12-membered heteroaryl; wherein A₁ is selected from a bond, —C(O) NH—, —NH—, —C(O)N(C₁-C₆ alkyl)-, and —C(O)O—; wherein R_{A} and Re, which may be the same or different, are each independently selected from hydrogen, halogen, C₁-C₆ alkyl, C₃-C₈ cycloalkyl, and oxo; or R_{A} and R_{B} together with the carbon atom(s) to which they are attached form a 3- to 6-membered cycloalkyl or a 3- to 6-membered heterocycle.

In some embodiments, R₁ is selected from hydroxy, amino, halogen, C₁-C₆ alkyl, C₃-C₆ cycloalkyl, C₁-C₆ haloalkyl, and C₁-C₆ alkoxy. In some embodiments. R₁ is hydroxy. In some embodiments, R₁ is selected from amino. In some embodiments, R₁ is selected from halogen.

In some embodiments. R₁ is selected from C₁-C₆ alkyl. In some embodiments, R₁ is selected from C₁ alkyl. In some embodiments, R₁ is selected from C₂ alkyl. In some embodiments, R₁ is selected from C₃ alkyl. In some embodiments, R₁ is selected from C₄ alkyl. In some embodiments, R₁ is selected from C₅ alkyl. In some embodiments, R₁ is selected from C₆ alkyl. In some embodiments. R₁ is selected from methyl and ethyl. In some embodiments, R₁ is methyl. In some embodiments, R₁ is ethyl.

In some embodiments, R₁ is selected from C₃-C₆ cycloalkyl. In some embodiments, R₁ is selected from C₃ cycloalkyl. In some embodiments, R₁ is selected from C₄ cycloalkyl. In some embodiments, R₁ is selected from C₅ cycloalkyl. In some embodiments, R₁ is selected from C₆ cycloalkyl.

In some embodiments, R₁ is selected from C₁-C₆ haloalkyl. In some embodiments, R₁ is selected from C₁ haloalkyl. In some embodiments, R₁ is selected from C₂ haloalkyl. In some embodiments, R₁ is selected from C₃ haloalkyl. In some embodiments, R₁ is selected from C₄ haloalkyl. In some embodiments, R₁ is selected from C₅ haloalkyl. In some embodiments, R₁ is selected from C₆ haloalkyl.

In some embodiments, R₁ is selected from C₁-C₆ alkoxy. In some embodiments, R₁ is selected from C₁ alkoxy. In some embodiments. R₁ is selected from C₂ alkoxy. In some embodiments, R₁ is selected from C₃ alkoxy. In some embodiments, R₁ is selected from C₄ alkoxy. In some embodiments, R₁ is selected from C₅ alkoxy. In some embodiments. R₁ is selected from C₆ alkoxy.

In some embodiments, R₂ is selected from hydrogen, C₁-C₆ alkyl, C₃-C₆ cycloalkyl, and C₁-C₆ haloalkyl. In some embodiments, R₂ is selected from hydrogen and C₁-C₆ alkyl. In some embodiments, R₂ is hydrogen.

In some embodiments, R₂ is selected from C₁-C₆ alkyl. In some embodiments, R₂ is selected from C₁ alkyl. In some embodiments, R₂ is selected from C₂ alkyl. In some embodiments, R₂ is selected from C₅ alkyl. In some embodiments, R₂ is selected from C₄ alkyl. In some embodiments, R₂ is selected from C₅ alkyl. In some embodiments, R₂ is selected from C₆ alkyl. In some embodiments, R₂ is selected from methyl and ethyl. In some embodiments, R₂ is methyl. In some embodiments, R₂ is ethyl. In some embodiments, R₂ is selected from hydrogen and methyl. In some embodiments, R₂ is selected from hydrogen and ethyl.

In some embodiments, R₂ is selected from C₃-C₆ cycloalkyl. In some embodiments, R₂ is selected from C₃ cycloalkyl. In some embodiments, R₂ is selected from C₄ cycloalkyl. In some embodiments, R₂ is selected from C₅ cycloalkyl. In some embodiments, R₂ is selected from C₆ cycloalkyl.

In some embodiments, R₂ is selected from C₁-C₆ haloalkyl. In some embodiments, R₂ is selected from C₁ haloalkyl. In some embodiments, R₂ is selected from C₂ haloalkyl. In some embodiments, R₂ is selected from C₃ haloalkyl. In some embodiments, R₂ is selected from C₄ haloalkyl. In some embodiments, R₂ is selected from C₅ haloalkyl. In some embodiments, R₂ is selected from C₆ haloalkyl.

In some embodiments, R₃ is selected from hydrogen, halogen, C₁-C₆ alkyl, C₃-C₆ cycloalkyl, C₁-C₆ haloalkyl, and C₁-C₆ alkoxy.

In some embodiments, R₃ is hydrogen. In some embodiments, R₃ is selected from halogen. In some embodiments, R₃ is F. In some embodiments, R₃ is Cl. In some embodiments, R₃ is Br. In some embodiments, R₃ is I.

In some embodiments, R₃ is selected from C₁-C₆ alkyl. In some embodiments, R₃ is selected from C₁ alkyl. In some embodiments, R₃ is selected from C₂ alkyl. In some embodiments, R₃ is selected from C₅ alkyl. In some embodiments, R₃ is selected from C₄ alkyl. In some embodiments, R₃ is selected from C₅ alkyl. In some embodiments, R₃ is selected from C₆ alkyl. In some embodiments, R₃ is methyl.

In some embodiments, R₃ is selected from C₃-C₆ cycloalkyl. In some embodiments, R₃ is selected from C₃ cycloalkyl. In some embodiments, R₃ is selected from C₄ cycloalkyl. In some embodiments, R₃ is selected from C₅ cycloalkyl. In some embodiments, R₃ is selected from C₆ cycloalkyl.

In some embodiments. R₃ is selected from C₁-C₆ haloalkyl. In some embodiments, R₃ is selected from C₁ haloalkyl. In some embodiments, R₃ is selected from C₂ haloalkyl. In some embodiments, R₃ is selected from Ca haloalkyl. In some embodiments, R₃ is selected from C₄ haloalkyl. In some embodiments, R₃ is selected from C₅ haloalkyl. In some embodiments, R₃ is selected from C₆ haloalkyl. In some embodiments, R₃ is trifluoromethyl.

In some embodiments, R₃ is selected from C₁-C₆ alkoxy. In some embodiments, R₃ is selected from C₁ alkoxy. In some embodiments, R₃ is selected from C₂ alkoxy. In some embodiments, R₃ is selected from C₃ alkoxy. In some embodiments, R₃ is selected from C₄ alkoxy. In some embodiments, R₃ is selected from C₅ alkoxy. In some embodiments. R₃ is selected from Ce alkoxy.

In some embodiments, R₃ is selected from hydrogen, halogen, C₁-C₆ alkyl, and C₁-C₆ haloalkyl. In some embodiments, R₃ is selected from Br, Cl, methyl, and trifluoromethyl.

In some embodiments, R₄ is selected from hydrogen, C₁-C₆ alkyl, C₃-C₆ cycloalkyl, C₁-C₄ alkenyl, C₁-C₅ alkoxy, C₃-C₆ cycloalkenyl, C₃-C₈ heterocyclyl, 5- to 10-membered heteroaryl, and 5- to 10-membered aryl; wherein each of the C₃-C₆ cycloalkyl, C₃-C₆ heterocyclyl, 5- to 10-membered heteroaryl, and 5- to 10-membered aryl is optionally substituted with 1-3 groups independently selected from halogen, hydroxy, amino, oxo, C₁-C₆ alkyl, C₃-C₆ cycloalkyl, and C₁-C₆ haloalkyl.

In some embodiments, R₄ is hydrogen. In some embodiments, R₄ is selected from halogen. In some embodiments, R₄ is F. In some embodiments, R₄ is C₁. In some embodiments, R₄ is I. In some embodiments. R₄ is Br.

In some embodiments, R₄ is selected from C₁-C₆ alkyl. In some embodiments, R₄ is selected from C₁ alkyl. In some embodiments, R₄ is selected from C₂ alkyl. In some embodiments, R₄ is selected from C₃ alkyl. In some embodiments, R₄ is selected from C₄ alkyl. In some embodiments, R₄ is selected from C₅ alkyl. In some embodiments, R₄ is selected from C₆ alkyl.

In some embodiments, R₄ is selected from C₃-C₆ cycloalkyl optionally substituted with 1-3 groups independently selected from halogen, hydroxy, amino, oxo, C₁-C₆ alkyl, C₃-C₆ cycloalkyl, and C₁-C₆ haloalkyl. In some embodiments, R₄ is selected from C₃-C₆ cycloalkyl. In some embodiments, R₄ is selected from C₃ cycloalkyl. In some embodiments. R₄ is selected from C₄ cycloalkyl. In some embodiments, R₄ is selected from C₅ cycloalkyl. In some embodiments, R₄ is selected from C₆ cycloalkyl.

In some embodiments, R₃ is selected from C₁-C₄ alkenyl. In some embodiments, R₄ is selected from C₁ alkenyl. In some embodiments, R₄ is selected from C₂ alkenyl. In some embodiments, R₄ is selected from C₃ alkenyl. In some embodiments, R₄ is selected from C₄ alkenyl.

In some embodiments, R₄ is selected from C₁-C₅ alkoxy. In some embodiments, R₄ is selected from C₁ alkoxy. In some embodiments, R₄ is selected from C₂ alkoxy. In some embodiments, R₄ is selected from C₃ alkoxy. In some embodiments, R₄ is selected from C₄ alkoxy. In some embodiments, R₄ is selected from C₅ alkoxy.

In some embodiments, R₄ is selected from C₃-C₆ cycloalkenyl. In some embodiments, R₄ is selected from C₃ cycloalkenyl. In some embodiments, R₄ is selected from C₄ cycloalkenyl. In some embodiments, R₄ is selected from C₅ cycloalkenyl. In some embodiments, R₄ is selected from C₆ cycloalkenyl.

In some embodiments, R₄ is selected from C₃-C₈ heterocyclyl optionally substituted with 1-3 groups independently selected from halogen, hydroxy, amino, oxo, C₁-C₆ alkyl, C₃-C₆ cycloalkyl, and C₁-C₆ haloalkyl. In some embodiments, R₄ is selected from C₃-C₈ heterocyclyl. In some embodiments, R₄ is selected from Ca heterocyclyl. In some embodiments, R₄ is selected from C₄ heterocyclyl. In some embodiments, R₄ is selected from C₅ heterocyclyl. In some embodiments, R₄ is selected from C₆ heterocyclyl. In some embodiments, R₄ is selected from C₂ heterocyclyl. In some embodiments. R₄ is selected from C₈ heterocyclyl.

In some embodiments, R₄ is selected from 5- to 10-membered heteroaryl optionally substituted with 1-3 groups independently selected from halogen, hydroxy, amino, oxo, C₁-C₆ alkyl, C₃-C₆ cycloalkyl, and C₁-C₆ haloalkyl. In some embodiments, R₄ is selected from 5- to 10-membered heteroaryl. In some embodiments, R₄ is selected from 5- to 10-membered heteroaryl substituted with 1-3 groups independently selected from halogen, hydroxy, amino, oxo, C₁-C₆ alkyl, C₃-C₆ cycloalkyl, and C₁-C₆ haloalkyl. In some embodiments, R₄ is selected from 5-membered heteroaryl. In some embodiments, R₄ is selected from 6-membered heteroaryl. In some embodiments, R₄ is selected from 7-membered heteroaryl. In some embodiments, R₄ is selected from 8-membered heteroaryl. In some embodiments, R₄ is selected from 9-membered heteroaryl. In some embodiments, R₄ is selected from 10-membered heteroaryl.

In some embodiments, R₄ is selected from 5- to 10-membered aryl optionally substituted with 1-3 groups independently selected from halogen, hydroxy, amino, oxo, C₁-C₆ alkyl, C₃-C₆ cycloalkyl, and C₁-C₆ haloalkyl. In some embodiments, R₄ is selected from 5- to 10-membered aryl. In some embodiments, R₄ is selected from 5- to 10-membered aryl substituted with 1-3 groups independently selected from halogen, hydroxy, amino, oxo, C₁-C₆ alkyl, C₃-C₆ cycloalkyl, and C₁-C₆ haloalkyl. In some embodiments. R₄ is selected from 5-membered aryl. In some embodiments, R₄ is selected from 6-membered aryl. In some embodiments, R₄ is selected from 7-membered aryl. In some embodiments, R₄ is selected from 8-membered aryl. In some embodiments, R₄ is selected from 9-membered aryl. In some embodiments, R₄ is selected from 10-membered aryl.

In some embodiments, R₄ is selected from hydrogen, halogen, C₁-C₆ alkyl, C₂-C₄ alkenyl, and 5- to 10-membered heteroaryl, wherein the 5- to 10-membered heteroaryl is optionally substituted with 1-2 groups independently selected from methyl, ethyl, propyl, butyl, and pentyl. In some embodiments, R₄ is selected from C₂-C₄ alkenyl. In some embodiments. R₄ is selected from 5- to 10-membered heteroaryl optionally substituted with 1-2 groups independently selected from methyl, ethyl, propyl, butyl, and pentyl. In some embodiments, R₄ is selected from 5- to 10-membered heteroaryl substituted with 1-2 groups independently selected from methyl, ethyl, propyl, butyl, and pentyl.

In some embodiments, R₄ is selected from methyl, ethyl, C₂ alkenyl,

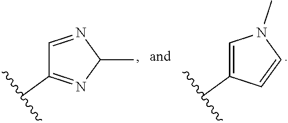

In some embodiments, R₄ is methyl. In some embodiments, R₄ is ethyl. In some embodiments, R₄ is C₂ alkenyl. In some embodiments, R₄ is

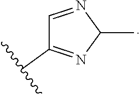 [SMILES: *C1=NC(C)N=C1]

In some embodiments, R₄ is

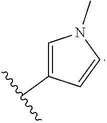 [SMILES: *c1ccn(C)c1]

In some embodiments, R₅ is selected from C₁-C₆ alkyl, deuterated C₁-C₆ alkyl, C₁-C₆ haloalkyl, C₃-C₆ cycloalkyl, deuterated C₁-C₆ alkoxy, and C₁-C₆ alkoxy.

In some embodiments, R₅ is selected from C₁-C₆ alkyl. In some embodiments, R₅ is selected from C₁ alkyl. In some embodiments, R₅ is selected from C₂ alkyl. In some embodiments, R₅ is selected from C₃ alkyl. In some embodiments, R₅ is selected from C₄ alkyl. In some embodiments, R₅ is selected from C₅ alkyl. In some embodiments, R₅ is selected from C₆ alkyl.

In some embodiments, R₅ is selected from deuterated C₁-C₆ alkyl. In some embodiments, R₅ is selected from deuterated C₁ alkyl. In some embodiments, R₅ is selected from deuterated C₂ alkyl. In some embodiments, R₅ is selected from deuterated C₃ alkyl. In some embodiments, R₅ is selected from deuterated C₄ alkyl. In some embodiments, R₅ is selected from deuterated C₅ alkyl. In some embodiments, R₅ is selected from deuterated C₆ alkyl.

In some embodiments. R₅ is selected from C₁-C₆ haloalkyl. In some embodiments, R₅ is selected from C₁ haloalkyl. In some embodiments, R₅ is selected from C₂ haloalkyl. In some embodiments, R₅ is selected from C₃ haloalkyl. In some embodiments, R₅ is selected from C₄ haloalkyl. In some embodiments, R₅ is selected from C₅ haloalkyl. In some embodiments, R₅ is selected from C₆ haloalkyl.

In some embodiments, R₅ is selected from C₃-C₆ cycloalkyl. In some embodiments, R₅ is selected from C₅ cycloalkyl. In some embodiments, R₅ is selected from C₄ cycloalkyl. In some embodiments, R₅ is selected from C₅ cycloalkyl. In some embodiments, R₅ is selected from C₆ cycloalkyl.

In some embodiments, R₅ is selected from deuterated C₁-C₆ alkoxy. In some embodiments. R₅ is selected from deuterated C₁ alkoxy. In some embodiments. R₅ is selected from deuterated C₂ alkoxy. In some embodiments, R₃ is selected from deuterated C₃ alkoxy. In some embodiments, R₅ is selected from deuterated C₄ alkoxy. In some embodiments, R₅ is selected from deuterated C₅ alkoxy. In some embodiments, R₅ is selected from deuterated C₆ alkoxy.

In some embodiments, R₅ is selected from C₁-C₆ alkoxy. In some embodiments, R₅ is selected from C₁ alkoxy. In some embodiments, R₅ is selected from C₂ alkoxy. In some embodiments, R₅ is selected from C₃ alkoxy. In some embodiments, R₅ is selected from C₄ alkoxy. In some embodiments, R₅ is selected from C₅ alkoxy. In some embodiments, R₅ is selected from C₆ alkoxy.

In some embodiments, R₅ is selected from C₁-C₆ haloalkoxy. In some embodiments, R₅ is selected from C₁ haloalkoxy. In some embodiments, R₅ is selected from C₂ haloalkoxy. In some embodiments, R₅ is selected from C₃ haloalkoxy. In some embodiments, R₅ is selected from C₄ haloalkoxy. In some embodiments, R₅ is selected from C₅ haloalkoxy. In some embodiments, R₅ is selected from Ce haloalkoxy.

In some embodiments, R₅ is selected from C₁-C₆ alkoxy and deuterated C₁-C₆ alkoxy. In some embodiments. R₅ is —OCH₃ or —OCD₃. In some embodiments, R₅ is —OCH₃. In some embodiments, R₅ is —OCD₃

In some embodiments, Q is

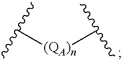

wherein each Q_{A} is independently selected from —C(H)(R_{Q})—, —O—, —N(R_{Q})—, —S(O)₂—, —C(O)—, —C(O)N(R_{Q})—, C₃-C₆ cycloalkyl, 3- to 12-membered heterocycle, 6- to 12-membered bridged heterocycle, 6- to 12-membered spiro heterocycle, and 6- to 12-membered spiro cycloalkyl; wherein each R_{Q} is independently selected from hydrogen, C₁-C₆ alkyl, and C₃-C₆ cycloalkyl.

In some embodiments, Q_{A} is selected from —C(H)(R_{Q})—. In some embodiments. Q_{A} is —O—. In some embodiments, Q_{A} is selected from —N(R_{Q})—. In some embodiments, Q_{A} is —S(O)₂—. In some embodiments, Q_{A} is —C(O)—. In some embodiments, Q_{A} is —NH—. In some embodiments, Q_{A} is selected from —C(O)N(R_{Q})—.

In some embodiments, Q_{A} is selected from C₃-C₆ cycloalkyl. In some embodiments, Q_{A} is selected from C₃ cycloalkyl. In some embodiments. Q_{A} is selected from C₄ cycloalkyl. In some embodiments, Q_{A} is selected from C₅ cycloalkyl. In some embodiments, Q_{A} is selected from C₆ cycloalkyl.

In some embodiments. Q_{A} is selected from 3- to 12-membered heterocycle. In some embodiments, Q_{A} is selected from 3-membered heterocycle. In some embodiments, Q_{A} is selected from 4-membered heterocycle. In some embodiments, Q_{A} is selected from 5-membered heterocycle. In some embodiments, Q_{A} is selected from 6-membered heterocycle. In some embodiments, Q_{A} is selected from 7-membered heterocycle. In some embodiments, Q_{A} is selected from 8-membered heterocycle. In some embodiments, Q_{A} is selected from 9-membered heterocycle. In some embodiments, Q_{A} is selected from 10-membered heterocycle. In some embodiments, Q_{A} is selected from 11-membered heterocycle. In some embodiments, Q_{A} is selected from 12-membered heterocycle.

In some embodiments, Q_{A} is selected from 6- to 12-membered bridged heterocycle. In some embodiments, Q_{A} is selected from 6-membered bridged heterocycle. In some embodiments, Q_{A} is selected from 7-membered bridged heterocycle. In some embodiments, Q_{A} is selected from 8-membered bridged heterocycle. In some embodiments, Q_{A} is selected from 9-membered bridged heterocycle. In some embodiments, Q_{A} is selected from 10-membered bridged heterocycle. In some embodiments, Q_{A} is selected from 11-membered bridged heterocycle. In some embodiments, Q_{A} is selected from 12-membered bridged heterocycle.

In some embodiments, Q_{A} is selected from 6- to 12-membered spiro heterocycle. In some embodiments, Q_{A} is selected from 6-membered spiro heterocycle. In some embodiments, Q_{A} is selected from 7-membered spiro heterocycle. In some embodiments, Q_{A} is selected from 8-membered spiro heterocycle. In some embodiments, Q_{A} is selected from 9-membered spiro heterocycle. In some embodiments, Q_{A} is selected from 10-membered spiro heterocycle. In some embodiments, Q_{A} is selected from 11-membered spiro heterocycle. In some embodiments, Q_{A} is selected from 12-membered spiro heterocycle.

In some embodiments, Q_{A} is selected from 6- to 12-membered spiro cycloalkyl. In some embodiments, Q_{A} is selected from 6-membered spiro cycloalkyl. In some embodiments, Q_{A} is selected from 7-membered spiro cycloalkyl. In some embodiments, Q_{A} is selected from 8-membered spiro cycloalkyl. In some embodiments, Q_{A} is selected from 9-membered spiro cycloalkyl. In some embodiments, Q_{A} is selected from 10-membered spiro cycloalkyl. In some embodiments, Q_{A} is selected from 11-membered spiro cycloalkyl. In some embodiments, Q_{A} is selected from 12-membered spiro cycloalkyl.

In some embodiments, Q_{A} is independently selected from —CH₂—, —O—, —C(O)—, C₃-C₆ cycloalkyl, 4- to 8-membered heterocycle, 7- to 11-membered spiro heterocycle, and 7- to 11-membered spiro cycloalkyl. In some embodiments, Q_{A} is —CH₂—. In some embodiments, Q_{A} is selected from 4- to 8-membered heterocycle. In some embodiments, Q_{A} is selected from 7- to 11-membered spiro heterocycle. In some embodiments, Q_{A} is selected from 7- to 11-membered spiro cycloalkyl.

In some embodiments, at least one R_{Q} is hydrogen. In some embodiments, at least one R_{Q} is hydroxyl. In some embodiments, at least one R_{Q} is selected from C₁-C₆ alkyl. In some embodiments, at least one R₃ is selected from C₁ alkyl. In some embodiments, at least one R_{Q} is selected from C₂ alkyl. In some embodiments, at least one R_{Q} is selected from C₆ alkyl. In some embodiments, at least one R_{Q} is selected from C₄ alkyl. In some embodiments, at least one R_{Q} is selected from C₅ alkyl. In some embodiments, at least one R_{Q} is selected from C₆ alkyl. In some embodiments, at least one R₃ is selected from C₃-C₆ cycloalkyl. In some embodiments, at least one R_{Q} is selected from C₃ cycloalkyl. In some embodiments, at least one R₃ is selected from C₄ cycloalkyl. In some embodiments, at least one R₃ is selected from C₅ cycloalkyl. In some embodiments, at least one R₃ is selected from C₆ cycloalkyl.

In some embodiments, each R₃ is hydrogen. In some embodiments, each R_{Q} is hydroxyl. In some embodiments, each R_{Q} is independently selected from C₁-C₆ alkyl. In some embodiments, each R_{Q} is selected from C₁ alkyl. In some embodiments, each R_{Q} is selected from C₂ alkyl. In some embodiments, each R_{Q} is selected from C₆ alkyl. In some embodiments, each R_{Q} is selected from C₄ alkyl. In some embodiments, each R_{Q} is selected from C₅ alkyl. In some embodiments, each R_{Q} is selected from C₆ alkyl. In some embodiments, each R_{Q} is independently selected from C₃-C₆ cycloalkyl. In some embodiments, each R_{Q} is selected from C₅ cycloalkyl. In some embodiments, each R_{Q} is selected from C₄ cycloalkyl. In some embodiments, each R_{Q} is selected from C₅ cycloalkyl. In some embodiments, each R_{Q} is selected from C₅ cycloalkyl. In some embodiments, each R_{Q} is independently selected from hydrogen, C₁-C₆ alkyl, and C₃-C₆ cycloalkyl.

In some embodiments, each Q_{A} is independently selected from —CH₂—, —O—, —C(O)—, —NH—, cyclopropyl, piperidinyl, piperazinyl, pyrrolidinyl, azetidinyl, 2,7-diazaspiro[3.5]nonanyl, 3,9-diazaspiro[5.5]undecanyl, 2,6-diazaspiro[3.3]heptanyl, 7-azaspiro[3.5]nonanyl, 3-azaspiro[5.5]undecanyl, 2-azaspiro[3.5]nonanyl, and 2-azaspiro[3.3]heptanyl. In some embodiments, at least one Q_{A} is independently selected from —CH₂—, —O—, —C(O)—, —NH—, cyclopropyl, piperidinyl, piperazinyl, pyrrolidinyl, azetidinyl, 2,7-diazaspiro[3.5]nonanyl, 3,9-diazaspiro[5.5]undecanyl, 2,6-diazaspiro[3.3]heptanyl, 7-azaspiro[3.5]nonanyl, 3-azaspiro[5.5]undecanyl, 2-azaspiro[3.5]nonanyl, and 2-azaspiro[3.3]heptanyl. In some embodiments, at least one Q_{A} is cyclopropyl. In some embodiments, at least one Q_{A} is piperidinyl. In some embodiments, at least one Q_{A} is piperazinyl. In some embodiments, at least one Q_{A} is pyrrolidinyl. In some embodiments, at least one Q_{A} is azetidinyl. In some embodiments, at least one Q_{A} is 2,7-diazaspiro[3.5]nonanyl. In some embodiments, at least one Q_{A} is 3,9-diazaspiro[5.5]undecanyl. In some embodiments, at least one Q_{A} is 2,6-diazaspiro[3.3]heptanyl. In some embodiments, at least one Q_{A} is 7-azaspiro[3.5]nonanyl. In some embodiments, at least one Q_{A} is 3-azaspiro[5.5]undecanyl. In some embodiments, at least one Q_{A} is 2-azaspiro[3.5]nonanyl. In some embodiments, at least one Q_{A} is 2-azaspiro[3.3]heptanyl.

In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10.

In some embodiments, Q is selected from

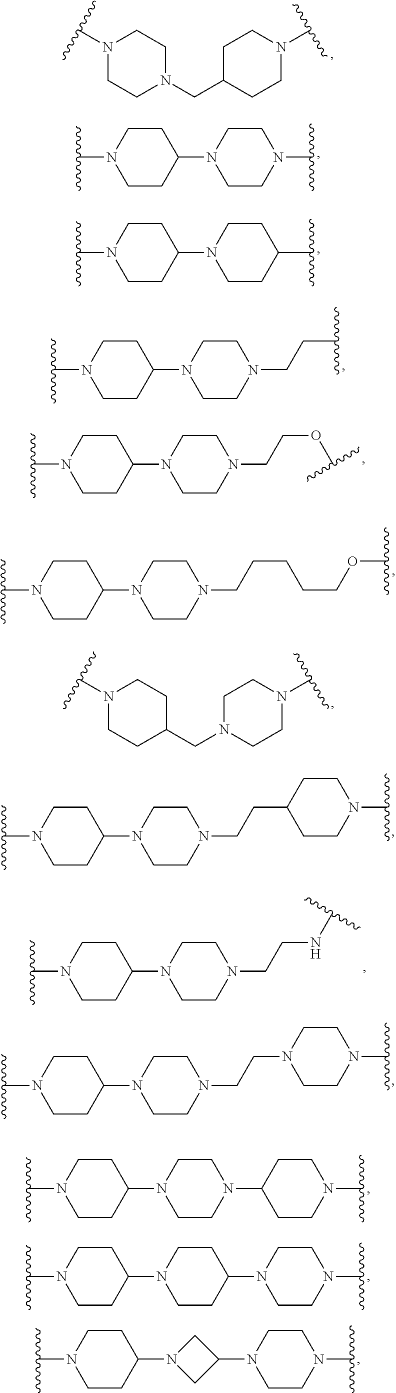

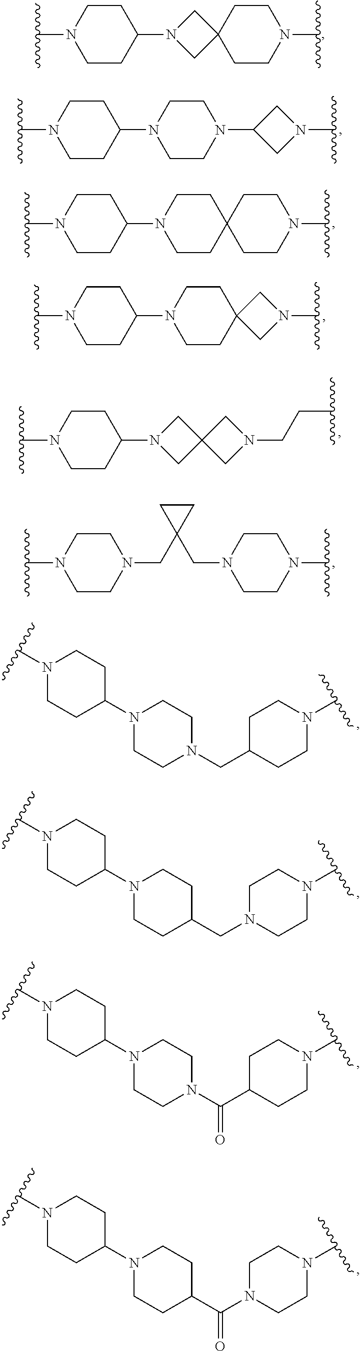

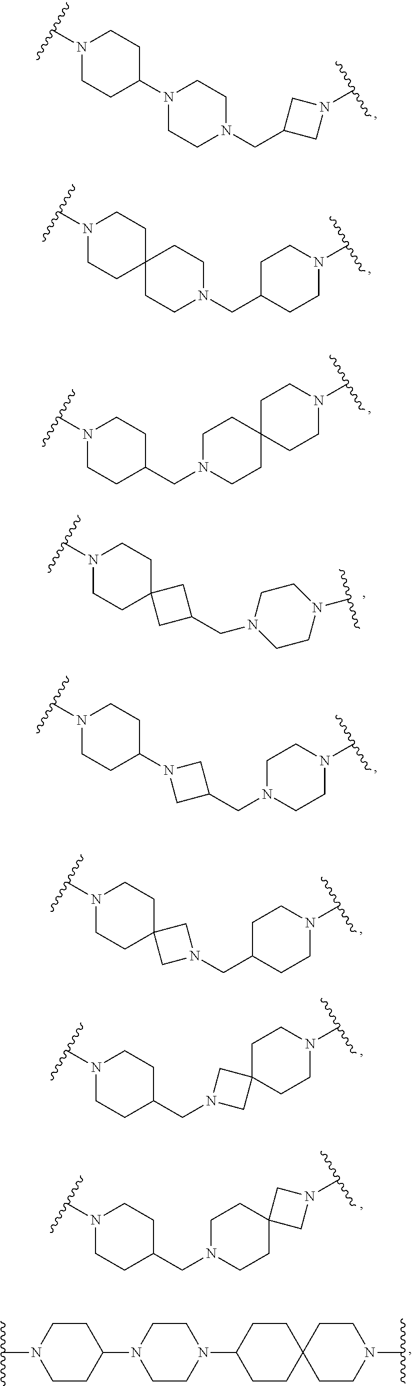

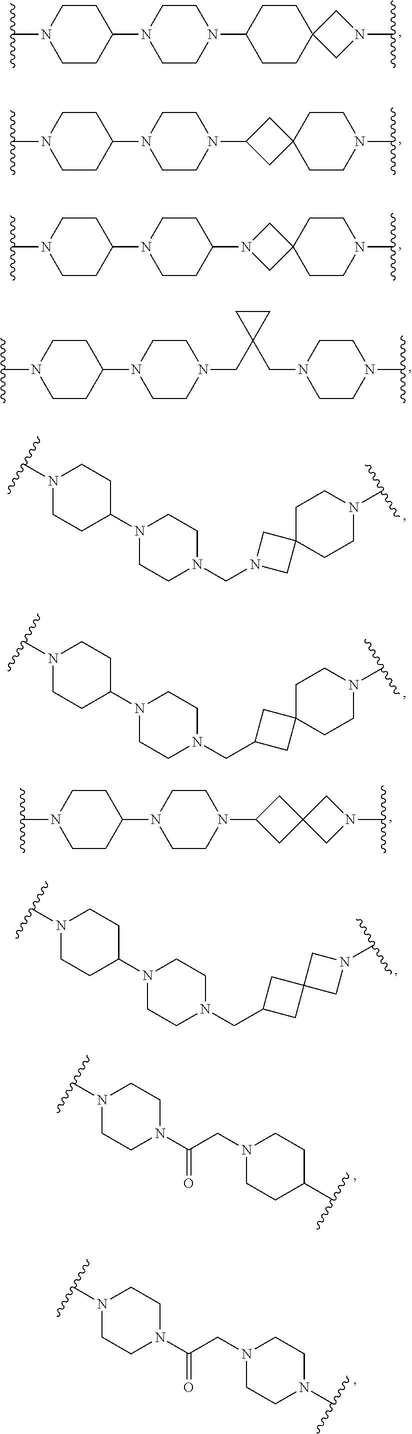

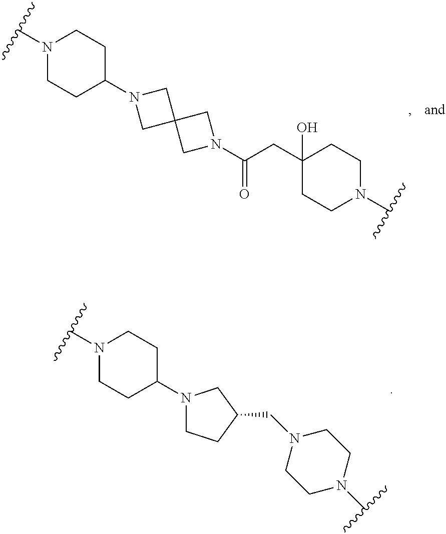 [SMILES: *N1CCC(N2CC3(CN(C(=O)CC4(O)CCN(*)CC4)C3)C2)CC1 ; *N1CCC(N2CC[C@@H](CN3CCN(*)CC3)C2)CC1]

In some embodiments, Q is selected from

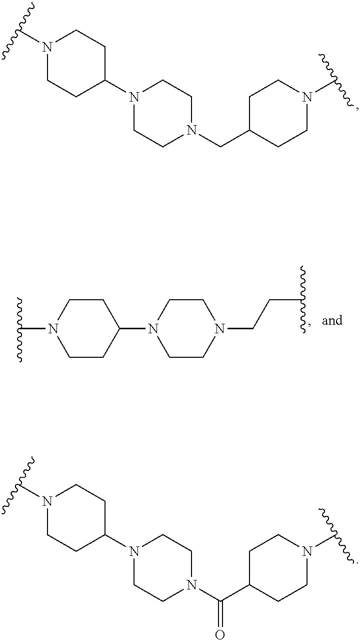 [SMILES: *N1CCC(CN2CCN(C3CCN(*)CC3)CC2)CC1 ; *CCN1CCN(C2CCN(*)CC2)CC1 ; *N1CCC(C(=O)N2CCN(C3CCN(*)CC3)CC2)CC1]

In some embodiments, Q is

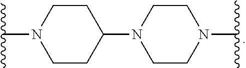 [SMILES: *N1CCC(N2CCN(*)CC2)CC1]

In some embodiments, Q is

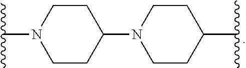 [SMILES: *C1CCN(C2CCN(*)CC2)CC1]

In some embodiments, Q is

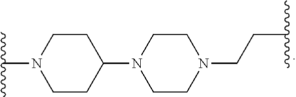 [SMILES: *CCN1CCN(C2CCN(*)CC2)CC1]

In some embodiments, Q is

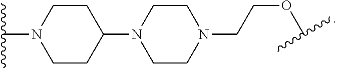 [SMILES: *OCCN1CCN(C2CCN(*)CC2)CC1]

In some embodiments, Q is

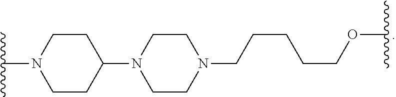 [SMILES: *OCCCCCN1CCN(C2CCN(*)CC2)CC1]

In some embodiments, Q is

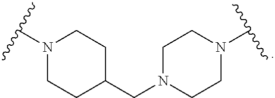 [SMILES: *N1CCC(CN2CCN(*)CC2)CC1]

In some embodiments, Q is

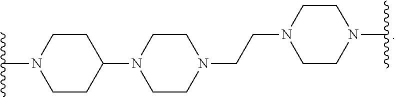 [SMILES: *N1CCC(N2CCN(CCN3CCN(*)CC3)CC2)CC1]

In some embodiments, Q is

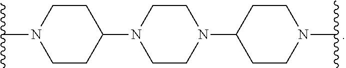 [SMILES: *N1CCC(N2CCN(C3CCN(*)CC3)CC2)CC1]

In some embodiments, Q is

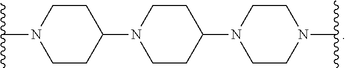 [SMILES: *N1CCC(N2CCC(N3CCN(*)CC3)CC2)CC1]

In some embodiments, Q is.

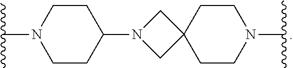 [SMILES: *N1CCC(N2CC3(CCN(*)CC3)C2)CC1]

In some embodiments, Q is

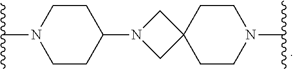 [SMILES: *N1CCC(N2CC3(CCN(*)CC3)C2)CC1]

In some embodiments, Q is

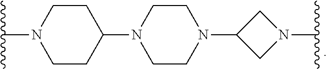 [SMILES: *N1CCC(N2CCN(C3CN(*)C3)CC2)CC1]

In some embodiments, Q is

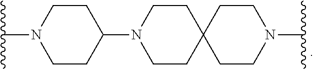 [SMILES: *N1CCC(N2CCC3(CCN(*)CC3)CC2)CC1]

In some embodiments, Q is

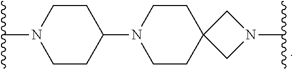 [SMILES: *N1CCC(N2CCC3(CC2)CN(*)C3)CC1]

In some embodiments, Q is

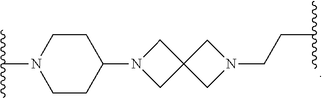 [SMILES: *CCN1CC2(C1)CN(C1CCN(*)CC1)C2]

In some embodiments, Q is

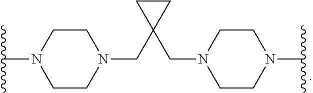 [SMILES: *N1CCN(CC2(CN3CCN(*)CC3)CC2)CC1]

In some embodiments, Q is

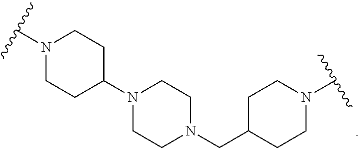 [SMILES: *N1CCC(CN2CCN(C3CCN(*)CC3)CC2)CC1]

In some embodiments, Q is

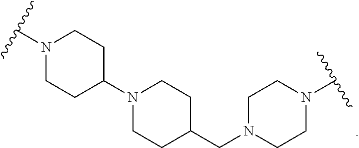 [SMILES: *N1CCC(N2CCC(CN3CCN(*)CC3)CC2)CC1]

In some embodiments, Q is

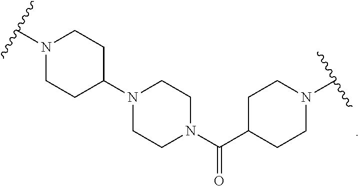 [SMILES: *N1CCC(C(=O)N2CCN(C3CCN(*)CC3)CC2)CC1]

In some embodiments, Q is

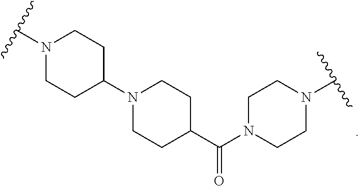 [SMILES: *N1CCC(N2CCC(C(=O)N3CCN(*)CC3)CC2)CC1]

In some embodiments, Q is

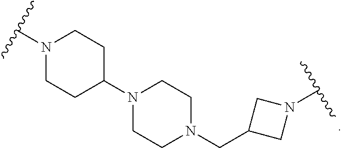 [SMILES: *N1CCC(N2CCN(CC3CN(*)C3)CC2)CC1]

In some embodiments, Q is

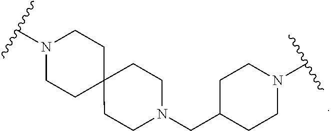 [SMILES: *N1CCC(CN2CCC3(CCN(*)CC3)CC2)CC1]

In some embodiments, Q is

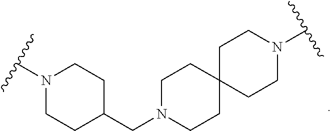 [SMILES: *N1CCC(CN2CCC3(CCN(*)CC3)CC2)CC1]

In some embodiments, Q is

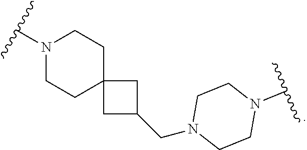 [SMILES: *N1CCN(CC2CC3(CCN(*)CC3)C2)CC1]

In some embodiments, Q is

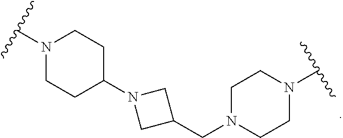 [SMILES: *N1CCC(N2CC(CN3CCN(*)CC3)C2)CC1]

In some embodiments, Q is

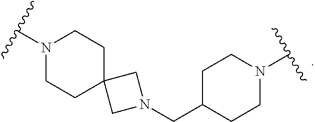 [SMILES: *N1CCC(CN2CC3(CCN(*)CC3)C2)CC1]

In some embodiments, Q is

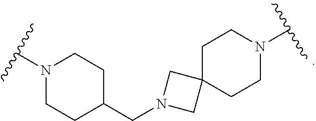 [SMILES: *N1CCC(CN2CC3(CCN(*)CC3)C2)CC1]

In some embodiments, Q is

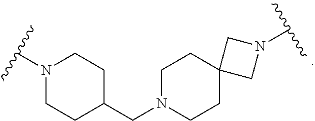 [SMILES: *N1CCC(CN2CCC3(CC2)CN(*)C3)CC1]

In some embodiments, Q is

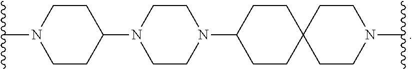 [SMILES: *N1CCC(N2CCN(C3CCC4(CC3)CCN(*)CC4)CC2)CC1]

In some embodiments, Q is

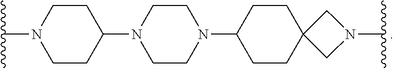 [SMILES: *N1CCC(N2CCN(C3CCC4(CC3)CN(*)C4)CC2)CC1]

In some embodiments, Q is

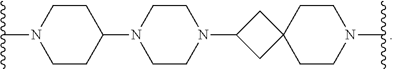 [SMILES: *N1CCC(N2CCN(C3CC4(CCN(*)CC4)C3)CC2)CC1]

In some embodiments, Q is

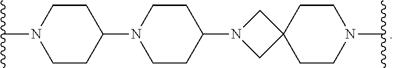 [SMILES: *N1CCC(N2CCC(N3CC4(CCN(*)CC4)C3)CC2)CC1]

In some embodiments, Q is

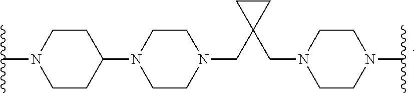 [SMILES: *N1CCC(N2CCN(CC3(CN4CCN(*)CC4)CC3)CC2)CC1]

In some embodiments, Q is

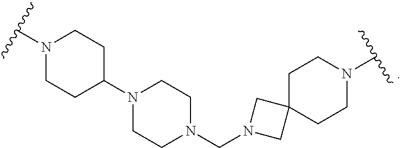 [SMILES: *N1CCC(N2CCN(CN3CC4(CCN(*)CC4)C3)CC2)CC1]

In some embodiments, Q S

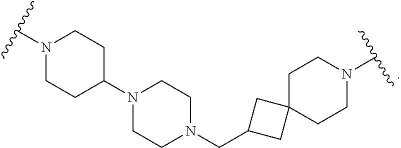 [SMILES: *N1CCC(N2CCN(CC3CC4(CCN(*)CC4)C3)CC2)CC1]

In some embodiments, Q is

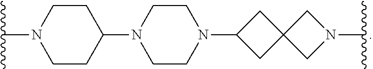 [SMILES: *N1CCC(N2CCN(C3CC4(C3)CN(*)C4)CC2)CC1]

In some embodiments, Q is

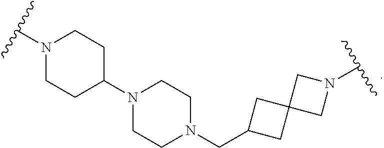 [SMILES: *N1CCC(N2CCN(CC3CC4(C3)CN(*)C4)CC2)CC1]

In some embodiments, Q is

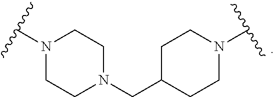 [SMILES: *N1CCC(CN2CCN(*)CC2)CC1]

In some embodiments, Q is

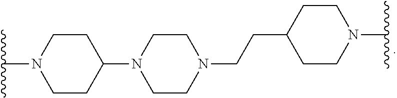 [SMILES: *N1CCC(CCN2CCN(C3CCN(*)CC3)CC2)CC1]

In some embodiments, Q is

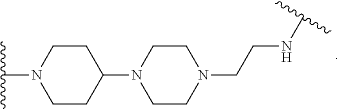 [SMILES: *NCCN1CCN(C2CCN(*)CC2)CC1]

In some embodiments, Q is

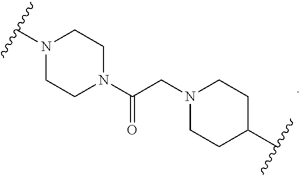 [SMILES: *C1CCN(CC(=O)N2CCN(*)CC2)CC1]

In some embodiments, Q is

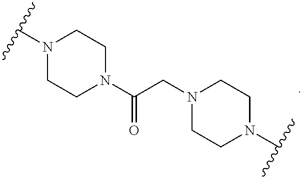 [SMILES: *N1CCN(CC(=O)N2CCN(*)CC2)CC1]

In some embodiments, Q is

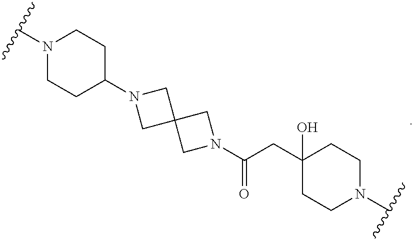 [SMILES: *N1CCC(N2CC3(CN(C(=O)CC4(O)CCN(*)CC4)C3)C2)CC1]

In some embodiments, Q is

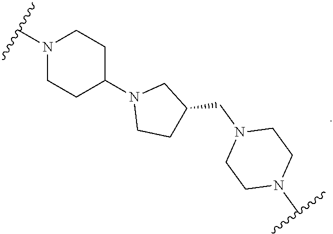 [SMILES: *N1CCC(N2CC[C@@H](CN3CCN(*)CC3)C2)CC1]

In some embodiments, W is

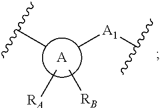

wherein Ring A is selected from 6- to 12-membered aryl and 5- to 12-membered heteroaryl; wherein A₁ is selected from a bond, —C(O) NH—, —C(O)N(C₁-C₆ alkyl)-, and —C(O)O—; wherein R_{A} and R_{B}, which may be the same or different, are each independently selected from hydrogen, halogen, C₁-C₆ alkyl, C₃-C₆ cycloalkyl, and oxo; or R_{A} and R_{B} together with the carbon atom(s) to which they are attached form a 3- to 6-membered cycloalkyl or a 3- to 6-membered heterocycle.

In some embodiments, Ring A is selected from 6- to 12-membered aryl. In some embodiments, Ring A is selected from 5- to 12-membered heteroaryl. In some embodiments, A₁ is a bond. In some embodiments, A₁ is a —C(O) NH—. In some embodiments, A₁ is selected from —C(O)N(C₁-C₆ alkyl)-. In some embodiments, A₁ is —C(O)O—.

In some embodiments, R_{A} is selected from hydrogen, halogen, C₁-C₈ alkyl, C₃-C₆ cycloalkyl, and oxo.

In some embodiments, R_{A} is hydrogen. In some embodiments, R_{A} is selected from halogen. In some embodiments, R₃ is F. In some embodiments. R_{A} is Br. In some embodiments, R_{A} is Cl. In some embodiments, R_{A} is I.

In some embodiments, R_{A} is selected from C₁-C₆ alkyl. In some embodiments, R_{A} is selected from C₁ alkyl. In some embodiments, R_{A} is selected from C₂ alkyl. In some embodiments, R_{A} is selected from C₅ alkyl. In some embodiments, R_{A} is selected from C₄ alkyl. In some embodiments, R_{A} is selected from C₅ alkyl. In some embodiments, R_{A} is selected from C₆ alkyl.

In some embodiments, R_{A} is selected from C₃-C₆ cycloalkyl. In some embodiments, R_{A} is selected from C₅ cycloalkyl. In some embodiments. R_{A} is selected from C₄ cycloalkyl. In some embodiments, R_{A} is selected from C₅ cycloalkyl. In some embodiments, R_{A} is selected from C₆ cycloalkyl.

In some embodiments. R_{A} is oxo.

In some embodiments, R_{B} is hydrogen. In some embodiments, R_{B} is selected from halogen. In some embodiments, R_{B} is F. In some embodiments, R_{B} is Br. In some embodiments, R_{B} is Cl. In some embodiments, R_{B} is I.

In some embodiments, R_{B} is selected from C₁-C₆ alkyl. In some embodiments, R₃ is selected from C₁ alkyl. In some embodiments, R_{B} is selected from C₂ alkyl. In some embodiments. R is selected from C₃ alkyl. In some embodiments. R_{B} is selected from C₄ alkyl. In some embodiments, R₃ is selected from C₅ alkyl. In some embodiments, R_{B} is selected from C₆ alkyl.

In some embodiments, R_{B} is selected from C₃-C₆ cycloalkyl. In some embodiments, R_{B} is selected from C₃ cycloalkyl. In some embodiments, R_{B} is selected from C₄ cycloalkyl. In some embodiments, R_{B} is selected from C₅ cycloalkyl. In some embodiments, R_{B} is selected from C₆ cycloalkyl.

In some embodiments, R_{B} is oxo.

In some embodiments, R_{A} and R_{B} together with the carbon atom(s) to which they are attached form a 3- to 6-membered cycloalkyl. In some embodiments, R_{A} and R_{B} together with the carbon atoms to which they are attached form a 3-membered cycloalkyl.

In some embodiments, R_{A} and R_{B} together with the carbon atom(s) to which they are attached form a 4-membered cycloalkyl. In some embodiments, R_{A} and R_{B} together with the carbon atom(s) to which they are attached form a 5-membered cycloalkyl. In some embodiments, R_{A} and R_{B} together with the carbon atom(s) to which they are attached form a 6-membered cycloalkyl.

In some embodiments, R_{A} and R_{B} together with the carbon atom(s) to which they are attached form a 3- to 6-membered heterocycle. In some embodiments, R_{A} and R_{B} together with the carbon atoms to which they are attached form a 3-membered heterocycle. In some embodiments, R_{A} and R_{B} together with the carbon atom(s) to which they are attached form a 4-membered heterocycle. In some embodiments, R_{A} and R_{B} together with the carbon atom(s) to which they are attached form a 5-membered heterocycle. In some embodiments, R_{A} and R_{B} together with the carbon atom(s) to which they are attached form a 6-membered heterocycle.

In some embodiments, W is

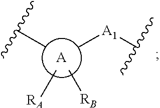

wherein Ring A is selected from phenyl and 9- to 12-membered heteroaryl; wherein A₁ is a bond, —NH—, or —C(O) NH—; and wherein R_{A} and R_{B}, which may be the same or different, are each independently selected from hydrogen, halogen, C₁-C₆ alkyl, C₃-C₆ cycloalkyl, and oxo; or R_{A} and R_{B} together with the carbon atom(s) to which they are attached form a 3- to 6-membered cycloalkyl or a 3- to 6-membered heterocycle.

In some embodiments, Ring A is phenyl.

In some embodiments, Ring A is selected from 9- to 12-membered heteroaryl. In some embodiments, Ring A is selected from a 9-membered heteroaryl. In some embodiments, Ring A is selected from a 10-membered heteroaryl. In some embodiments, Ring A is selected from a 11-membered heteroaryl. In some embodiments, Ring A is selected from a 12-membered heteroaryl.

In some embodiments, Ring A is selected from

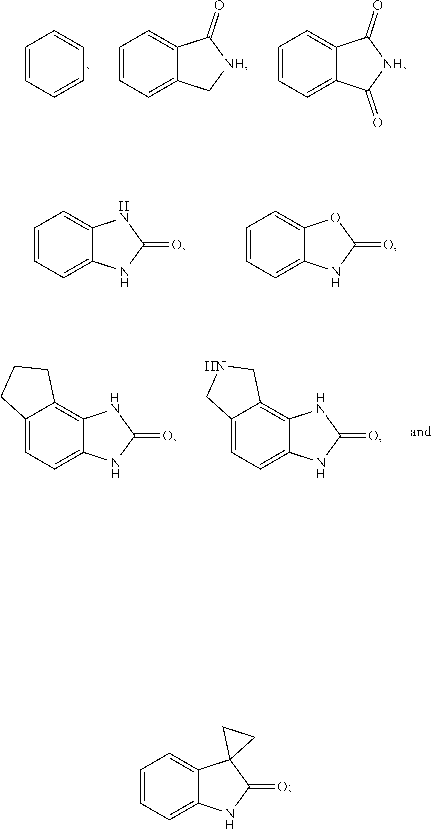

and wherein A₁ is a bond, —NH—, or —C(O) NH—.

In some embodiments, Ring A is

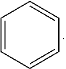

In some embodiments, Ring A is

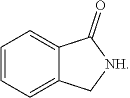

In some embodiments, Ring A is

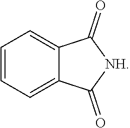 [SMILES: O=C1NC(=O)c2ccccc21]

In some embodiments, Ring A is

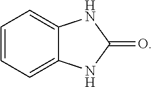

In some embodiments, Ring A is

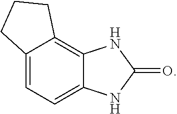 [SMILES: O=c1[nH]c2ccc3c(c2[nH]1)CCC3]

In some embodiments, Ring A is

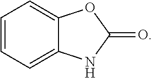

In some embodiments, Ring A is

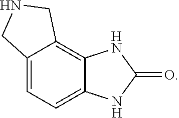 [SMILES: O=c1[nH]c2ccc3c(c2[nH]1)CNC3]

In some embodiments, Ring A is

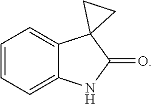 [SMILES: O=C1Nc2ccccc2C12CC2]

In some embodiments, A₁ is a bond. In some embodiments, A₁ is —NH—. In some embodiments, A₁ is —C(O) NH—.

In some embodiments, W is selected from

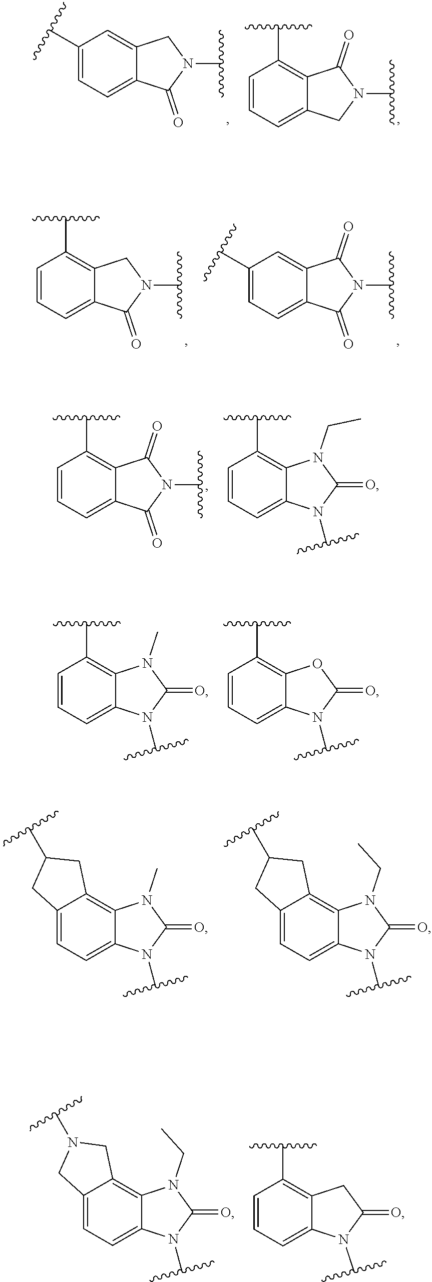

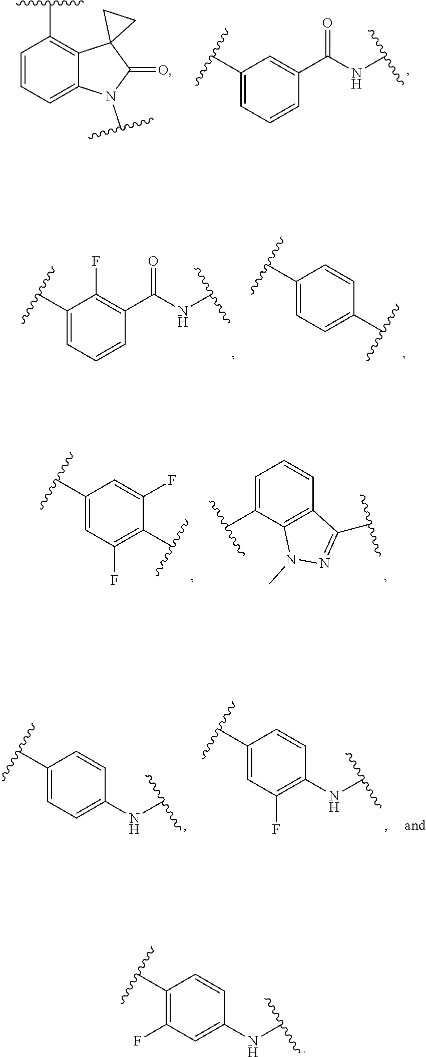

In some embodiments, W is selected from

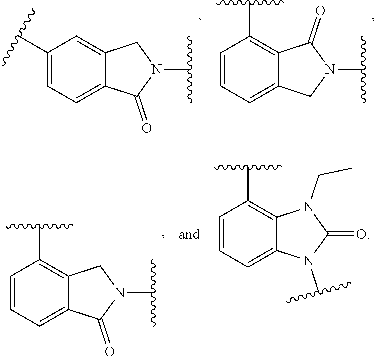

In some embodiments, W is

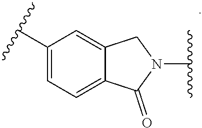 [SMILES: *c1ccc2c(c1)CN(*)C2=O]

In some embodiments, W is

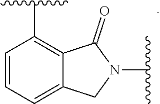 [SMILES: *c1cccc2c1C(=O)N(*)C2]

In some embodiments, W is

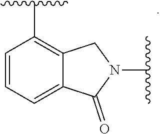 [SMILES: *c1cccc2c1CN(*)C2=O]

In some embodiments, W is

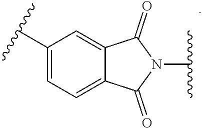 [SMILES: *c1ccc2c(c1)C(=O)N(*)C2=O]

In some embodiments, W is

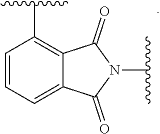 [SMILES: *c1cccc2c1C(=O)N(*)C2=O]

In some embodiments, W is

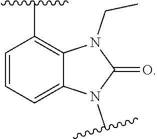 [SMILES: *c1cccc2c1n(CC)c(=O)n2*]

In some embodiments, W is

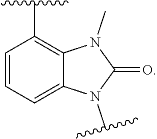 [SMILES: *c1cccc2c1n(C)c(=O)n2*]

In some embodiments, W is

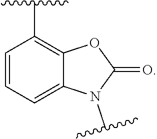 [SMILES: *c1cccc2c1oc(=O)n2*]

In some embodiments, W is

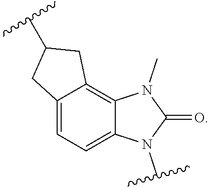 [SMILES: *C1Cc2ccc3c(c2C1)n(C)c(=O)n3*]

In some embodiments, W is

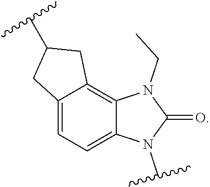 [SMILES: *C1Cc2ccc3c(c2C1)n(CC)c(=O)n3*]

In some embodiments, W is

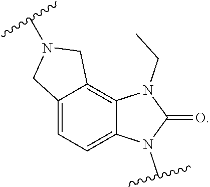 [SMILES: *N1Cc2ccc3c(c2C1)n(CC)c(=O)n3*]

In some embodiments, W is

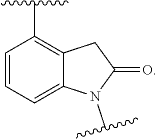 [SMILES: *c1cccc2c1CC(=O)N2*]

In some embodiments, W is

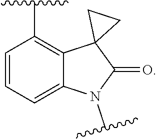 [SMILES: *c1cccc2c1C1(CC1)C(=O)N2*]

In some embodiments, W is

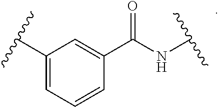 [SMILES: *NC(=O)c1cccc(*)c1]

In some embodiments, W is

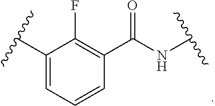 [SMILES: *NC(=O)c1cccc(*)c1F]

In some embodiments, W is

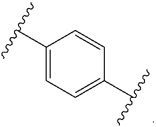 [SMILES: *c1ccc(*)cc1]

In some embodiments, W is

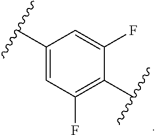 [SMILES: *c1cc(F)c(*)c(F)c1]

In some embodiments, W is

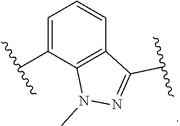 [SMILES: *c1nn(C)c2c(*)cccc12]

In some embodiments, W is

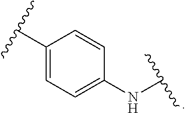 [SMILES: *Nc1ccc(*)cc1]

In some embodiments, W is

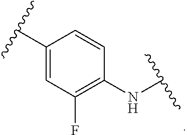 [SMILES: *Nc1ccc(*)cc1F]

In some embodiments, W is

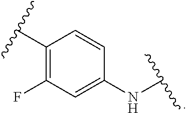 [SMILES: *Nc1ccc(*)c(F)c1]

Exemplary compounds of the present disclosure are listed in Table 2.

TABLE 2
Exemplary Compounds

| Cmpd | |
|---|---|
| # | Chemical Structure |
| 1 | 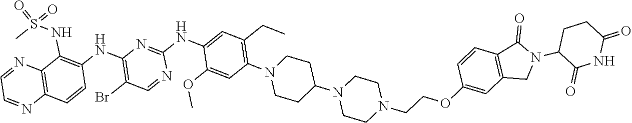 [SMILES: CCc1cc(Nc2ncc(Br)c(Nc3ccc4nccnc4c3NS(C)(=O)=O)n2)c(OC)cc1N1CCC(N2CCN(CCOc3ccc4c(c3)CN(C3CCC(=O)NC3=O)C4=O)CC2)CC1] |
| | IUPAC Nomenclature |
| | N-(6-((5-bromo-2-((4-(4-(4-(2-((2-(2,6-dioxopiperidin-3-yl)-1- |
| | oxoisoindolin-5-yl)oxy)ethyl)piperazin-1-yl)piperidin-1-yl)-5-ethyl- |
| | 2-methoxyphenyl)amino)pyrimidin-4-yl)amino)quinoxalin-5- |
| | yl)methanesulfonamide |
| 2 | 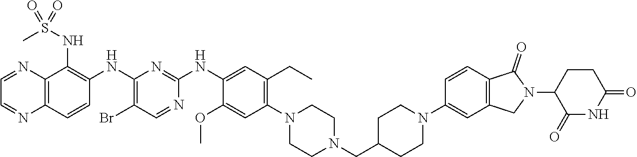 [SMILES: CCc1cc(Nc2ncc(Br)c(Nc3ccc4nccnc4c3NS(C)(=O)=O)n2)c(OC)cc1N1CCN(CC2CCN(c3ccc4c(c3)CN(C3CCC(=O)NC3=O)C4=O)CC2)CC1] |
| | N-(6-((5-bromo-2-((4-(4-((1-(2-(2,6-dioxopiperidin-3-yl)-1- |
| | oxoisoindolin-5-yl)piperidin-4-yl)methyl)piperazin-1-yl)-5-ethyl-2- |
| | methoxyphenyl)amino)pyrimidin-4-yl)amino)quinoxalin-5- |
| | yl)methanesulfonamide |
| 3 | 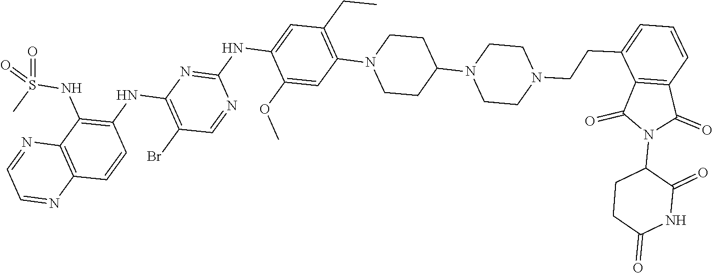 [SMILES: CCc1cc(Nc2ncc(Br)c(Nc3ccc4nccnc4c3NS(C)(=O)=O)n2)c(OC)cc1N1CCC(N2CCN(CCc3cccc4c3C(=O)N(C3CCC(=O)NC3=O)C4=O)CC2)CC1] |
| | N-(6-((5-bromo-2-((4-(4-(4-(2-(2-(2,6-dioxopiperidin-3-yl)-1,3- |
| | dioxoisoindolin-4-yl)ethyl)piperazin-1-yl)piperidin-1-yl)-5-ethyl-2- |
| | methoxyphenyl)amino)pyrimidin-4-yl)amino)quinoxalin-5- |
| | yl)methanesulfonamide |
| 4 | 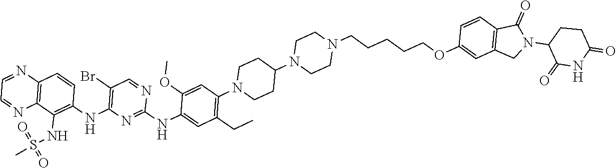 [SMILES: CCc1cc(Nc2ncc(Br)c(Nc3ccc4nccnc4c3NS(C)(=O)=O)n2)c(OC)cc1N1CCC(N2CCN(CCCCCOc3ccc4c(c3)CN(C3CCC(=O)NC3=O)C4=O)CC2)CC1] |
| | N-(6-((5-bromo-2-((4-(4-(4-(5-((2-(2,6-dioxopiperidin-3-yl)-1- |
| | oxoisoindolin-5-yl)oxy)pentyl)piperazin-1-yl)piperidin-1-yl)-5-ethyl- |
| | 2-methoxyphenyl)amino)pyrimidin-4-yl)amino)quinoxalin-5- |
| | yl)methanesulfonamide |
| 5 | 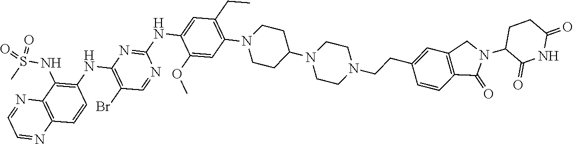 [SMILES: CCc1cc(Nc2ncc(Br)c(Nc3ccc4nccnc4c3NS(C)(=O)=O)n2)c(OC)cc1N1CCC(N2CCN(CCc3ccc4c(c3)CN(C3CCC(=O)NC3=O)C4=O)CC2)CC1] |
| | N-(6-((5-bromo-2-((4-(4-(4-(2-(2-(2,6-dioxopiperidin-3-yl)-1- |
| | oxoisoindolin-5-ylethyl)piperazin-1-yl)piperidin-1-yl)-5-ethyl-2- |
| | methoxyphenyl)amino)pyrimidin-4-yl)amino)quinoxalin-5- |
| | yl)methanesulfonamide |
| 6 | 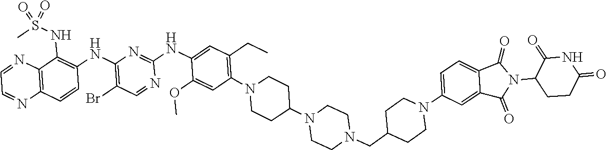 [SMILES: CCc1cc(Nc2ncc(Br)c(Nc3ccc4nccnc4c3NS(C)(=O)=O)n2)c(OC)cc1N1CCC(N2CCN(CC3CCN(c4ccc5c(c4)C(=O)N(C4CCC(=O)NC4=O)C5=O)CC3)CC2)CC1] |
| | N-(6-((5-bromo-2-((4-(4-(4-((1-(2-(2,6-dioxopiperidin-3-yl)-1,3- |
| | dioxoisoindolin-5-yl)piperidin-4-yl)methyl)piperazin-1-yl)piperidin- |
| | 1-yl)-5-ethyl-2-methoxyphenyl)amino)pyrimidin-4- |
| | yl)amino)quinoxalin-5-yl)methanesulfonamide |
| 7 | 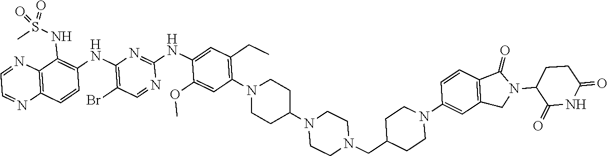 [SMILES: CCc1cc(Nc2ncc(Br)c(Nc3ccc4nccnc4c3NS(C)(=O)=O)n2)c(OC)cc1N1CCC(N2CCN(CC3CCN(c4ccc5c(c4)CN(C4CCC(=O)NC4=O)C5=O)CC3)CC2)CC1] |
| | N-(6-((5-bromo-2-((4-(4-(4-((1-(2-(2,6-dioxopiperidin-3-yl)-1- |
| | oxoisoindolin-5-yl)piperidin-4-yl)methyl)piperazin-1-yl)piperidin-1- |
| | yl)-5-ethyl-2-methoxyphenyl)amino)pyrimidin-4~ |
| | yl)amino)quinoxalin-5-yl)methanesulfonamide |
| 8 | 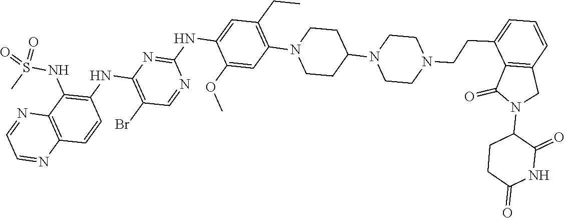 [SMILES: CCc1cc(Nc2ncc(Br)c(Nc3ccc4nccnc4c3NS(C)(=O)=O)n2)c(OC)cc1N1CCC(N2CCN(CCc3cccc4c3C(=O)N(C3CCC(=O)NC3=O)C4)CC2)CC1] |
| | N-(6-((5-bromo-2-((4-(4-(4-(2-(2-(2,6-dioxopiperidin-3-yl)-3- |
| | oxoisoindolin-4-yl)ethyl)piperazin-1-yl)piperidin-1-yl)-5-ethyl-2- |
| | methoxyphenyl)amino)pyrimidin-4-yl)amino)quinoxalin-5- |
| | yl)methanesulfonamide |
| 9 | 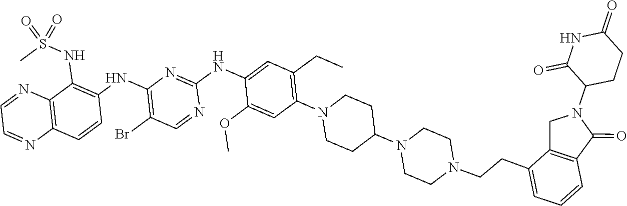 [SMILES: CCc1cc(Nc2ncc(Br)c(Nc3ccc4nccnc4c3NS(C)(=O)=O)n2)c(OC)cc1N1CCC(N2CCN(CCc3cccc4c3CN(C3CCC(=O)NC3=O)C4=O)CC2)CC1] |
| | N-(6-((5-bromo-2-((4-(4-(4-(2-(2-(2,6-dioxopiperidin-3-yl)-1- |
| | oxoisoindolin-4-yl)ethyl)piperazin-1-yl)piperidin-1-yl)-5-ethyl-2- |
| | methoxyphenyl)amino)pyrimidin-4-yl)amino)quinoxalin-5- |
| | yl)methanesulfonamide |
| 10 | 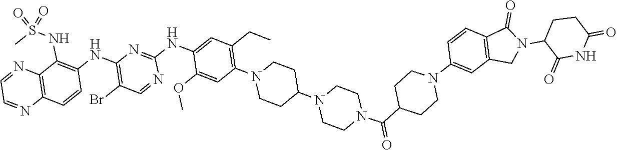 [SMILES: CCc1cc(Nc2ncc(Br)c(Nc3ccc4nccnc4c3NS(C)(=O)=O)n2)c(OC)cc1N1CCC(N2CCN(C(=O)C3CCN(c4ccc5c(c4)CN(C4CCC(=O)NC4=O)C5=O)CC3)CC2)CC1] |
| | N-(6-((5-bromo-2-((4-(4-(4-(1-(2-(2,6-dioxopiperidin-3-yl)-1- |
| | oxoisoindolin-5-yl)piperidine-4-carbonyl)piperazin-1-yl)piperidin-1- |
| | yl)-5-ethyl-2-methoxyphenyl)amino)pyrimidin-4- |
| | yl)amino)quinoxalin-5-yl)methanesulfonamide |
| 11 | 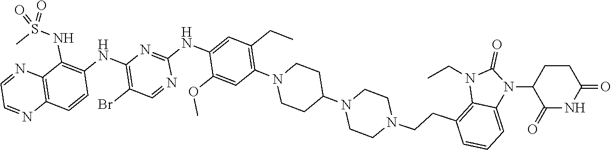 [SMILES: CCc1cc(Nc2ncc(Br)c(Nc3ccc4nccnc4c3NS(C)(=O)=O)n2)c(OC)cc1N1CCC(N2CCN(CCc3cccc4c3n(CC)c(=O)n4C3CCC(=O)NC3=O)CC2)CC1] |
| | N-(6-((5-bromo-2-((4-(4-(4-(2-(1-(2,6-dioxopiperidin-3-yl)-3-ethyl- |
| | 2-oxo-2,3-dihydro-1H-benzo[d]imidazol-4-yl)ethyl)piperazin-1- |
| | yl)piperidin-1-yl)-5-ethyl-2-methoxyphenyl)amino)pyrimidin-4- |
| | yl)amino)quinoxalin-5-yl)methanesulfonamide |
| 12 | 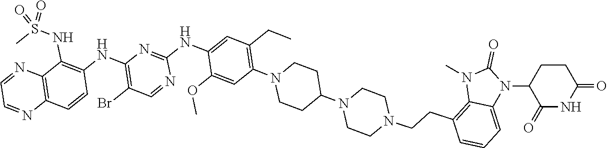 [SMILES: CCc1cc(Nc2ncc(Br)c(Nc3ccc4nccnc4c3NS(C)(=O)=O)n2)c(OC)cc1N1CCC(N2CCN(CCc3cccc4c3n(C)c(=O)n4C3CCC(=O)NC3=O)CC2)CC1] |
| | N-(6-((5-bromo-2-((4-(4-(4-(2-(1-(2,6-dioxopiperidin-3-yl)-3- |
| | methyl-2-oxo-2,3-dihydro-1H-benzo[d]imidazol-4- |
| | yl)ethyl)piperazin-1-yl)piperidin-1-yl)-5-ethyl-2- |
| | methoxyphenyl)amino)pyrimidin-4-yl)amino)quinoxalin-5- |
| | yl)methanesulfonamide |
| 13 | 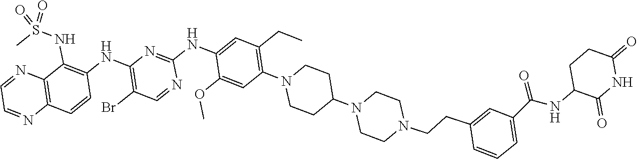 [SMILES: CCc1cc(Nc2ncc(Br)c(Nc3ccc4nccnc4c3NS(C)(=O)=O)n2)c(OC)cc1N1CCC(N2CCN(CCc3cccc(C(=O)NC4CCC(=O)NC4=O)c3)CC2)CC1] |
| | 3-(2-(4-(1-(4-((5-bromo-4-((5-(methylsulfonamido)quinoxalin-6- |
| | yl)amino)pyrimidin-2-yl)amino)-2-ethyl-5- |
| | methoxyphenyl)piperidin-4-yl)piperazin-1-yl)ethyl)-N-(2,6- |
| | dioxopiperidin-3-yl)benzamide |
| 14 | 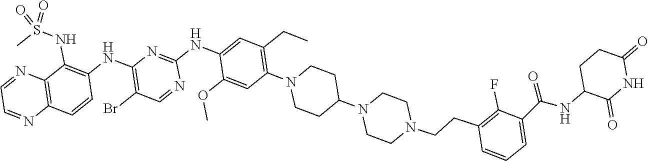 [SMILES: CCc1cc(Nc2ncc(Br)c(Nc3ccc4nccnc4c3NS(C)(=O)=O)n2)c(OC)cc1N1CCC(N2CCN(CCc3cccc(C(=O)NC4CCC(=O)NC4=O)c3F)CC2)CC1] |
| | 3-(2-(4-(1-(4-((5-bromo-4-((5-(methylsulfonamido)quinoxalin-6- |
| | yl)amino)pyrimidin-2-yl)amino)-2-ethyl-5- |
| | methoxyphenyl)piperidin-4-yl)piperazin-1-yl)ethyl)-N-(2,6- |
| | dioxopiperidin-3-yl)-2-fluorobenzamide |
| 15 | 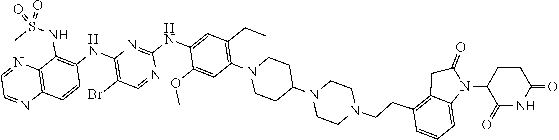 [SMILES: CCc1cc(Nc2ncc(Br)c(Nc3ccc4nccnc4c3NS(C)(=O)=O)n2)c(OC)cc1N1CCC(N2CCN(CCc3cccc4c3CC(=O)N4C3CCC(=O)NC3=O)CC2)CC1] |
| | N-(6-((5-bromo-2-((4-(4-(4-(2-(1-(2,6-dioxopiperidin-3-yl)-2- |
| | oxoindolin-4-yl)ethyl)piperazin-1-yl)piperidin-1-yl)-5-ethyl-2- |
| | methoxyphenyl)amino)pyrimidin-4-yl)amino)quinoxalin-5- |
| | yl)methanesulfonamide |
| 16 | 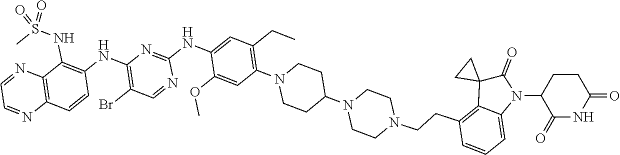 [SMILES: CCc1cc(Nc2ncc(Br)c(Nc3ccc4nccnc4c3NS(C)(=O)=O)n2)c(OC)cc1N1CCC(N2CCN(CCc3cccc4c3C3(CC3)C(=O)N4C3CCC(=O)NC3=O)CC2)CC1] |
| | N-(6-((5-bromo-2-((4-(4-(4-(2-(1′-(2,6-dioxopiperidin-3-yl)-2′- |
| | oxospiro[cyclopropane-1,3′-indolin]-4′-yl)ethyl)piperazin-1- |
| | yl)piperidin-1-yl)-5-ethyl-2-methoxyphenyl)amino)pyrimidin-4- |
| | yl)amino)quinoxalin-5-yl)methanesulfonamide |
| 17 | 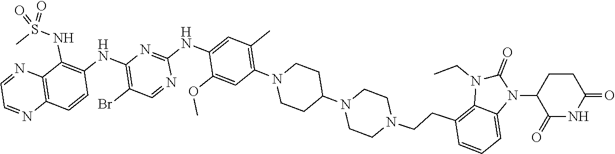 [SMILES: CCn1c(=O)n(C2CCC(=O)NC2=O)c2cccc(CCN3CCN(C4CCN(c5cc(OC)c(Nc6ncc(Br)c(Nc7ccc8nccnc8c7NS(C)(=O)=O)n6)cc5C)CC4)CC3)c21] |
| | N-(6-((5-bromo-2-((4-(4-(4-(2-(1-(2,6-dioxopiperidin-3-yl)-3-ethyl- |
| | 2-oxo-2,3-dihydro-1H-benzo[d]imidazol-4-yl)ethyl)piperazin-1- |
| | yl)piperidin-1-yl)-2-methoxy-5-methylphenyl)amino)pyrimidin-4- |
| | yl)amino)quinoxalin-5-yl)methanesulfonamide |
| 18 | 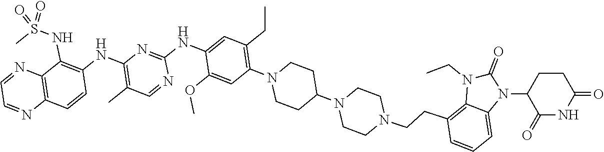 [SMILES: CCc1cc(Nc2ncc(C)c(Nc3ccc4nccnc4c3NS(C)(=O)=O)n2)c(OC)cc1N1CCC(N2CCN(CCc3cccc4c3n(CC)c(=O)n4C3CCC(=O)NC3=O)CC2)CC1] |
| | N-(6-((2-((4-(4-(4-(2-(1-(2,6-dioxopiperidin-3-yl)-3-ethyl-2-oxo-2,3- |
| | dihydro-1H-benzo[d]imidazol-4-yl)ethyl)piperazin-1-yl)piperidin-1- |
| | yl)-5-ethyl-2-methoxyphenyl)amino)-5-methylpyrimidin-4- |
| | yl)amino)quinoxalin-5-yl)methanesulfonamide |
| 19 | 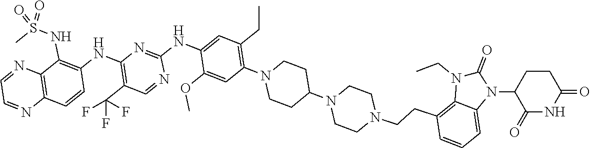 [SMILES: CCc1cc(Nc2ncc(C(F)(F)F)c(Nc3ccc4nccnc4c3NS(C)(=O)=O)n2)c(OC)cc1N1CCC(N2CCN(CCc3cccc4c3n(CC)c(=O)n4C3CCC(=O)NC3=O)CC2)CC1] |
| | N-(6-((2-((4-(4-(4-(2-(1-(2,6-dioxopiperidin-3-yl)-3-ethyl-2-oxo-2,3- |
| | dihydro-1H-benzo[d]imidazol-4-yl)ethyl)piperazin-1-yl)piperidin-1- |
| | yl)-5-ethyl-2-methoxyphenyl)amino)-5-(trifluoromethyl)pyrimidin-4- |
| | yl)amino)quinoxalin-5-yl)methanesulfonamide |
| 20 | 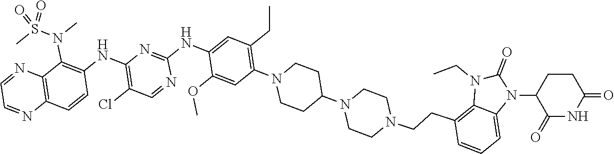 [SMILES: CCc1cc(Nc2ncc(Cl)c(Nc3ccc4nccnc4c3N(C)S(C)(=O)=O)n2)c(OC)cc1N1CCC(N2CCN(CCc3cccc4c3n(CC)c(=O)n4C3CCC(=O)NC3=O)CC2)CC1] |
| | N-(6-((5-chloro-2-((4-(4-(4-(2-(1-(2,6-dioxopiperidin-3-yl)-3-ethyl- |
| | 2-oxo-2,3-dihydro-1H-benzo[d]imidazol-4-yl)ethyl)piperazin-1- |
| | yl)piperidin-1-yl)-5-ethyl-2-methoxyphenyl)amino)pyrimidin-4- |
| | yl)amino)quinoxalin-5-yl)methanesulfonamide |
| 21 | 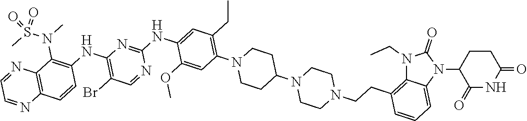 [SMILES: CCc1cc(Nc2ncc(Br)c(Nc3ccc4nccnc4c3N(C)S(C)(=O)=O)n2)c(OC)cc1N1CCC(N2CCN(CCc3cccc4c3n(CC)c(=O)n4C3CCC(=O)NC3=O)CC2)CC1] |
| | N-(6-((5-bromo-2-((4-(4-(4-(2-(1-(2,6-dioxopiperidin-3-yl)-3-ethyl- |
| | 2-oxo-2,3-dihydro-1H-benzo[d]imidazol-4-yl)ethyl)piperazin-1- |
| | yl)piperidin-1-yl)-5-ethyl-2-methoxyphenyl)amino)pyrimidin-4- |
| | yl)amino)quinoxalin-5-yl)-N-methylmethanesulfonamide |
| 22 | 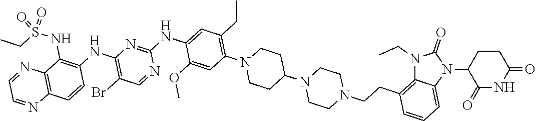 [SMILES: CCc1cc(Nc2ncc(Br)c(Nc3ccc4nccnc4c3NS(=O)(=O)CC)n2)c(OC)cc1N1CCC(N2CCN(CCc3cccc4c3n(CC)c(=O)n4C3CCC(=O)NC3=O)CC2)CC1] |
| | N-(6-((5-bromo-2-((4-(4-(4-(2-(1-(2,6-dioxopiperidin-3-yl)-3-ethyl- |
| | 2-oxo-2,3-dihydro-1H-benzo[d]imidazol-4-yl)ethyl)piperazin-1- |
| | yl)piperidin-1-yl)-5-ethyl-2-methoxyphenyl)amino)pyrimidin-4- |
| | yl)amino)quinoxalin-5-yl)ethanesulfonamide |
| 23 | 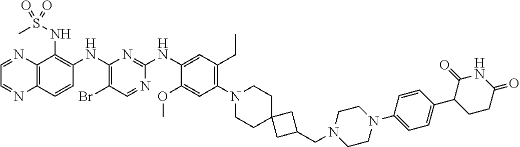 [SMILES: CCc1cc(Nc2ncc(Br)c(Nc3ccc4nccnc4c3NS(C)(=O)=O)n2)c(OC)cc1N1CCC2(CC1)CC(CN1CCN(c3ccc(C4CCC(=O)NC4=O)cc3)CC1)C2] |
| | N-(6-((5-bromo-2-((4-(2-((4-(4-(2,6-dioxopiperidin-3- |
| | yl)phenyl)piperazin-1-yl)methyl)-7-azaspiro[3.5]nonan-7-yl)-5- |
| | ethyl-2-methoxyphenyl)amino)pyrimidin-4-yl)amino)quinoxalin-5- |
| | yl)methanesulfonamide |
| 24 | 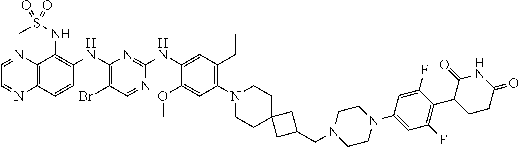 [SMILES: CCc1cc(Nc2ncc(Br)c(Nc3ccc4nccnc4c3NS(C)(=O)=O)n2)c(OC)cc1N1CCC2(CC1)CC(CN1CCN(c3cc(F)c(C4CCC(=O)NC4=O)c(F)c3)CC1)C2] |
| | N-(6-((5-bromo-2-((4-(2-((4-(4-(2,6-dioxopiperidin-3-yl)-3,5- |
| | difluorophenyl)piperazin-1-yl)methyl)-7-azaspiro[3.5]nonan-7-yl)- |
| | 5-ethyl-2-methoxyphenyl)amino)pyrimidin-4-yl)amino)quinoxalin- |
| | 5-yl)methanesulfonamide |
| 25 | 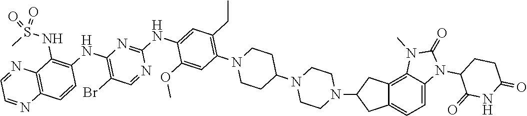 [SMILES: CCc1cc(Nc2ncc(Br)c(Nc3ccc4nccnc4c3NS(C)(=O)=O)n2)c(OC)cc1N1CCC(N2CCN(C3Cc4ccc5c(c4C3)n(C)c(=O)n5C3CCC(=O)NC3=O)CC2)CC1] |
| | N-(6-((5-bromo-2-((4-(4-(4-(3-(2,6-dioxopiperidin-3-yl)-1-methyl-2- |
| | oxo-1,2,3,6,7,8-hexahydroindeno[4,5-d]imidazol-7-yl)piperazin-1- |
| | yl)piperidin-1-yl)-5-ethyl-2-methoxyphenyl)amino)pyrimidin-4- |
| | yl)amino)quinoxalin-5-yl)methanesulfonamide |
| 26 | 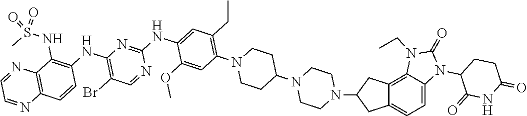 [SMILES: CCc1cc(Nc2ncc(Br)c(Nc3ccc4nccnc4c3NS(C)(=O)=O)n2)c(OC)cc1N1CCC(N2CCN(C3Cc4ccc5c(c4C3)n(CC)c(=O)n5C3CCC(=O)NC3=O)CC2)CC1] |
| | N-(6-((5-bromo-2-((4-(4-(4-(3-(2,6-dioxopiperidin-3-yl)-1-ethyl-2- |
| | oxo-1,2,3,6,7,8-hexahydroindeno[4,5-d]imidazol-7-yl)piperazin-1- |
| | yl)piperidin-1-yl)-5-ethyl-2-methoxyphenyl)amino)pyrimidin-4- |
| | yl)amino)quinoxalin-5-yl)methanesulfonamide |
| 27 | 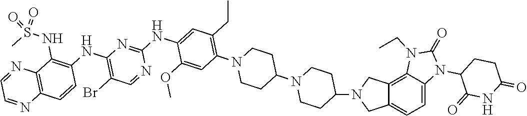 [SMILES: CCc1cc(Nc2ncc(Br)c(Nc3ccc4nccnc4c3NS(C)(=O)=O)n2)c(OC)cc1N1CCC(N2CCC(N3Cc4ccc5c(c4C3)n(CC)c(=O)n5C3CCC(=O)NC3=O)CC2)CC1] |
| | N-(6-(5-bromo-2-((4-(4-(3-(2,6-dioxopiperidin-3-yl)-1-ethyl-2-oxo- |
| | 2,3,6,8-tetrahydroimidazo[4,5-e]isoindol-7(1H)-yl)-[1,4′- |
| | bipiperidin]-1′-yl)-5-ethyl-2-methoxyphenyl)amino)pyrimidin-4- |
| | yl)amino)quinoxalin-5-yl)methanesulfonamide |
| 28 | 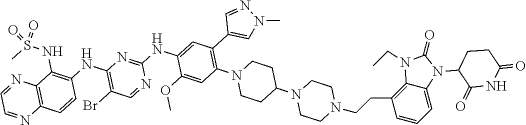 [SMILES: CCn1c(=O)n(C2CCC(=O)NC2=O)c2cccc(CCN3CCN(C4CCN(c5cc(OC)c(Nc6ncc(Br)c(Nc7ccc8nccnc8c7NS(C)(=O)=O)n6)cc5-c5cnn(C)c5)CC4)CC3)c21] |
| | N-(6-((5-bromo-2-((4-(4-(4-(2-(1-(2,6-dioxopiperidin-3-yl)-3-ethyl- |
| | 2-oxo-2,3-dihydro-1H-benzo[d]imidazol-4-yl)ethyl)piperazin-1- |
| | yl)piperidin-1-yl)-2-methoxy-5-(1-methyl-1H-pyrazol-4- |
| | yl)phenyl)amino)pyrimidin-4-yl)amino)quinoxalin-5- |
| | yl)methanesulfonamide |
| 29 | 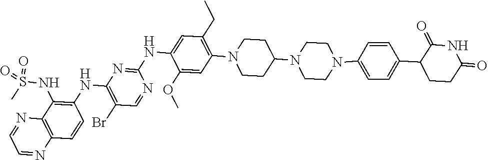 [SMILES: CCc1cc(Nc2ncc(Br)c(Nc3ccc4nccnc4c3NS(C)(=O)=O)n2)c(OC)cc1N1CCC(N2CCN(c3ccc(C4CCC(=O)NC4=O)cc3)CC2)CC1] |
| | N-(6-((5-bromo-2-((4-(4-(4-(4-(2,6-dioxopiperidin-3- |
| | yl)phenyl)piperazin-1-yl)piperidin-1-yl)-5-ethyl-2- |
| | methoxyphenyl)amino)pyrimidin-4-yl)amino)quinoxalin-5- |
| | yl)methanesulfonamide |
| 30 | 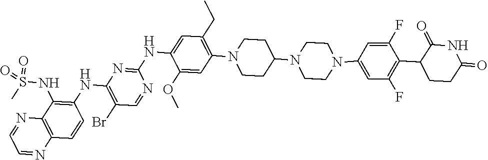 [SMILES: CCc1cc(Nc2ncc(Br)c(Nc3ccc4nccnc4c3NS(C)(=O)=O)n2)c(OC)cc1N1CCC(N2CCN(c3cc(F)c(C4CCC(=O)NC4=O)c(F)c3)CC2)CC1] |
| | N-(6-((5-bromo-2-((4-(4-(4-(4-(2,6-dioxopiperidin-3-yl)-3,5- |
| | difluorophenyl)piperazin-1-yl)piperidin-1-yl)-5-ethyl-2- |
| | methoxyphenyl)amino)pyrimidin-4-yl)amino)quinoxalin-5- |
| | yl)methanesulfonamide |
| 31 | 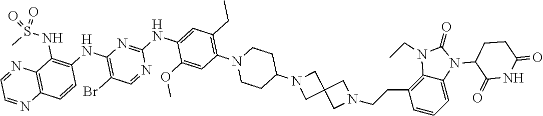 [SMILES: CCc1cc(Nc2ncc(Br)c(Nc3ccc4nccnc4c3NS(C)(=O)=O)n2)c(OC)cc1N1CCC(N2CC3(CN(CCc4cccc5c4n(CC)c(=O)n5C4CCC(=O)NC4=O)C3)C2)CC1] |
| | N-(6-((5-bromo-2-((4-(4-(6-(2-(1-(2,6-dioxopiperidin-3-yl)-3-ethyl- |
| | 2-oxo-2,3-dihydro-1H-benzo[d]imidazol-4-yl)ethyl)-2,6- |
| | diazaspiro[3.3]heptan-2-yl)piperidin-1-yl)-5-ethyl-2- |
| | methoxyphenyl)amino)pyrimidin-4-yl)amino)quinoxalin-5- |
| | yl)methanesulfonamide |
| 32 | 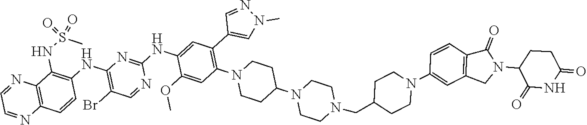 [SMILES: COc1cc(N2CCC(N3CCN(CC4CCN(c5ccc6c(c5)CN(C5CCC(=O)NC5=O)C6=O)CC4)CC3)CC2)c(-c2cnn(C)c2)cc1Nc1ncc(Br)c(Nc2ccc3nccnc3c2NS(C)(=O)=O)n1] |
| | N-(6-((5-bromo-2-((4-(4-(4-((1-(2-(2,6-dioxopiperidin-3-yl)-1- |
| | oxoisoindolin-5-yl)piperidin-4-yl)methyl)piperazin-1-yl)piperidin-1- |
| | yl)-2-methoxy-5-(1-methyl-1H-pyrazol-4- |
| | yl)phenyl)amino)pyrimidin-4-yl)amino)quinoxalin-5- |
| | yl)methanesulfonamide |
| 33 | 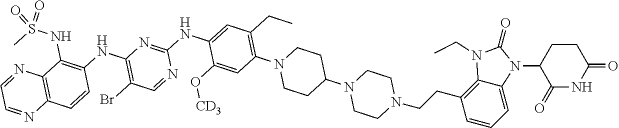 [SMILES: [2H]C([2H])([2H])Oc1cc(N2CCC(N3CCN(CCc4cccc5c4n(CC)c(=O)n5C4CCC(=O)NC4=O)CC3)CC2)c(CC)cc1Nc1ncc(Br)c(Nc2ccc3nccnc3c2NS(C)(=O)=O)n1] |
| | N-(6-((5-bromo-2-((4-(4-(4-(2-(1-(2,6-dioxopiperidin-3-yl)-3-ethyl- |
| | 2-oxo-2,3-dihydro-1H-benzo[d]imidazol-4-yl)ethyl)piperazin-1- |
| | yl)piperidin-1-yl)-5-ethyl-2-(methoxy-d3)phenyl)amino)pyrimidin-4- |
| | yl)amino)quinoxalin-5-yl)methanesulfonamide |
| 34 | 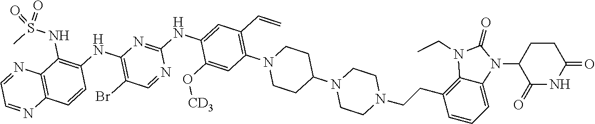 [SMILES: [2H]C([2H])([2H])Oc1cc(N2CCC(N3CCN(CCc4cccc5c4n(CC)c(=O)n5C4CCC(=O)NC4=O)CC3)CC2)c(C=C)cc1Nc1ncc(Br)c(Nc2ccc3nccnc3c2NS(C)(=O)=O)n1] |
| | N-(6-((5-bromo-2-((4-(4-(4-(2-(1-(2,6-dioxopiperidin-3-yl)-3-ethyl- |
| | 2-oxo-2,3-dihydro-1H-benzo[d]imidazol-4-yl)ethyl)piperazin-1- |
| | yl)piperidin-1-yl)-2-(methoxy-d3)-5-vinylphenyl)amino)pyrimidin-4- |
| | yl)amino)quinoxalin-5-yl)methanesulfonamide |
| 35 | 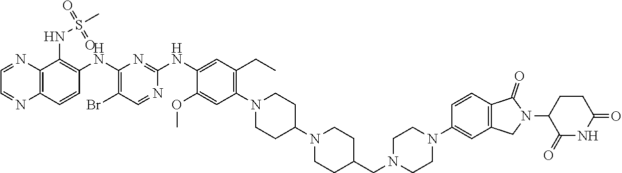 [SMILES: CCc1cc(Nc2ncc(Br)c(Nc3ccc4nccnc4c3NS(C)(=O)=O)n2)c(OC)cc1N1CCC(N2CCC(CN3CCN(c4ccc5c(c4)CN(C4CCC(=O)NC4=O)C5=O)CC3)CC2)CC1] |
| | (S)-N-(6-((5-bromo-2-((4-(4-((4-(2-(2,6-dioxopiperidin-3-yl)-1- |
| | oxoisoindolin-5-yl)piperazin-1-yl)methyl) [1,4′-bipiperidin]-1′-yl)-5- |
| | ethyl-2-methoxyphenyl)amino)pyrimidin-4-yl)amino)quinoxalin-5- |
| | yl)methanesulfonamide |
| 36 | 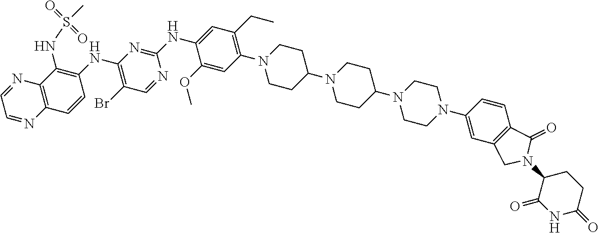 [SMILES: CCc1cc(Nc2ncc(Br)c(Nc3ccc4nccnc4c3NS(C)(=O)=O)n2)c(OC)cc1N1CCC(N2CCC(N3CCN(c4ccc5c(c4)CN([C@H]4CCC(=O)NC4=O)C5=O)CC3)CC2)CC1] |
| | (S)-N-(6-((5-bromo-2-((4-(4-(4-(2-(2,6-dioxopiperidin-3-yl)-1- |
| | oxoisoindolin-5-yl)piperazin-1-yl)-[1,4′-bipiperidin]-1′-yl)-5-ethyl-2- |
| | methoxyphenyl)amino)pyrimidin-4-yl)amino)quinoxalin-5- |
| | yl)methanesulfonamide |
| 37 | 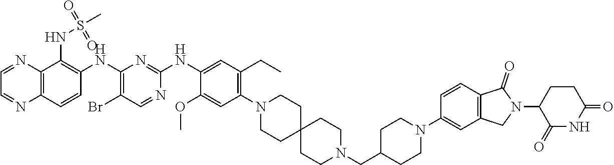 [SMILES: CCc1cc(Nc2ncc(Br)c(Nc3ccc4nccnc4c3NS(C)(=O)=O)n2)c(OC)cc1N1CCC2(CCN(CC3CCN(c4ccc5c(c4)CN(C4CCC(=O)NC4=O)C5=O)CC3)CC2)CC1] |
| | N-(6-((5-bromo-2-((4-(9-((1-(2-(2,6-dioxopiperidin-3-yl)-1- |
| | oxoisoindolin-5-yl)piperidin-4-yl)methyl)-3,9- |
| | diazaspiro[5.5]undecan-3-yl)-5-ethyl-2- |
| | methoxyphenyl)amino)pyrimidin-4-yl)amino)quinoxalin-5- |
| | yl)methanesulfonamide |
| 38 | 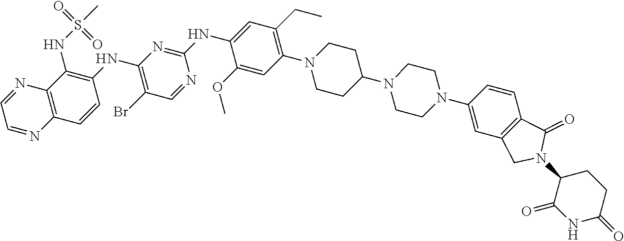 [SMILES: CCc1cc(Nc2ncc(Br)c(Nc3ccc4nccnc4c3NS(C)(=O)=O)n2)c(OC)cc1N1CCC(N2CCN(c3ccc4c(c3)CN([C@H]3CCC(=O)NC3=O)C4=O)CC2)CC1] |
| | (S)-N-(6-((5-bromo-2-((4-(4-(4-(2-(2,6-dioxopiperidin-3-yl)-1- |
| | oxoisoindolin-5-yl)piperazin-1-yl)piperidin-1-yl)-5-ethyl-2- |
| | methoxyphenyl)amino)pyrimidin-4-yl)amino)quinoxalin-5~ |
| | yl)methanesulfonamide |
| 39 | 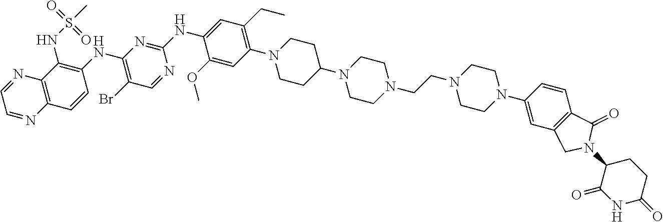 [SMILES: CCc1cc(Nc2ncc(Br)c(Nc3ccc4nccnc4c3NS(C)(=O)=O)n2)c(OC)cc1N1CCC(N2CCN(CCN3CCN(c4ccc5c(c4)CN([C@H]4CCC(=O)NC4=O)C5=O)CC3)CC2)CC1] |
| | (S)-N-(6-((5-bromo-2-((4-(4-(4-(2-(4-(2-(2,6-dioxopiperidin-3-yl)-1- |
| | oxoisoindolin-5-yl)piperazin-1-yl)ethyl)piperazin-1-yl)piperidin-1- |
| | yl)-5-ethyl-2-methoxyphenyl)amino)pyrimidin-4- |
| | yl)amino)quinoxalin-5-yl)methanesulfonamide |
| 40 | 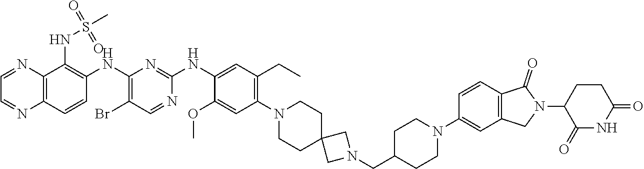 [SMILES: CCc1cc(Nc2ncc(Br)c(Nc3ccc4nccnc4c3NS(C)(=O)=O)n2)c(OC)cc1N1CCC2(CC1)CN(CC1CCN(c3ccc4c(c3)CN(C3CCC(=O)NC3=O)C4=O)CC1)C2] |
| | N-(6-((5-bromo-2-((4-(2-((1-(2-(2,6-dioxopiperidin-3-yl)-1- |
| | oxoisoindolin-5-yl)piperidin-4-yl)methyl)-2,7-diazaspiro[3.5]nonan- |
| | 7-yl)-5-ethyl-2-methoxyphenyl)amino)pyrimidin-4- |
| | yl)amino)quinoxalin-5-yl)methanesulfonamide |
| 41 | 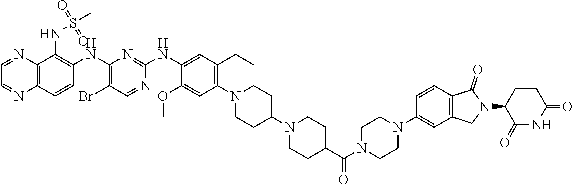 [SMILES: CCc1cc(Nc2ncc(Br)c(Nc3ccc4nccnc4c3NS(C)(=O)=O)n2)c(OC)cc1N1CCC(N2CCC(C(=O)N3CCN(c4ccc5c(c4)CN([C@H]4CCC(=O)NC4=O)C5=O)CC3)CC2)CC1] |
| | (S)-N-(6-((5-bromo-2-((4-(4-(4-(2-(2,6-dioxopiperidin-3-yl)-1- |
| | oxoisoindolin-5-yl)piperazine-1-carbonyl)-[1,4′-bipiperidin]-1′-yl)-5- |
| | ethyl-2-methoxyphenyl)amino)pyrimidin-4-yl)amino)quinoxalin-5- |
| | yl)methanesulfonamide |
| 42 | 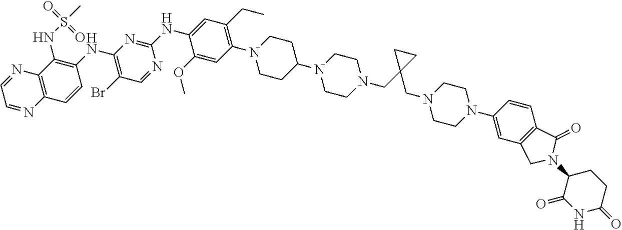 [SMILES: CCc1cc(Nc2ncc(Br)c(Nc3ccc4nccnc4c3NS(C)(=O)=O)n2)c(OC)cc1N1CCC(N2CCN(CC3(CN4CCN(c5ccc6c(c5)CN([C@H]5CCC(=O)NC5=O)C6=O)CC4)CC3)CC2)CC1] |
| | (S)-N-(6-((5-bromo-2-((4-(4-(4-((1-((4-(2-(2,6-dioxopiperidin-3-yl)- |
| | 1-oxoisoindolin-5-yl)piperazin-1- |
| | yl)methyl)cyclopropyl)methyl)piperazin-1-yl)piperidin-1-yl)-5-ethyl- |
| | 2-methoxyphenyl)amino)pyrimidin-4-yl)amino)quinoxalin-5- |
| | yl)methanesulfonamide |
| 43 | 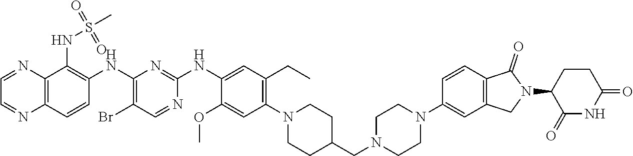 [SMILES: CCc1cc(Nc2ncc(Br)c(Nc3ccc4nccnc4c3NS(C)(=O)=O)n2)c(OC)cc1N1CCC(CN2CCN(c3ccc4c(c3)CN([C@H]3CCC(=O)NC3=O)C4=O)CC2)CC1] |
| | (S)-N-(6-((5-bromo-2-((4-(4-((4-(2-(2,6-dioxopiperidin-3-yl)-1- |
| | oxoisoindolin-5-yl)piperazin-1-yl)methyl)piperidin-1-yl)-5-ethyl-2- |
| | methoxyphenyl)amino)pyrimidin-4-yl)amino)quinoxalin-5- |
| | yl)methanesulfonamide |
| 44 | 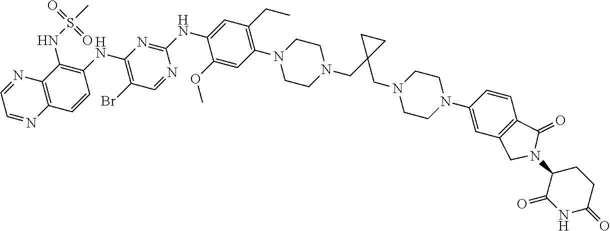 [SMILES: CCc1cc(Nc2ncc(Br)c(Nc3ccc4nccnc4c3NS(C)(=O)=O)n2)c(OC)cc1N1CCN(CC2(CN3CCN(c4ccc5c(c4)CN([C@H]4CCC(=O)NC4=O)C5=O)CC3)CC2)CC1] |
| | (S)-N-(6-((5-bromo-2-((4-(4-((1-((4-(2-(2,6-dioxopiperidin-3-yl)-1- |
| | oxoisoindolin-5-yl)piperazin-1- |
| | yl)methyl)cyclopropyl)methyl)piperazin-1-yl)-5-ethyl-2- |
| | methoxyphenyl)amino)pyrimidin-4-yl)amino)quinoxalin-5- |
| | yl)methanesulfonamide |
| 45 | 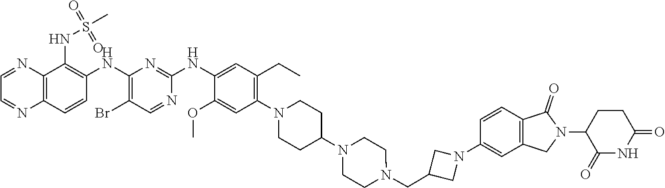 [SMILES: CCc1cc(Nc2ncc(Br)c(Nc3ccc4nccnc4c3NS(C)(=O)=O)n2)c(OC)cc1N1CCC(N2CCN(CC3CN(c4ccc5c(c4)CN(C4CCC(=O)NC4=O)C5=O)C3)CC2)CC1] |
| | N-(6-((5-bromo-2-((4-(4-(4-((1-(2-(2,6-dioxopiperidin-3-yl)-1- |
| | oxoisoindolin-5-yl)azetidin-3-yl)methyl)piperazin-1-yl)piperidin-1- |
| | yl)-5-ethyl-2-methoxyphenyl)amino)pyrimidin-4- |
| | yl)amino)quinoxalin-5-yl)methanesulfonamide |
| 46 | 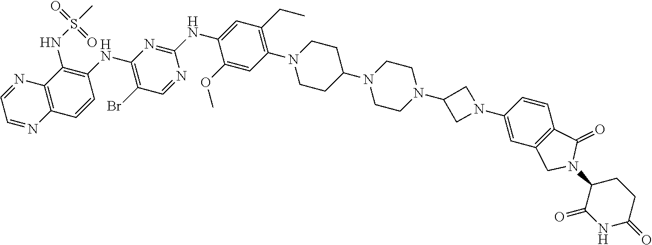 [SMILES: CCc1cc(Nc2ncc(Br)c(Nc3ccc4nccnc4c3NS(C)(=O)=O)n2)c(OC)cc1N1CCC(N2CCN(C3CN(c4ccc5c(c4)CN([C@H]4CCC(=O)NC4=O)C5=O)C3)CC2)CC1] |
| | N-(6-((5-bromo-2-((4-(4-(4-(1-(2-(2,6-dioxopiperidin-3-yl)-1- |
| | oxoisoindolin-5-yl)azetidin-3-yl)piperazin-1-yl)piperidin-1-yl)-5- |
| | ethyl-2-methoxyphenyl)amino)pyrimidin-4-yl)amino)quinoxalin-5- |
| | yl)methanesulfonamide |
| 47 | 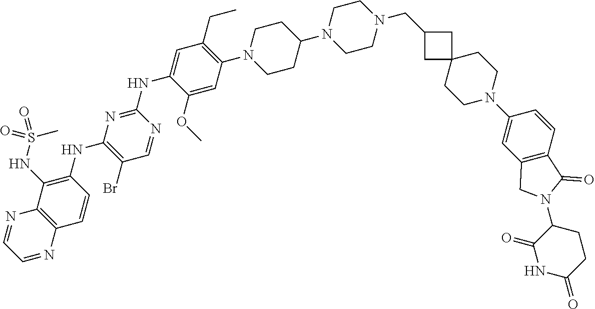 [SMILES: CCc1cc(Nc2ncc(Br)c(Nc3ccc4nccnc4c3NS(C)(=O)=O)n2)c(OC)cc1N1CCC(N2CCN(CC3CC4(CCN(c5ccc6c(c5)CN(C5CCC(=O)NC5=O)C6=O)CC4)C3)CC2)CC1] |
| | N-(6-((5-bromo-2-((4-(4-(4-((7-(2-(2,6-dioxopiperidin-3-yl)-1- |
| | oxoisoindolin-5-yl)-7-azaspiro[3.5]nonan-2-yl)methyl)piperazin-1- |
| | yl)piperidin-1-yl)-5-ethyl-2-methoxyphenyl)amino)pyrimidin-4- |
| | yl)amino)quinoxalin-5-yl)methanesulfonamide |
| 48 | 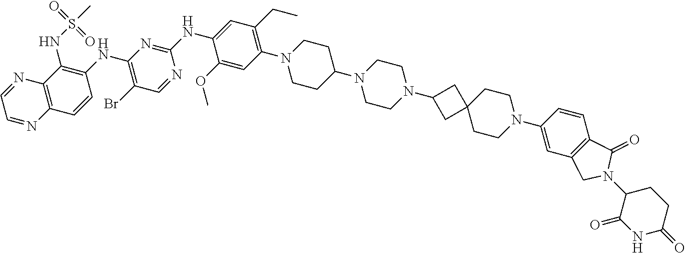 [SMILES: CCc1cc(Nc2ncc(Br)c(Nc3ccc4nccnc4c3NS(C)(=O)=O)n2)c(OC)cc1N1CCC(N2CCN(C3CC4(CCN(c5ccc6c(c5)CN(C5CCC(=O)NC5=O)C6=O)CC4)C3)CC2)CC1] |
| | N-(6-((5-bromo-2-((4-(4-(4-(7-(2-(2,6-dioxopiperidin-3-yl)-1- |
| | oxoisoindolin-5-yl)-7-azaspiro[3.5]nonan-2-yl)piperazin-1- |
| | yl)piperidin-1-yl)-5-ethyl-2-methoxyphenyl)amino)pyrimidin-4- |
| | yl)amino)quinoxalin-5-yl)methanesulfonamide |
| 49 | 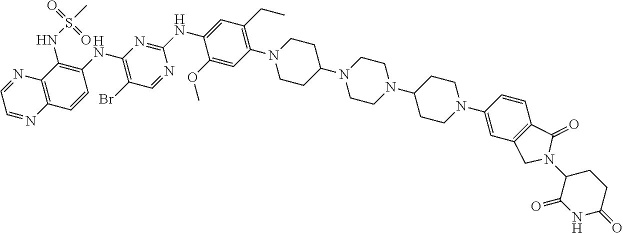 [SMILES: CCc1cc(Nc2ncc(Br)c(Nc3ccc4nccnc4c3NS(C)(=O)=O)n2)c(OC)cc1N1CCC(N2CCN(C3CCN(c4ccc5c(c4)CN(C4CCC(=O)NC4=O)C5=O)CC3)CC2)CC1] |
| | N-(6-((5-bromo-2-((4-(4-(4-(1-(2-(2,6-dioxopiperidin-3-yl)-1- |
| | oxoisoindolin-5-yl)piperidin-4-yl)piperazin-1-yl)piperidin-1-yl)-5- |
| | ethyl-2-methoxyphenyl)amino)pyrimidin-4-yl)amino)quinoxalin-5- |
| | yl)methanesulfonamide |
| 50 | 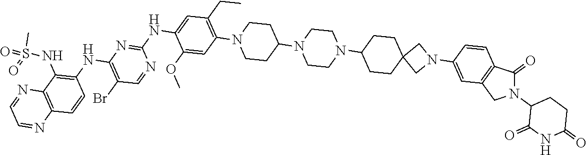 [SMILES: CCc1cc(Nc2ncc(Br)c(Nc3ccc4nccnc4c3NS(C)(=O)=O)n2)c(OC)cc1N1CCC(N2CCN(C3CCC4(CC3)CN(c3ccc5c(c3)CN(C3CCC(=O)NC3=O)C5=O)C4)CC2)CC1] |
| | N-(6-((5-bromo-2-((4-(4-(4-(2-(2-(2,6-dioxopiperidin-3-yl)-1- |
| | oxoisoindolin-5-yl)-2-azaspiro[3.5]nonan-7-yl)piperazin-1- |
| | yl)piperidin-1-yl)-5-ethyl-2-methoxyphenyl)amino)pyrimidin-4- |
| | yl)amino)quinoxalin-5-yl)methanesulfonamide |
| 51 | 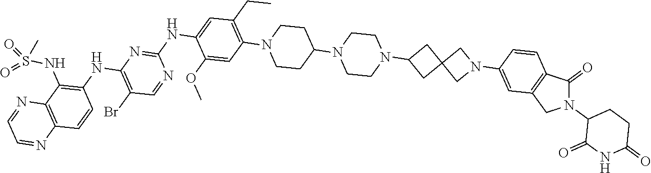 [SMILES: CCc1cc(Nc2ncc(Br)c(Nc3ccc4nccnc4c3NS(C)(=O)=O)n2)c(OC)cc1N1CCC(N2CCN(C3CC4(C3)CN(c3ccc5c(c3)CN(C3CCC(=O)NC3=O)C5=O)C4)CC2)CC1] |
| | N-(6-((5-bromo-2-((4-(4-(4-(2-(2-(2,6-dioxopiperidin-3-yl)-1- |
| | oxoisoindolin-5-yl)-2-azaspiro[3.3]heptan-6-yl)piperazin-1- |
| | yl)piperidin-1-yl)-5-ethyl-2-methoxyphenyl)amino)pyrimidin-4- |
| | yl)amino)quinoxalin-5-yl)methanesulfonamide |
| 52 | 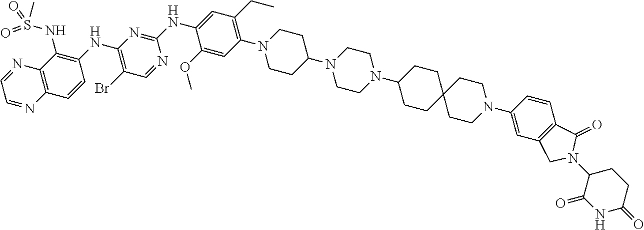 [SMILES: CCc1cc(Nc2ncc(Br)c(Nc3ccc4nccnc4c3NS(C)(=O)=O)n2)c(OC)cc1N1CCC(N2CCN(C3CCC4(CC3)CCN(c3ccc5c(c3)CN(C3CCC(=O)NC3=O)C5=O)CC4)CC2)CC1] |
| | N-(6-((5-bromo-2-((4-(4-(4-(3-(2-(2,6-dioxopiperidin-3-yl)-1- |
| | oxoisoindolin-5-yl)-3-azaspiro[5.5]undecan-9-yl)piperazin-1- |
| | yl)piperidin-1-yl)-5-ethyl-2-methoxyphenyl)amino)pyrimidin-4- |
| | yl)amino)quinoxalin-5-yl)methanesulfonamide |
| 53 | 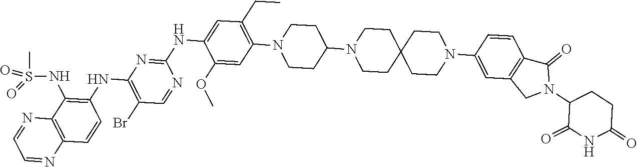 [SMILES: CCc1cc(Nc2ncc(Br)c(Nc3ccc4nccnc4c3NS(C)(=O)=O)n2)c(OC)cc1N1CCC(N2CCC3(CCN(c4ccc5c(c4)CN(C4CCC(=O)NC4=O)C5=O)CC3)CC2)CC1] |
| | N-(6-((5-bromo-2-((4-(4-(9-(2-(2,6-dioxopiperidin-3-yl)-1- |
| | oxoisoindolin-5-yl)-3,9-diazaspiro[5.5]undecan-3-yl)piperidin-1- |
| | yl)-5-ethyl-2-methoxyphenyl)amino)pyrimidin-4- |
| | yl)amino)quinoxalin-5-yl)methanesulfonamide |
| 54 | 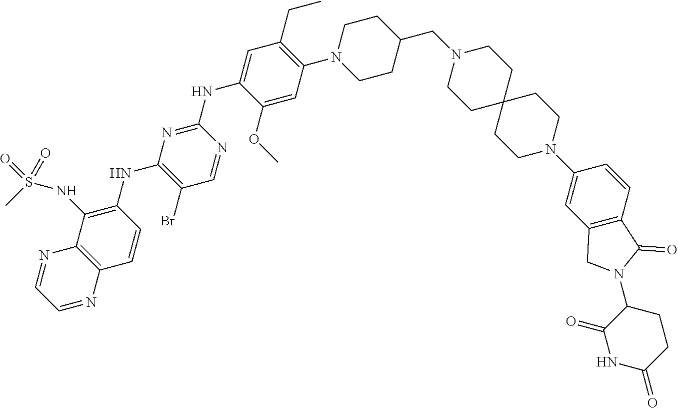 [SMILES: CCc1cc(Nc2ncc(Br)c(Nc3ccc4nccnc4c3NS(C)(=O)=O)n2)c(OC)cc1N1CCC(CN2CCC3(CC2)CCN(c2ccc4c(c2)CN(C2CCC(=O)NC2=O)C4=O)CC3)CC1] |
| | N-(6-((5-bromo-2-((4-(4-((9-(2-(2,6-dioxopiperidin-3-yl)-1- |
| | oxoisoindolin-5-yl)-3,9-diazaspiro[5.5]undecan-3- |
| | yl)methyl)piperidin-1-yl)-5-ethyl-2- |
| | methoxyphenyl)amino)pyrimidin-4-yl)amino)quinoxalin-5- |
| | yl)methanesulfonamide |
| 55 | 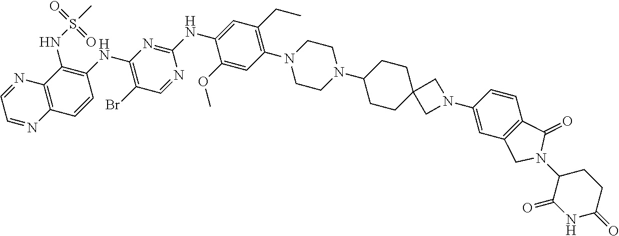 [SMILES: CCc1cc(Nc2ncc(Br)c(Nc3ccc4nccnc4c3NS(C)(=O)=O)n2)c(OC)cc1N1CCN(C2CCC3(CC2)CN(c2ccc4c(c2)CN(C2CCC(=O)NC2=O)C4=O)C3)CC1] |
| | N-(6-((5-bromo-2-((4-(4-(2-(2-(2,6-dioxopiperidin-3-yl)-1- |
| | oxoisoindolin-5-yl)-2,7-diazaspiro[3.5]nonan-7-yl)piperidin-1-yl)-5- |
| | ethyl-2-methoxyphenyl)amino)pyrimidin-4-yl)amino)quinoxalin-5- |
| | yl)methanesulfonamide |
| 56 | 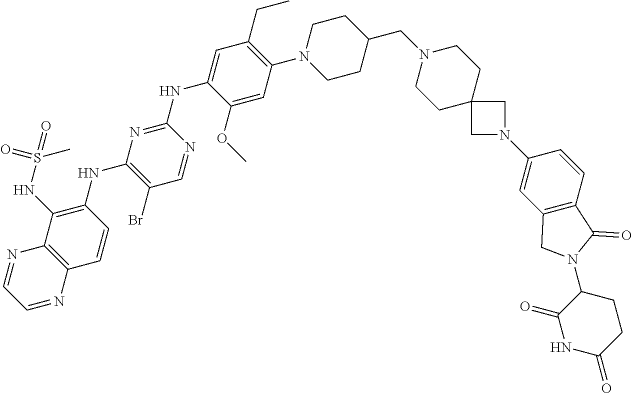 [SMILES: CCc1cc(Nc2ncc(Br)c(Nc3ccc4nccnc4c3NS(C)(=O)=O)n2)c(OC)cc1N1CCC(CN2CCC3(CC2)CN(c2ccc4c(c2)CN(C2CCC(=O)NC2=O)C4=O)C3)CC1] |
| | N-(6-((5-bromo-2-((4-(4-((2-(2-(2,6-dioxopiperidin-3-yl)-1~ |
| | oxoisoindolin-5-yl)-2,7-diazaspiro[3.5]nonan-7-yl)methyl)piperidin- |
| | 1-yl)-5-ethyl-2-methoxyphenyl)amino)pyrimidin-4- |
| | yl)amino)quinoxalin-5-yl)methanesulfonamide |
| 57 | 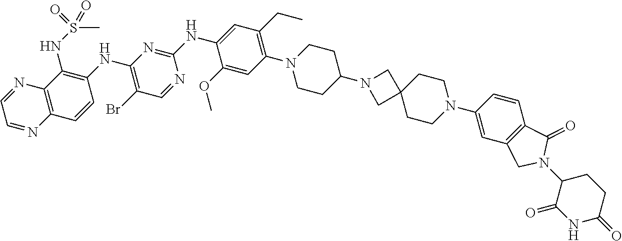 [SMILES: CCc1cc(Nc2ncc(Br)c(Nc3ccc4nccnc4c3NS(C)(=O)=O)n2)c(OC)cc1N1CCC(N2CC3(CCN(c4ccc5c(c4)CN(C4CCC(=O)NC4=O)C5=O)CC3)C2)CC1] |
| | N-(6-((5-bromo-2-((4-(4-(7-(2-(2,6-dioxopiperidin-3-yl)-1~ |
| | oxoisoindolin-5-yl)-2,7-diazaspiro[3.5]nonan-2-yl)piperidin-1-yl)-5- |
| | ethyl-2-methoxyphenyl)amino)pyrimidin-4-yl)amino)quinoxalin-5- |
| | yl)methanesulfonamide |
| 58 | 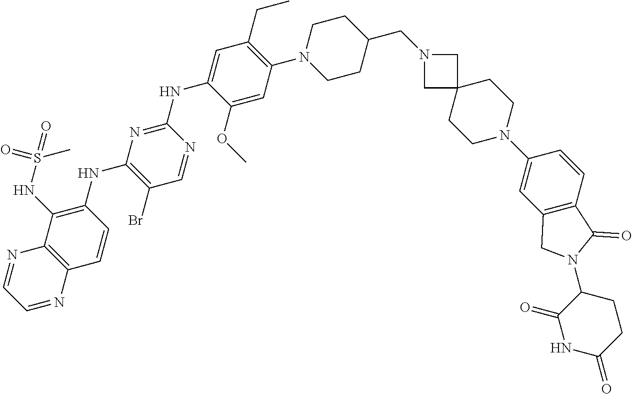 [SMILES: CCc1cc(Nc2ncc(Br)c(Nc3ccc4nccnc4c3NS(C)(=O)=O)n2)c(OC)cc1N1CCC(CN2CC3(CCN(c4ccc5c(c4)CN(C4CCC(=O)NC4=O)C5=O)CC3)C2)CC1] |
| | N-(6-((5-bromo-2-((4-(4-((7-(2-(2,6-dioxopiperidin-3-yl)-1- |
| | oxoisoindolin-5-yl)-2,7-diazaspiro[3.5]nonan-2-yl)methyl)piperidin- |
| | 1-yl)-5-ethyl-2-methoxyphenyl)amino)pyrimidin-4- |
| | yl)amino)quinoxalin-5-yl)methanesulfonamide |
| 59 | 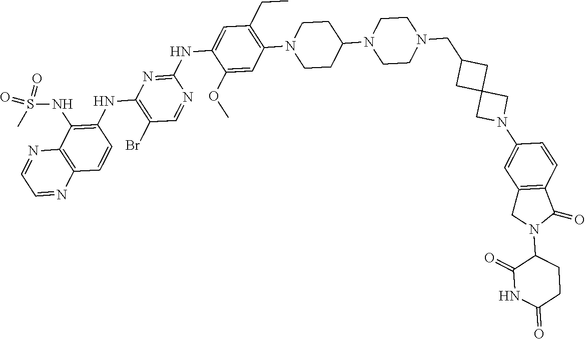 [SMILES: CCc1cc(Nc2ncc(Br)c(Nc3ccc4nccnc4c3NS(C)(=O)=O)n2)c(OC)cc1N1CCC(N2CCN(CC3CC4(C3)CN(c3ccc5c(c3)CN(C3CCC(=O)NC3=O)C5=O)C4)CC2)CC1] |
| | N-(6-((5-bromo-2-((4-(4-(4-((2-(2-(2,6-dioxopiperidin-3-yl)-1- |
| | oxoisoindolin-5-yl)-2-azaspiro[3.3]heptan-6-yl)methyl)piperazin-1- |
| | yl)piperidin-1-yl)-5-ethyl-2-methoxyphenyl)amino)pyrimidin-4- |
| | yl)amino)quinoxalin-5-yl)methanesulfonamide |
| 60 | 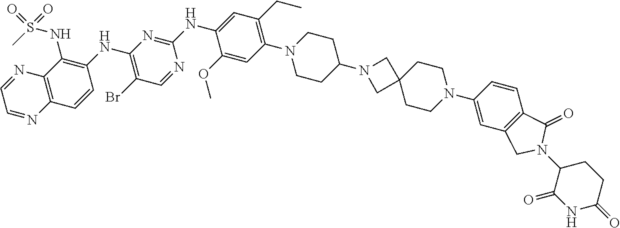 [SMILES: CCc1cc(Nc2ncc(Br)c(Nc3ccc4nccnc4c3NS(C)(=O)=O)n2)c(OC)cc1N1CCC(N2CC3(CCN(c4ccc5c(c4)CN(C4CCC(=O)NC4=O)C5=O)CC3)C2)CC1] |
| | N-(6-((5-bromo-2-((4-(4-(7-(2-(2,6-dioxopiperidin-3-yl)-1- |
| | oxoisoindolin-5-yl)-2,7-diazaspiro[3.5]nonan-2-yl)piperidin-1-yl)-5- |
| | ethyl-2-methoxyphenyl)amino)pyrimidin-4-yl)amino)quinoxalin-5- |
| | yl)methanesulfonamide |
| 61 | 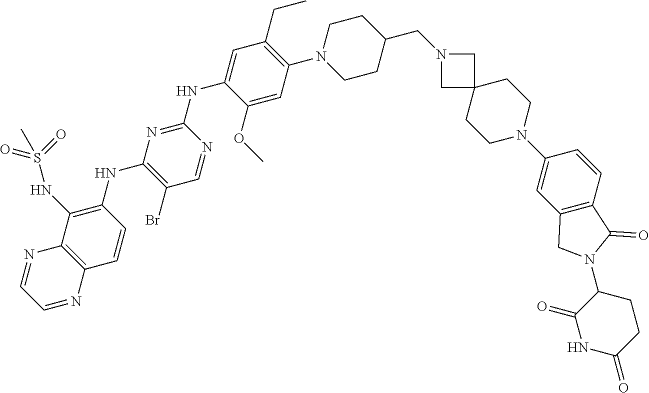 [SMILES: CCc1cc(Nc2ncc(Br)c(Nc3ccc4nccnc4c3NS(C)(=O)=O)n2)c(OC)cc1N1CCC(CN2CC3(CCN(c4ccc5c(c4)CN(C4CCC(=O)NC4=O)C5=O)CC3)C2)CC1] |
| | N-(6-((5-bromo-2-((4-(4-((7-(2-(2,6-dioxopiperidin-3-yl)-1- |
| | oxoisoindolin-5-yl)-2,7-diazaspiro[3.5]nonan-2-yl)methyl)piperidin- |
| | 1-yl)-5-ethyl-2-methoxyphenyl)amino)pyrimidin-4- |
| | yl)amino)quinoxalin-5-yl)methanesulfonamide |
| 62 | 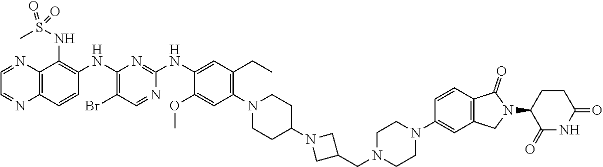 [SMILES: CCc1cc(Nc2ncc(Br)c(Nc3ccc4nccnc4c3NS(C)(=O)=O)n2)c(OC)cc1N1CCC(N2CC(CN3CCN(c4ccc5c(c4)CN([C@H]4CCC(=O)NC4=O)C5=O)CC3)C2)CC1] |
| | (S)-N-(6-((5-bromo-2-((4-(4-(3-((4-(2-(2,6-dioxopiperidin-3-yl)-1- |
| | oxoisoindolin-5-yl)piperazin-1-yl)methyl)azetidin-1-yl)piperidin-1- |
| | yl)-5-ethyl-2-methoxyphenyl)amino)pyrimidin-4- |
| | yl)amino)quinoxalin-5-yl)methanesulfonamide |
| 63 | 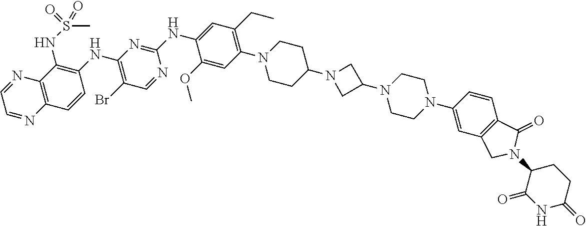 [SMILES: CCc1cc(Nc2ncc(Br)c(Nc3ccc4nccnc4c3NS(C)(=O)=O)n2)c(OC)cc1N1CCC(N2CC(N3CCN(c4ccc5c(c4)CN([C@H]4CCC(=O)NC4=O)C5=O)CC3)C2)CC1] |
| | (S)-N-(6-((5-bromo-2-((4-(4-(3-(4-(2-(2,6-dioxopiperidin-3-yl)-1- |
| | oxoisoindolin-5-yl)piperazin-1-yl)azetidin-1-yl)piperidin-1-yl)-5- |
| | ethyl-2-methoxyphenyl)amino)pyrimidin-4-yl)amino)quinoxalin-5- |
| | yl)methanesulfonamide |
| 64 | 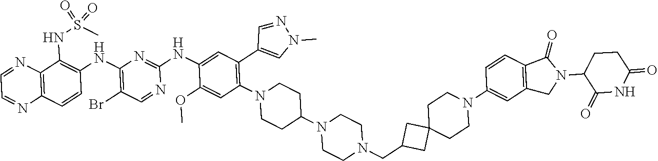 [SMILES: COc1cc(N2CCC(N3CCN(CC4CC5(CCN(c6ccc7c(c6)CN(C6CCC(=O)NC6=O)C7=O)CC5)C4)CC3)CC2)c(-c2cnn(C)c2)cc1Nc1ncc(Br)c(Nc2ccc3nccnc3c2NS(C)(=O)=O)n1] |
| | N-(6-((5-bromo-2-((4-(4-(4-((7-(2-(2,6-dioxopiperidin-3-yl)-1- |
| | oxoisoindolin-5-yl)-7-azaspiro[3.5]nonan-2-yl)methyl)piperazin-1- |
| | yl)piperidin-1-yl)-2-methoxy-5-(1-methyl-1H-pyrazol-4- |
| | yl)phenyl)amino)pyrimidin-4-yl)amino)quinoxalin-5- |
| | yl)methanesulfonamide |
| 65 | 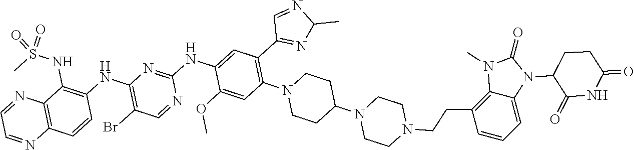 [SMILES: COc1cc(N2CCC(N3CCN(CCc4cccc5c4n(C)c(=O)n5C4CCC(=O)NC4=O)CC3)CC2)c(C2=NC(C)N=C2)cc1Nc1ncc(Br)c(Nc2ccc3nccnc3c2NS(C)(=O)=O)n1] |
| | N-(6-((5-bromo-2-((4-(4-(4-(2-(1-(2,6-dioxopiperidin-3-yl)-3- |
| | methyl-2-oxo-2,3-dihydro-1H-benzo[d]imidazol-4- |
| | yl)ethyl)piperazin-1-yl)piperidin-1-yl)-2-methoxy-5-(1-methyl-1H- |
| | pyrazol-4-yl)phenyl)amino)pyrimidin-4-yl)amino)quinoxalin-5- |
| | yl)methanesulfonamide |
| 66 | 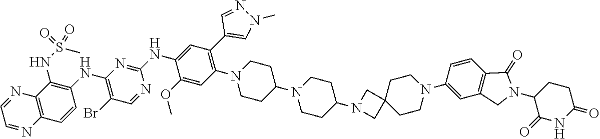 [SMILES: COc1cc(N2CCC(N3CCC(N4CC5(CCN(c6ccc7c(c6)CN(C6CCC(=O)NC6=O)C7=O)CC5)C4)CC3)CC2)c(-c2cnn(C)c2)cc1Nc1ncc(Br)c(Nc2ccc3nccnc3c2NS(C)(=O)=O)n1] |
| | N-(6-((5-bromo-2-((4-(4-(7-(2-(2,6-dioxopiperidin-3-yl)-1~ |
| | oxoisoindolin-5-yl)-2,7-diazaspiro[3.5]nonan-2-yl)-[1,4′- |
| | bipiperidin]-1′-yl)-2-methoxy-5-(1-methyl-1H-pyrazol-4- |
| | yl)phenyl)amino)pyrimidin-4-yl)amino)quinoxalin-5- |
| | yl)methanesulfonamide |
| 67 | 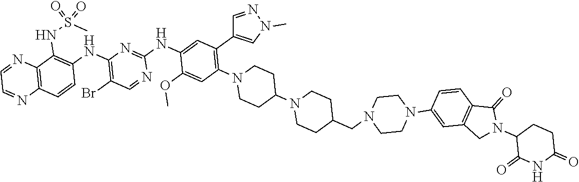 [SMILES: COc1cc(N2CCC(N3CCC(CN4CCN(c5ccc6c(c5)CN(C5CCC(=O)NC5=O)C6=O)CC4)CC3)CC2)c(-c2cnn(C)c2)cc1Nc1ncc(Br)c(Nc2ccc3nccnc3c2NS(C)(=O)=O)n1] |
| | N-(6-((5-bromo-2-((4-(4-((4-(2-(2,6-dioxopiperidin-3-yl)-1~ |
| | oxoisoindolin-5-yl)piperazin-1-yl)methyl)-[1,4′-bipiperidin]-1′-yl)-2- |
| | methoxy-5-(1-methyl-1H-pyrazol-4-yl)phenyl)amino)pyrimidin-4- |
| | yl)amino)quinoxalin-5-yl)methanesulfonamide |
| 68 | 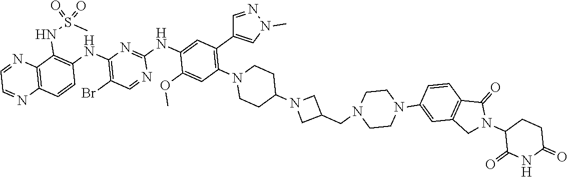 [SMILES: COc1cc(N2CCC(N3CC(CN4CCN(c5ccc6c(c5)CN(C5CCC(=O)NC5=O)C6=O)CC4)C3)CC2)c(-c2cnn(C)c2)cc1Nc1ncc(Br)c(Nc2ccc3nccnc3c2NS(C)(=O)=O)n1] |
| | N-(6-((5-bromo-2-((4-(4-(3-((4-(2-(2,6-dioxopiperidin-3-yl)-1- |
| | oxoisoindolin-5-yl)piperazin-1-yl)methyl)azetidin-1-yl)piperidin-1- |
| | yl)-2-methoxy-5-(1-methyl-1H-pyrazol-4- |
| | yl)phenyl)amino)pyrimidin-4-yl)amino)quinoxalin-5- |
| | yl)methanesulfonamide |
| 69 | 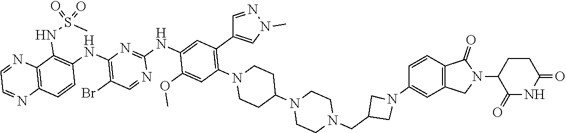 [SMILES: COc1cc(N2CCC(N3CCN(CC4CN(c5ccc6c(c5)CN(C5CCC(=O)NC5=O)C6=O)C4)CC3)CC2)c(-c2cnn(C)c2)cc1Nc1ncc(Br)c(Nc2ccc3nccnc3c2NS(C)(=O)=O)n1] |
| | N-(6-((5-bromo-2-((4-(4-(4-((1-(2-(2,6-dioxopiperidin-3-yl)-1- |
| | oxoisoindolin-5-yl)azetidin-3-yl)methyl)piperazin-1-yl)piperidin-1- |
| | yl)-2-methoxy-5-(1-methyl-1H-pyrazol-4- |
| | yl)phenyl)amino)pyrimidin-4-yl)amino)quinoxalin-5- |
| | yl)methanesulfonamide |
| 70 | 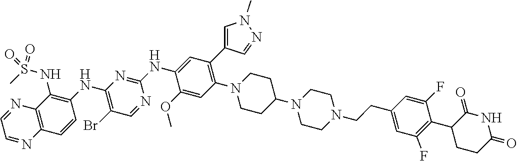 [SMILES: COc1cc(N2CCC(N3CCN(CCc4cc(F)c(C5CCC(=O)NC5=O)c(F)c4)CC3)CC2)c(-c2cnn(C)c2)cc1Nc1ncc(Br)c(Nc2ccc3nccnc3c2NS(C)(=O)=O)n1] |
| | N-(6-((5-bromo-2-((4-(4-(4-(4-(2,6-dioxopiperidin-3-yl)-3,5- |
| | difluorophenethyl)piperazin-1-yl)piperidin-1-yl)-2-methoxy-5-(1- |
| | methyl-1H-pyrazol-4-yl)phenyl)amino)pyrimidin-4- |
| | yl)amino)quinoxalin-5-yl)methanesulfonamide |
| 71 | 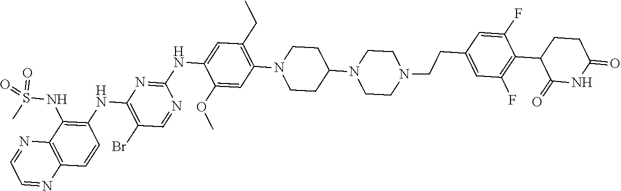 [SMILES: CCc1cc(Nc2ncc(Br)c(Nc3ccc4nccnc4c3NS(C)(=O)=O)n2)c(OC)cc1N1CCC(N2CCN(CCc3cc(F)c(C4CCC(=O)NC4=O)c(F)c3)CC2)CC1] |
| | N-(6-((5-bromo-2-((4-(4-(4-(4-(2,6-dioxopiperidin-3-yl)-3,5~ |
| | difluorophenethyl)piperazin-1-yl)piperidin-1-yl)-5-ethyl-2- |
| | methoxyphenyl)amino)pyrimidin-4-yl)amino)quinoxalin-5- |
| | yl)methanesulfonamide |
| 72 | 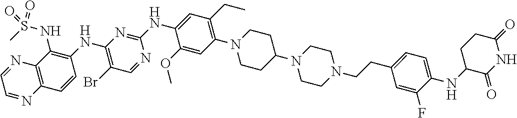 [SMILES: CCc1cc(Nc2ncc(Br)c(Nc3ccc4nccnc4c3NS(C)(=O)=O)n2)c(OC)cc1N1CCC(N2CCN(CCc3ccc(NC4CCC(=O)NC4=O)c(F)c3)CC2)CC1] |
| | N-(6-((5-bromo-2-((4-(4-(4-(4-((2,6-dioxopiperidin-3-yl)amino)-3- |
| | fluorophenethyl)piperazin-1-yl)piperidin-1-yl)-5-ethyl-2- |
| | methoxyphenyl)amino)pyrimidin-4-yl)amino)quinoxalin-5- |
| | yl)methanesulfonamide |
| 73 | 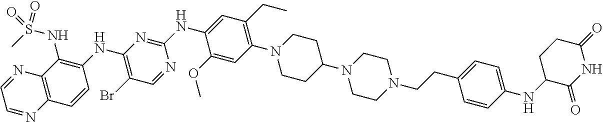 [SMILES: CCc1cc(Nc2ncc(Br)c(Nc3ccc4nccnc4c3NS(C)(=O)=O)n2)c(OC)cc1N1CCC(N2CCN(CCc3ccc(NC4CCC(=O)NC4=O)cc3)CC2)CC1] |
| | N-(6-((5-bromo-2-((4-(4-(4-(4-((2,6-dioxopiperidin-3- |
| | yl)amino)phenethyl)piperazin-1-yl)piperidin-1-yl)-5-ethyl-2- |
| | methoxyphenyl)amino)pyrimidin-4-yl)amino)quinoxalin-5- |
| | yl)methanesulfonamide |
| 74 | 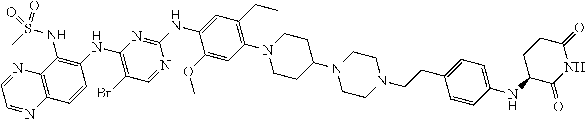 [SMILES: CCc1cc(Nc2ncc(Br)c(Nc3ccc4nccnc4c3NS(C)(=O)=O)n2)c(OC)cc1N1CCC(N2CCN(CCc3ccc(N[C@H]4CCC(=O)NC4=O)cc3)CC2)CC1] |
| | (S)-N-(6-((5-bromo-2-((4-(4-(4-(4-((2,6-dioxopiperidin-3- |
| | yl)amino)phenethyl)piperazin-1-yl)piperidin-1-yl)-5-ethyl-2- |
| | methoxyphenyl)amino)pyrimidin-4-yl)amino)quinoxalin-5- |
| | yl)methanesulfonamide |
| 75 | 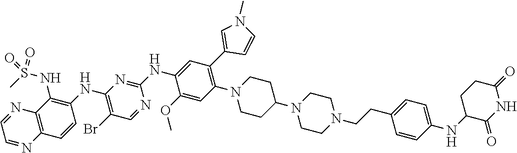 [SMILES: COc1cc(N2CCC(N3CCN(CCc4ccc(NC5CCC(=O)NC5=O)cc4)CC3)CC2)c(-c2ccn(C)c2)cc1Nc1ncc(Br)c(Nc2ccc3nccnc3c2NS(C)(=O)=O)n1] |
| | N-(6-((5-bromo-2-((4-(4-(4-(4-((2,6-dioxopiperidin-3- |
| | yl)amino)phenethyl)piperazin-1-yl)piperidin-1-yl)-2-methoxy-5-(1- |
| | methyl-1H-pyrrol-3-yl)phenyl)amino)pyrimidin-4- |
| | yl)amino)quinoxalin-5-yl)methanesulfonamide |
| 76 | 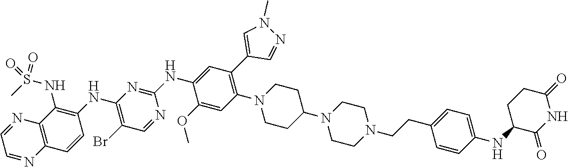 [SMILES: COc1cc(N2CCC(N3CCN(CCc4ccc(N[C@H]5CCC(=O)NC5=O)cc4)CC3)CC2)c(-c2cnn(C)c2)cc1Nc1ncc(Br)c(Nc2ccc3nccnc3c2NS(C)(=O)=O)n1] |
| | (S)-N-(6-((5-bromo-2-((4-(4-(4-(4-((2,6-dioxopiperidin-3- |
| | yl)amino)phenethyl)piperazin-1-yl)piperidin-1-yl)-2-methoxy-5-(1- |
| | methyl-1H-pyrazol-4-yl)phenyl)amino)pyrimidin-4- |
| | yl)amino)quinoxalin-5-yl)methanesulfonamide |
| 77 | 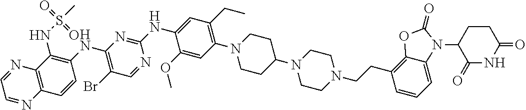 [SMILES: CCc1cc(Nc2ncc(Br)c(Nc3ccc4nccnc4c3NS(C)(=O)=O)n2)c(OC)cc1N1CCC(N2CCN(CCc3cccc4c3oc(=O)n4C3CCC(=O)NC3=O)CC2)CC1] |
| | N-(6-((5-bromo-2-((4-(4-(4-(2-(3-(2,6-dioxopiperidin-3-yl)-2-oxo- |
| | 2,3-dihydrobenzo[d]oxazol-7-yl)ethyl)piperazin-1-yl)piperidin-1- |
| | yl)-5-ethyl-2-methoxyphenyl)amino)pyrimidin-4- |
| | yl)amino)quinoxalin-5-yl)methanesulfonamide |
| 78 | 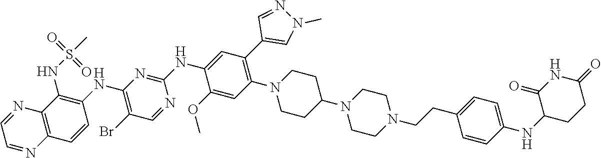 [SMILES: COc1cc(N2CCC(N3CCN(CCc4ccc(NC5CCC(=O)NC5=O)cc4)CC3)CC2)c(-c2cnn(C)c2)cc1Nc1ncc(Br)c(Nc2ccc3nccnc3c2NS(C)(=O)=O)n1] |
| | N-(6-((5-bromo-2-((4-(4-(4-(4-((2,6-dioxopiperidin-3- |
| | yl)amino)phenethyl)piperazin-1-yl)piperidin-1-yl)-2-methoxy-5-(1- |
| | methyl-1H-pyrazol-4-yl)phenyl)amino)pyrimidin-4- |
| | yl)amino)quinoxalin-5-yl)methanesulfonamide |
| 79 | 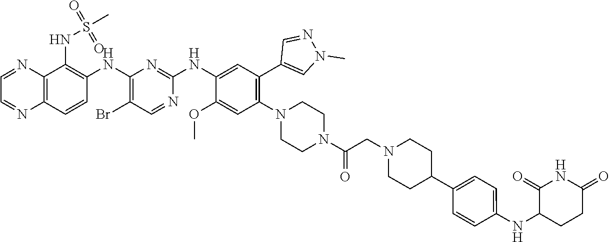 [SMILES: COc1cc(N2CCN(C(=O)CN3CCC(c4ccc(NC5CCC(=O)NC5=O)cc4)CC3)CC2)c(-c2cnn(C)c2)cc1Nc1ncc(Br)c(Nc2ccc3nccnc3c2NS(C)(=O)=O)n1] |
| | N-(6-((5-bromo-2-((4-(4-(2-(4-(4-((2,6-dioxopiperidin-3- |
| | yl)amino)phenyl)piperidin-1-yl)acetyl)piperazin-1-yl)-2-methoxy-5- |
| | (1-methyl-1H-pyrazol-4-yl)phenyl)amino)pyrimidin-4- |
| | yl)amino)quinoxalin-5-yl)methanesulfonamide |
| 80 | 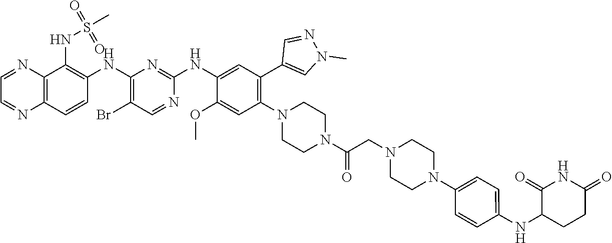 [SMILES: COc1cc(N2CCN(C(=O)CN3CCN(c4ccc(NC5CCC(=O)NC5=O)cc4)CC3)CC2)c(-c2cnn(C)c2)cc1Nc1ncc(Br)c(Nc2ccc3nccnc3c2NS(C)(=O)=O)n1] |
| | N-(6-((5-bromo-2-((4-(4-(2-(4-(4-((2,6-dioxopiperidin-3- |
| | yl)amino)phenyl)piperazin-1-yl)acetyl)piperazin-1-yl)-2-methoxy- |
| | 5-(1-methyl-1H-pyrazol-4-yl)phenyl)amino)pyrimidin-4- |
| | yl)amino)quinoxalin-5-yl)methanesulfonamide |
| 81 | 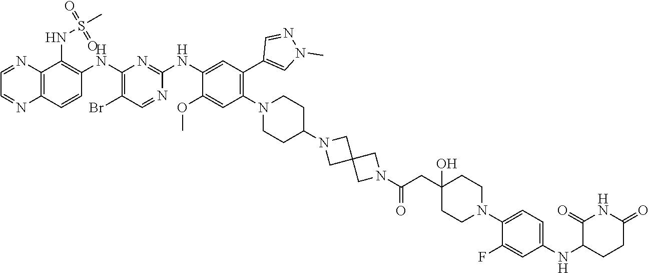 [SMILES: COc1cc(N2CCC(N3CC4(CN(C(=O)CC5(O)CCN(c6ccc(NC7CCC(=O)NC7=O)cc6F)CC5)C4)C3)CC2)c(-c2cnn(C)c2)cc1Nc1ncc(Br)c(Nc2ccc3nccnc3c2NS(C)(=O)=O)n1] |
| | N-(6-((5-bromo-2-((4-(4-(6-(2-(1-(4-((2,6-dioxopiperidin-3- |
| | yl)amino)-2-fluorophenyl)-4-hydroxypiperidin-4-yl)acetyl)-2,6- |
| | diazaspiro[3.3]heptan-2-yl)piperidin-1-yl)-2-methoxy-5-(1-methyl- |
| | 1H-pyrazol-4-yl)phenyl)amino)pyrimidin-4-yl)amino)quinoxalin-5- |
| | yl)methanesulfonamide |
| 82 | 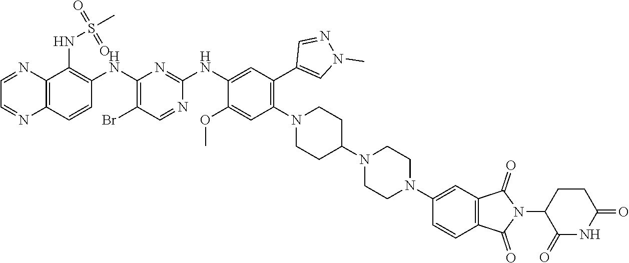 [SMILES: COc1cc(N2CCC(N3CCN(c4ccc5c(c4)C(=O)N(C4CCC(=O)NC4=O)C5=O)CC3)CC2)c(-c2cnn(C)c2)cc1Nc1ncc(Br)c(Nc2ccc3nccnc3c2NS(C)(=O)=O)n1] |
| | N-(6-((5-bromo-2-((4-(4-(4-(2-(2,6-dioxopiperidin-3-yl)-1,3- |
| | dioxoisoindolin-5-yl)piperazin-1-yl)piperidin-1-yl)-2-methoxy-5-(1- |
| | methyl-1H-pyrazol-4-yl)phenyl)amino)pyrimidin-4- |
| | yl)amino)quinoxalin-5-yl)methanesulfonamide |
| 83 | 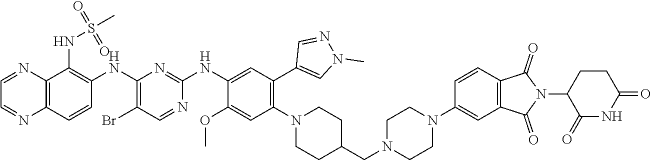 [SMILES: COc1cc(N2CCC(CN3CCN(c4ccc5c(c4)C(=O)N(C4CCC(=O)NC4=O)C5=O)CC3)CC2)c(-c2cnn(C)c2)cc1Nc1ncc(Br)c(Nc2ccc3nccnc3c2NS(C)(=O)=O)n1] |
| | N-(6-((5-bromo-2-((4-(4-((4-(2-(2,6-dioxopiperidin-3-yl)-1,3- |
| | dioxoisoindolin-5-yl)piperazin-1-yl)methyl)piperidin-1-yl)-2- |
| | methoxy-5-(1-methyl-1H-pyrazol-4-yl)phenyl)amino)pyrimidin-4- |
| | yl)amino)quinoxalin-5-yl)methanesulfonamide |
| 84 | 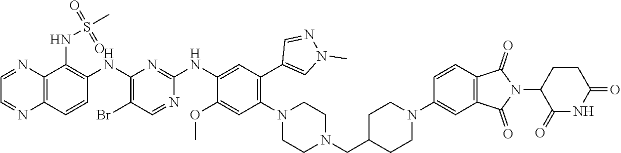 [SMILES: COc1cc(N2CCN(CC3CCN(c4ccc5c(c4)C(=O)N(C4CCC(=O)NC4=O)C5=O)CC3)CC2)c(-c2cnn(C)c2)cc1Nc1ncc(Br)c(Nc2ccc3nccnc3c2NS(C)(=O)=O)n1] |
| | N-(6-((5-bromo-2-((4-(4-((1-(2-(2,6-dioxopiperidin-3-yl)-1,3- |
| | dioxoisoindolin-5-yl)piperidin-4-yl)methyl)piperazin-1-yl)-2- |
| | methoxy-5-(1-methyl-1H-pyrazol-4-yl)phenyl)amino)pyrimidin-4- |
| | yl)amino)quinoxalin-5-yl)methanesulfonamide |
| 85 | 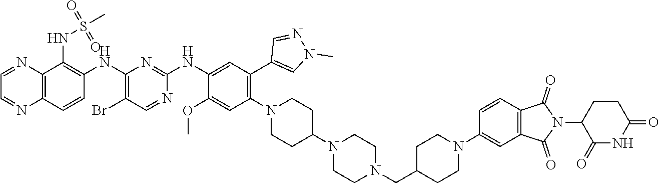 [SMILES: COc1cc(N2CCC(N3CCN(CC4CCN(c5ccc6c(c5)C(=O)N(C5CCC(=O)NC5=O)C6=O)CC4)CC3)CC2)c(-c2cnn(C)c2)cc1Nc1ncc(Br)c(Nc2ccc3nccnc3c2NS(C)(=O)=O)n1] |
| | N-(6-((5-bromo-2-((4-(4-(4-((1-(2-(2,6-dioxopiperidin-3-yl)-1,3- |
| | dioxoisoindolin-5-yl)piperidin-4-yl)methyl)piperazin-1-yl)piperidin- |
| | 1-yl)-2-methoxy-5-(1-methyl-1H-pyrazol-4- |
| | yl)phenyl)amino)pyrimidin-4-yl)amino)quinoxalin-5- |
| | yl)methanesulfonamide |
| 86 | 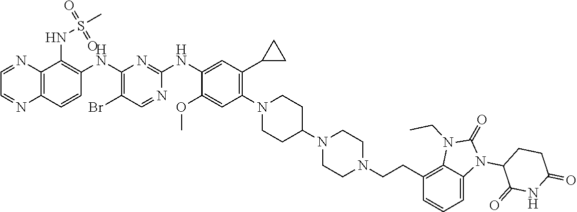 [SMILES: CCn1c(=O)n(C2CCC(=O)NC2=O)c2cccc(CCN3CCN(C4CCN(c5cc(OC)c(Nc6ncc(Br)c(Nc7ccc8nccnc8c7NS(C)(=O)=O)n6)cc5C5CC5)CC4)CC3)c21] |
| | N-(6-((5-bromo-2-((5-cyclopropyl-4-(4-(4-(2-(1~(2,6-dioxopiperidin- |
| | 3-yl)-3-ethyl-2-oxo-2,3-dihydro-1H-benzo[d]imidazol-4- |
| | yl)ethyl)piperazin-1-yl)piperidin-1-yl)-2- |
| | methoxyphenyl)amino)pyrimidin-4-yl)amino)quinoxalin-5- |
| | yl)methanesulfonamide |
| 87 | 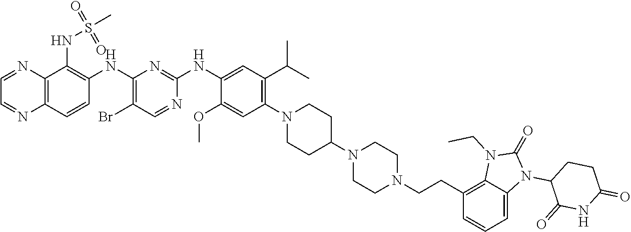 [SMILES: CCn1c(=O)n(C2CCC(=O)NC2=O)c2cccc(CCN3CCN(C4CCN(c5cc(OC)c(Nc6ncc(Br)c(Nc7ccc8nccnc8c7NS(C)(=O)=O)n6)cc5C(C)C)CC4)CC3)c21] |
| | N-(6-((5-bromo-2-((4-(4-(4-(2-(1-(2,6-dioxopiperidin-3-yl)-3-ethyl- |
| | 2-oxo-2,3-dihydro-1H-benzo[d]imidazol-4-yl)ethyl)piperazin-1- |
| | yl)piperidin-1-yl)-5-isopropyl-2-methoxyphenyl)amino)pyrimidin-4- |
| | yl)amino)quinoxalin-5-yl)methanesulfonamide |
| 88 | 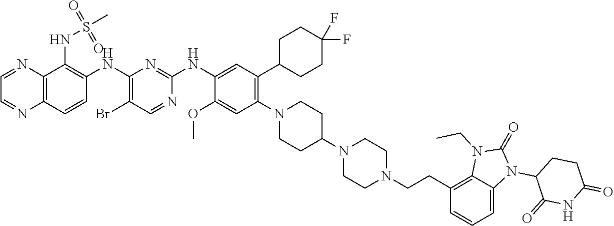 [SMILES: CCn1c(=O)n(C2CCC(=O)NC2=O)c2cccc(CCN3CCN(C4CCN(c5cc(OC)c(Nc6ncc(Br)c(Nc7ccc8nccnc8c7NS(C)(=O)=O)n6)cc5C5CCC(F)(F)CC5)CC4)CC3)c21] |
| | N-(6-((5-bromo-2-((5-(4,4-difluorocyclohexyl)-4-(4-(4-(2-(1-(2,6- |
| | dioxopiperidin-3-yl)-3-ethyl-2-oxo-2,3-dihydro-1H- |
| | benzo[d]imidazol-4-yl)ethyl)piperazin-1-yl)piperidin-1-yl)-2- |
| | methoxyphenyl)amino)pyrimidin-4-yl)amino)quinoxalin-5- |
| | yl)methanesulfonamide |
| 89 | 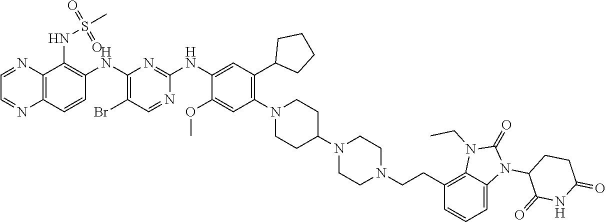 [SMILES: CCn1c(=O)n(C2CCC(=O)NC2=O)c2cccc(CCN3CCN(C4CCN(c5cc(OC)c(Nc6ncc(Br)c(Nc7ccc8nccnc8c7NS(C)(=O)=O)n6)cc5C5CCCC5)CC4)CC3)c21] |
| | N-(6-((5-bromo-2-((5-cyclopentyl-4-(4-(4-(2-(1-(2,6-dioxopiperidin- |
| | 3-yl)-3-ethyl-2-oxo-2,3-dihydro-1H-benzo[d]imidazol-4- |
| | yl)ethyl)piperazin-1-yl)piperidin-1-yl)-2- |
| | methoxyphenyl)amino)pyrimidin-4-yl)amino)quinoxalin-5- |
| | yl)methanesulfonamide |
| 90 | 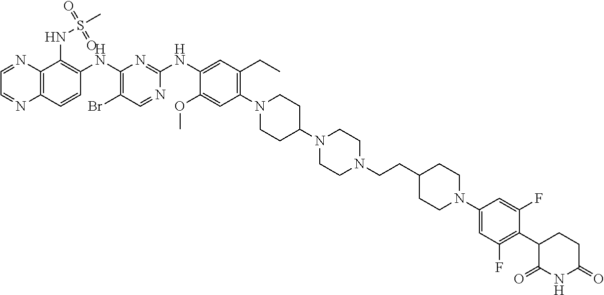 [SMILES: CCc1cc(Nc2ncc(Br)c(Nc3ccc4nccnc4c3NS(C)(=O)=O)n2)c(OC)cc1N1CCC(N2CCN(CCC3CCN(c4cc(F)c(C5CCC(=O)NC5=O)c(F)c4)CC3)CC2)CC1] |
| | N-(6-((5-bromo-2-((4-(4-(4-(2-(1-(4-(2,6-dioxopiperidin-3-yl)-3,5- |
| | difluorophenyl)piperidin-4-yl)ethyl)piperazin-1-yl)piperidin-1-yl)-5- |
| | ethyl-2-methoxyphenyl)amino)pyrimidin-4-yl)amino)quinoxalin-5- |
| | yl)methanesulfonamide |
| 91 | 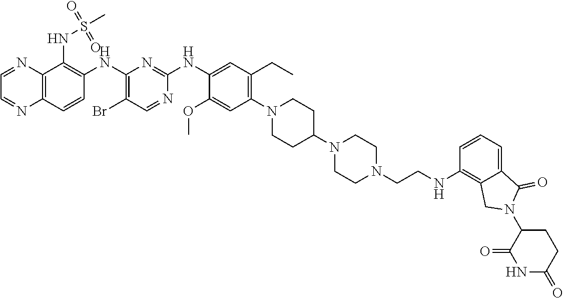 [SMILES: CCc1cc(Nc2ncc(Br)c(Nc3ccc4nccnc4c3NS(C)(=O)=O)n2)c(OC)cc1N1CCC(N2CCN(CCNc3cccc4c3CN(C3CCC(=O)NC3=O)C4=O)CC2)CC1] |
| | N-(6-((5-bromo-2-((4-(4-(4-(2-((2-(2,6-dioxopiperidin-3-yl)-1- |
| | oxoisoindolin-4-yl)amino)ethyl)piperazin-1-yl)piperidin-1-yl)-5- |
| | ethyl-2-methoxyphenyl)amino)pyrimidin-4-yl)amino)quinoxalin-5- |
| | yl)methanesulfonamide |
| 92 | 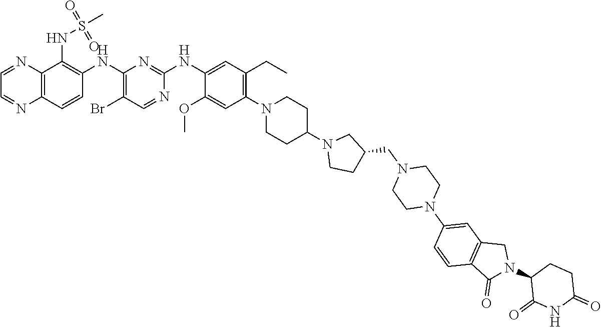 [SMILES: CCc1cc(Nc2ncc(Br)c(Nc3ccc4nccnc4c3NS(C)(=O)=O)n2)c(OC)cc1N1CCC(N2CC[C@@H](CN3CCN(c4ccc5c(c4)CN([C@H]4CCC(=O)NC4=O)C5=O)CC3)C2)CC1] |
| | N-(6-(5-bromo-2-(4-(4-((S)-3-(4-(2-(S)-2,6-dioxopiperidin-3-yl)- |
| | 1-oxoisoindolin-5-yl)piperazin-1-yl)methyl)pyrrolidin-1-yl)piperidin- |
| | 1-yl)-5-ethyl-2-methoxyphenyl)amino)pyrimidin-4- |
| | yl)amino)quinoxalin-5-yl)methanesulfonamide |
| 93 | 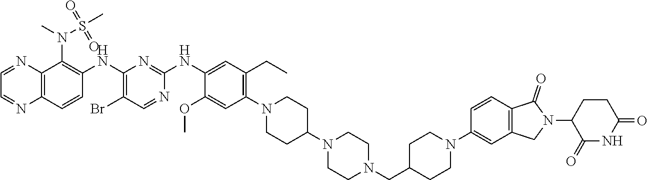 [SMILES: CCc1cc(Nc2ncc(Br)c(Nc3ccc4nccnc4c3N(C)S(C)(=O)=O)n2)c(OC)cc1N1CCC(N2CCN(CC3CCN(c4ccc5c(c4)CN(C4CCC(=O)NC4=O)C5=O)CC3)CC2)CC1] |
| | N-(6-((5-bromo-2-((4-(4-(4-((1-(2-(2,6-dioxopiperidin-3-yl)-1- |
| | oxoisoindolin-5-yl)piperidin-4-yl)methyl)piperazin-1-yl)piperidin-1- |
| | yl)-5-ethyl-2-methoxyphenyl)amino)pyrimidin-4- |
| | yl)amino)quinoxalin-5-yl)-N-methylmethanesulfonamide |
| 94 | 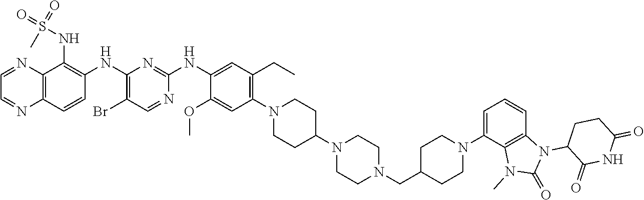 [SMILES: CCc1cc(Nc2ncc(Br)c(Nc3ccc4nccnc4c3NS(C)(=O)=O)n2)c(OC)cc1N1CCC(N2CCN(CC3CCN(c4cccc5c4n(C)c(=O)n5C4CCC(=O)NC4=O)CC3)CC2)CC1] |
| | N-(6-((5-bromo-2-((4-(4-(4-((1-(1-(2,6-dioxopiperidin-3-yl)-3- |
| | methyl-2-oxo-2,3-dihydro-1H-benzo[d]imidazol-4-yl)piperidin-4- |
| | yl)methyl)piperazin-1-yl)piperidin-1-yl)-5-ethyl-2- |
| | methoxyphenyl)amino)pyrimidin-4-yl)amino)quinoxalin-5- |
| | yl)methanesulfonamide |
| 95 | 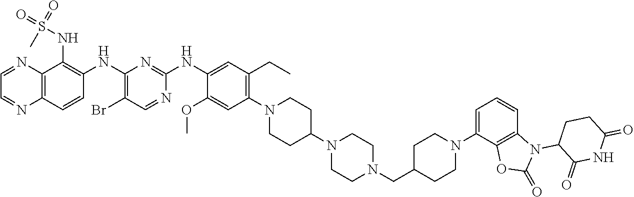 [SMILES: CCc1cc(Nc2ncc(Br)c(Nc3ccc4nccnc4c3NS(C)(=O)=O)n2)c(OC)cc1N1CCC(N2CCN(CC3CCN(c4cccc5c4oc(=O)n5C4CCC(=O)NC4=O)CC3)CC2)CC1] |
| | N-(6-((5-bromo-2-((4-(4-(4-((1-(3-(2,6-dioxopiperidin-3-yl)-2-oxo- |
| | 2,3-dihydrobenzo[d]oxazol-7-yl)piperidin-4-yl)methyl)piperazin-1- |
| | yl)piperidin-1-yl)-5-ethyl-2-methoxyphenyl)amino)pyrimidin-4- |
| | yl)amino)quinoxalin-5-yl)methanesulfonamide |

### Pharmaceutical Compositions

Pharmaceutical compositions of the present disclosure comprise at least one entity chosen from the compounds of Formula (I), or tautomers thereof, stereoisomers or a mixture of stereoisomers thereof, pharmaceutically acceptable salts thereof, hydrates thereof, and deuterated derivatives thereof, with at least one pharmaceutically acceptable carrier. These formulations include those suitable for oral, rectal, topical, buccal, and parenteral (e.g., subcutaneous, intramuscular, intradermal, or intravenous) administration. The most suitable form of administration in any given case will depend on the degree and severity of the condition being treated and on the nature of the particular compound being used.

Formulations suitable for oral administration may be presented in discrete units, such as capsules, cachets, lozenges, or tablets, each containing a predetermined amount of a compound of the present disclosure as powder or granules; as a solution or a suspension in an aqueous or non-aqueous liquid; or as an oil-in-water or water-in-oil emulsion. As indicated, such formulations may be prepared by any suitable method of pharmacy which includes the step of bringing into association at least one entity of the present disclosure as the active compound and a carrier or excipient (which may constitute one or more accessory ingredients). The carrier must be acceptable in the sense of being compatible with the other ingredients of the formulation and must not be deleterious to the recipient. The carrier may be a solid or a liquid, or both, and may be formulated with at least one compound described herein as the active compound in a unit-dose formulation, for example, a tablet, which may contain from about 0.05% to about 95% by weight of the at least one active compound. Other pharmacologically active substances may also be present including other compounds. The formulations of the present disclosure may be prepared by any of the well-known techniques of pharmacy consisting essentially of admixing the components.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by, for example, dissolving or dispersing, at least entity of the present disclosure as described herein and optional pharmaceutical adjuvants in an excipient, such as, for example, water, saline, aqueous dextrose, glycerol, ethanol, and the like, to thereby form a solution or suspension. In general, suitable formulations may be prepared by uniformly and intimately admixing the at least one active compound of the present disclosure with a liquid or finely divided solid carrier, or both, and then, if necessary, shaping the product. For example, a tablet may be prepared by compressing or molding a powder or granules of at least entity of the present disclosure, which may be optionally combined with one or more accessory ingredients. Compressed tablets may be prepared by compressing, in a suitable machine, at least entity of the present disclosure in a free-flowing form, such as a powder or granules, which may be optionally mixed with a binder, lubricant, inert diluent and/or surface active/dispersing agent(s). Molded tablets may be made by molding, in a suitable machine, where the powdered form of at least one compound of the present disclosure is moistened with an inert liquid diluent.

Formulations suitable for buccal (sub-lingual) administration include lozenges comprising at least one entity of the present disclosure in a flavored base, usually sucrose and acacia or tragacanth, and pastilles comprising the at least one compound in an inert base such as gelatin and glycerin or sucrose and acacia.

Formulations of the present disclosure suitable for parenteral administration comprise sterile aqueous preparations of at least one entity chosen from the compounds of Formula (I), or tautomers thereof, stereoisomers or a mixture of stereoisomers thereof, pharmaceutically acceptable salts thereof, hydrates thereof, and deuterated derivatives thereof, which are approximately isotonic with the blood of the intended recipient. These preparations are administered intravenously, although administration may also be affected by means of subcutaneous, intramuscular, or intradermal injection. Such preparations may conveniently be prepared by admixing at least one compound described herein with water and rendering the resulting solution sterile and isotonic with the blood. Injectable compositions according to the present disclosure may contain from about 0.1 to about 5% w/w of the active compound.

Formulations suitable for rectal administration are presented as unit-dose suppositories. These may be prepared by admixing at least one entity as described herein with one or more conventional solid carriers, for example, cocoa butter, and then shaping the resulting mixture.

Formulations suitable for topical application to the skin may take the form of an ointment, cream, lotion, paste, gel, spray, aerosol, or oil. Carriers and excipients which may be used include Vaseline, lanoline, polyethylene glycols, alcohols, and combinations of two or more thereof. The active compound (i.e., at least one entity chosen from the compounds of Formula (I), or tautomers thereof, stereoisomers or a mixture of stereoisomers thereof, pharmaceutically acceptable salts thereof, hydrates thereof, and deuterated derivatives thereof) is generally present at a concentration of from about 0.1% to about 15% w/w of the composition, for example, from about 0.5 to about 2%.

The amount of active compound administered may be dependent on the subject being treated, the subject's weight, the manner of administration and the judgment of the prescribing physician. For example, a dosing schedule may involve the daily or semi-daily administration of the encapsulated compound at a perceived dosage of about 1 μg to about 1000 mg. In another embodiment, intermittent administration, such as on a monthly or yearly basis, of a dose of the encapsulated compound may be employed.

Encapsulation facilitates access to the site of action and allows the administration of the active ingredients simultaneously, in theory producing a synergistic effect. In accordance with standard dosing regimens, physicians will readily determine optimum dosages and will be able to readily modify administration to achieve such dosages.

A therapeutically effective amount of a compound or composition disclosed herein can be measured by the therapeutic effectiveness of the compound. The dosages, however, may be varied depending upon the requirements of the patient, the severity of the condition being treated, and the compound being used. In one embodiment, the therapeutically effective amount of a disclosed compound is sufficient to establish a maximal plasma concentration. Preliminary doses as, for example, determined according to animal tests, and the scaling of dosages for human administration is performed according to art-accepted practices.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferable.

Data obtained from cell culture assays or animal studies can be used in formulating a range of dosage for use in humans. Therapeutically effective dosages achieved in one animal model may be converted for use in another animal, including humans, using conversion factors known in the art (see, e.g., Freireich et al., Cancer Chemother. Reports 50 (4): 219-244 (1966) and the following table (Table 3) for Equivalent Surface Area Dosage Factors).

TABLE 3
Equivalent Surface Area Dosage Factors.

To:

| | Mouse | Rat | Monkey | Dog | Human |
|---|---|---|---|---|---|
| From: | (20 g) | (150 g) | (3.5 kg) | (8 kg) | (60 kg) |
| Mouse | 1 | 1/2 | 1/4 | 1/6 | 1/12 |
| Rat | 2 | 1 | 1/2 | 1/4 | 1/7 |
| Monkey | 4 | 2 | 1 | 3/5 | 1/3 |
| Dog | 6 | 4 | 3/5 | 1 | 1/2 |
| Human | 12 | 7 | 3 | 2 | 1 |

The dosage of such compounds lies preferably within a range of circulating concentrations that include the ED₅₀ with little or no toxicity. The dosage may vary within this range depending upon the dosage form employed and the route of administration utilized. Generally, a therapeutically effective amount may vary with the subject's age, condition, and gender, as well as the severity of the medical condition in the subject. The dosage may be determined by a physician and adjusted, as necessary, to suit observed effects of the treatment.

### Methods of Treatment

In some embodiments, at least one entity chosen from the compounds of Formula (I), or tautomers thereof, stereoisomers or a mixture of stereoisomers thereof, pharmaceutically acceptable salts thereof, hydrates thereof, and deuterated derivatives thereof, is administered to treat cancer in a subject in need thereof.

In some embodiments, the cancer is associated with an EGFR or Her2 exon20 insertion mutation.

In some embodiments, the cancer is selected from breast cancer, lung cancer, pancreatic cancer, colon cancer, head and neck cancer, renal cell carcinoma, squamous cell carcinoma, thyroid cancer, gall bladder cancer, thyroid cancer, bile duct cancer, ovarian cancer, endometrial cancer, prostate cancer, or esophageal cancer.

In some embodiments, the cancer is lung cancer. In a further embodiment, the cancer is non-small cell lung cancer. In some embodiments, the cancer is pancreatic cancer. In some embodiments, the cancer is colon cancer. In some embodiments, the cancer is breast cancer.

In some embodiments, at least one entity chosen from the compounds of Formula (I), or tautomers thereof, stereoisomers or a mixture of stereoisomers thereof, pharmaceutically acceptable salts thereof, hydrates thereof, and deuterated derivatives thereof, is administered as a pharmaceutical composition.

The concentration and route of administration to the patient will vary depending on the cancer to be treated.

In some embodiments, at least one entity from the compounds of Formula (I), or tautomers thereof, stereoisomers or a mixture of stereoisomers thereof, pharmaceutically acceptable salts thereof, hydrates thereof, and deuterated derivatives thereof, is administered in combination with another therapeutic agent, e.g., chemotherapy, or used in combination with other treatments, such as radiation or surgical intervention, either as an adjuvant prior to surgery or post-operatively. In some embodiments, the therapeutic agent is chosen from gemcitabine, cisplatin, erlotinib, gefitinib, pemetrexed, bevacizumab, cetuximab, trastuzumab, pertuzumab, sorafenib, lapatinib, cobimetinib, selumetinib, and everolimus.

In some embodiments, provided herein is at least one entity chosen from the compounds of Formula (I), or tautomers, stereoisomers or a mixture of stereoisomers thereof, pharmaceutically acceptable salts thereof, hydrates thereof, and deuterated derivatives thereof, or a pharmaceutical composition thereof as defined herein for use in therapy.

In some embodiments, provided herein is at least one entity chosen from the compounds of Formula (I), or tautomers thereof, stereoisomers or a mixture of stereoisomers thereof, pharmaceutically acceptable salts thereof, hydrates thereof, and deuterated derivatives thereof, or a pharmaceutical composition thereof as defined herein for use in the treatment of cancer.

In some embodiments, provided herein is at least one entity chosen from the compounds of Formula (I), or tautomers thereof, stereoisomers or a mixture of stereoisomers thereof, pharmaceutically acceptable salts thereof, hydrates thereof, and deuterated derivatives thereof, for use in the inhibition of EGFR.

In some embodiments, provided herein is at least one entity from the compounds of Formula (I), or tautomers thereof, stereoisomers or a mixture of stereoisomers thereof, pharmaceutically acceptable salts thereof, hydrates thereof, and deuterated derivatives thereof, or a pharmaceutical composition thereof as defined herein, for use in the treatment of a disease or disorder associated with an EGFR or HER2 exon 20 insertion mutation.

In some embodiments, provided herein is the use of at least one entity chosen from the compounds of Formula (I), or tautomers thereof, stereoisomers or a mixture of stereoisomers thereof, pharmaceutically acceptable salts thereof, hydrates thereof, and deuterated derivatives thereof, as defined herein in the manufacture of a medicament for the treatment of cancer. In some embodiments, the cancer is lung cancer. In some embodiments, the cancer is breast cancer. In some embodiments, the lung cancer is non-small cell lung cancer (NSCLC) or small cell lung cancer (SCLC). In some embodiments, the NSCLC is selected from adenocarcinoma, squamous cell carcinoma, and large cell carcinoma.

In some embodiments, provided herein is a use of at least one entity chosen from the compounds of Formula (I), or tautomers thereof, stereoisomers or a mixture of stereoisomers thereof, pharmaceutically acceptable salts thereof, hydrates thereof, and deuterated derivatives thereof, as defined herein in the manufacture of a medicament for the inhibition of activity of EGFR.

In some embodiments, provided herein is the use of at least one entity chosen from the compounds of Formula (I), or tautomers thereof, stereoisomers or a mixture of stereoisomers thereof, pharmaceutically acceptable salts thereof, hydrates thereof, and deuterated derivatives thereof, as defined herein, in the manufacture of a medicament for the treatment of a disease or disorder associated with an EGFR or HER2 mutations including EGFR exon19 deletion, L858R, T790M, C797S, EGFR or HER2 exon20 insertion.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

In some embodiments, provided herein is a method of inhibiting EGFR kinase activity in a cell comprising contacting the cell with an effective amount of an EGFR degrader. In some embodiments, the administered amount is a therapeutically effective amount and the inhibition of EGFR kinase activity further results in the inhibition of the growth of the cell. In some embodiments, the cell is a cancer cell.

Inhibition of cell proliferation is measured using methods known to those skilled in the art. For example, a convenient assay for measuring cell proliferation is the CellTiter-Glo™ Luminescent Cell Viability Assay, which is commercially available from Promega (Madison, Wis.). That assay determines the number of viable cells in culture based on quantitation of ATP present, which is an indication of metabolically active cells. See Crouch et al (1993) J. Immunol. Meth. 160:81-88, U.S. Pat. No. 6,602,677. The assay may be conducted in 96- or 384-well format, making it amenable to automated high throughput screening (HTS). See Cree et al (1995) AntiCancer Drugs 6:398-404. The assay procedure involves adding a single reagent (CellTiter-Glo® Reagent) directly to cultured cells. This results in cell lysis and generation of a luminescent signal produced by a luciferase reaction. The luminescent signal is proportional to the amount of ATP present, which is directly proportional to the number of viable cells present in culture. Data can be recorded by luminometer or CCD camera imaging device. The luminescence output is expressed as relative light units (RLU). Inhibition of cell proliferation may also be measured using colony formation assays known in the art.

Furthermore, the present disclosure provides for methods of treating a condition associated with an EGFR or HER2 mutations including EGFR exon19 deletion, L858R, T790M, C797S, EGFR or HER2 exon20 insertion in a subject suffering therefrom, comprising administering to the subject a therapeutically effective amount of an EGFR or HER2 degrader. In one embodiment, the condition is a cell proliferative disease.

Treatment of the cell proliferative disorder by administration of an EGFR degrader results in an observable and/or measurable reduction in or absence of one or more of the following: reduction in the number of cancer cells or absence of the cancer cells; reduction in the tumor size; inhibition of cancer cell infiltration into peripheral organs including the spread of cancer into soft tissue and bone; inhibition of tumor metastasis; inhibition, to some extent, of tumor growth; and/or relief to some extent, one or more of the symptoms associated with the specific cancer; reduced morbidity and mortality, and improvement in quality of life issues. To the extent the EGFR degrader may prevent growth and/or kill existing cancer cells, it may be cytostatic and/or cytotoxic. Reduction of these signs or symptoms may also be felt by the patient.

### Examples

The examples and preparations provided below further illustrate and exemplify the compounds as disclosed herein and methods of preparing such compounds. It is to be understood that the scope of the present disclosure is not limited in any way by the scope of the following examples and preparations.

The chemical entities described herein can be synthesized according to one or more illustrative schemes herein and/or techniques well known in the art. Unless specified to the contrary, the reactions described herein take place at atmospheric pressure, generally within a temperature range from about-10° C. to about 200° C. Further, except as otherwise specified, reaction times and conditions are intended to be approximate, e.g., taking place at about atmospheric pressure within a temperature range of about-10° C. to about 200° C. over a period that can be, for example, about 1 to about 24 hours; reactions left to run overnight in some embodiments can average a period of about 16 hours.

Isolation and purification of the chemical entities and intermediates described herein can be affected, if desired, by any suitable separation or purification procedure such as, for example, filtration, extraction, crystallization, column chromatography, thin-layer chromatography or thick-layer chromatography, or a combination of these procedures. See, e.g., Carey et al. Advanced Organic Chemistry, 3rd Ed., 1990 New York: Plenum Press; Mundy et al., Name Reaction and Reagents in Organic Synthesis, 2nd Ed., 2005 Hoboken, NJ: J. Wiley & Sons. Specific illustrations of suitable separation and isolation procedures are given by reference to the examples hereinbelow. However, other equivalent separation or isolation procedures can also be used.

In all the methods, it is well understood that protecting groups for sensitive or reactive groups may be employed where necessary, in accordance with general principles of chemistry. Protecting groups are manipulated according to standard methods of organic synthesis (T. W. Greene and P. G. M. Wuts (1999) Protective Groups in Organic Synthesis, 3rd Ed., John Wiley & Sons). These groups may be removed at a convenient stage of the compound synthesis using methods that are readily apparent to those skilled in the art.

The compounds described herein can be optionally contacted with a pharmaceutically acceptable acid to form the corresponding acid addition salts. Also, the compounds described herein can be optionally contacted with a pharmaceutically acceptable base to form the corresponding basic addition salts.

In some embodiments, disclosed compounds can generally be synthesized by an appropriate combination of generally well-known synthetic methods. Techniques useful in synthesizing these chemical entities are both readily apparent and accessible to those of skill in the relevant art, based on the instant disclosure. Many of the optionally substituted starting compounds and other reactants are commercially available, e.g., from Millipore Sigma or can be readily prepared by those skilled in the art using commonly employed synthetic methodology.

The discussion below is offered to illustrate certain of the diverse methods available for use in making the disclosed compounds and is not intended to limit the scope of reactions or reaction sequences that can be used in preparing the compounds provided herein. The skilled artisan will understand that standard atom valences apply to all compounds disclosed herein in genus or named compound for unless otherwise specified.

### Abbreviations

The following abbreviations have the meanings set forth below:
Ac Acetyl
ACN Acetonitrile
Boc tert-Butyloxycarbonyl
Cbz Benzyloxycarbonyl
CDI 1,1′-Carbonyldiimidazole
DCM Dichloromethane
DIEA N,N-Diisopropylethylamine
DMF Dimethylformamide
DMP Dess-Martin Periodinane
DMSO Dimethyl sulfoxide
Dppf 1,1′-bis(diphenylphosphino) ferrocene
EA Ethyl acetate
EDCl 1-Ethyl-3-(3-dimethylaminopropyl) carbodiimide
Equiv Equivalent
FA Formic acid
H Hours
HATU 1-[Bis(dimethylamino)methylene]-1H-1,2,3-triazolo[4,5-b]pyridinium 3-oxide hexafluorophosphate
HOBt Hydroxybenzotriazole
LCMS Liquid chromatography mass spectrometry
LiHMDS Lithium bis(trimethylsilyl)amide
Min Minutes
MS Mol sieves
Ms Methanesulfonyl
NMR Nuclear magnetic resonance
dppf 1,1′-bis(diphenylphosphino) ferrocene
PCy₃ Tricyclohexylphosphine
Pin Pinacolato
PE Petroleum ether
Prep-TLC Preparative thin layer chromatography
STAB Sodium triacetoxyborohydride
TEA Triethylamine
TFA Trifluoroacetic acid
THF Tetrahydrofuran
TLC Thin layer chromatography
TMS Trimethylsilyl
TsOH p-Toluenesulfonic acid

### General Synthesis Schemes

The claimed entities can be prepared according to the following schemes. The following schemes represent the general methods used in preparing these compounds. However, the synthesis of these entities is not limited to these representative methods, as they can also be prepared through various other methods by those skilled in the art of synthetic chemistry.

### Preparation of Representative Intermediates

### Intermediate-1: Preparation of benzyl 4-(1-(4-amino-2-ethyl-5-methoxyphenyl)piperidin-4-yl)piperazine-1-carboxylate (Int-1

### Step 1: Preparation of 8-(2-bromo-5-methoxy-4-nitrophenyl)-1,4-dioxa-8-azaspiro[4.5]decane

1-bromo-2-fluoro-4-methoxy-5-nitrobenzene (25 g, 100 mmol, 1.0 equiv), 1,4-dioxa-8-azaspiro[4.5]decane (21.4 g, 150 mmol, 1.5 equiv), and K₂CO₃ (27.6 g, 200 mmol, 2.0 equiv) were dissolved in MeCN (500 mL). The reaction mixture was heated to 80° C. and allowed to stir overnight. LCMS indicated complete consumption of starting material and formation of product with desired mass (90% peak area). The resulting mixture was filtered, and the filtrate was concentrated to give crude residue (37 g) which was used directly in the next step without further purification. LCMS: [M+H+]=373

### Step 2: Preparation of 8-(5-methoxy-4-nitro-2-vinylphenyl)-1,4-dioxa-8-azaspiro[4.5]decane

8-(2-Bromo-5-methoxy-4-nitrophenyl)-1,4-dioxa-8-azaspiro[4.5]decane (37 g, mmol, 1.0 eq), trifluoro (vinyl)-14-borane, potassium salt (20.1 g, 150 mmol, 1.5 eq), Pd (dppf) Cl₂ (7.2 g, 10 mmol, 0.1 eq) and K₃PO⁴ (42.4 g, 200 mmol, 2.0 eq) were dissolved in DMF (500 mL). The reaction mixture was heated to 100° C. and allowed to stir overnight. LCMS indicated complete consumption of starting material and formation of product with desired mass (90% peak area). Then the resulting mixture was concentrated. The crude residue was purified via column chromatography (EA/PE=60%) to give 8-(5-methoxy-4-nitro-2-vinylphenyl)-1,4-dioxa-8-azaspiro[4.5]decane (25 g, 78% yield) as a yellow oil. LCMS: [M+H+]=321.

### Step 3: Preparation of 5-ethyl-2-methoxy-4-(1,4-dioxa-8-azaspiro[4.5]decan-8-yl)aniline

8-(5-Methoxy-4-nitro-2-vinylphenyl)-1,4-dioxa-8-azaspiro[4.5]decane (25 g, 78 mmol, 1.0 eq) and Pd/C (2.5 g, 10%, wet) were dissolved in THF (300 mL). The reaction mixture was stirred at 25° C. under H2 balloon for 16 h. LCMS indicated complete consumption of starting material and formation of product with desired mass (90% peak area). Then the reaction mixture was filtered, and filtrate was concentrated to give crude residue (18 g) which was used directly for next step. LCMS: [M+H+]=293.

### Step 4: Preparation of 1-(4-amino-2-ethyl-5-methoxyphenyl)piperidin-4-one

5-Ethyl-2-methoxy-4-(1,4-dioxa-8-azaspiro[4.5]decan-8-yl)aniline (18 g, 56 mmol, 1.0 eq) were dissolved in acetone (140 mL) and 2N HCl (140 mL). The reaction mixture was heated to 50° C. and allowed to stir overnight. LCMS indicated complete consumption of starting material and formation of product with desired mass (70% peak area). The reaction mixture was neutralized with saturated aqueous sodium bicarbonate solution. The resulting mixture was extracted with ethyl acetate (50 mL×3) and washed with water, saturated brine. The organic phase was concentrated to give 1-(4-amino-2-ethyl-5-methoxyphenyl)piperidin-4-one (14 g) which was used directly for next step. LCMS: [M+H+]=249.

### Step 5: Preparation of tert-butyl (5-ethyl-2-methoxy-4-(4-oxopiperidin-1-yl)phenyl) carbamate

1-(4-Amino-2-ethyl-5-methoxyphenyl)piperidin-4-one (14 g, 56 mmol, 1.0 eq), (Boc)₂₀ (24.4 g, 112 mmol, 2.0 eq) and TEA (11.3 g, 112 mmol, 2.0 eq) were dissolved in DCM (280 mL). The reaction mixture was heated to 35° C. and allowed to stir overnight. LCMS indicated complete consumption of starting material and formation of product with desired mass (50% peak area). Then reaction mixture was concentrated and the crude residue was purified via column chromatography (EA/PE=25%) to give tert-butyl (5-ethyl-2-methoxy-4-(4-oxopiperidin-1-yl)phenyl) carbamate (7 g, 36% yield) as a white solid. LCMS: [M+H+]=349.

### Step 6: Preparation of benzyl 4-(1-(4-((tert-butoxycarbonyl)amino)-2-ethyl-5-methoxyphenyl)piperidin-4-yl)piperazine-1-carboxylate

tert-Butyl (5-ethyl-2-methoxy-4-(4-oxopiperidin-1-yl)phenyl) carbamate (7 g, 20 mmol, 1.0 eq), benzyl piperazine-1-carboxylate (6.6 g, 30 mmol, 1.5 eq) and AcOH (1.2 g, 20 mmol, 1.0 eq) were dissolved in MeOH (100 mL). Then the reaction mixture was stirred at 25° C. for 1 h. Then NaBH₃CN (1.5 g, 24 mmol, 1.2 eq) was added and the reaction mixture was stirred for another 16 h. LCMS indicated complete consumption of starting material and formation of product with desired mass (50% peak area). The resulting mixture was concentrated and the crude residue was purified via column chromatography (EA/PE=3/2) to give benzyl 4-(1-(4-((tert-butoxycarbonyl)amino)-2-ethyl-5-methoxyphenyl)piperidin-4-yl)piperazine-1-carboxylate (7 g, 64% yield) as a colorless oil. LCMS: [M+H+]=553.

### Step 7: Preparation of benzyl 4-(1-(4-amino-2-ethyl-5-methoxyphenyl)piperidin-4-yl) piperazine-1-carboxylate

4-(1-(4-((tert-Butoxycarbonyl)amino)-2-ethyl-5-methoxyphenyl)piperidin-4-yl) piperazine-1-carboxylate (7 g, 12.7 mmol, 1.0 eq) were dissolved in HCl/dioxane (50 mL, 4M). The reaction mixture was heated to 40° C. and allowed to stir overnight. LCMS indicated complete consumption of starting material and formation of product with desired mass (90% peak area). The reaction mixture was concentrated to give benzyl 4-(1-(4-amino-2-ethyl-5-methoxyphenyl)piperidin-4-yl)piperazine-1-carboxylate (6.2 g) as HCl salt which was used directly for next step. LCMS: [M+H+]=453.

### Preparation of N-(6-((5-bromo-2-chloropyrimidin-4-yl)amino)quinoxalin-5-yl) methanesulfonamide (Int-2

### Step 1: Preparation of 5-nitroquinoxalin-6-amine

Quinoxalin-6-amine (50 g, 344.8 mmol, 1.0 equiv) was dissolved in conc. H₂SO₄ (500 mL) and cooled to 0° C. Then, NaNO₃ (44 g, 517.2 mmol, 1.5 equiv) was added slowly at this temperature. The reaction mixture was heated to 25° C. and allowed to stir for 3 h. LCMS indicated complete consumption of starting material and formation of product with desired mass (90% peak area). Then, the resulting mixture was poured into ice water. The reaction mixture was neutralized with saturated aqueous sodium bicarbonate solution. The resulting mixture was extracted with EA (500 mL×3) and washed with water and saturated brine. The organic phase was dried over sodium sulfate and concentrated under reduced pressure to give crude residue which was recrystallized by DCM/PE to give 5-nitroquinoxalin-6-amine (30 g, 46% yield) as a yellow solid. LCMS: [M+H+]=191. ¹H NMR (400 MHZ, DMSO-d₆) δ: 8.81 (s, 1H), 8.65 (s, 1H), 7.95 (d, J=2.0 Hz, 1H), 7.47 (d, J=2.0 Hz, 1H), 7.41 (brs, 2H).

### Step 2: Preparation of N-(5-bromo-2-chloropyrimidin-4-yl)-5-nitroquinoxalin-6-amine

5-nitroquinoxalin-6-amine (30 g, 157.9 mmol, 1.0 equiv), 5-bromo-2,4-dichloropyrimidine (72 g, 315.8 mmol, 2.0 equiv), and K₂CO₃ (43.6 g, 315.8 mmol, 2.0 equiv) were dissolved in DMF (700 mL). The reaction mixture was heated to 100° C. and allowed to stir overnight. LCMS indicated complete consumption of starting material and formation of product with desired mass (70% peak area). Then, the resulting mixture was concentrated, and the crude residue was purified via column chromatography (EA/PE=4/1) to give N-(5-bromo-2-chloropyrimidin-4-yl)-5-nitroquinoxalin-6-amine (20 g, 33% yield) as a yellow solid. LCMS: [M+H+]=383.

### Step 3: Preparation of N6-(5-bromo-2-chloropyrimidin-4-yl)quinoxaline-5,6-diamine

N-(5-bromo-2-chloropyrimidin-4-yl)-5-nitroquinoxalin-6-amine (20 g, 52.5 mmol, 1.0 equiv), Fe powder (14.7 g, 262.5 mmol, 5.0 equiv), and NH₄Cl (14 g, 262.5 mmol, 5.0 equiv) were dissolved in EtOH (200 mL) and H₂O (50 mL). The reaction mixture was heated to 40° C. and allowed to stir overnight. LCMS indicated complete consumption of starting material and formation of product with desired mass (60% peak area). Then, the resulting mixture was filtered, and the filter cake was washed with EA. The filtrate was extracted with EA (100 mL×3). The organic layers were combined and washed with water and brine. The organic phase was dried over anhydrous sodium sulfate and concentrated under reduced pressure to give crude residue, which was purified via column chromatography (EA/DCM=1/1) to give N6-(5-bromo-2-chloropyrimidin-4-yl)quinoxaline-5,6-diamine (10 g, 54% yield) as a yellow solid. LCMS: [M+H+]=353.

### Step 4: Preparation of N-(6-((5-bromo-2-chloropyrimidin-4-yl)amino)quinoxalin-5-yl) methanesulfonamide

To a solution of N6-(5-bromo-2-chloro-pyrimidin-4-yl)quinoxaline-5,6-diamine (3 g, 8.53 mmol, 1 equiv) in pyridine (30 mL) was added a solution of methanesulfonic anhydride (2.97 g, 17.07 mmol, 2 equiv) in DCM (20 mL) in a dropwise manner at 0° C. The mixture was allowed to stir at 25° C. for 1 h. LC-MS showed ˜2% of N6-(5-bromo-2-chloro-pyrimidin-4-yl)quinoxaline-5,6-diamine remained. Several new peaks appeared on LC-MS and ˜22% of desired compound was detected. The reaction was concentrated under reduced pressure to give a residue. Then, a solution of the residue in sodium hydroxide (3 M, 100 mL) was allowed to stir at 25° C. for 3 h, adjusted to pH=5 with conc. HCl, extracted with EA (200 mL×3), dried over anhydrous sodium sulfate, and concentrated under reduced pressure to give a brown solid. The brown solid was further triturated with EA (50 mL) for 12 h at 25° C. to give the desired product (2.7 g, 6.10 mmol, 71.43% yield, 97% purity) as a brown solid.

### Preparation of 2-(1-(2,6-dioxopiperidin-3-yl)-3-ethyl-2-oxo-2,3-dihydro-1H-benzo[d]imidazol-4-yl) acetaldehyde (Int-3

### Step 1: Preparation of 2-bromo-N-ethyl-6-nitroaniline

To a solution of 1-bromo-2-fluoro-3-nitro-benzene (25.0 g, 113.64 mmol) in THF (250 mL) was added K₂CO₃ (15.71 g, 113.64 mmol) at 20° C. under N₂. Then, ethanamine hydrochloride (10.19 g, 125.00 mmol) was added portion-wise at 0° C. The resulting mixture was allowed to stir at 20° C. for 16 h under N₂. LCMS showed 86% peak with desired mass. The reaction mixture was poured into water (200 mL) and extracted with EA (200 mL×3). The combined organic layers were washed with brine (150 mL), dried over Na₂SO₄, filtered, and concentrated under reduced pressure to give 2-bromo-N-ethyl-6-nitro-aniline (29 g, crude) as yellow oil. ¹H NMR (400 MHZ, DMSO-d₆) δ 7.82-7.80 (m, 2H), 6.80-6.76 (m, 1H), 5.93 (s, 1H), 3.12-3.07 (m, 2H), 1.13-1.09 (m, 3H).

### Step 2: Preparation of 6-bromo-N1-ethylbenzene-1,2-diamine

A solution of 2-bromo-N-ethyl-6-nitro-aniline (29 g, 118.33 mmol) in EtOAc (300 mL), H₂O (10 mL), and AcOH (100 mL) was allowed to stir at 50° C., then Fe (26.43 g, 473.33 mmol) was added portion-wise. The mixture was allowed to stir at 80° C. for 4 h. LCMS showed 87% peak with desired mass. After cooling to room temperature, the reaction mixture was quenched by saturated solution of Na₂CO₃ (600 mL) and extracted with EA (200 mL×3). The combined organic layers were washed with brine (300 mL×2), dried over Na₂SO₄, filtered, and concentrated to give 6-bromo-N1-ethylbenzene-1,2-diamine (26.61 g, crude) as brown oil.

### Step 3: Preparation of 4-bromo-3-ethyl-1H-benzimidazol-2-one

To a solution of 3-bromo-N2-ethyl-benzene-1,2-diamine (26.6 g, 123.67 mmol) in ACN (400 mL) was added di(imidazol-1-yl) methanone (24.06 g, 148.40 mmol). The reaction mixture was allowed to stir at 80° C. for 16 h. LCMS showed 74% peak with desired mass. After cooling to room temperature, the reaction mixture was concentrated under reduced pressure to give a residue. The residue was triturated with water (1200 mL) at 20° C. for 1 h, then filtered, and the filter cake was collected to give 4-bromo-3-ethyl-1H-benzimidazol-2-one (23 g, 77.14%) as a brown solid. ¹H NMR (400 MHZ, DMSO-d₆) δ 11.19 (s, 1H), 7.18-7.16 (m, 1H), 7.00-6.99 (m, 1H), 6.94-6.90 (m, 1H), 4.14-4.09 (m, 2H), 1.25-1.21 (m, 3H).

### Step 4: Preparation of 3-(4-bromo-3-ethyl-2-oxo-2,3-dihydro-1H-benzo[d]imidazol-1-yl) piperidine-2,6-dione

To a mixture of 4-bromo-3-ethyl-1H-benzimidazol-2-one (2 g, 8.30 mmol) in THF (20 mL) cooled to 0° C. under N₂, LIHMDS (26.40 mL, 1 M in THF) was added dropwise at 0° C. under N₂. After 15 min, a mixture of 3-bromopiperidine-2,6-dione (2.39 g, 12.44 mmol) in THF (4 mL) was added to the reaction mixture. The mixture was allowed to stir at 70° C. for 12 h under N₂. LCMS showed 19% peak with desired mass. The reaction mixture was cooled to 0° C. and quenched with H₂O (30 mL). The mixture was adjusted to PH ˜2-3 using an aqueous solution of HCl (1 M). The mixture was filtered, and the filtrate was concentrated under reduced pressure to give a residue. The residue was purified by column (SiO₂, EA) to give 3-(4-bromo-3-ethyl-2-oxo-benzimidazol-1-yl) piperidine-2,6-dione (250 mg) as a yellow solid. ¹H NMR (400 MHZ, DMSO-d₆) δ 11.13 (s, 1H), 7.27-7.25 (m, 1H), 7.19-7.17 (m, 1H), 7.01-6.97 (m, 1H), 5.44-5.39 (m, 1H), 4.19-4.16 (m, 2H), 2.76-2.54 (m, 2H), 2.05-1.78 (m, 2H), 1.28-1.24 (m, 3H).

### Step 5: Preparation of (Z)-3-(4-(2-ethoxyvinyl)-3-ethyl-2-oxo-2,3-dihydro-1H-benzo[d]imidazol-1-yl)piperidine-2,6-dione

To a mixture of 3-(4-bromo-3-ethyl-2-oxo-benzimidazol-1-yl)piperidine-2,6-dione (200 mg, 567.89 μmol) and 2-[(E)-2-ethoxyvinyl]-4,4,5,5-tetramethyl-1,3,2-dioxaborolane (134.98 mg, 681.46 μmol) in dioxane (2 mL) and H₂O (0.4 mL) was added Pd (dppf) Cl₂ (41.55 mg, 56.79 μmol) and K₂CO₃ (156.97 mg, 1.14 mmol) under N₂. Then, the mixture was allowed to stir at 80° C. for 2 h under N₂. LCMS (EC13404-153-P1A₃) showed 34% peak with desired mass. After cooling to room temperature, the reaction mixture was poured into water (30 mL) and extracted with EA (20 mL×3). The combined organic layers were washed with brine (10 mL×2), dried over Na₂SO₄, filtered, and concentrated under reduced pressure to give a residue. The residue was purified by prep-HPLC (FA conditions) to give 3-[4-[(Z)-2-ethoxyvinyl]-3-ethyl-2-oxo-benzimidazol-1-yl]piperidine-2,6-dione (38 mg, 19.49%) as a white solid. ¹H NMR (400 MHZ, DMSO-d6 400 MHZ) δ 11.08 (s, 1H), 7.00-6.90 (m, 4H), 6.26-6.23 (m, 1H), 5.38-5.34 (m, 1H), 4.07-3.92 (m, 4H), 3.30 (s, 1H), 2.90 (m, 1H), 2.72-2.65 (m, 1H), 2.03-2.00 (m, 1H), 1.31-1.19 (m, 6H).

### Step 6: Preparation of 2-(1-(2,6-dioxopiperidin-3-yl)-3-ethyl-2-oxo-2,3-dihydro-1H-benzo[d]imidazol-4-yl) acetaldehyde

A mixture of 3-[4-[(Z)-2-ethoxyvinyl]-3-ethyl-2-oxo-benzimidazol-1-yl]piperidine-2,6-dione (38 mg, 110.67 μmol) in HCOOH (1 mL) was allowed to stir at 25° C. for 1.5 h. LCMS (EC13404-161-P1A₂) showed 87% peak with desired mass. The reaction mixture was concentrated under reduced pressure to give 2-[1-(2,6-dioxo-3-piperidyl)-3-ethyl-2-oxo-benzimidazol-4-yl]acetaldehyde (32 mg, 91.70%) as a light yellow solid. ¹H NMR (400 MHz, DMSO-d₆) δ 11.10 (s, 1H), 9.77 (s, 1H), 7.09-7.01 (m, 2H), 6.94-6.89 (m, 1H), 5.41-5.36 (m, 1H), 4.09 (s, 2H), 3.91-3.86 (m, 2H), 2.89-2.64 (m, 1H), 2.61-2.59 (m, 1H), 2.03-1.99 (m, 2H), 1.17-1.13 (m, 3H).

### Preparation of N-(6-((5-bromo-2-((5-ethyl-2-methoxy-4-(4-(piperazin-1-yl)piperidin-1-yl)phenyl)amino)pyrimidin-4-yl)amino)quinoxalin-5-yl) methanesulfonamide (Int-4

### Step 1: Preparation of benzyl 4-(1-(4-((5-bromo-4-((5-(methylsulfonamido)quinoxalin-6-yl)amino)pyrimidin-2-yl)amino)-2-ethyl-5-methoxyphenyl)piperidin-4-yl)piperazine-1-carboxylate

N-(6-((5-bromo-2-chloropyrimidin-4-yl)amino)quinoxalin-5-yl) methanesulfonamide (600 mg, 1.4 mmol, 1.0 equiv), benzyl 4-(1-(4-amino-2-ethyl-5-methoxyphenyl)piperidin-4-yl)piperazine-1-carboxylate (682 mg, 1.4 mmol, 1.0 equiv), and MsOH (403 mg, 4.2 mmol, 3.0 equiv) were dissolved in t-BuOH (20 mL). The reaction mixture was degassed with N₂3 times, heated to 90° C., and allowed to stir overnight. LCMS indicated complete consumption of starting material and formation of product with desired mass (40% peak area). Then, the resulting mixture was concentrated and the crude residue was purified via column chromatography (MeOH/DCM=10/1) to give benzyl 4-(1-(4-((5-bromo-4-((5-(methylsulfonamido)quinoxalin-6-yl)amino)pyrimidin-2-yl)amino)-2-ethyl-5-methoxyphenyl)piperidin-4-yl)piperazine-1-carboxylate (1 g, 83% yield) as a yellow solid. LCMS: [M+H+]=847.

### Step 2: Preparation of N-(6-((5-bromo-2-((5-ethyl-2-methoxy-4-(4-(piperazin-1-yl) piperidin-1-yl)phenyl)amino)pyrimidin-4-yl)amino)quinoxalin-5-yl) methanesulfonamide

4-(1-(4-((5-bromo-4-((5-(methylsulfonamido)quinoxalin-6-yl)amino)pyrimidin-2-yl)amino)-2-ethyl-5-methoxyphenyl)piperidin-4-yl)piperazine-1-carboxylate (1 g, 1.2 mmol, 1.0 equiv) was dissolved in HBr (30% in AcOH, 10 mL). The reaction mixture stirred 2 h at 25° C. LCMS indicated complete consumption of starting material and formation of product with desired mass (90% peak area). The resulting mixture was concentrated to give N-(6-((5-bromo-2-((5-ethyl-2-methoxy-4-(4-(piperazin-1-yl)piperidin-1-yl)phenyl)amino)pyrimidin-4-yl)amino)quinoxalin-5-yl) methanesulfonamide as HBr salt (1.1 g) which was used directly in the next step. LCMS: [M+H+]=713.

### Preparation of N-(6-((5-bromo-2-((5-ethyl-2-methoxy-4-(4-oxopiperidin-1-yl)phenyl)amino)pyrimidin-4-yl)amino)quinoxalin-5-yl) methanesulfonamide, Int-5

### Step 1: Preparation of N-(6-((5-bromo-2-((5-ethyl-2-methoxy-4-(1,4-dioxa-8-azaspiro[4.5]decan-8-yl)phenyl)amino)pyrimidin-4-yl)amino)quinoxalin-5-yl) methanesulfonamide

5-Ethyl-2-methoxy-4-(1,4-dioxa-8-azaspiro[4.5]decan-8-yl)aniline (1 g, 3.4 mmol, 1.0 eq), N-(6-((5-bromo-2-chloropyrimidin-4-yl)amino)quinoxalin-5-yl) methanesulfonamide (1.4 g, 3.4 mmol, 1.0 eq), and TFA (1.2 g, 10.2 mmol, 3.0 eq) were dissolved in t-BuOH (10 mL). The reaction mixture was heated to 90° C. and allowed to stir for 5 h. LCMS analysis indicated complete consumption of starting material and formation of product with desired mass. The reaction mixture was concentrated under reduced pressure. The crude residue was purified via column chromatography (DCM:MeOH=10:1) to give N-(6-((5-bromo-2-((5-ethyl-2-methoxy-4-(1,4-dioxa-8-azaspiro[4.5]decan-8-yl)phenyl)amino)pyrimidin-4-yl)amino)quinoxalin-5-yl) methanesulfonamide (1 g, 61 yield) as a yellow solid. LCMS: [M+H+]=687.

### Step 2: Preparation of N-(6-((5-bromo-2-((5-ethyl-2-methoxy-4-(4-oxopiperidin-1-yl)phenyl)amino)pyrimidin-4-yl)amino)quinoxalin-5-yl) methanesulfonamide

N-(6-((5-Bromo-2-((5-ethyl-2-methoxy-4-(1,4-dioxa-8-azaspiro[4.5]decan-8-yl)phenyl)amino)pyrimidin-4-yl)amino)quinoxalin-5-yl) methanesulfonamide (200 mg, 0.29 mmol, 1.0 eq) was dissolved in 2N HCl/THF (2 mL/2 mL). The reaction mixture was heated to 50° C. and allowed to stir 16 h. LCMS analysis indicated complete consumption of starting material and formation of product with desired mass. The reaction mixture was neutralized and extracted with ethyl acetate (10 mL×3). The organic layers were combined and washed with water and brine. The organic phase was dried over anhydrous sodium sulfate and concentrated under reduced pressure to give N-(6-((5-bromo-2-((5-ethyl-2-methoxy-4-(4-oxopiperidin-1-yl)phenyl)amino)pyrimidin-4-yl)amino)quinoxalin-5-yl) methanesulfonamide (170 mg crude) as a yellow oil which was used for next step without further purification. LCMS: [M+H+]=643.

### Preparation of N-(6-((5-bromo-2-((5-ethyl-4-(4-formylpiperidin-1-yl)-2-methoxyphenyl)amino)pyrimidin-4-yl)amino)quinoxalin-5-yl) methanesulfonamide, Int-6

### Step 1: Preparation of 1-(2-bromo-5-methoxy-4-nitrophenyl)-4-(dimethoxymethyl) piperidine

1-Bromo-2-fluoro-4-methoxy-5-nitrobenzene (10 g, 40 mmol, 1.0 eq), 4-(dimethoxymethyl) piperidine (5.7 g, 40 mmol, 1.0 eq), and K₂CO₃ (11 g, 80 mmol, 2.0 eq) were dissolved in MeCN (100 mL). The reaction mixture was stirred at 80° C. for 16 h. LCMS analysis indicated complete consumption of starting material and formation of product with the desired mass. The reaction mixture was filtered. The filtrate was concentrated to give 1-(2-bromo-5-methoxy-4-nitrophenyl)-4-(dimethoxymethyl) piperidine (15 g crude) as a brown solid which was used directly in next step without further purification. LCMS: [M+H]⁺=389.2.

### Step 2: Preparation of 4-(dimethoxymethyl)-1-(5-methoxy-4-nitro-2-vinylphenyl)piperidine

1-(2-Bromo-5-methoxy-4-nitrophenyl)-4-(dimethoxymethyl) piperidine (15 g, 40.2 mmol, 1.0 eq), trifluoro (vinyl)-borane, potassium salt (5.4 g, 40.2 mmol, 1.0 eq), Pd (dppf) Cl₂ (2.9 g, 0.1 mmol, 0.1 eq) and K₃PO₄ (17 g, 80.4 mmol, 2.0 eq) were dissolved in DMF/H₂O (120 mL/30 mL). The reaction mixture was stirred at 100° C. for 16 h under N2 atmosphere. LCMS analysis indicated complete consumption of starting material and formation of product with the desired mass. The reaction mixture was concentrated under reduced pressure and the crude residue was purified via column chromatography (PE:EA=3:1) to give 4-(dimethoxymethyl)-1-(5-methoxy-4-nitro-2-vinylphenyl)piperidine (10 g, 78% yield) as a yellow solid. LCMS: [M+H]⁺=337.3.

### Step 3: Preparation of 4-(4-(dimethoxymethyl) piperidin-1-yl)-5-ethyl-2-methoxyaniline

4-(Dimethoxymethyl)-1-(5-methoxy-4-nitro-2-vinylphenyl)piperidine (10 g, 31.2 mmol, 1.0 eq) and Pd/C (1 g, 10% wet) were dissolved in THF (100 mL). The reaction mixture was stirred at 40° C. for 16 h under H₂ atmosphere. LCMS analysis indicated complete consumption of starting material and formation of product with the desired mass. The reaction mixture was filtered. The filtrate was concentrated to give 4-(4-(dimethoxymethyl) piperidin-1-yl)-5-ethyl-2-methoxyaniline (8 g crude) as a yellow solid which was used directly in next step without further purification. LCMS: [M+H]⁺=309.2.

### Step 4: Preparation of N-(6-((5-bromo-2-((4-(4-(dimethoxymethyl) piperidin-1-yl)-5-ethyl-2-methoxyphenyl)amino)pyrimidin-4-yl)amino)quinoxalin-5-yl) methanesulfonamide

4-(4-(Dimethoxymethyl) piperidin-1-yl)-5-ethyl-2-methoxyaniline (1 g, 3.4 mmol, 1.0 eq), N-(6-((5-bromo-2-chloropyrimidin-4-yl)amino)quinoxalin-5-yl) methanesulfonamide (1.4 g, 3.4 mmol, 1.0 eq) and TFA (1.2 g, 10.2 mmol, 3.0 eq) were dissolved in t-BuOH (10 mL). The reaction mixture was stirred at 90° C. for 5 h. LCMS analysis indicated complete consumption of starting material and formation of product with the desired mass. The reaction mixture was concentrated under reduced pressure. The crude residue was purified via column chromatography (DCM:MeOH=10:1) to give N-(6-((5-bromo-2-((4-(4-(dimethoxymethyl) piperidin-1-yl)-5-ethyl-2-methoxyphenyl)amino)pyrimidin-4-yl)amino)quinoxalin-5-yl) methanesulfonamide (1 g, 61 yield) as a yellow solid. LCMS: [M+H]⁺=701.2.

### Step 5: Preparation of N-(6-((5-bromo-2-((5-ethyl-4-(4-formylpiperidin-1-yl)-2-methoxyphenyl)amino)pyrimidin-4-yl)amino)quinoxalin-5-yl) methanesulfonamide

N-(6-((5-Bromo-2-((4-(4-(dimethoxymethyl) piperidin-1-yl)-5-ethyl-2-methoxyphenyl)amino)pyrimidin-4-yl)amino)quinoxalin-5-yl) methanesulfonamide (200 mg, 0.29 mmol, 1.0 eq) was dissolved in 2N HCl/THF (2 mL/2 mL). The reaction mixture was stirred at 50° C. for 16 h. LCMS analysis indicated complete consumption of starting material and formation of product with the desired mass. The reaction mixture was neutralized and extracted with ethyl acetate (10 mL×3). The organic layers were combined, washed with water and brine. The organic phase was dried over anhydrous sodium sulfate, concentrated under reduced pressure to give N-(6-((5-bromo-2-((5-ethyl-4-(4-formylpiperidin-1-yl)-2-methoxyphenyl)amino)pyrimidin-4-yl)amino)quinoxalin-5-yl) methanesulfonamide (170 mg) as a yellow oil which was used directly in next step without further purification. LCMS: [M+H]⁺=657.3.

### Preparation of Exemplary Compounds

### Example 1: Preparation of N-(6-((5-bromo-2-((4-(4-(4-(2-((2-(2,6-dioxopiperidin-3-yl)-1-oxoisoindolin-5-yl)oxy) ethyl) piperazin-1-yl)piperidin-1-yl)-5-ethyl-2-methoxyphenyl)amino)pyrimidin-4-yl)amino)quinoxalin-5-yl) methanesulfonamide (Compound 1

### Step 1: Preparation of 3-(5-(2-bromoethoxy)-1-oxoisoindolin-2-yl)piperidine-2,6-dione

To a solution of 3-(5-hydroxy-1-oxo-isoindolin-2-yl)piperidine-2,6-dione (150 mg, 576.38 μmol) in DMF (1 mL) was added K₂CO₃ (79.66 mg, 576.38 μmol) and 1,2-dibromoethane (541.40 mg, 2.88 mmol). The mixture was allowed to stir at 70° C. for 16 h. LCMS showed 15% peak area with desired mass. After cooling to room temperature, the reaction mixture was poured into water (20 mL) and extracted with DCM (20 mL×2). The combined organic layers were washed with brine (40 mL), dried over Na₂SO₄, filtered, and concentrated under reduced pressure to give a residue. The residue was purified by prep-TLC (SiO₂, DCM/MeOH=10/1) to afford 3-[5-(2-bromoethoxy)-1-oxo-isoindolin-2-yl]piperidine-2,6-dione (20.0 mg, 9.45% yield) as a white solid. ¹H NMR (400 MHZ, DMSO-d₆) δ 10.97 (s, 1H), 7.95 (s, 1H), 7.64 (d, J=8.4 Hz, 1H), 7.08 (dd, J=2.0, 8.4 Hz, 1H), 5.08 (dd, J=5.2, 13.2 Hz, 1H), 4.42 (t, J=5.6 Hz, 2H), 4.38-4.28 (m, 2H), 3.84 (t, J=5.6 Hz, 2H), 3.39-3.36 (m, 2H), 2.52-2.51 (m, 2H).

### Step 2: Preparation of N-(6-((5-bromo-2-((4-(4-(4-(2-((2-(2,6-dioxopiperidin-3-yl)-1-oxoisoindolin-5-yl)oxy) ethyl) piperazin-1-yl)piperidin-1-yl)-5-ethyl-2-methoxyphenyl)amino)pyrimidin-4-yl)amino)quinoxalin-5-yl) methanesulfonamide

To a solution of 3-[5-(2-bromoethoxy)-1-oxo-isoindolin-2-yl]piperidine-2,6-dione (20.0 mg, 54.47 μmol) in DMF (0.5 mL) was added N-[6-[[5-bromo-2-[5-ethyl-2-methoxy-4-(4-piperazin-1-yl-1-piperidyl)anilino]pyrimidin-4-yl]amino]quinoxalin-5-yl]methanesulfonamide (38.76 mg, 54.47 μmol), K₂CO₃ (7.53 mg, 54.47 μmol), and KI (904.17 μg, 5.45 μmol). The mixture was allowed to stir at 60° C. for 16 h. LCMS showed 43% peak with desired mass. After cooling to room temperature, the reaction mixture was purified by prep-HPLC (FA conditions) to afford N-(6-((5-bromo-2-((4-(4-(4-(2-((2-(2,6-dioxopiperidin-3-yl)-1-oxoisoindolin-5-yl)oxy) ethyl) piperazin-1-yl)piperidin-1-yl)-5-ethyl-2-methoxyphenyl)amino)pyrimidin-4-yl)amino)quinoxalin-5-yl) methanesulfonamide (5.0 mg, 8.79% yield) as a yellow solid. ¹H NMR (400 MHZ, DMSO-d₆) δ 8.99 (s, 1H), 8.89 (s, 1H), 8.64 (d, J=8.8 Hz, 1H), 8.23 (s, 1H), 7.97 (d, J=9.6 Hz, 1H), 7.75 (d, J=8.8 Hz, 1H), 7.64 (s, 1H), 7.19 (s, 1H), 7.13 (d, J=7.6 Hz, 1H), 6.78 (s, 1H), 5.16-5.12 (m, 1H), 4.82-4.75 (m, 1H), 4.48-4.29 (m, 3H), 4.29 (s, 2H), 3.85 (s, 3H), 3.10-3.02 (m, 3H), 2.99-2.77 (m, 15H), 2.52-2.41 (m, 3H), 2.24-2.09 (m, 3H), 1.79-1.78 (m, 2H), 0.77-0.73 (m, 3H).

### Example 2: Preparation of N-(6-((5-bromo-2-((4-(4-((1-(2-(2,6-dioxopiperidin-3-yl)-1-oxoisoindolin-5-yl)piperidin-4-yl)methyl) piperazin-1-yl)-5-ethyl-2-methoxyphenyl)amino)pyrimidin-4-yl)amino)quinoxalin-5-yl) methanesulfonamide (Compound 2

### Step 1: Preparation of methyl 2-cyano-4-(4-(hydroxymethyl) piperidin-1-yl)benzoate

To a solution of methyl 2-cyano-4-fluorobenzoate (2 g, 11.16 mmol) and piperidin-4-yl, methanol (1.93 g, 16.75 mmol) in DMSO (20 mL) was added DIEA (4.33 g, 33.49 mmol) at 20° C. The mixture was allowed to stir at 110° C. for 2 h. LCMS showed 57% peak with desired mass. After cooling to room temperature, the reaction mixture was poured into water (100 mL) and extracted with EA (100 mL×3). The combined organic layers were washed with brine (200 mL×2), dried over Na₂SO₄, filtered, and concentrated under reduced pressure to give crude product. The crude product was purified by column chromatography (SiO₂, PE/EA= 10/1 to ½) to afford methyl 2-cyano-4-(4-(hydroxymethyl) piperidin-1-yl)benzoate (3.41 g) as yellow oil. ¹H NMR (400 MHZ, DMSO-d₆) δ 7.87 (d, J=9.2 Hz, 1H), 7.39 (d, J=2.8 Hz, 1H), 7.20 (dd, J=2.8, 9.2 Hz, 1H), 4.50 (t, J=5.2 Hz, 1H), 4.05-3.98 (m, 2H), 3.82 (s, 3H), 3.26 (t, J=6.0 Hz, 2H), 2.89 (dt, J=2.4, 12.8 Hz, 2H), 1.73-1.67 (m, 2H), 1.67-1.57 (m, 1H), 1.20-1.12 (m, 2H).

### Step 2: Preparation of methyl 2-formyl-4-(4-(hydroxymethyl) piperidin-1-yl)benzoate

To a solution of methyl 2-cyano-4-(4-(hydroxymethyl) piperidin-1-yl)benzoate (1 g, 3.65 mmol) in H₂O (5 mL) and py (10 mL) was added Raney-Ni (434.13 mg, 5.07 mmol) and sodium hypophosphite monohydrate (5.03 g, 36.45 mmol) at 25° C. The mixture was allowed to stir at 25° C. for 20 min. After that, AcOH (5 mL) was added at 25° C. The mixture was allowed to stir at 70° C. for 16 h under N₂. LCMS showed about 59% peak of desired mass. After cooling to room temperature, the reaction mixture was filtered, and the filtrate was concentrated under reduced pressure to give a residue. The residue was poured into water (100 mL) and extracted with EA (100 mL×3). The combined organic layers were washed with brine (200 mL×2), dried over Na₂SO₄, filtered, and concentrated under reduced pressure to give crude product. The crude product was purified by column chromatography (SiO₂, Petroleum ether/EA=20/1 to 1/1) to afford methyl 2-formyl-4-(4-(hydroxymethyl) piperidin-1-yl)benzoate (847 mg, 83% yield) as yellow oil. ¹H NMR (400 MHz, DMSO-d₆) δ10.50 (s, 1H), 7.80 (d, J=8.8 Hz, 1H), 7.21-7.12 (m, 2H), 3.95 (d, J=12.8 Hz, 2H), 3.84 (s, 3H), 3.27 (t, J=5.6 Hz, 2H), 2.87 (dt, J=2.4, 12.8 Hz, 2H), 1.73 (d, J=12.8 Hz, 2H), 1.67-1.56 (m, 1H), 1.20-1.14 (m, 2H).

### Step 3: Preparation of 3-(5-(4-(hydroxymethyl) piperidin-1-yl)-1-oxoisoindolin-2-yl) piperidine-2,6-dione

To a solution of methyl 2-formyl-4-(4-(hydroxymethyl) piperidin-1-yl)benzoate (400 mg, 1.44 mmol) in DCM (24 mL) was added 3-aminopiperidine-2,6-dione hydrochloride (237.40 mg, 1.44 mmol), AcOH (840.21 mg, 13.99 mmol), and DIEA (356.06 mg, 2.75 mmol) at 25° C. The mixture was allowed to stir at 35° C. for 16 h. Then, NaBH(OAc)₃ (920.17 mg, 4.34 mmol) was added to the mixture at 25° C. The mixture was allowed to stir at 25° C. for 16 h. LCMS showed 64% peak of desired mass. The mixture was diluted with water (80 mL), adjusted to pH=8 with saturated NaHCO₃, and extracted with EA (100 mL×3). The combined organic layers were washed with brine (200 mL), dried over Na₂SO₄, filtered, and concentrated under reduced pressure to give a residue. The residue was purified through column chromatography (SiO₂, DCM/MeOH=50/1 to 9/1) to afford 3-(5-(4-(hydroxymethyl) piperidin-1-yl)-1-oxoisoindolin-2-yl) piperidine-2,6-dione (110 mg, 21% yield) as a green solid.

### Step 4: Preparation of 1-(2-(2,6-dioxopiperidin-3-yl)-1-oxoisoindolin-5-yl)piperidine-4-carbaldehyde

To a mixture of 3-(5-(4-(hydroxymethyl) piperidin-1-yl)-1-oxoisoindolin-2-yl) piperidine-2,6-dione (110 mg, 307.78 μmol) in DCM (30 mL) was slowly added DMP (261.08 mg, 615.55 μmol) at 20° C. The mixture was allowed to stir at 35° C. for 1 h. TLC (DCM/MeOH=8/1) showed a major new spot. After cooling to room temperature, the reaction was filtered under vacuum to give a filter head and a filtrate. The filter head was washed with MeOH (20 mL), and the combined filtrate was concentrated under vacuum to give a residue. The residue was purified by prep-TLC (DCM/MeOH=8/1) to afford 1-(2-(2,6-dioxopiperidin-3-yl)-1-oxoisoindolin-5-yl)piperidine-4-carbaldehyde (50 mg, 45% yield) as a brown solid.

### Step 5: Preparation of N-(6-((5-bromo-2-((4-(4-((1-(2-(2,6-dioxopiperidin-3-yl)-1-oxoisoindolin-5-yl)piperidin-4-yl)methyl) piperazin-1-yl)-5-ethyl-2-methoxyphenyl)amino)pyrimidin-4-yl)amino)quinoxalin-5-yl) methanesulfonamide hydrochloride

To a mixture of N-(6-((5-bromo-2-((5-ethyl-2-methoxy-4-(piperazin-1-yl)phenyl)amino)pyrimidin-4-yl)amino)quinoxalin-5-yl) methanesulfonamide (20 mg, 30.08 μmol, HCl) in THF (1 mL) was added TEA (6.09 mg, 60.15 μmol). The mixture was allowed to stir at 20° C. for 30 min. To the mixture was added 1-(2-(2,6-dioxopiperidin-3-yl)-1-oxoisoindolin-5-yl)piperidine-4-carbaldehyde (10.69 mg, 30.08 μmol) and AcOH (3.61 mg, 60.15 μmol). The mixture was allowed to stir at 20° C. for 10 h. To the mixture was added another batch of NaBH(OAc)₃ (31.87 mg, 150.38 μmol). The mixture was allowed to stir at 20° C. for 10 h. LCMS showed about 50% peak with desired mass. The reaction mixture was concentrated under vacuum to give a residue. The residue was purified by prep-HPLC (HCl conditions) to afford N-(6-((5-bromo-2-((4-(4-((1-(2-(2,6-dioxopiperidin-3-yl)-1-oxoisoindolin-5-yl)piperidin-4-yl)methyl) piperazin-1-yl)-5-ethyl-2-methoxyphenyl)amino)pyrimidin-4-yl)amino)quinoxalin-5-yl) methanesulfonamide (9.2 mg, 2HCl salt, 29% yield) as a white solid. ¹H NMR (400 MHZ, DMSO-d₆) δ 10.96 (s, 1H), 10.25-10.06 (m, 1H), 9.98 (s, 1H), 9.27-9.13 (m, 1H), 9.10-8.93 (m, 2H), 8.90-8.34 (m, 3H), 7.94 (d, J=8.8 Hz, 1H), 7.64-7.40 (m, 2H), 7.13 (s, 2H), 6.76 (s, 1H), 5.06 (dd, J=5.2, 13.2 Hz, 1H), 4.32-4.24 (m, 2H), 3.93 (d, J=12.8 Hz, 2H), 3.82 (s, 3H), 3.62-3.60 (m, 2H), 3.37-3.02 (m, 13H), 2.93-2.91 (m, 3H), 2.39-2.37 (m, 2H), 2.22-2.09 (m, 1H), 2.03-1.87 (m, 3H), 1.47-1.31 (m, 2H), 0.85-0.78 (m, 3H).

### Example 3: Preparation of N-(6-((5-bromo-2-((4-(4-(4-(2-(2-(2,6-dioxopiperidin-3-yl)-1,3-dioxoisoindolin-4-yl)ethyl) piperazin-1-yl)piperidin-1-yl)-5-ethyl-2-methoxyphenyl)amino)pyrimidin-4-yl)amino)quinoxalin-5-yl) methanesulfonamide (Compound 3

### Step 1: Preparation of (E)-2-(2,6-dioxopiperidin-3-yl)-4-(2-ethoxyvinyl) isoindoline-1,3-dione

To a mixture of 4-bromo-2-(2,6-dioxo-3-piperidyl) isoindoline-1,3-dione (300 mg, 889.88 μmol) and 2-[(E)-2-ethoxyvinyl]-4,4,5,5-tetramethyl-1,3,2-dioxaborolane (211.51 mg, 1.07 mmol) in dioxane (3 mL) and H₂O (0.6 mL) was added Pd (dppf) Cl₂ (65.11 mg, 88.99 μmol) and K₂CO₃ (245.97 mg, 1.78 mmol) under N₂. Then, the mixture was allowed to stir at 80° C. for 2 h under N₂. LCMS showed 47% peak with desired mass. After cooling to room temperature, the reaction mixture was poured into water (30 mL) and extracted with EA (20 mL×3). The combined organic layers were washed with brine (20 ml×2), dried over Na₂SO₄, filtered, and concentrated under reduced pressure to give a residue. The residue was purified by prep-HPLC (NH₄HCO₃ conditions) to give (E)-2-(2,6-dioxopiperidin-3-yl)-4-(2-ethoxyvinyl) isoindoline-1,3-dione (17 mg, 5.82% yield) as a yellow solid. ¹H NMR (400 MHz, DMSO-d₆) δ 11.09 (m, 1H), 8.02-8.00 (m, 1H), 7.75-7.67 (m, 2H), 7.62-7.60 (m, 1H), 6.78-6.75 (m, 1H), 5.14-5.09 (m, 1H), 4.03-3.97 (m, 2H), 2.90-2.84 (m, 1H), 2.61-2.53 (m, 2H), 2.05-2.03 (m, 1H), 1.31-1.28 (m, 3H).

### Step 2: Preparation of 2-(2-(2,6-dioxopiperidin-3-yl)-1,3-dioxoisoindolin-4-yl) acetaldehyde

A solution of (E)-2-(2,6-dioxopiperidin-3-yl)-4-(2-ethoxyvinyl) isoindoline-1,3-dione (17 mg, 51.78 μmol) in HCOOH (1 mL) was allowed to stir at 25° C. for 2 h. LCMS showed 96% peak with desired mass. The reaction mixture was concentrated under reduced pressure to give 2-(2-(2,6-dioxopiperidin-3-yl)-1,3-dioxoisoindolin-4-yl) acetaldehyde (13 mg, 83.62%) as a yellow solid.

### Step 3: Preparation of N-(6-((5-bromo-2-((4-(4-(4-(2-(2-(2,6-dioxopiperidin-3-yl)-1,3-dioxoisoindolin-4-yl)ethyl) piperazin-1-yl)piperidin-1-yl)-5-ethyl-2-methoxyphenyl)amino)pyrimidin-4-yl)amino)quinoxalin-5-yl) methanesulfonamide

To a mixture of N-[6-[[5-bromo-2-[5-ethyl-2-methoxy-4-(4-piperazin-1-yl-1-piperidyl)anilino]pyrimidin-4-yl]amino]quinoxalin-5-yl]methanesulfonamide (24.92 mg, 33.30 μmol, HCl salt) in THF (1 mL) was added TEA (10.11 mg, 99.91 μmol). The mixture was allowed to stir at 25° C. for 0.5 h. Then, 2-[2-(2,6-dioxo-3-piperidyl)-1,3-dioxo-isoindolin-4-yl]acetaldehyde (10 mg, 33.30 μmol), AcOH (8.00 mg, 133.22 μmol), and 4 ÅMS (20 mg) was added. The mixture was allowed to stir at 25° C. for 16 h, before NaBH(OAc)₃ (35.29 mg, 166.52 μmol) was added, and the mixture was allowed to stir at 25° C. for another 2 h. After that, a second batch of NaBH(OAc)₃ (7.06 mg, 33.30 μmol) was added and the mixture was allowed to stir at 25° C. for another 1 h. LCMS showed 62% peak with desired mass. The reaction mixture was concentrated under reduced pressure to afford a residue. The residue was purified by prep-HPLC (FA conditions) to give N-(6-((5-bromo-2-((4-(4-(4-(2-(2-(2,6-dioxopiperidin-3-yl)-1,3-dioxoisoindolin-4-yl)ethyl) piperazin-1-yl)piperidin-1-yl)-5-ethyl-2-methoxyphenyl)amino)pyrimidin-4-yl)amino)quinoxalin-5-yl) methanesulfonamide (1.3 mg, FA salt, 8.36% yield) as a yellow solid. ¹H NMR (400 MHZ, DMSO-d₆) δ 11.13 (s, 1H), 8.96-8.93 (m, 3H), 8.67-8.65 (m, 1H), 8.27-8.19 (m, 3H), 7.80-7.73 (m, 5H), 7.40 (s, 1H), 6.78 (s, 1H), 5.16-5.12 (m, 1H), 3.76 (s, 3H), 3.24-3.01 (m, 2H), 2.68-2.67 (m, 6H), 2.59-2.58 (m, 11H), 2.46-2.42 (m, 4H), 2.36-2.30 (m, 1H), 1.89-1.83 (m, 1H), 1.84-1.83 (m, 2H), 1.58-1.55 (m, 2H), 0.87-0.84 (m, 3H).

### Example 4: Preparation of N-(6-((5-bromo-2-((4-(4-(4-(5-((2-(2,6-dioxopiperidin-3-yl)-1-oxoisoindolin-5-yl)oxy) pentyl) piperazin-1-yl)piperidin-1-yl)-5-ethyl-2-methoxyphenyl)amino)pyrimidin-4-yl)amino)quinoxalin-5-yl) methanesulfonamide (Compound 4

### Step 1: Preparation of 3-(5-((5-bromopentyl)oxy)-1-oxoisoindolin-2-yl)piperidine-2,6-dione

To a solution of 3-(5-hydroxy-1-oxo-isoindolin-2-yl)piperidine-2,6-dione (250 mg, 960.63 μmol) in DMF (3 mL) was added K₂CO₃ (132.76 mg, 960.63 μmol) and 1,5-dibromopentane (1.10 g, 4.80 mmol, 650.44 μL). The mixture was allowed to stir at 70° C. for 16 h. LCMS showed 23% peak with desired mass. The reaction mixture was poured into water (10 mL) and extracted with EA (10 mL×2). The combined organic layers were washed with brine (10 mL×2), dried over Na₂SO₄, filtered, and concentrated. The residue was purified by column chromatography (SiO₂, DCM/MeOH=99/1 to 10/1) to afford 3-(5-((5-bromopentyl)oxy)-1-oxoisoindolin-2-yl)piperidine-2,6-dione (85.0 mg, 21.62% yield) as yellow oil.

### Step 2: Preparation of N-(6-((5-bromo-2-((4-(4-(4-(5-((2-(2,6-dioxopiperidin-3-yl)-1-oxoisoindolin-5-yl)oxy) pentyl) piperazin-1-yl)piperidin-1-yl)-5-ethyl-2-methoxyphenyl)amino)pyrimidin-4-yl)amino)quinoxalin-5-yl) methanesulfonamide

To a solution of 3-[5-(5-bromopentoxy)-1-oxo-isoindolin-2-yl]piperidine-2,6-dione (25 mg, 61.08 μmol) in DMF (0.5 mL) was added N-[6-[[5-bromo-2-[5-ethyl-2-methoxy-4-(4-piperazin-1-yl-1-piperidyl)anilino]pyrimidin-4-yl]amino]quinoxalin-5-yl]methanesulfonamide (43.47 mg, 61.08 μmol, HCl salt), K₂CO₃ (8.44 mg, 61.08 μmol), and KI (1.01 mg, 6.11 μmol). The mixture was allowed to stir at 60° C. for 4 h. LCMS showed 25% peak with desired mass. After cooling to room temperature and filtration, the filtrate was purified by prep-HPLC (FA conditions) to afford N-(6-((5-bromo-2-((4-(4-(4-(5-((2-(2,6-dioxopiperidin-3-yl)-1-oxoisoindolin-5-yl)oxy) pentyl) piperazin-1-yl)piperidin-1-yl)-5-ethyl-2-methoxyphenyl)amino)pyrimidin-4-yl)amino)quinoxalin-5-yl) methanesulfonamide (3.1 mg, 4.66% yield) as a yellow solid. ¹H NMR (400 MHZ, DMSO-d₆) δ 10.97 (br, 1H), 8.97-8.95 (m, 2H), 8.62 (s, 2H), 8.33 (s, 1H), 8.28 (d, J=3.2 Hz, 1H), 8.20 (s, 1H), 7.97-7.90 (m, 1H), 7.55-7.52 (m, 1H), 7.41 (s, 1H), 6.94 (s, 1H), 6.84-6.82 (m, 1H), 6.75 (s, 1H), 5.06 (dd, J=5.2, 13.2 Hz, 1H), 4.31-4.23 (m, 2H), 4.04-3.99 (m, 1H), 3.74-3.73 (m, 6H), 3.17 (s, 3H), 3.03-2.87 (m, 5H), 2.79-2.75 (m, 4H), 2.60-2.50 (m, 4H), 2.32-2.22 (m, 3H), 1.99-1.94 (m, 1H), 1.85-1.78 (m, 2H), 1.63-1.44 (m, 4H), 1.41-1.26 (m, 6H), 0.75-0.60 (m, 3H).

### Example 5: Preparation of N-(6-((5-bromo-2-((4-(4-(4-(2-(2-(2,6-dioxopiperidin-3-yl)-1-oxoisoindolin-5-yl)ethyl) piperazin-1-yl)piperidin-1-yl)-5-ethyl-2-methoxyphenyl)amino)pyrimidin-4-yl)amino)quinoxalin-5-yl) methanesulfonamide (Compound 5

### Step 1: Preparation of (E)-3-(5-(2-ethoxyvinyl)-1-oxoisoindolin-2-yl)piperidine-2,6-dione

To a solution of 2-[(E)-2-ethoxyvinyl]-4,4,5,5-tetramethyl-1,3,2-dioxaborolane (122.59 mg, 618.92 μmol) in DMF (2 mL) was added 3-(5-bromo-1-oxo-isoindolin-2-yl) piperidine-2,6-dione (200 mg, 618.92 μmol), K₃PO₄ (157.65 mg, 742.71 μmol), and Pd (dppf) Cl₂ (54.34 mg, 74.27 μmol). The reaction was degassed and purged with N₂ and then stirred at 90° C. for 16 h. LCMS showed 62% peak with desired mass. After cooling to room temperature, the reaction was diluted with water (30 mL) and extracted with EA (10 mL×3). The combined organic layers were washed with brine (10 mL×2) and dried over Na₂SO₄, filtered, and concentrated under reduced pressure to give a residue. The residue was purified by prep-HPLC (FA conditions) to give (E)-3-(5-(2-ethoxyvinyl)-1-oxoisoindolin-2-yl)piperidine-2,6-dione (80 mg, 41.12%) as a white solid.

### Step 2: Preparation of 2-(2-(2,6-dioxopiperidin-3-yl)-1-oxoisoindolin-5-yl) acetaldehyde

A mixture of 3-[5-[(E)-2-ethoxyvinyl]-1-oxo-isoindolin-2-yl]piperidine-2,6-dione (20 mg, 63.63 μmol) in HCOOH (1 mL) was allowed to stir at 25° C. for 2 h. LCMS showed 94% peak with desired mass. The reaction mixture was concentrated under reduced pressure to give 2-(2-(2,6-dioxopiperidin-3-yl)-1-oxoisoindolin-5-yl) acetaldehyde (18 mg, 98.82%) as a yellow solid. ¹H NMR (400 MHZ, DMSO-d₆) δ 9.83 (s, 1H), 7.93-7.91 (m, 2H), 7.38-7.36 (m, 2H), 5.28-5.23 (m, 1H), 4.56-4.36 (m, 2H), 3.87 (s, 2H), 2.94-2.86 (m, 2H), 2.41-2.24 (m, 2H).

### Step 3: Preparation of N-(6-((5-bromo-2-((4-(4-(4-(2-(2-(2,6-dioxopiperidin-3-yl)-1-oxoisoindolin-5-yl)ethyl) piperazin-1-yl)piperidin-1-yl)-5-ethyl-2-methoxyphenyl)amino)pyrimidin-4-yl)amino)quinoxalin-5-yl) methanesulfonamide

To a mixture of N-(6-((5-bromo-2-((5-ethyl-2-methoxy-4-(4-(piperazin-1-yl) piperidin-1-yl)phenyl)amino)pyrimidin-4-yl)amino)quinoxalin-5-yl) methanesulfonamide (47.04 mg, 62.88 μmol, HCl salt) in THF (1 mL) was added TEA (19.09 mg, 188.63 μmol, 26.25 μL), and the mixture was allowed to stir at 25° C. for 0.5 h. Then, 2-[2-(2,6-dioxo-3-piperidyl)-1-oxo-isoindolin-5-yl]acetaldehyde (18 mg, 62.88 μmol), HOAc (15.10 mg, 251.50 μmol, 14.40 L), and 4 Å MS (20 mg) was added. The mixture was allowed to stir at 25° C. for 12 h. Then, NaBH(OAc)₃ (66.63 mg, 314.38 μmol) was added, and the mixture was allowed to stir at 25° C. for further 2 h. LCMS showed 49% peak with desired mass. The reaction mixture was concentrated under reduced pressure to give a residue. The residue was purified by prep-HPLC (FA conditions) to give N-(6-((5-bromo-2-((4-(4-(4-(2-(2-(2,6-dioxopiperidin-3-yl)-1-oxoisoindolin-5-yl)ethyl) piperazin-1-yl)piperidin-1-yl)-5-ethyl-2-methoxyphenyl)amino)pyrimidin-4-yl)amino)quinoxalin-5-yl) methanesulfonamide (13.3 mg, FA salt) as a yellow solid. ¹H NMR (400 MHZ, DMSO-d₆) δ 10.98 (s, 1H), 8.98-8.89 (m, 3H), 8.67-8.65 (m, 1H), 8.28-21 (m, 2H), 8.20 (s, 1H), 7.86-7.84 (m, 1H), 7.65-7.63 (m, 1H), 7.47-7.37 (m, 3H), 6.78 (s, 1H), 5.13-5.08 (m, 1H), 4.45-4.27 (m, 2H), 3.76 (s, 3H), 3.02-2.98 (m, 6H), 2.88-2.87 (m, 3H), 2.68-2.55 (m, 11H), 2.46-2.43 (m, 4H), 2.45-2.35 (m, 1H), 1.99-1.87 (m, 1H), 1.90-1.80 (m, 2H), 1.58-1.56 (m, 2H), 0.85 (s, 3H).

### Example 6: Preparation of N-(6-((5-bromo-2-((4-(4-(4-((1-(2-(2,6-dioxopiperidin-3-yl)-1,3-dioxoisoindolin-5-yl)piperidin-4-yl)methyl) piperazin-1-yl)piperidin-1-yl)-5-ethyl-2-methoxyphenyl)amino)pyrimidin-4-yl)amino)quinoxalin-5-yl) methanesulfonamide (Compound 6

### Step 1: Preparation of 5-(4-(dimethoxymethyl) piperidin-1-yl)-2-(2,6-dioxopiperidin-3-yl) isoindoline-1,3-dione

2-(2,6-dioxopiperidin-3-yl)-5-fluoroisoindoline-1,3-dione (1.000 g, 3.620 mmol, 1 equiv) and 4-(dimethoxymethyl) piperidine (864.691 mg, 5.430 mmol, 1.5 equiv) were dissolved in DMSO (20 ml) at room temperature. Then, DIEA (935.848 mg, 7.241 mmol, equiv) was slowly added. After degassing with N₂ for three times, the reaction mixture was allowed to stir at 120° C. overnight. TLC and LCMS analysis indicated no starting material remained and formation of desired product. The reaction was cooled to room temperature, extracted three times with EA, and washed with water. The combined organic layer were washed once with saturated brine, dried with anhydrous sodium sulfate, concentrated in vacuo, and passed through column chromatographic purification (PE:EA=4:1) to give 5-(4-(dimethoxymethyl) piperidin-1-yl)-2-(2,6-dioxopiperidin-3-yl) isoindoline-1,3-dione (1.300 g, yield 86.44%) as a yellow solid.

### Step 2: Preparation of 1-(2-(2,6-dioxopiperidin-3-yl)-1,3-dioxoisoindolin-5-yl)piperidine-4-carbaldehyde

5-(4-(dimethoxymethyl) piperidin-1-yl)-2-(2,6-dioxopiperidin-3-yl) isoindoline-1,3-dione (500 mg, 1.204 mmol, 1 equiv) was dissolved in 2 N hydrochloride (10 mL) and tetrahydrofuran (10 ml) solution and heated to 60° C. for 5 h. The reaction was monitored by TLC and LCMS. The target compound was produced and no raw materials remained. Then, the pH was adjusted to neutral with saturated aqueous sodium bicarbonate solution, extracted three times with EA and water, washed with saturated brine, and the organic phase was collected. Drying over sodium sulfate and concentrating in vacuo to give 1-(2-(2,6-dioxopiperidin-3-yl)-1,3-dioxoisoindolin-5-yl)piperidine-4-carbaldehyde (400 mg, 90% yield) as a white solid.

### Step 3: Preparation of N-(6-((5-bromo-2-((4-(4-(4-(1-(2-(2,6-dioxopiperidin-3-yl)-1,3-dioxoisoindoline-5-yl)piperidin-4-yl)methyl) piperazin-1-yl)piperidin-1-yl)-5-ethyl-2-methoxybenzene yl)amino)pyrimidin-4-yl)amino)quinoxalin-5-yl) methanesulfonamide

N-(6-((5-bromo-2-((5-ethyl-2-methoxy-4-(4-(piperazin-1-yl)piperidin-1-yl)phenyl)amino)pyrimidin-4-yl)amino)quinoxalin-5-yl) methanesulfonamide (214.576 mg, 0.271 mmol, 1 equiv) and DIEA (34.991 mg, 0.271 mmol, 1 equiv) were dissolved in MeOH (10 ml) at 15° C. for 15 min. 1-(2-(2,6-Dioxopiperidin-3-yl)-1,3-dioxoisoindolin-5-yl) piperidine-4-carbaldehyde (120 mg, 0.325 mmol, 1.2 equiv) and AcOH (40.642 mg, 0.677 mmol, 2.5 equiv) were added and stirred for 1 h. Then, NaBH₃CN (33.976 mg, 0.541 mmol, 2 equiv) was added and stirred for 3 h. The reaction was monitored by TLC and LCMS. The reaction was stopped after no starting material remained. Volatiles were removed under reduced pressure, and the residue was purified by Prep-HPLC to obtain N-(6-((5-bromo-2-((4-(4-(4-(1-(2-(2,6-dioxopiperidin-3-yl)-1,3-dioxoisoindoline-5-yl) piperidin-4-yl)methyl) piperazin-1-yl)piperidin-1-yl)-5-ethyl-2-methoxybenzene yl)amino)pyrimidin-4-yl)amino)quinoxalin-5-yl) methanesulfonamide (46 mg, yield 18.25%) as a yellow solid. LCMS: [M+H+]=1065.06. H₁NMR (400 MHZ, DMSO-d₆) δ 11.09 (s, 1H), 9.01 (d, J=1.6 Hz, 2H), 8.94 (d, J=1.6 Hz, 1H), 8.66 (s, 1H), 8.27 (d, J=26.4 Hz, 2H), 7.98 (d, J=8.4 Hz, 1H), 7.88 (d, J=9.2 Hz, 1H), 7.66 (d, J=8.4 Hz, 2H), 7.40 (s, 1H), 7.32-7.23 (m, 1H), 6.79 (s, 1H), 5.10-5.06 (m, 2H), 4.05 (d. J=12.0 Hz, 3H), 3.77 (s, 3H), 3.03 (s, 6H), 3.02-2.84 (m, 2H), 2.70 (t, J=11.2 Hz, 2H), 2.57 (d, J=6.8 Hz, 3H), 2.46-2.34 (m, 6H), 2.15 (d, J=6.8 Hz, 2H), 2.07-1.98 (m, 2H), 1.84 (d. J=17.6 Hz, 5H), 1.59 (t, J=11.2 Hz, 2H), 1.25 (s, 1H), 1.15 (d, J=12.4 Hz, 2H), 0.89-0.81 (m, 3H).

### Example 7: Preparation of N-(6-((5-bromo-2-((4-(4-(4-((1-(2-(2,6-dioxopiperidin-3-yl)-1-oxoisoindolin-5-yl)piperidin-4-yl)methyl) piperazin-1-yl)piperidin-1-yl)-5-ethyl-2-methoxyphenyl)amino)pyrimidin-4-yl)amino)quinoxalin-5-yl) methanesulfonamide (Compound 7

### Step 1: Preparation of 1-(2-(2,6-dioxopiperidin-3-yl)-1-oxoisoindolin-5-yl)piperidine-4-carbaldehyde

3-(5-(4-(dimethoxymethyl) piperidin-1-yl)-1-oxoisoindolin-2-yl)piperidine-2,6-dione (600 mg, 1.50 mmol, 1.0 equiv) was dissolved in 2 N hydrochloride/THF (10 mL/10 mL). The reaction mixture was allowed to stir at 25° C. for 16 h. The reaction mixture was neutralized with saturated aqueous sodium bicarbonate solution. The resulting mixture was extracted with EA (20 mL×3) and washed with water and saturated brine. The organic phase was dried over sodium sulfate and concentrated in vacuo to give 1-(2-(2,6-dioxopiperidin-3-yl)-1-oxoisoindolin-5-yl)piperidine-4-carbaldehyde (500 mg, 94% yield) as a brown solid.

### Step 2: Preparation of N-(6-((5-bromo-2-((4-(4-(4-((1-(2-(2,6-dioxopiperidin-3-yl)-1-oxoisoindolin-5-yl)piperidin-4-yl)methyl) piperazin-1-yl)piperidin-1-yl)-5-ethyl-2-methoxyphenyl)amino)pyrimidin-4-yl)amino)quinoxalin-5-yl) methanesulfonamide

To a solution of 1-(2-(2,6-dioxopiperidin-3-yl)-1-oxoisoindolin-5-yl)piperidine-4-carbaldehyde (500 mg, 1.41 mmol, 1.0 equiv) in DCM (10 mL) were added N-(6-((5-bromo-2-((5-ethyl-2-methoxy-4-(4-(piperazin-1-yl)piperidin-1-yl)phenyl)amino)pyrimidin-4-yl)amino)quinoxalin-5-yl) methanesulfonamide (1 g, 1.41 mmol, 1.0 equiv) and DIEA (273 mg, 2.12 mmol, 1.5 equiv). Then, the reaction mixture was allowed to stir at 25° C. for 0.5 h. Then, AcOH (85 mg, 1.41 mmol, 1.0 equiv) was added, and the reaction mixture was allowed to stir for another 0.5 h. Then, NaBH(OAc)₃ (600 mg, 2.82 mmol, 2.0 equiv) was added, and the reaction mixture was allowed to stir for another 15 h. LCMS indicated complete consumption of starting material and formation of product with desired mass (65% peak area). The reaction mixture was concentrated under vacuum and the resulting crude residue was purified by flash column (DCM/MeOH=10/1) to give N-(6-((5-bromo-2-((4-(4-(4-((1-(2-(2,6-dioxopiperidin-3-yl)-1-oxoisoindolin-5-yl)piperidin-4-yl)methyl) piperazin-1-yl)piperidin-1-yl)-5-ethyl-2-methoxyphenyl)amino)pyrimidin-4-yl)amino)quinoxalin-5-yl) methanesulfonamide (300 mg, 20% yield) as a yellow solid. LCMS: [M+H+]=1050.45. ¹H NMR (400 MHZ, DMSO-d₆) § 10.95 (s, 1H), 8.99 (d, J=2.0 Hz, 1H), 8.95-8.84 (m, 2H), 8.64 (s, 1H), 8.29 (s, 1H), 8.20 (d, J=13.2 Hz, 1H), 7.86 (d, J=9.2 Hz, 1H), 7.53-7.47 (m, 1H), 7.39 (s, 1H), 7.07-7.00 (m, 2H), 6.78 (s, 1H), 5.04 (dd, J=13.2, 5.2 Hz, 1H), 4.32 (d, J=16.8 Hz, 1H), 4.24-4.11 (m, 1H), 3.87 (d, J=12.0 Hz, 2H), 3.76 (s, 3H), 3.04-3.01 (m, 5H), 2.91-2.80 (m, 3H), 2.70-2.65 (m, 2H), 2.59-2.52 (m, 4H), 2.47-2.26 (m, 8H), 2.14 (d, J=6.4 Hz, 2H), 2.04-1.90 (m, 1H), 1.90-1.82 (m, 2H), 1.81-1.66 (m, 4H), 1.63-1.50 (m, 2H), 1.36-1.09 (m, 3H), 0.85 (q, J=7.2 Hz, 3H).

### Example 8: Preparation of N-(6-((5-bromo-2-((4-(4-(4-(2-(2-(2,6-dioxopiperidin-3-yl)-3-oxoisoindolin-4-yl)ethyl) piperazin-1-yl)piperidin-1-yl)-5-ethyl-2-methoxyphenyl)amino)pyrimidin-4-yl)amino)quinoxalin-5-yl) methanesulfonamide (Compound 8

### Step 1: Preparation of (Z)-3-(7-(2-ethoxyvinyl)-1-oxoisoindolin-2-yl)piperidine-2,6-dione

3-(7-bromo-1-oxoisoindolin-2-yl)piperidine-2,6-dione (1.000 g, 3.095 mmol, 1 equiv) was dissolved in dioxane/water (6 mL/3 mL). Subsequently, (E)-2-(2-ethoxyvinyl)-4,4,5,5-tetramethyl-1,3,2-dioxaborolane (919.431 mg, 3.095 mmol, 1 equiv), Pd (dppf) Cl₂ (226.434 mg, 0.309 mmol, 0.1 equiv), and K₂CO₃ (1.281 g, 9.284 mmol, 3 equiv) were added under N₂. The reaction mixture was allowed to stir for 6 h at 65° C. under nitrogen atmosphere and then concentrated under vacuum. The so-obtained residue was purified by flash column chromatography on silica gel, eluting with EA/PE (1:3) to give (Z)-3-(7-(2-ethoxyvinyl)-1-oxoisoindolin-2-yl)piperidine-2,6-dione (800 mg, yield 82%) as an orange solid. LCMS: [M+H+]=315.

### Step 2: Preparation of 2-(2-(2,6-dioxopiperidin-3-yl)-3-oxoisoindolin-4-yl) acetaldehyde

(Z)-3-(7-(2-ethoxyvinyl)-1-oxoisoindolin-2-yl)piperidine-2,6-dione (400 mg, 1.273 mmol, 1 equiv) was dissolved in HCOOH (2 mL), and the resulting solution was allowed to stir for 2 h at room temperature. LCMS indicated complete consumption of starting material and formation of product with desired mass (85% peak area). The reaction solution was evaporated to dryness to afford product 2-(2-(2,6-dioxopiperidin-3-yl)-3-oxoisoindolin-4-yl) acetaldehyde (300 mg, yield 82%) as a brown solid, which was used directly in the next step without further purification. LCMS: [M+H+]=287.

### Step 3: Preparation of N-(6-((5-bromo-2-((4-(4-(4-(2-(2-(2,6-dioxopiperidin-3-yl)-3-oxoisoindolin-4-yl)ethyl) piperazin-1-yl)piperidin-1-yl)-5-ethyl-2-methoxyphenyl)amino)pyrimidin-4-yl)amino)quinoxalin-5-yl) methanesulfonamide

A mixture of N-(6-((5-bromo-2-((5-ethyl-2-methoxy-4-(4-(piperazin-1-yl)piperidin-1-yl)phenyl)amino)pyrimidin-4-yl)amino)quinoxalin-5-yl) methanesulfonamide (745.761 mg, 1.048 mmol, 1.5 equiv) and TEA (211.673 mg, 2.096 mmol, 3 equiv) in MeOH (5 mL) was allowed to stir at 25° C. for 20 min. Then, AcOH (62.873 mg, 1.048 mmol, 1.5 equiv) and 2-(2-(2,6-dioxopiperidin-3-yl)-3-oxoisoindolin-4-yl) acetaldehyde (200 mg, 0.699 mmol, 1 equiv) were added. The mixture was allowed to stir at 25° C. for 0.5 h. Then, NaBH₃CN (57.487 mg, 0.908 mmol, 1.3 equiv) was added, and the mixture was allowed to stir at 25° C. for another 1 h. LCMS indicated complete consumption of starting material and formation of product with desired mass (35% peak area). The reaction mixture was concentrated under reduced pressure to afford a residue. The residue was purified by Prep-HPLC to give N-(6-((5-bromo-2-((4-(4-(4-(2-(2-(2,6-dioxopiperidin-3-yl)-3-oxoisoindolin-4-yl)ethyl) piperazin-1-yl)piperidin-1-yl)-5-ethyl-2-methoxyphenyl)amino)pyrimidin-4-yl)amino)quinoxalin-5-yl) methanesulfonamide (34.100 mg, yield 4.9%) as a yellow solid. LCMS: [M+H+]=981. ¹H NMR (400 MHZ, DMSO-d₆) δ 11.00 (s, 1H), 9.01 (d, J=2.0 Hz, 1H), 8.94 (d, J=2.0 Hz, 1H), 8.87 (s, 1H), 8.65 (s, 1H), 8.30 (s, 1H), 8.22 (s, 1H), 7.88 (d, J=9.2 Hz, 1H), 7.53 (t, J=7.2 Hz, 1H), 7.45-7.38 (m, 2H), 7.33 (d, J=7.2 Hz, 1H), 6.79 (s, 1H), 5.11 (dd, J=13.2, 5.2 Hz, 1H), 4.41 (d, J=17.2 Hz, 1H), 4.28 (d, J=17.2 Hz, 1H), 3.77 (s, 3H), 3.08-2.89 (m, 7H), 2.74-2.52 (m, 13H), 2.49-2.35 (m, 6H), 2.06-1.99 (m, 1H), 1.92-1.75 (m, 2H), 1.66-1.54 (m, 2H), 0.85 (s, 3H).

### Example 9: Preparation of N-(6-((5-bromo-2-((4-(4-(4-(2-(2-(2,6-dioxopiperidin-3-yl)-1-oxoisoindolin-4-yl)ethyl) piperazin-1-yl)piperidin-1-yl)-5-ethyl-2-methoxyphenyl)amino)pyrimidin-4-yl)amino)quinoxalin-5-yl) methanesulfonamide (Compound 9

### Step 1: Preparation of (Z)-3-(4-(2-ethoxyvinyl)-1-oxoisoindolin-2-yl)piperidine-2,6-dione

3-(4-bromo-1-oxoisoindolin-2-yl)piperidine-2,6-dione (1.000 g, 3.095 mmol, 1 equiv) and (E)-2-(2-ethoxyvinyl)-4,4,5,5-tetramethyl-1,3,2-dioxaborolane (919.431 mg, 4.642 mmol, 1.5 equiv) were dissolved in dioxane (45 ml) and H₂O (5 ml) at room temperature, K₂CO₃ (1.283 g, 9.284 mmol, 3 equiv), and Pd (dppf) Cl₂ (229.557 mg, 0.309 mmol, 0.1 equiv) were added, and the reaction mixture was degassed with N₂ for three times. The mixture was allowed to stir at 50° C. for 5 h. TLC (MeOH/DCM= 1/10) showed the starting material was consumed completely and a newly generated spot was detected. The solvent was concentrated under vacuum, and the residue was purified by flash column chromatography on silica gel (12 g), eluting with MeOH/DCM ( 1/20) to give (Z)-3-(4-(2-ethoxyvinyl)-1-oxoisoindolin-2-yl)piperidine-2,6-dione (900 mg, yield 92.52%) as a yellow solid. LCMS: [M+H+]=315.34.

### Step 2: Preparation of 2-(2-(2,6-dioxopiperidin-3-yl)-1-oxoisoindolin-4-yl) acetaldehyde

(Z)-3-(4-(2-ethoxyvinyl)-1-oxoisoindolin-2-yl)piperidine-2,6-dione (500 mg, 1.591 mmol, 1 equiv) was dissolved in TFA/DCM (v/v=¼, 10 ml) at room temperature, and the reaction mixture was allowed to stir at this temperature for 5 min. LCMS indicated complete consumption of starting material and formation of product with desired mass. The reaction mixture was neutralized with saturated aqueous sodium carbonate solution. The resulting mixture was extracted with EA (20 mL×3) and washed with water, saturated brine, dried over Na₂SO₄, filtered, and concentrated to give 2-(2-(2,6-dioxopiperidin-3-yl)-1-oxoisoindolin-4-yl) acetaldehyde (300 mg) as a yellow solid. LCMS: [M+H+]=287.29.

### Step 3: Preparation of N-(6-((5-bromo-2-((4-(4-(4-(2-(2-(2,6-dioxopiperidin-3-yl)-1-oxoisoindolin-4-yl)ethyl) piperazin-1-yl)piperidin-1-yl)-5-ethyl-2-methoxyphenyl)amino)pyrimidin-4-yl)amino)quinoxalin-5-yl) methanesulfonamide

N-(6-((5-bromo-2-((5-ethyl-2-methoxy-4-(4-(piperazin-1-yl)piperidin-1-yl)phenyl)amino)pyrimidin-4-yl)amino)quinoxalin-5-yl) methanesulfonamide (700 mg, 0.883 mmol, 1 equiv) and DIEA (114.150 mg, 0.883 mmol, 1 equiv) were dissolved in DCM (20 ml) and MeOH (20 ml) at stirred at room temperature for 1.5 h. AcOH (106.069 mg, 1.766 mmol, 2 equiv) and 2-(2-(2,6-dioxopiperidin-3-yl)-1-oxoisoindolin-4-yl) acetaldehyde (278.127 mg, 0.971 mmol, 1.1 equiv) were added to the solution and stirred at room temperature. The reaction mixture was allowed to stir for 0.5 h. Then, NaBH₃CN (83.128 mg, 1.325 mmol, 1.5 equiv) was added to the solution and stirred at room temperature for another 0.5 h. LCMS indicated complete consumption of starting material and formation of product with desired mass (40% peak area). The reaction mixture was concentrated under vacuum and the residue was purified by preparative chromatography, eluting with MeCN/H₂O (3/2) to give N-(6-((5-bromo-2-((4-(4-(4-(2-(2-(2,6-dioxopiperidin-3-yl)-1-oxoisoindolin-4-yl)ethyl) piperazin-1-yl)piperidin-1-yl)-5-ethyl-2-methoxyphenyl)amino)pyrimidin-4-yl)amino)quinoxalin-5-yl) methanesulfonamide (55 mg, yield 6.34%) as a white solid after lyophilization. LCMS: [M+H+]=982.97. ¹H NMR (DMSO-d6, 400 MHZ) δ 11.00 (s, 1H), 9.00 (d, J=1.6 Hz, 1H), 8.93 (d, J=2.0 Hz, 1H), 8.86 (s, 1H), 8.29 (s, 1H), 8.21 (s, 1H), 8.16 (s, 1H), 8.13 (s, 0.6H), 7.88 (d, J=9.2 Hz 1H), 7.59 (d, J=8.0 Hz, 1H), 7.52-7.45 (m, 2H), 7.39 (s, 1H), 6.78 (s, 1H), 5.17-5.12 (m, 1H), 4.55-4.35 (m, 2H), 3.76 (s, 3H), 3.18-3.02 (m, 3H), 2.99 (m, 2H), 2.95-2.90 (m, 2H), 2.85-2.81 (t, J=7.6 Hz, 2H), 2.72-2.54 (m, 12H), 2.46-2.39 (m, 4H), 2.07-1.99 (m, 1H), 1.90-1.87 (m, 2H), 1.63-1.55 (m, 2H), 0.85-0.84 (m, 3H).

### Example 10: Preparation of N-(6-((5-bromo-2-((4-(4-(4-(1-(2-(2,6-dioxopiperidin-3-yl)-1-oxoisoindolin-5-yl)piperidine-4-carbonyl) piperazin-1-yl)piperidin-1-yl)-5-ethyl-2-methoxyphenyl)amino)pyrimidin-4-yl)amino)quinoxalin-5-yl) methanesulfonamide (Compound 10

### Step 1: Preparation of tert-butyl 1-(3-bromo-4-(methoxycarbonyl)phenyl)piperidine-4-carboxylate

2-bromo-4-fluorobenzoate (1.2 g, 5.15 mmol, 1.0 equiv) and tert-butyl piperidine-4-carboxylate (950 mg, 5.15 mmol, 1.0 equiv) were dissolved in MeCN (25 mL) at room temperature. K₂CO₃ (1.4 g, 10.3 mmol, 2.0 equiv) was slowly added, and the reaction mixture was degassed with N₂ for three times. The reaction mixture was heated to 80° C. and allowed to stir overnight under nitrogen atmosphere. LCMS analysis indicated complete consumption of starting material and formation of product with desired mass. The reaction mixture was cooled to room temperature and filtered, and the filtrate was concentrated in vacuo to give tert-butyl 1-(3-bromo-4-(methoxycarbonyl)phenyl)piperidine-4-carboxylate as a yellow solid. LCMS: [M+H+]=398.30.

### Step 2: Preparation of tert-butyl 1-(3-formyl-4-(methoxycarbonyl)phenyl)piperidine-4-carboxylate

tert-butyl 1-(3-bromo-4-(methoxycarbonyl)phenyl)piperidine-4-carboxylate (800 mg, 2.01 mmol, 1.0 equiv), tert-butylisocyanide (332 mg, 4.02 mmol, 2.0 equiv), Pd(OAc)₂ (45 mg, 0.2 mmol, 0.1 equiv), PCy₃ (56 mg, 0.2 mmol, 0.1 equiv), Et₃SiH (700 mg, 6.03 mmol, 3.0 equiv), and Na₂CO₃ (1.2 g, 4.02 mmol, 2.0 equiv) were dissolved in DMF (10 ml) at room temperature. The reaction mixture was degassed with N₂ for three times. The reaction mixture was warmed to 65° C. and stirred for 12 h. The reaction was monitored by LCMS to monitor product formation and stopped after no starting material remained. The reaction mixture was cooled to room temperature, poured into water, and extracted with EA (20 mL×3). The organic layers were combined, washed with water and brine. The organic phase was dried over anhydrous sodium sulfate and concentrated in vacuo. The crude residue was purified via column chromatography (PE:EA=5:1) to give tert-butyl 1-(3-formyl-4-(methoxycarbonyl)phenyl)piperidine-4-carboxylate (500 mg, 71% yield) as a yellow solid. LCMS: [M+H+]=347.41.

### Step 3: Preparation of tert-butyl 1-(2-(2,6-dioxopiperidin-3-yl)-1-oxoisoindolin-5-yl) piperidine-4-carboxylate

3-Aminopiperidine-2,6-dione (277 mg, 2.16 mmol, 1.5 equiv) and AcONa (392 mg, 2.88 mmol, 2.0 equiv) were dissolved in MeOH (10 ml). The mixture was allowed to stir at room temperature for 15 min. Then, tert-Butyl 1-(3-formyl-4-(methoxycarbonyl)phenyl)piperidine-4-carboxylate (500 mg, 1.44 mmol, 1.0 equiv) and AcOH (86 mg, 1.44 mmol, 1.0 equiv) were added to the above solution, and the reaction mixture was allowed to stir for another 1 h at 15° C. Then, NaBH₃CN (184 mg, 2.88 mmol, 2.0 equiv) was added at 0° C., and, upon complete addition, the reaction mixture was allowed to warm to 35° C. and stir overnight. LCMS monitored the product formation and no starting material remained. The reaction mixture was filtered, the filter cake was washed with MeOH and collected to give tert-butyl 1-(2-(2,6-dioxopiperidin-3-yl)-1-oxoisoindolin-5-yl)piperidine-4-carboxylate (220 mg, 36% yield) as a light blue solid. LCMS: [M+H+]=427.50.

### Step 4: Preparation of 1-(2-(2,6-dioxopiperidin-3-yl)-1-oxoisoindolin-5-yl)piperidine-4-carboxylic acid

tert-Butyl 1-(2-(2,6-dioxopiperidin-3-yl)-1-oxoisoindolin-5-yl)piperidine-4-carboxylate (220 mg, 0.52 mmol, 1.0 equiv) was dissolved in DCM/TFA (4/1, 5 mL). The reaction mixture was allowed to stir at 25° C. for 16 h. LCMS indicated complete consumption of starting material and formation of product with desired mass. The reaction mixture was concentrated to give 1-(2-(2,6-dioxopiperidin-3-yl)-1-oxoisoindolin-5-yl) piperidine-4-carboxylic acid (150 mg), which was used in the next step without further purification. LCMS: [M+H+]=371.39.

### Step 5: Preparation of N-(6-((5-bromo-2-((4-(4-(4-(1-(2-(2,6-dioxopiperidin-3-yl)-1-oxoisoindolin-5-yl)piperidine-4-carbonyl) piperazin-1-yl)piperidin-1-yl)-5-ethyl-2-methoxyphenyl)amino)pyrimidin-4-yl)amino)quinoxalin-5-yl) methanesulfonamide

1-(2-(2,6-Dioxopiperidin-3-yl)-1-oxoisoindolin-5-yl)piperidine-4-carboxylic acid (50 mg, 0.13 mmol, 1.0 equiv), N-(6-((5-bromo-2-((5-ethyl-2-methoxy-4-(4-(piperazin-1-yl) piperidin-1-yl)phenyl)amino)pyrimidin-4-yl)amino)quinoxalin-5-yl) methanesulfonamide (95 mg, 0.13 mmol, 1.0 equiv), DIEA (50 mg, 0.39 mmol, 3.0 equiv), and HATU (59 mg, 0.16 mmol, 1.2 equiv) were dissolved in THF (5 ml) and stirred at 25° C. under N₂ for 16 h. LCMS indicated complete consumption of starting material and formation of product with desired mass. The reaction mixture was concentrated and the crude was purified by Prep-HPLC to give N-(6-((5-bromo-2-((4-(4-(4-(1-(2-(2,6-dioxopiperidin-3-yl)-1-oxoisoindolin-5-yl)piperidine-4-carbonyl) piperazin-1-yl)piperidin-1-yl)-5-ethyl-2-methoxyphenyl)amino)pyrimidin-4-yl)amino)quinoxalin-5-yl) methanesulfonamide (9 mg, 6% yield) as a yellow solid. LCMS: [M+H+]=1065.06. ¹H NMR (400 MHZ, DMSO-d₆) δ10.94 (s, 1H), 9.92 (s, 1H), 9.00 (t, J=1.6 Hz, 1H), 8.93 (d, J=1.6 Hz, 1H), 8.85 (s, 1H), 8.64 (d, J=9.2 Hz, 1H), 8.29 (d, J=0.8 Hz, 1H), 8.21 (d, J=1.6 Hz, 1H), 7.89 (t. J=8.8 Hz, 1H), 7.57-7.44 (m, 1H), 7.41-7.37 (m, 1H), 7.07 (s, 1H), 6.78 (s, 1H), 5.04 (dd, J=13.2, 5.2 Hz, 1H), 4.33 (d. J=16.8 Hz, 1H), 4.21 (d, J=16.8 Hz, 1H), 3.91 (d, J=12.4 Hz, 2H), 3.76 (s, 3H), 3.71-3.51 (m, 5H), 3.21-2.85 (m, 8H), 2.75-2.52 (m, 6H), 2.49-2.24 (m, 5H), 2.05-1.91 (m, 1H), 1.90-1.83 (m, 2H), 1.80-1.41 (m, 6H), 1.24 (s, 1H), 0.85 (s, 3H).

### Example 11: Preparation of N-(6-((5-bromo-2-((4-(4-(4-(2-(1-(2,6-dioxopiperidin-3-yl)-3-ethyl-2-oxo-2,3-dihydro-1H-benzo[d]imidazol-4-yl)ethyl) piperazin-1-yl) piperidin-1-yl)-5-ethyl-2-methoxyphenyl)amino)pyrimidin-4-yl)amino)quinoxalin-5-yl) methanesulfonamide (Compound 11

2-(1-(2,6-dioxopiperidin-3-yl)-3-ethyl-2-oxo-2,3-dihydro-1H-benzo[d]imidazol-4-yl) acetaldehyde (500 mg, 1.59 mmol, 1.0 equiv), N-(6-((5-bromo-2-((5-ethyl-2-methoxy-4-(4-(piperazin-1-yl)piperidin-1-yl)phenyl)amino)pyrimidin-4-yl)amino)quinoxalin-5-yl) methanesulfonamide (1.2 g, 1.59 mmol, 1.0 equiv), and DIEA (308 mg, 2.39 mmol, 1.5 equiv) were dissolved in DCM (10 mL). The reaction mixture was allowed to stir at 25° C. for 0.5 h. Then, AcOH (95 mg, 1.59 mmol, 1.0 equiv) was added to the reaction mixture, which was allowed to stir for another 0.5 h. Then, NaBH(OAc)₃ (674 mg, 3.18 mmol, 2.0 equiv) was added to the reaction mixture and stirred for another 15 h. LCMS indicated complete consumption of starting material and formation of product with desired mass (70% peak area). The resulting mixture was concentrated and crude residue was purified by Prep-HPLC to give N-(6-((5-bromo-2-((4-(4-(4-(2-(1-(2,6-dioxopiperidin-3-yl)-3-ethyl-2-oxo-2,3-dihydro-1H-benzo[d]imidazol-4-yl)ethyl) piperazin-1-yl)piperidin-1-yl)-5-ethyl-2-methoxyphenyl)amino)pyrimidin-4-yl)amino)quinoxalin-5-yl) methanesulfonamide (150 mg, 19% yield) as a yellow solid. LCMS: [M+H+]=1012.1H NMR (400 MHZ, DMSO-d₆) δ 11.12 (s, 1H), 9.01 (s, 1H), 8.94 (s, 1H), 8.88 (s, 1H), 8.65 (s, 1H), 8.30 (s, 1H), 8.24 (s, 1H), 7.89 (d, J=9.0 Hz, 1H), 7.40 (s, 1H), 7.05-6.91 (m, 3H), 6.80 (s, 1H), 5.42-5.35 (m, 1H), 4.10-4.00 (m, 2H), 3.77 (s, 3H), 3.04-3.01 (m, 7H), 2.92-2.86 (m, 1H), 2.78-2.65 (m, 5H), 2.53-2.51 (m, 6H), 2.49-2.32 (m, 4H), 2.10-1.96 (m, 2H), 1.94-1.81 (m, 2H), 1.65-1.55 (m, 2H), 1.31-1.22 (m, 5H), 0.91-0.85 (m, 3H).

### Example 12: Preparation of N-(6-((5-bromo-2-((4-(4-(4-(2-(3-(2,6-dioxopiperidin-3-yl)-2-oxo-2,3-dihydrobenzo[d]oxazol-7-yl)ethyl) piperazin-1-yl)piperidin-1-yl)-5-ethyl-2-methoxyphenyl)amino)pyrimidin-4-yl)amino)quinoxalin-5-yl) methanesulfonamide (Compound 77

### Step 1: Preparation of 3-(7-bromo-2-oxobenzo[d]oxazol-3 (2H)-yl)piperidine-2,6-dione

A mixture of 7-bromobenzo[d]oxazol-2 (3H)-one (500 mg, 2.336 mmol, 1 equiv), 3-bromopiperidine-2,6-dione (897.159 mg, 4.672 mmol, 2 equiv), and Cs₂CO₃ (1.519 g, 4.672 mmol, 2 equiv) in DMF (10 mL) was allowed to stir in a round bottom flask at 50° C. for 16 h. The reaction was quenched with water (30 mL), and the mixture was extracted with EA (20 mL×3). The combined organic layers were washed with brine (30 ml×3), dried over anhydrous Na₂SO₄, filtered, and concentrated under reduced pressure. The residue was purified by re-crystallization from MeOH to afford 3-(7-bromo-2-oxobenzo[d]oxazol-3 (2H)-yl)piperidine-2,6-dione (400 mg, yield 52.66%) as a white solid. LCMS: [M+H+]=325.

### Step 2: Preparation of (E)-3-(7-(2-ethoxyvinyl)-2-oxobenzo[d]oxazol-3 (2H)-yl)piperidine-2,6-dione

To a solution of 3-(7-bromo-2-oxobenzo[d]oxazol-3 (2H)-yl)piperidine-2,6-dione (400 mg, 1.538 mmol, 1 equiv) and (E)-2-(2-ethoxyvinyl)-4,4,5,5-tetramethyl-1,3,2-dioxaborolane (395.994 mg, 1.999 mmol, 1.3 equiv) in dioxane (8 mL)/H₂O (2 mL) was added Pd (dppf) Cl₂ (112.420 mg, 0.154 mmol, 0.1 equiv) and K₂CO₃ (636.688 mg, 4.614 mmol, 3 equiv). The resulting solution was heated to 65° C. and allowed to stir under N₂ atmosphere for 3 h. LCMS indicated complete consumption of starting material and formation of product with desired mass. Then, the reaction solution was washed with brine and extracted with EA (10 mL×3). The organic phase was dried over Na₂SO₄, and volatiles were evaporated to give (E)-3-(7-(2-ethoxyvinyl)-2-oxobenzo[d]oxazol-3 (2H)-yl) piperidine-2,6-dione (400 mg, yield 82.23%) as a brown solid, which was used directly in the next step without further purification. LCMS: [M+H+]=317.

### Step 3: Preparation of 2-(3-(2,6-dioxopiperidin-3-yl)-2-oxo-2,3-dihydrobenzo[d]oxazol-7-yl) acetaldehyde

(E)-3-(7-(2-ethoxyvinyl)-2-oxobenzo[d]oxazol-3 (2H)-yl)piperidine-2,6-dione (400 mg, 1.265 mmol, 1 equiv) was dissolved in HCOOH (2 mL), and the reaction mixture was allowed to stir for 2 h at room temperature. LCMS indicated complete consumption of starting material and formation of product with desired mass (90% peak area). The reaction solution was evaporated to give product 2-(3-(2,6-dioxopiperidin-3-yl)-2-oxo-2,3-dihydrobenzo[d]oxazol-7-yl) acetaldehyde as a brown solid, which was used directly in the next step without further purification. LCMS: [M+H+]=289.

### Step 4: Preparation of N-(6-((5-bromo-2-((4-(4-(4-(2-(3-(2,6-dioxopiperidin-3-yl)-2-oxo-2,3-dihydrobenzo[d]oxazol-7-yl)ethyl) piperazin-1-yl)piperidin-1-yl)-5-ethyl-2-methoxyphenyl)amino)pyrimidin-4-yl)amino)quinoxalin-5-yl) methanesulfonamide

A mixture of N-(6-((5-bromo-2-((5-ethyl-2-methoxy-4-(4-(piperazin-1-yl)piperidin-1-yl)phenyl)amino)pyrimidin-4-yl)amino)quinoxalin-5-yl) methanesulfonamide (103.124 mg, 0.145 mmol, 1 equiv) and TEA (43.905 mg, 0.435 mmol, 3 equiv) in MeOH (5 ml) was allowed to stir at 25° C. for 20 min. Then, AcOH (26.082 mg, 0.435 mmol, 3 equiv) and 2-(3-(2,6-dioxopiperidin-3-yl)-2-oxo-2,3-dihydrobenzo[d]oxazol-7-yl) acetaldehyde (91.384 mg, 0.290 mmol, 2 equiv) were added. The mixture was allowed to stir at 25° C. for another 0.5 h. Then, NaBH₃CN (10.955 mg, 0.174 mmol, 1.2 equiv) was added, and the mixture was allowed to stir at 25° C. for another 1 h. LCMS indicated complete consumption of starting material and formation of product with desired mass. The reaction mixture was concentrated under reduced pressure and the residue was purified through Prep-HPLC to give N-(6-((5-bromo-2-((4-(4-(4-(2-(3-(2,6-dioxopiperidin-3-yl)-2-oxo-2,3-dihydrobenzo[d]oxazol-7-yl)ethyl) piperazin-1-yl)piperidin-1-yl)-5-ethyl-2-methoxyphenyl)amino)pyrimidin-4-yl)amino)quinoxalin-5-yl) methanesulfonamide (49.500 mg, purity 92%, yield 13.34%) as a yellow solid. LCMS: [M+H+]=983. ¹H NMR (400 MHZ, DMSO-d₆) δ 11.22 (s, 1H), 9.01 (d. J=2.0 Hz, 1H), 8.94 (d, J=2.0 Hz, 1H), 8.87 (s, 1H), 8.66 (s, 1H), 8.30 (s, 1H), 8.15 (s, 1H), 7.89 (d, J=9.2 Hz, 1H), 7.40 (d, J=5.2 Hz, 1H), 7.23-7.08 (m, 4H), 6.79 (s, 1H), 5.41-5.37 (m, 1H), 3.78 (s, 3H), 3.13-2.98 (m, 5H), 2.98-2.85 (m, 4H), 2.77-2.62 (m, 12H), 2.49-2.39 (m, 3H), 2.24-2.13 (m, 2H), 1.98-1.89 (m, 2H), 1.65-1.55 (m, 2H), 0.85 (s, 3H).

### Example 13: Preparation of(S)-N-(6-((5-bromo-2-((4-(4-((4-(2-(2,6-dioxopiperidin-3-yl)-1-oxoisoindolin-5-yl)piperazin-1-yl)methyl)-[1,4′-bipiperidin]-1′-yl)-5-ethyl-2-methoxyphenyl)amino)pyrimidin-4-yl)amino)quinoxalin-5-yl) methanesulfonamide (compound 35

### Step 1: Preparation of tert-butyl(S)-4-((4-(2-(2,6-dioxopiperidin-3-yl)-1-oxoisoindolin-5-yl) piperazin-1-yl)methyl) piperidine-1-carboxylate

A mixture of(S)-3-(1-oxo-5-(piperazin-1-yl) isoindolin-2-yl)piperidine-2,6-dione (1.000 g, 1.784 mmol, 1 eq) and CH₃COONa (146.147 mg, 1.784 mmol, 1 eq) in DCM (100 mL) and MeOH (10 mL) was stirred at room temperature for 0.5 hour, then CH₃₀₀H (214.208 mg, 3.567 mmol, 2 eq) and tert-butyl 4-formylpiperidine-1-carboxylate (418.442 mg, 1.962 mmol, 1.1 eq) were added to the solution and stirred at room temperature for 0.5 hours. NaBH₃CN (123.112 mg, 1.962 mmol, 1.1 eq) was added to the solution and stirred at room temperature for another 0.5 hours. LCMS showed a peak of 95% peak area with desired mass. The reaction mixture was quenched with sodium bicarbonate aqueous solution. The organic layer was separated and washed with water twice, dried over anhydrous magnesium sulfate, and concentrated under reduced pressure to give tert-butyl(S)-4-((4-(2-(2,6-dioxopiperidin-3-yl)-1-oxoisoindolin-5-yl)piperazin-1-yl)methyl) piperidine-1-carboxylate (900 mg, yield 96.00%) as a white solid. LCMS: [M+H]⁺=526.30.

### Step 2: Preparation of(S)-3-(1-oxo-5-(4-(piperidin-4-ylmethyl) piperazin-1-yl) isoindolin-2-yl) piperidine-2,6-dione

A mixture of Tert-butyl(S)-4-((4-(2-(2,6-dioxopiperidin-3-yl)-1-oxoisoindolin-5-yl) piperazin-1-yl)methyl) piperidine-1-carboxylate (900.000 mg, 1.712 mmol, 1 eq) in HCl/Dioxane (10 mL) was stirred at room temperature for 2 hours. LCMS analysis indicated complete consumption of starting material and formation of product with desired mass. The solvent was concentrated under reduced pressure to give(S)-3-(1-oxo-5-(4-(piperidin-4-ylmethyl) piperazin-1-yl) isoindolin-2-yl)piperidine-2,6-dione (600 mg, yield 96.15%) as a white solid. LCMS: [M+H+]=426.24.

### Step 3: Preparation of(S)-N-(6-((5-bromo-2-((4-(4-((4-(2-(2,6-dioxopiperidin-3-yl)-1-oxoisoindolin-5-yl)piperazin-1-yl)methyl)-[1,4′-bipiperidin]-1′-yl)-5-ethyl-2-methoxyphenyl)amino)pyrimidin-4-yl)amino)quinoxalin-5-yl) methanesulfonamide

A mixture of(S)-3-(1-oxo-5-(4-(piperidin-4-ylmethyl) piperazin-1-yl) isoindolin-2-yl) piperidine-2,6-dione (108.017 mg, 0.234 mmol, 1.5 eq), TEA (47.318 mg, 0.468 mmol, eq) in DCM (50 mL) and MeOH (5 mL) was stirred at room temperature for 0.5 hour. Then, CH₃COOH (14.040 mg, 0.234 mmol, 1.5 eq), N-(6-((5-bromo-2-((5-ethyl-2-methoxy-4-(4-oxopiperidin-1-yl)phenyl)amino)pyrimidin-4-yl)amino)quinoxalin-5-yl) methanesulfonamide (100.000 mg, 0.156 mmol, 1 eq) were added to the solution. The reaction mixture was stirred at room temperature for 1 hour. NaBH₃CN (14.440 mg, 0.234 mmol, 1.5 eq) was added to the solution and stirred at room temperature for 48 hours. LCMS analysis indicated complete consumption of starting material and formation of product with desired mass. The reaction mixture was concentrated under reduced pressure and the resulting crude residue was purified by preparative chromatography to afford (S)-N-(6-((5-bromo-2-((4-(4-((4-(2-(2,6-dioxopiperidin-3-yl)-1-oxoisoindolin-5-yl) piperazin-1-yl)methyl)-[1,4′-bipiperidin]-1′-yl)-5-ethyl-2-methoxyphenyl)amino)pyrimidin-4-yl)amino)quinoxalin-5-yl) methanesulfonamide (3.000 mg, yield 1.83%) as a white solid. LCMS: [M+H+]=1052.08.1H NMR (400 MHZ, DMSO-d₆) § 10.95 (s, 1H), 9.02 (s, 1H), 8.98 (s, 1H), 8.86 (s, 1H), 8.65 (s, 1H), 8.30 (s, 1H), 8.22 (s, 1H), 8.18 (s, 1H), 7.88 (d, J=9.2 Hz, 1H), 7.53 (d, J=8.4 Hz, 1H), 7.40 (s, 1H), 7.08 (s, 1H), 7.06 (s, 1H), 6.79 (s, 1H), 5.06 (dd. J=13.2, 5.2 Hz, 1H), 4.34 (d. J=16.8 Hz, 1H), 4.22 (d, J=16.8 Hz, 1H), 3.77 (s, 3H), 3.05-2.98 (m, 7H), 2.97-2.85 (m, 3H), 2.78-2.54 (m, 5H), 2.46-2.28 (m, 6H), 2.20 (d, J=7.2 Hz, 2H), 2.01-1.73 (m, 6H), 1.73-1.50 (m, 4H), 1.24-1.15 (m, 2H), 1.01-0.93 (m, 1H), 0.85 (t, J=7.6 Hz, 3H).

### Example 14: Preparation of(S)-N-(6-((5-bromo-2-((4-(4-(4-(2-(2,6-dioxopiperidin-3-yl)-1-oxoisoindolin-5-yl)piperazin-1-yl)-[1,4′-bipiperidin]-1′-yl)-5-ethyl-2-methoxyphenyl)amino)pyrimidin-4-yl)amino)quinoxalin-5-yl) methanesulfonamide (compound 36

### Step 1: Preparation of tert-butyl(S)-4-(4-(2-(2,6-dioxopiperidin-3-yl)-1-oxoisoindolin-5-yl) piperazin-1-yl)piperidine-1-carboxylate

A mixture of(S)-3-(1-oxo-5-(piperazin-1-yl) isoindolin-2-yl)piperidine-2,6-dione (2.000 g, 3.567 mmol, 1 eq) and CH₃COONa (292.293 mg, 3.567 mmol, 1 eq) in DCM (100 mL) and MeOH (10 mL) was stirred at room temperature for 0.5 hour. CH₃COOH (428.416 mg, 7.134 mmol, 2 eq), tert-butyl 4-oxopiperidine-1-carboxylate (781.832 mg, 3.924 mmol, 1.1 eq) were added and the reaction mixture was stirred at room temperature for 0.5 hour. NaBH₃CN (246.233 mg, 3.924 mmol, 1.1 eq) was added and the mixture was stirred at room temperature for 0.5 hours. LCMS indicated complete consumption of starting material and formation of product with the desired mass. After quenching the reaction solution with sodium bicarbonate aqueous solution, the organic layer was separated, washed with brine, dried over anhydrous magnesium sulfate, filtered and concentrated under reduced pressure to give tert-butyl(S)-4-(4-(2-(2,6-dioxopiperidin-3-yl)-1-oxoisoindolin-5-yl)piperazin-1-yl)piperidine-1-carboxylate (1.500 g, yield 82.19%) as a white solid. LCMS: [M+H]⁺=512.28.

### Step 2: Preparation of(S)-3-(1-oxo-5-(4-(piperidin-4-yl)piperazin-1-yl) isoindolin-2-yl) piperidine-2,6-dione

A mixture of Tert-butyl(S)-4-(4-(2-(2,6-dioxopiperidin-3-yl)-1-oxoisoindolin-5-yl) piperazin-1-yl)piperidine-1-carboxylate (1.400 g, 2.736 mmol, 1 eq) in HCl/Dioxane (10 mL) was stirred at room temperature for 2 hours. LCMS indicated complete consumption of starting material and formation of product with the desired mass. The solvent was removed under reduced pressure to give(S)-3-(1-oxo-5-(4-(piperidin-4-yl)piperazin-1-yl) isoindolin-2-yl)piperidine-2,6-dione. (1.000 g, yield 81.58%) as a white solid which was used directly in next step without further purification. LCMS: [M+H]⁺=412.23.

### Step 3: Preparation of(S)-N-(6-((5-bromo-2-((4-(4-(4-(2-(2,6-dioxopiperidin-3-yl)-1-oxoisoindolin-5-yl)piperazin-1-yl)-[1,4′-bipiperidin]-1′-yl)-5-ethyl-2-methoxyphenyl)amino)pyrimidin-4-yl)amino)quinoxalin-5-yl) methanesulfonamide

A mixture of(S)-3-(1-oxo-5-(4-(piperidin-4-yl)piperazin-1-yl) isoindolin-2-yl) piperidine-2,6-dione (104.737 mg, 0.234 mmol, 1.5 eq), TEA (47.318 mg, 0.468 mmol, eq) in DCM (10 mL) and MeOH (5 mL) was stirred at room temperature for 0.5 hour. CH₃COOH (14.040 mg, 0.234 mmol, 1.5 eq) and N-(6-((5-bromo-2-((5-ethyl-2-methoxy-4-(4-oxopiperidin-1-yl)phenyl)amino)pyrimidin-4-yl)amino)quinoxalin-5-yl) methanesulfonamide (100.000 mg, 0.156 mmol, 1 eq) were added and the mixture was stirred at room temperature for another 1 hour. NaBH₃CN (14.440 mg, 0.234 mmol, 1.5 eq) was added and the reaction mixture was stirred at room temperature for 2 hours. LCMS indicated complete consumption of starting material and formation of product with the desired mass (90% peak area). The reaction mixture was concentrated under reduced pressure and the resulting crude residue was purified by preparative chromatography to afford (S)-N-(6-((5-bromo-2-((4-(4-(4-(2-(2,6-dioxopiperidin-3-yl)-1-oxoisoindolin-5-yl) piperazin-1-yl)-[1,4′-bipiperidin]-1′-yl)-5-ethyl-2-methoxyphenyl)amino)pyrimidin-4-yl)amino)quinoxalin-5-yl) methanesulfonamide (12.800 mg, yield 7.92%) as a white solid. LCMS: [M+H+]=1038.05. ¹H NMR (400 MHZ, DMSO-d₆) δ 10.94 (s, 1H), 9.92 (s, 1H), 9.05 (s, 1H), 8.95 (s, 1H), 8.84 (s, 1H), 8.64 (d, J=9.2 Hz, 1H), 8.26 (s, 1H), 8.24 (s, 1H), 7.89 (d, J=9.2 Hz, 1H), 7.58 (s, 1H), 7.42 (s, 1H), 7.23-7.15 (m, 2H), 6.79 (s, 1H), 5.06 (dd, J=13.2, 5.2 Hz, 1H), 4.35 (d, J=17.2 Hz, 1H), 4.24 (d, J=17.2 Hz, 1H), 4.23-3.95 (m, 2H), 3.78 (s, 3H), 3.52-3.50 (m, 1H), 3.26-3.16 (m, 3H), 3.12-3.05 (m, 3H), 3.05-3.01 (m, 3H), 2.96-2.86 (m, 2H), 2.82-2.55 (m, 5H), 2.48-2.31 (m, 4H), 2.62-2.01 (m, 5H), 2.01-1.71 (m, 5H), 1.19 (t, J=7.2 Hz, 1H), 1.00 (t. J=7.2 Hz, 1H), 0.94-0.76 (m, 3H).

### Example 15: Preparation of N-(6-((5-bromo-2-((4-(9-((1-(2-(2,6-dioxopiperidin-3-yl)-1-oxoisoindolin-5-yl)piperidin-4-yl)methyl)-3,9-diazaspiro[5.5]undecan-3-yl)-5-ethyl-2-methoxyphenyl)amino)pyrimidin-4-yl)amino)quinoxalin-5-yl) methanesulfonamide (Compound 37

### Step 1: Preparation of 3-benzyl 9-(tert-butyl)_{3,9}-diazaspiro[5.5]undecane-3,9-dicarboxylate

CbzCl (2.112 g, 12.378 mmol, 1.2 eq) was added dropwise to a mixture of tert-butyl 3,9-diazaspiro[5.5]undecane-3-carboxylate hydrochloride (3.000 g, 10.315 mmol, 1 eq) and DIEA (4.000 g, 30.946 mmol, 3 eq) in DCM (50 mL), then the mixture was stirred at room temperature for 15 hours. TLC showed the starting material was consumed completely. The mixture was poured into water, separated and the organic layer was washed with HCl (1 N), dried over anhydrous Na₂SO₄, filtered and concentrated to give 3-benzyl 9-(tert-butyl)_{3,9}-diazaspiro[5.5]undecane-3,9-dicarboxylate (4.200 g, yield 104.80%) (crude) as a yellow solid which was used directly in next step without further purification.

### Step 2: Preparation of benzyl 3,9-diazaspiro[5.5]undecane-3-carboxylate hydrochloride

HCl/dioxane (10 mL) (4 M) was added to a mixture of 3-benzyl 9-(tert-butyl)_{3,9}-diazaspiro[5.5]undecane-3,9-dicarboxylate (4.200 g, 10.811 mmol, 1 eq) in EA (50 mL), then the mixture was stirred at 30° C. for 18 hours. TLC showed the starting material was consumed completely. The mixture was filtered and the filter cake was collected to give benzyl 3,9-diazaspiro[5.5]undecane-3-carboxylate hydrochloride (3.400 g, yield 96.81%) as a white solid which was used directly in next step without further purification.

### Step 3: Preparation of benzyl 9-(2-bromo-5-methoxy-4-nitrophenyl)-3,9-diazaspiro[5.5]undecane-3-carboxylate

A mixture of benzyl 3,9-diazaspiro[5.5]undecane-3-carboxylate hydrochloride (3.500 g, 10.774 mmol, 1 eq), 1-Bromo-2-fluoro-4-methoxy-5-nitrobenzene (2.694 g, 10.774 mmol, 1 eq) and K₂CO₃ (4.467 g, 32.323 mmol, 3 eq) in MeCN (50 mL) was stirred at 80° C. for 16 hours. TLC showed the starting material remained and desired compound was detected. The mixture was filtered and the filtrate was concentrated. The residue was dissolved in DCM and PE was added. The mixture was filtered and the filter cake was collected to give benzyl 9-(2-bromo-5-methoxy-4-nitrophenyl)-3,9-diazaspiro[5.5]undecane-3-carboxylate (4.200 g, yield 75.20%) as a yellow solid which was used directly in next step without further purification.

### Step 4: Preparation of benzyl 9-(5-methoxy-4-nitro-2-vinylphenyl)-3,9-diazaspiro[5.5]undecane-3-carboxylate

A mixture of benzyl 9-(2-bromo-5-methoxy-4-nitrophenyl)-3,9-diazaspiro[5.5]undecane-3-carboxylate (4.200 g, 8.102 mmol, 1 eq), potassium trifluoro (vinyl) borate (1.628 g, 12.153 mmol, 1.5 eq), K₃PO₄ (3.439 g, 16.204 mmol, 2 eq) and Pd (dppf) Cl₂ (600.987 mg, 0.810 mmol, 0.1 eq) in DMF (50 mL) and H₂O (10 mL) was stirred at 100° C. under N₂ for 16 hours. TLC showed the starting material was consumed completely. The mixture was filtered and the filtrate was extracted with EA. The combined organic layer was dried over anhydrous Na₂SO₄, filtered and concentrated. The crude residue was purified by column chromatography on silica gel to give benzyl 9-(5-methoxy-4-nitro-2-vinylphenyl)-3,9-diazaspiro[5.5]undecane-3-carboxylate (900.000 mg, yield 23.86%) as a yellow solid.

### Step 5: Preparation of benzyl 9-(4-amino-2-ethyl-5-methoxyphenyl)-3,9-diazaspiro[5.5]undecane-3-carboxylate

A mixture of benzyl 9-(5-methoxy-4-nitro-2-vinylphenyl)-3,9-diazaspiro[5.5]undecane-3-carboxylate (900.000 mg, 1.933 mmol, 1 eq) and Pd/C (102.8 mg, 0.097 mmol, 0.05 eq, purity 10%) in MeOH (40 mL) was stirred at room temperature under H₂ balloon for 16 hours. TLC and LCMS showed the starting material was consumed completely. The mixture was filtered and the filtrate was concentrated to give benzyl 9-(4-amino-2-ethyl-5-methoxyphenyl)-3,9-diazaspiro[5.5]undecane-3-carboxylate (800.000 mg, yield 94.57%) as a brown oil which was used directly in next step without further purification. LCMS: [M+H]⁺=438.3.

### Step 6: Preparation of benzyl 9-(4-((5-bromo-4-((5-(methylsulfonamido)quinoxalin-6-yl)amino)pyrimidin-2-yl)amino)-2-ethyl-5-methoxyphenyl)-3,9-diazaspiro[5.5]undecane-3-carboxylate

A mixture of benzyl 9-(4-amino-2-ethyl-5-methoxyphenyl)-3,9-diazaspiro[5.5]undecane-3-carboxylate (200.000 mg, 0.457 mmol, 1 eq), N-(6-((5-bromo-2-chloropyrimidin-4-yl)amino)quinoxalin-5-yl) methanesulfonamide (196.389 mg, 0.457 mmol, 1 eq) and MsOH (131.770 mg, 1.371 mmol, 3 eq) in t-BuOH (10 mL) was stirred at 90° C. under N₂ in a seal tube for 16 hours. LCMS showed the starting material remained and desired compound was detected. The mixture was concentrated and the residue was purified by column chromatography on silica gel (DCM/MeOH=20/1) to give benzyl 9-(4-((5-bromo-4-((5-(methylsulfonamido)quinoxalin-6-yl)amino)pyrimidin-2-yl)amino)-2-ethyl-5-methoxyphenyl)-3,9-diazaspiro[5.5]undecane-3-carboxylate (250.000 mg, yield 65.84%) as a yellow solid. LCMS: [M+H]⁺=830.3.

### Step 7: Preparation of N-(6-((5-bromo-2-((5-ethyl-2-methoxy-4-(3,9-diazaspiro[5.5]undecan-3-yl)phenyl)amino)pyrimidin-4-yl)amino)quinoxalin-5-yl) methanesulfonamide

A mixture of benzyl 9-(4-((5-bromo-4-((5-(methylsulfonamido)quinoxalin-6-yl)amino)pyrimidin-2-yl)amino)-2-ethyl-5-methoxyphenyl)-3,9-diazaspiro[5.5]undecane-3-carboxylate (200.000 mg, 0.241 mmol, 1 eq) in HBr/AcOH (2 mL, purity 30%) was stirred at room temperature for 1 hour. LCMS indicated complete consumption of starting material and formation of product with desired mass. The mixture was concentrated to give N-(6-(5-bromo-2-((5-ethyl-2-methoxy-4-(3,9-diazaspiro[5.5]undecan-3-yl)phenyl)amino)pyrimidin-4-yl)amino)quinoxalin-5-yl) methanesulfonamide (250.000 mg, yield 106.84%) (crude) as a yellow solid which was used directly in next step without further purification. LCMS: [M+H]⁺=696.2.

### Step 8: Preparation of N-(6-((5-bromo-2-((4-(9-((1-(2-(2,6-dioxopiperidin-3-yl)-1-oxoisoindolin-5-yl)piperidin-4-yl)methyl)-3,9-diazaspiro[5.5]undecan-3-yl)-5-ethyl-2-methoxyphenyl)amino)pyrimidin-4-yl)amino)quinoxalin-5-yl) methanesulfonamide

A mixture of N-(6-((5-bromo-2-((5-ethyl-2-methoxy-4-(3,9-diazaspiro[5.5]undecan-3-yl)phenyl)amino)pyrimidin-4-yl)amino)quinoxalin-5-yl) methanesulfonamide (250.000 mg, 0.322 mmol, 1 eq), 1-(2-(2,6-dioxopiperidin-3-yl)-1-oxoisoindolin-5-yl)piperidine-4-carbaldehyde (114.262 mg, 0.322 mmol, 1 eq), DIEA (62.333 mg, 0.482 mmol, 1.5 eq) and AcOH (19.307 mg, 0.322 mmol, 1 eq) in DCM (10 mL) was stirred at room temperature for 1 hour. NaBH(OAc)₃ (136.224 mg, 0.643 mmol, eq) was added and the mixture was stirred for another 12 hours. LCMS showed the starting material was consumed completely and desired compound was detected. The mixture was concentrated and the residue was purified by Prep-HPLC (FA) to give N-(6-((5-bromo-2-((4-(9-((1-(2-(2,6-dioxopiperidin-3-yl)-1-oxoisoindolin-5-yl)piperidin-4-yl)methyl)-3,9-diazaspiro[5.5]undecan-3-yl)-5-ethyl-2-methoxyphenyl)amino)pyrimidin-4-yl)amino)quinoxalin-5-yl) methanesulfonamide (27.000 mg, yield 8.11%) as a yellow solid. LCMS: [M+H]⁺=1037.9.1H NMR (400 MHZ, DMSO-d₆) § 10.95 (s, 1H), 9.01 (s, 1H), 8.95 (s, 1H), 8.88 (s, 1H), 8.64 (d, J=8.0 Hz, 1H), 8.28 (s, 1H), 8.18 (s, 1H), 7.85 (d, J=8.0 Hz, 1H), 7.49 (d, J=8.0 Hz, 1H), 7.38 (s, 1H), 7.05 (s, 1H), 7.03 (d. J=8.0 Hz, 1H), 6.83 (s, 1H), 5.04 (dd, J=12.0, 4.0 Hz, 1H), 4.32 (d, J=16.8 Hz, 1H), 4.19 (d, J=16.8 Hz, 1H), 3.86 (d, J=11.6 Hz, 2H), 3.77 (s, 3H), 3.02 (s, 3H), 2.94-2.76 (m, 7H), 2.62-2.55 (m, 1H), 2.46-2.29 (m, 7H), 2.21-2.15 (m, 2H), 1.99-1.92 (m, 1H), 1.82-1.71 (m, 3H), 1.61-1.48 (m, 8H), 1.25-1.09 (m, 2H), 0.84 (t, J=8.0 Hz, 3H).

### Example 16: Preparation of(S)-N-(6-((5-bromo-2-((4-(4-(4-(2-(2,6-dioxopiperidin-3-yl)-1-oxoisoindolin-5-yl)piperazin-1-yl)piperidin-1-yl)-5-ethyl-2-methoxyphenyl)amino)pyrimidin-4-yl)amino)quinoxalin-5-yl) methanesulfonamide (Compound 38

N-(6-((5-Bromo-2-((5-ethyl-2-methoxy-4-(4-oxopiperidin-1-yl)phenyl)amino)pyrimidin-4-yl)amino)quinoxalin-5-yl) methanesulfonamide (85 mg, 0.13 mmol, 1.0 eq), (S)-3-(1-oxo-5-(piperazin-1-yl) isoindolin-2-yl)piperidine-2,6-dione (43 mg, 0.13 mmol, 1.0 eq) and DIEA (17 mg, 0.13 mmol, 1.0 eq) were dissolved in DCM/MeOH (3 mL/1 mL). The reaction mixture was allowed to stir 0.5 h at 25° C. AcOH (8 mg, 0.13 mmol, 1.0 eq) was added and the reaction mixture was stirred for another 0.5 h. NaBH(OAc)₃ (34 mg, 0.16 mmol, 1.2 eq) was added and the mixture was stirred for another 3 h. LCMS analysis indicated complete consumption of starting material and formation of product with desired mass. The reaction mixture was concentrated under reduced pressure and the resulting crude residue was purified by Prep-HPLC (FA) to afford (S)-N-(6-((5-bromo-2-((4-(4-(4-(2-(2,6-dioxopiperidin-3-yl)-1-oxoisoindolin-5-yl) piperazin-1-yl)piperidin-1-yl)-5-ethyl-2-methoxyphenyl)amino)pyrimidin-4-yl)amino)quinoxalin-5-yl) methanesulfonamide (3 mg, 2.4% yield) as a yellow solid. LCMS: [M+H+]=955.2.1H NMR (400 MHZ, DMSO-d₆) δ 10.94 (s, 1H), 9.90 (s, 1H), 9.00 (d. J=2.0 Hz, 1H), 8.93 (d, J=2.0 Hz, 1H), 8.86 (s, 1H), 8.65 (d, J=9.2 Hz, 1H), 8.29 (s, 1H), 8.22 (s, 1H), 7.88 (d, J=9.2 Hz, 1H), 7.54 (d, J=8.4 Hz, 1H), 7.40 (s, 1H), 7.08 (d, J=7.6 Hz, 2H), 6.79 (s, 1H), 5.05 (dd, J=13.2, 5.2 Hz, 1H), 4.34 (d, J=16.8 Hz, 1H), 4.22 (d, J=16.8 Hz, 1H), 3.77 (s, 3H), 3.28-3.25 (m, 1H), 3.08-3.01 (m, 5H), 2.99-2.82 (m, 1H), 2.80-2.64 (m, 6H), 2.63-2.56 (m, 1H), 2.47-2.33 (m, 4H), 2.04-1.89 (m, 4H), 1.72-1.58 (m, 2H), 1.34-1.26 (m, 2H), 0.85 (t, J=6.8 Hz, 3H).

| Compd # | Characterizations |
|---|---|
| 53 | LCMS: [M + H]⁺ = 1022.04. ¹H NMR (400 MHz, DMSO-d₆) δ 10.95 |
| | (s, 1H), 9.85 (br, 1H), 9.15 (s, 1H), 9.00 (s, 1H), 8.83 (s, 1H), 8.74- |
| | 8.56 (m, 1H), 8.37 (s, 1H), 8.27 (s, 1H), 7.89 (d, J = 9.6 Hz, 1H), |
| | 7.57-7.47 (m, 1H), 7.43 (s, 1H), 7.08 (d, J = 8.0 Hz, 2H), 6.80 (s, |
| | 1H), 5.10-4.99 (m, 1H), 4.38-4.32 (m, 1H), 4.25-4.14 (m, 1H), |
| | 3.79 (s, 3H), 3.13-3.05 (m, 4H), 3.04 (s, 3H), 2.83-2.70 (m, 3H), |
| | 2.53-2.48 (m, 8H), 2.23-2.18 (m, 2H), 1.83-1.71 (m, 6H), 1.65-1.55 |
| | (m, 6H), 1.19 (t, J = 7.2 Hz, 2H), 0.91-0.81 (m, 3H). |
| 55 | LCMS: [M + H]⁺ = 993.98. ¹H NMR (400 MHz, DMSO-d₆) δ 10.94 |
| | (s, 1H), 9.91 (br, 1H), 9.02 (d, J = 2.0 Hz, 1H), 8.94 (d, J = 2.0 |
| | Hz, 1H), 8.87 (s, 1H), 8.66 (d, J = 9.6 Hz, 1H), 8.30 (d, J = 3.6 |
| | Hz, 1H), 8.24 (s, 1H), 7.89 (d, J = 9.6 Hz, 1H), 7.52 (d, J = 8.4 |
| | Hz, 1H), 7.41 (d, J = 10.4 Hz, 1H), 6.92-6.73 (m, 2H), 6.53 (s, |
| | 1H), 5.05 (dd, J = 13.2, 5.2 Hz, 1H), 4.33 (d, J = 17.2 Hz, 1H), |
| | 4.20 (d, J = 16.8 Hz, 1H), 3.77 (s, 3H), 3.75-3.68 (m, 4H), 3.18- |
| | 3.02 (m, 6H), 2.97-2.86 (m, 2H), 2.77-2.61 (m, 4H), 2.48-2.33 |
| | (m, 4H), 2.12-1.82 (m, 8H), 1.76-1.69 (m, 2H), 0.88-0.76 (m, 3H). |
| 57 | ¹H NMR (400 MHz, DMSO-d₆) δ 11.05 (s, 1H), 9.11 (d, J = 2.0 |
| | Hz, 1H), 9.05 (d, J = 2.0 Hz, 1H), 8.99 (s, 1H), 8.77 (d, J = 9.2 |
| | Hz, 1H), 8.41 (s, 1H), 8.32 (s, 1H), 7.99 (d, J = 9.2 Hz, 1H), 7.63 |
| | (d, J = 8.4 Hz, 1H), 7.52 (s, 1H), 7.24 (s, 1H), 7.22 (s, 1H), 6.89 |
| | (s, 1H), 5.16 (dd, J = 13.2, 5.2 Hz, 1H), 4.45 (d, J = 16.8 Hz, |
| | 1H), 4.33 (d, J = 16.8 Hz, 1H), 3.89 (s, 3H), 3.27-3.24 (m, 5H), |
| | 3.23-3.21 (m, 3H), 3.11-3.06 (m, 3H), 3.01-2.95 (m, 1H), 2.81- |
| | 2.75 (m, 2H), 2.75-2.68 (m, 1H), 2.57-2.45 (m, 4H), 2.11-2.07 |
| | (m, 1H), 1.99-1.86 (m, 6H), 1.51-1.47 (m, 2H), 1.45-1.31 (m, |
| | 2H), 0.97 (t, J = 7.2 Hz, 3H). |
| 62 | LCMS: [M + H]⁺ = 1024.02. ¹H NMR (400 MHz, DMSO-d₆) δ 10.93 |
| | (s, 1H), 9.94 (br, 1H), 9.01 (s, 1H), 8.95 (s, 1H), 8.85 (s, 1H), |
| | 8.65 (d, J = 9.2 Hz, 1H), 8.26 (s, 1H), 8.24 (s, 1H), 7.88 (d, J = |
| | 9.2 Hz, 1H), 7.53 (d, J = 8.8 Hz, 1H), 7.42 (s, 1H), 7.09 (s, 1H), |
| | 7.07 (d, J = 7.6 Hz, 1H), 6.77 (s, 1H), 5.05 (dd, J = 13.2, 5.2 Hz, |
| | 1H), 4.33 (d, J = 17.2 Hz, 1H), 4.23 (d, J = 16.8 Hz, 1H), 3.77 (s, |
| | 3H), 3.47-3.32 (m, 5H), 3.14-3.00 (m, 6H), 2.98-2.85 (m, 2H), |
| | 2.81-2.52 (m, 10H), 2.43-2.32 (m, 3H), 2.11-1.92 (m, 3H), 1.63- |
| | 1.42 (m, 3H), 1.23-1.12 (m, 1H), 0.86 (t, J = 7.2 Hz, 3H). |
| 63 | LCMS: [M + H]⁺ = 1009.00. ¹H NMR (400 MHz, DMSO-d₆) δ 10.94 |
| | (s, 1H), 9.93 (br, 1H), 9.05 (s, 1H), 8.95 (s, 1H), 8.82 (s, 1H), |
| | 8.65 (d, J = 9.2 Hz, 1H), 8.26 (s, 1H), 8.24 (s, 1H), 7.88 (d, J = |
| | 9.2 Hz, 1H), 7.55 (d, J = 8.4 Hz, 1H), 7.42 (s, 1H), 7.15 (s, 1H), |
| | 7.11 (d, J = 8.4 Hz, 1H), 6.77 (s, 1H), 5.05 (dd, J = 13.2, 5.2 Hz, |
| | 1H), 4.33 (d, J = 17.2 Hz, 1H), 4.23 (d, J = 16.8 Hz, 1H), 4.23- |
| | 3.81 (m, 4H), 3.77 (s, 3H), 3.50-3.26 (m, 7H), 3.18-2.98 (m, 5H), |
| | 2.95-2.86 (m, 1H), 2.70 (t, J = 11.6 Hz, 2H), 2.58-2.52 (m, 4H), |
| | 2.45-2.35 (m, 3H), 2.12-1.91 (m, 3H), 1.60-1.41 (m, 2H), 0.86 (t, |
| | J = 7.2 Hz, 3H). |

Compounds 53, 55, 57, 62, and 63 were synthesized via a procedure similar to Compound 38.

### Example 17: Preparation of(S)-N-(6-((5-bromo-2-((4-(4-(4-(2-(4-(2-(2,6-dioxopiperidin-3-yl)-1-oxoisoindolin-5-yl)piperazin-1-yl)ethyl) piperazin-1-yl) piperidin-1-yl)-5-ethyl-2-methoxyphenyl)amino)pyrimidin-4-yl)amino)quinoxalin-5-yl) methanesulfonamide (Compound 39

### Step 1: Preparation of N-(6-((5-bromo-2-((4-(4-(4-(2-chloroethyl) piperazin-1-yl)piperidin-1-yl)-5-ethyl-2-methoxyphenyl)amino)pyrimidin-4-yl)amino)quinoxalin-5-yl) methanesulfonamide

N-(6-((5-bromo-2-((5-ethyl-2-methoxy-4-(4-(piperazin-1-yl)piperidin-1-yl)phenyl)amino)pyrimidin-4-yl)amino)quinoxalin-5-yl) methanesulfonamide (200.000 mg, 0.281 mmol, 1 eq) and 1-bromo-2-chloroethane (48.362 mg, 0.337 mmol, 1.2 eq) were dissolved in DMF (10 mL) at room temperature. DIEA (43.587 mg, 0.337 mmol, 1.2 eq) was added slowly and the reaction mixture was degassed 3 times with N2. The reaction mixture was stirred at 100° C. under a nitrogen atmosphere overnight. LCMS analysis showed complete consumption of starting material and formation of product with the desired mass. The reaction mixture was cooled to room temperature and extracted with ethyl acetate (15 mL×3). The organic layers were combined and washed with water and brine. The organic phase was dried over anhydrous sodium sulfate and concentrated under reduced pressure. The crude residue was purified by column chromatography (DCM:MeOH=4:1) to give N-(6-((5-bromo-2-((4-(4-(4-(2-chloroethyl) piperazin-1-yl) piperidin-1-yl)-5-ethyl-2-methoxyphenyl)amino)pyrimidin-4-yl)amino)quinoxalin-5-yl) methanesulfonamide (150.000 mg, yield 68.95%) as a yellow solid. LCMS: [M+H+]=774.18.

### Step 2: Preparation of(S)-N-(6-((5-bromo-2-((4-(4-(4-(2-(4-(2-(2,6-dioxopiperidin-3-yl)-1-oxoisoindolin-5-yl)piperazin-1-yl)ethyl) piperazin-1-yl)piperidin-1-yl)-5-ethyl-2-methoxyphenyl)amino)pyrimidin-4-yl)amino)quinoxalin-5-yl) methanesulfonamide

N-(6-((5-bromo-2-((4-(4-(4-(2-chloroethyl) piperazin-1-yl)piperidin-1-yl)-5-ethyl-2-methoxyphenyl)amino)pyrimidin-4-yl)amino)quinoxalin-5-yl) methanesulfonamide (100.000 mg, 0.129 mmol, 1 eq) and(S)-3-(1-oxo-5-(piperazin-1-yl) isoindolin-2-yl) piperidine-2,6-dione (50.898 mg, 0.155 mmol, 1.2 eq) were dissolved in DMSO (15 mL) at room temperature. DIEA (20.034 mg, 0.155 mmol, 1.2 eq) was slowly added and the reaction mixture was stirred at 65° C. overnight under nitrogen atmosphere. LCMS indicated complete consumption of starting material and formation of product with the desired mass. The reaction mixture was concentrated under reduced pressure. The resulting crude residue was purified by Prep-HPLC (FA) to afford (S)-N-(6-((5-bromo-2-((4-(4-(4-(2-(4-(2-(2,6-dioxopiperidin-3-yl)-1-oxoisoindolin-5-yl)piperazin-1-yl)ethyl) piperazin-1-yl)piperidin-1-yl)-5-ethyl-2-methoxyphenyl)amino)pyrimidin-4-yl)amino)quinoxalin-5-yl) methanesulfonamide (60.000 mg, yield 43.57%) as a yellow solid. LCMS: [M+H]⁺=1066.09. ¹H NMR (400 MHZ, DMSO-d₆) δ 10.92 (s, 1H), 8.99 (d, J=2.0 Hz, 1H), 8.92 (d, J=2.0 Hz, 1H), 8.87 (s, 1H), 8.64 (d, J=9.2 Hz, 1H), 8.28 (s, 1H), 8.18 (d, J=5.2 Hz, 2H), 7.86 (d. J=9.2 Hz, 1H), 7.52 (d, J=8.4 Hz, 1H), 7.39 (s, 1H), 7.11-7.03 (m, 2H), 6.77 (s, 1H), 5.04 (dd, J=13.2, 5.2 Hz, 1H), 4.33 (d, J=16.8 Hz, 1H), 4.21 (d, J=16.8 Hz, 1H), 3.76 (s, 3H), 3.29-3.25 (m, 8H), 3.06-2.81 (m, 8H), 2.75-2.55 (m, 10H), 2.43-2.30 (m, 6H), 1.98-1.82 (m, 4H), 1.62-1.50 (m, 2H), 0.89-0.81 (m, 3H).

### Example 18: Preparation of N-(6-((5-bromo-2-((4-(2-((1-(2-(2,6-dioxopiperidin-3-yl)-1-oxoisoindolin-5-yl)piperidin-4-yl)methyl)-2,7-diazaspiro[3.5]nonan-7-yl)-5-ethyl-2-methoxyphenyl)amino)pyrimidin-4-yl)amino)quinoxalin-5-yl) methanesulfonamide (Compound 40

### Step 1: Preparation of 2-benzyl 7-(tert-butyl)_{2,7}-diazaspiro[3.5]nonane-2,7-dicarboxylate

Tert-butyl 2,7-diazaspiro[3.5]nonane-7-carboxylate (5.000 g, 19.027 mmol, 1 eq) and TEA (5.776 g, 57.082 mmol, 3 eq) were dissolved in DCM (100 mL) at room temperature, then N-(Benzyloxycarbonyloxy) succinimide was slowly added (4.869 g, 28.541 mmol, 1.5 eq). The mixture was stirred overnight under nitrogen. LCMS indicated complete consumption of starting material and formation of product with the desired mass. The mixture was poured into water, extracted with dichloromethane (50 ml×3), washed with saturated brine. The organic phase was dried with anhydrous sodium sulfate, filtered and concentrated under reduced pressure, the residue was purified by column chromatography (PE:EA=4:1) to afford 2-benzyl 7-(tert-butyl)_{2,7}-diazaspiro[3.5]nonane-2,7-dicarboxylate (5.800 g, yield 84.57%) as a white solid. LCMS: [M+H]⁺=360.45.

### Step 2: Preparation of benzyl 2,7-diazaspiro[3.5]nonane-2-carboxylate

2-Benzyl 7-(tert-butyl)_{2,7}-diazaspiro[3.5]nonane-2,7-dicarboxylate (5.000 g, 13.872 mmol, 1 eq) was dissolved in ethyl acetate (20 mL) in hydrogen chloride solution. The mixture was stirred at room temperature for 30 minutes, LCMS indicated complete consumption of starting material and formation of product with the desired mass. The reaction was concentrated under reduced pressure to afford benzyl 2,7-diazaspiro[3.5]nonane-2-carboxylate (3.200 g, yield 88.61%) as a white solid which was directly used in the next step without further purification. LCMS: [M+H]⁺=260.34.

### Step 3: Preparation of benzyl 7-(2-bromo-5-methoxy-4-nitrophenyl)-2,7-diazaspiro[3.5]nonane-2-carboxylate

Benzyl 2,7-diazaspiro[3.5]nonane-2-carboxylate (5.762 g, 23.047 mmol, 1.2 eq) and 1-bromo-2-fluoro-4-methoxy-5-nitrobenzene (5.000 g, 19.206 mmol, 1 eq) were dissolved in DMSO (150 mL) at room temperature. DIEA (9.929 g, 76.823 mmol, 4 eq) was slowly added and the reaction mixture was degassed with N₂ for 3 times. The reaction mixture was stirred at 120 overnight under nitrogen atmosphere. LCMS analysis indicated complete consumption of starting material and formation of product with the desired mass. The reaction mixture was cooled to room temperature, extracted with ethyl acetate (100 ml×3). The organic layers were combined, washed with water and brine. The organic phase was dried over anhydrous sodium sulfate, concentrated under reduced pressure. The crude residue was purified via column chromatography (PE:EA=4:1) to give benzyl 7-(2-bromo-5-methoxy-4-nitrophenyl)-2,7-diazaspiro[3.5]nonane-2-carboxylate (2.500 g, yield 26.67%) as a yellow solid. LCMS: [M+H]⁺=490.35.

### Step 4: Preparation of benzyl 7-(5-methoxy-4-nitro-2-vinylphenyl)-2,7-diazaspiro[3.5]nonane-2-carboxylate

Benzyl 7-(2-bromo-5-methoxy-4-nitrophenyl)-2,7-diazaspiro[3.5]nonane-2-carboxylate (2.000 g, 4.079 mmol, 1 eq), Potassium vinyltrifluoroborate (1.093 g, 8.157 mmol, 2 eq), K₃PO₄ (1.731 g, 8.157 mmol, 2 eq) and Pd (dppf) Cl₂ (295.568 mg, 0.408 mmol, 0.1 eq) were dissolved in DMF:H₂O (50:5 mL). The reaction mixture was stirred at 100° C. under nitrogen atmosphere for 3 hours. TLC indicated complete consumption of the starting material. The reaction mixture was cooled to room temperature, extracted with dichloromethane (50 ml×3). The organic phase was collected and dried over anhydrous sodium sulfate, filtered. The filtrate was concentrated and the residue was purified by column chromatography on silica gel (PE:EA=3:1) to give benzyl 7-(5-methoxy-4-nitro-2-vinylphenyl)-2,7-diazaspiro[3.5]nonane-2-carboxylate (1.500 g, yield 84.06%) as a yellow solid. LCMS: [M+H]⁺=437.50.

### Step 5: Preparation of benzyl 7-(4-amino-2-ethyl-5-methoxyphenyl)-2,7-diazaspiro[3.5]nonane-2-carboxylate

Benzyl 7-(5-methoxy-4-nitro-2-vinylphenyl)-2,7-diazaspiro[3.5]nonane-2-carboxylate (1.500 g, 3.429 mmol, 1 eq) was dissolved in MeOH (30 mL). Pd/C (36.488 mg, 0.343 mmol, 0.1 eq, 10%, wet) was slowly added and the mixture was stirred at room temperature under H₂ atmosphere overnight. LCMS indicated complete consumption of starting material and formation of product with the desired mass. The reaction solution was filtered and washed with EA. The filtrate was concentrated to give benzyl 7-(4-amino-2-ethyl-5-methoxyphenyl)-2,7-diazaspiro[3.5]nonane-2-carboxylate (1.300 g, yield 92.58%) as a yellow solid which was used directly in next step without further purification. LCMS: [M+H]⁺=409.53.

### Step 6: Preparation of benzyl 7-(4-((5-bromo-4-((5-(methylsulfonamido)quinoxalin-6-yl)amino)pyrimidin-2-yl)amino)-2-ethyl-5-methoxyphenyl)-2,7-diazaspiro[3.5]nonane-2-carboxylate

Benzyl 7-(4-amino-2-ethyl-5-methoxyphenyl)-2,7-diazaspiro[3.5]nonane-2-carboxylate (500.000 mg, 1.816 mmol, 1 eq) and N-(6-((5-bromo-2-chloropyrimidin-4-yl)amino)quinoxalin-5-yl) methanesulfonamide (936.156 mg, 2.179 mmol, 1.2 eq) were dissolved in 20 mL of n-butanol and methanesulfonic acid (348.960 mg, 3.631 mmol, eq). The mixture was stirred at 120° C. for 10 hours. TLC indicated complete consumption of the starting material. The reaction mixture was cooled to room temperature, extracted with dichloromethane (10 mL×3). The organic phase was collected and dried over anhydrous sodium sulfate, concentrated and purified by column chromatography on silica gel (DCM; MeOH=4:1) to afford benzyl 7-(4-((5-bromo-4-((5-(methylsulfonamido)quinoxalin-6-yl)amino)pyrimidin-2-yl)amino)-2-ethyl-5-methoxyphenyl)-2,7-diazaspiro[3.5]nonane-2-carboxylate (1.000 g, yield 82.38%) as a yellow solid. LCMS: [M+H]⁺=802.75.

### Step 7: Preparation of N-(6-((5-bromo-2-((5-ethyl-2-methoxy-4-(2,7-diazaspiro[3.5]nonan-7-yl)phenyl)amino)pyrimidin-4-yl)amino)quinoxalin-5-yl) methanesulfonamide

Benzyl 7-(4-((5-bromo-4-((5-(methylsulfonamido)quinoxalin-6-yl)amino)pyrimidin-2-yl)amino)-2-ethyl-5-methoxyphenyl)-2,7-diazaspiro[3.5]nonane-2-carboxylate (1.000 g, 1.246 mmol, 1 eq) was dissolved in EtOH (50 ml). Pd/C (265.139 mg, 0.249 mmol, 0.2 eq, purity 10%, wet) was slowly added and the mixture was stirred at 65° C. under H₂ atmosphere overnight. LCMS indicated complete consumption of starting material and formation of product with the desired mass. The reaction solution was filtered and washed with EA, and the organic phase was concentrated under reduced pressure to afford N-(6-((5-bromo-2-((5-ethyl-2-methoxy-4-(2,7-diazaspiro[3.5]nonan-7-yl)phenyl)amino)pyrimidin-4-yl)amino)quinoxalin-5-yl) methanesulfonamide (820.000 mg, yield 98.45%) as a white solid which was used directly in next step without further purification. LCMS: [M+H]⁺=668.62.

### Step 8: Preparation of N-(6-((5-bromo-2-((4-(2-((1-(2-(2,6-dioxopiperidin-3-yl)-1-oxoisoindolin-5-yl)piperidin-4-yl)methyl)-2,7-diazaspiro[3.5]nonan-7-yl)-5-ethyl-2-methoxyphenyl)amino)pyrimidin-4-yl)amino)quinoxalin-5-yl) methanesulfonamide

1-(2-(2,6-Dioxopiperidin-3-yl)-1-oxoisoindolin-5-yl)piperidine-4-carbaldehyde (70.327 mg, 0.179 mmol, 1.2 eq) and DIEA (38.662 mg, 0.299 mmol, 2 eq) were dissolved in MeOH (10 mL) at 15° C. N-(6-((5-bromo-2-((5-ethyl-2-methoxy-4-(2,7-diazaspiro[3.5]nonan-7-yl)phenyl)amino)pyrimidin-4-yl)amino)quinoxalin-5-yl) methanesulfonamide (100.000 mg, 0.150 mmol, 1 eq) and AcOH (35.925 mg, 0.598 mmol, 4 eq) were added and the reaction mixture was stirred for 1 h. NaBH₃CN (18.770 mg, 0.299 mmol, 2 eq) was added and the reaction mixture was stirred for another 3 h. LCMS indicated complete consumption of starting material and formation of product with the desired mass. The reaction mixture was concentrated under reduced pressure and the resulting crude residue was purified by Prep-HPLC (FA) to afford N-(6-((5-bromo-2-((4-(2-((1-(2-(2,6-dioxopiperidin-3-yl)-1-oxoisoindolin-5-yl)piperidin-4-yl)methyl)-2,7-diazaspiro[3.5]nonan-7-yl)-5-ethyl-2-methoxyphenyl)amino)pyrimidin-4-yl)amino)quinoxalin-5-yl) methanesulfonamide (50.000 mg, yield 33.17%) as a white solid. LCMS: [M+H]⁺=1008.01.1H NMR (400 MHZ, DMSO-d₆) δ 10.95 (s, 1H), 9.00 (d, J=2.0 Hz, 1H), 8.93 (d, J=2.0 Hz, 2H), 8.65 (d, J=9.2 Hz, 1H), 8.29 (s, 1H), 8.25 (s, 1H), 7.86 (d, J=9.6 Hz, 1H), 7.51 (d, J=8.4 Hz, 1H), 7.41 (s, 1H), 7.07-7.04 (m, 2H), 6.77 (s, 1H), 5.05 (dd, J=13.2, 5.2 Hz, 1H), 4.33 (d, J=16.8 Hz, 1H), 4.21 (d, J=16.8 Hz, 1H), 3.88 (d, J=12.4 Hz, 2H), 3.77 (s, 3H), 3.06-3.02 (m, 8H), 2.87-2.67 (m, 8H), 2.50-2.32 (m, 5H), 2.00-1.92 (m, 1H), 1.92-1.74 (m, 6H), 1.25-1.22 (m, 2H), 0.92-0.76 (m, 3H).

### Example 19: Preparation of(S)-N-(6-((5-bromo-2-((4-(4-(4-(2-(2,6-dioxopiperidin-3-yl)-1-oxoisoindolin-5-yl)piperazine-1-carbonyl)-[1,4′-bipiperidin]-1′-yl)-5-ethyl-2-methoxyphenyl)amino)pyrimidin-4-yl)amino)quinoxalin-5-yl) methanesulfonamide (Compound 41

### Step 1: Preparation of benzyl(S)-4-(4-(2-(2,6-dioxopiperidin-3-yl)-1-oxoisoindolin-5-yl) piperazine-1-carbonyl) piperidine-1-carboxylate

(S)-3-(1-oxo-5-(piperazin-1-yl) isoindolin-2-yl)piperidine-2,6-dione (1.033 g, 3.924 mmol, 1.1 eq) and HATU (1.632 g, 4.281 mmol, 1.2 eq) were dissolved in DCM (100 mL), then DIEA (1.383 g, 10.701 mmol, 3 eq) was added slowly and the reaction mixture was stirred for 0.5 h, 1-((benzyloxy) carbonyl) piperidine-4-carboxylic acid (2.000 g, 3.567 mmol, 1 eq) was added, and the reaction mixture was stirred for another 1 h at room temperature. LCMS analysis indicated complete consumption of starting material and formation of product with the desired mass. The reaction mixture was concentrated under reduced pressure. The crude residue was purified via column chromatography MeOH/DCM (2/98) to give Benzyl(S)-4-(4-(2-(2,6-dioxopiperidin-3-yl)-1-oxoisoindolin-5-yl) piperazine-1-carbonyl) piperidine-1-carboxylate (2.000 g, yield 97.74%) as a white solid. LCMS: [M+H]⁺=574.26.

### Step 2: Preparation of(S)-3-(1-oxo-5-(4-(piperidine-4-carbonyl) piperazin-1-yl) isoindolin-2-yl)piperidine-2,6-dione

A mixture of Benzyl(S)-4-(4-(2-(2,6-dioxopiperidin-3-yl)-1-oxoisoindolin-5-yl) piperazine-1-carbonyl) piperidine-1-carboxylate (2.000 g, 3.486 mmol, 1 eq) in HBr/AcOH (10 eq) was stirred at room temperature for 2 hours. LCMS analysis indicated complete consumption of starting material and formation of product with desired mass. The solvent was concentrated under reduced pressure to give(S)-3-(1-oxo-5-(4-(piperidine-4-carbonyl) piperazin-1-yl) isoindolin-2-yl)piperidine-2,6-dione (1.500 g, yield 97.89%) as a white solid which was used directly in next step without further purification. LCMS: [M+H]⁺=440.22.

### Step 3: Preparation of(S)-N-(6-((5-bromo-2-((4-(4-(4-(2-(2,6-dioxopiperidin-3-yl)-1-oxoisoindolin-5-yl)piperazine-1-carbonyl)-[1,4′-bipiperidin]-1′-yl)-5-ethyl-2-methoxyphenyl)amino)pyrimidin-4-yl)amino)quinoxalin-5-yl) methanesulfonamide

A mixture of(S)-3-(1-oxo-5-(4-(piperidine-4-carbonyl) piperazin-1-yl) isoindolin-2-yl) piperidine-2,6-dione (77.874 mg, 0.150 mmol, 1.2 eq), TEA (37.855 mg, 0.374 mmol, eq) in DCM (20 mL) and MeOH (5 mL) was stirred at room temperature for 0.5 hour. CH₃COOH (7.488 mg, 0.125 mmol, 1 eq), N-(6-((5-bromo-2-((5-ethyl-2-methoxy-4-(4-oxopiperidin-1-yl)phenyl)amino)pyrimidin-4-yl)amino)quinoxalin-5-yl) methanesulfonamide (80.000 mg, 0.125 mmol, 1 eq) were added and stirred at room temperature for 1 hour. NaBH₃CN (11.552 mg, 0.187 mmol, 1.5 eq) was added and the reaction mixture was stirred at room temperature for another 48 hours. LCMS analysis indicated complete consumption of starting material and formation of product with the desired mass. The reaction mixture was concentrated under reduced pressure and the resulting crude residue was purified by silica-gel column chromatograph (DCM:MeOH=97:3) to afford (S)-N-(6-((5-bromo-2-((4-(4-(4-(2-(2,6-dioxopiperidin-3-yl)-1-oxoisoindolin-5-yl)piperazine-1-carbonyl)-[1,4′-bipiperidin]-1′-yl)-5-ethyl-2-methoxyphenyl)amino)pyrimidin-4-yl)amino)quinoxalin-5-yl) methanesulfonamide (11.500 mg, yield 8.66%) as a white solid. LCMS: [M+H]⁺=1066.06.1H NMR (400 MHZ, DMSO-d₆) δ 10.95 (s, 1H), 9.91 (s, 1H), 9.01 (d, J=2.0 Hz, 1H), 8.94 (d, J=2.0 Hz, 1H), 8.87 (s, 1H), 8.65 (d. J=9.4 Hz, 1H), 8.30 (s, 1H), 8.22 (s, 1H), 7.89 (d, J=9.4 Hz, 1H), 7.57 (d, J=8.4 Hz, 1H), 7.41 (s, 1H), 7.17 (s, 1H), 7.11 (d, J=8.4 Hz, 1H), 6.79 (s, 1H), 5.06 (dd, J=13.2, 5.2 Hz, 1H), 4.33 (d. J=17.2 Hz, 1H), 4.24 (d. J=16.8 Hz, 1H), 3.78 (s, 3H), 3.75-3.51 (m, 4H), 3.26-3.23 (m, 3H), 3.21-3.15 (m, 2H), 3.05-2.9 (m, 5H), 2.97-2.81 (m, 2H), 2.78-2.65 (m, 3H), 2.64-2.55 (m, 2H), 2.48-2.31 (m, 3H), 2.04-1.88 (m, 3H), 1.76-1.65 (m, 5H), 1.38-1.21 (m, 3H), 0.86-0.75 (m, 3H).

### Example 20: Preparation of N-(6-((5-bromo-2-((4-(4-(4-((7-(2-(2,6-dioxopiperidin-3-yl)-1-oxoisoindolin-5-yl)-7-azaspiro[3.5]nonan-2-yl)methyl) piperazin-1-yl)piperidin-1-yl)-5-ethyl-2-methoxyphenyl)amino)pyrimidin-4-yl)amino)quinoxalin-5-yl) methanesulfonamide (Compound 47

### Step 1: Preparation of tert-butyl 2-(dimethoxymethyl)-7-azaspiro[3.5]nonane-7-carboxylate

tert-Butyl 2-formyl-7-azaspiro[3.5]nonane-7-carboxylate (5 g, 19.8 mmol, 1.0 eq), Trimethoxymethane (4.2 g, 39.6 mmol, 2.0 eq) and TsOH (3.4 g, 39.6 mmol, 2.0 eq) were dissolved in MeOH (50 mL). The reaction mixture was allowed to stir at 25° C. for 16 h. LCMS analysis indicated complete consumption of starting material and formation of product with the desired mass. The reaction mixture was concentrated under reduced pressure and the crude residue was purified via column chromatography (PE:EA=3:1) to give tert-butyl 2-(dimethoxymethyl)-7-azaspiro[3.5]nonane-7-carboxylate (4.5 g, 76% yield) as a white solid. LCMS: [M+H]⁺=300.2.

### Step 2: Preparation of 2-(dimethoxymethyl)-7-azaspiro[3.5]nonane

tert-Butyl 2-(dimethoxymethyl)-7-azaspiro[3.5]nonane-7-carboxylate (2 g, 6.7 mmol, 1.0 eq) and TMSI (1.9 g, 13.4 mmol, 2.0 eq) were dissolved in MeCN (20 mL). The reaction mixture was allowed to stir at 25° C. for 2 h. LCMS analysis indicated complete consumption of starting material and formation of product with the desired mass. The reaction mixture was concentrated to give crude 2-(dimethoxymethyl)-7-azaspiro[3.5]nonane (1 g) as a brown oil which was used directly in next step without further purification. LCMS: [M+H]⁺=200.3.

### Step 3: Preparation of methyl 2-bromo-5-(2-(dimethoxymethyl)-7-azaspiro[3.5]nonan-7-yl)benzoate

2-(Dimethoxymethyl)-7-azaspiro[3.5]nonane (800 mg, 4 mmol, 1.0 eq), methyl 2-bromo-5-fluorobenzoate (930 mg, 4 mmol, 1.0 eq) and DIEA (1.5 g, 12 mmol, 3.0 eq) were dissolved in DMSO (10 mL) and the reaction mixture was stirred at 120° C. for 16 h. LCMS analysis indicated complete consumption of starting material and formation of product with the desired mass. The reaction mixture was poured into water (10 mL) and extracted with EA (10 mL*3). The organic layers were combined, washed with water and brine. The organic phase was dried over anhydrous sodium sulfate, concentrated under reduced pressure. The crude residue was purified via column chromatography (PE:EA=3:1) to give methyl 2-bromo-5-(2-(dimethoxymethyl)-7-azaspiro[3.5]nonan-7-yl)benzoate (1.3 g, 81% yield) as a yellow solid. LCMS: [M+H]⁺=412.3.

### Step 4: Preparation of methyl 5-(2-(dimethoxymethyl)-7-azaspiro[3.5]nonan-7-yl)-2-formylbenzoate

Methyl 2-bromo-5-(2-(dimethoxymethyl)-7-azaspiro[3.5]nonan-7-yl)benzoate (1.3 g, 3.2 mmol, 1.0 eq), t-BuNC (530 mg, 6.4 mmol, 2.0 eq), Et₃SiH (1.1 g, 9.6 mmol, 3.0 eq), Pd(OAc)₂ (72 mg, 0.32 mmol, 0.1 eq), P(cy)₃ (180 mg, 0.64 mmol, 0.2 eq) and Na₂CO₃ (678 mg, 6.4 mmol, 2.0 eq) were dissolved in DMF (10 mL). The reaction mixture was stirred at 65° C. for 16 h. LCMS analysis indicated complete consumption of starting material and formation of product with the desired mass. The reaction mixture was concentrated under reduced pressure. The crude residue was purified via column chromatography (PE:EA=2:1) to give 5-(2-(dimethoxymethyl)-7-azaspiro[3.5]nonan-7-yl)-2-formylbenzoate (800 mg, 71% yield) as a yellow solid. LCMS: [M+H]⁺=362.3.

### Step 5: Preparation of 3-(5-(2-(dimethoxymethyl)-7-azaspiro[3.5]nonan-7-yl)-1-oxoisoindolin-2-yl)piperidine-2,6-dione

5-(2-(Dimethoxymethyl)-7-azaspiro[3.5]nonan-7-yl)-2-formylbenzoate (800 mg, 2.2 mmol, 1.0 eq), 3-aminopiperidine-2,6-dione (333 mg, 2.6 mmol, 1.2 eq) and NaOAc (361 mg, 4.4 mmol, 2.0 eq) were dissolved in MeOH (10 mL). The reaction mixture was allowed to stir at 25° C. for 0.5 h. AcOH (66 mg, 11 mmol, 5.0 eq) was added and the reaction mixture was stirred for another 0.5 h. NaBH₃CN (164 mg, 2.6 mmol, 1.2 eq) was added and the mixture was stirred for another 15 h. LCMS analysis indicated complete consumption of starting material and formation of product with the desired mass. The reaction mixture was filtered and the filter cake was washed with MeOH to give 3-(5-(2-(dimethoxymethyl)-7-azaspiro[3.5]nonan-7-yl)-1-oxoisoindolin-2-yl)piperidine-2,6-dione (300 mg) as a blue solid which was used directly in nest step without further purification. LCMS: [M+H]⁺=442.3.

### Step 6: Preparation of 7-(2-(2,6-dioxopiperidin-3-yl)-1-oxoisoindolin-5-yl)-7-azaspiro[3.5]nonane-2-carbaldehyde

3-(5-(2-(Dimethoxymethyl)-7-azaspiro[3.5]nonan-7-yl)-1-oxoisoindolin-2-yl) piperidine-2,6-dione (150 mg, 0.34 mmol, 1.0 eq) was dissolved in 2N HCl/THF (2 mL/2 mL). The reaction mixture was allowed to stir at 25° C. for 16 h. LCMS analysis indicated complete consumption of starting material and formation of product with the desired mass. The reaction mixture was neutralized and extracted with ethyl acetate (10 ml×3). The organic layers were combined, washed with water and brine. The organic phase was dried over anhydrous sodium sulfate, concentrated in vacuo to give crude residue 7-(2-(2,6-dioxopiperidin-3-yl)-1-oxoisoindolin-5-yl)-7-azaspiro[3.5]nonane-2-carbaldehyde (120 mg) as a off-white oil. LCMS: [M+H]⁺=396.2.

### Step 7: Preparation of N-(6-((5-bromo-2-((4-(4-(4-((7-(2-(2,6-dioxopiperidin-3-yl)-1-oxoisoindolin-5-yl)-7-azaspiro[3.5]nonan-2-yl)methyl) piperazin-1-yl)piperidin-1-yl)-5-ethyl-2-methoxyphenyl)amino)pyrimidin-4-yl)amino)quinoxalin-5-yl) methanesulfonamide

7-(2-(2,6-Dioxopiperidin-3-yl)-1-oxoisoindolin-5-yl)-7-azaspiro[3.5]nonane-2-carbaldehyde (120 mg, 0.3 mmol, 1.0 eq), N-(6-((5-bromo-2-((5-ethyl-2-methoxy-4-(4-(piperazin-1-yl)piperidin-1-yl)phenyl)amino)pyrimidin-4-yl)amino)quinoxalin-5-yl) methanesulfonamide (214 mg, 0.3 mmol, 1.0 eq) and DIEA (39 mg, 0.3 mmol, 1.0 eq) were dissolved in MeOH (10 mL). The reaction mixture was allowed to stir at 25° C. for 0.5 h. AcOH (18 mg, 0.3 mmol, 1.0 eq) was added and the reaction mixture was stirred for another 0.5 h. NaBH₃CN (23 mg, 0.36 mmol, 1.2 eq) was added and reaction mixture was stirred for another 15 h. LCMS analysis indicated complete consumption of starting material and formation of product with desired mass. The reaction mixture was concentrated under reduced pressure and the resulting crude residue was purified by Prep-HPLC to afford N-(6-((5-bromo-2-((4-(4-(4-((7-(2-(2,6-dioxopiperidin-3-yl)-1-oxoisoindolin-5-yl)-7-azaspiro[3.5]nonan-2-yl)methyl) piperazin-1-yl)piperidin-1-yl)-5-ethyl-2-methoxyphenyl)amino)pyrimidin-4-yl)amino)quinoxalin-5-yl) methanesulfonamide (170 mg, 52% yield) as a yellow solid. LCMS: [M+H]⁺=1092.00.1H NMR (400 MHZ, DMSO-d₆) δ 10.93 (s, 1H), 9.00 (d, J=2.0 Hz, 1H), 8.93 (d, J=2.0 Hz, 1H), 8.86 (s, 1H), 8.65 (d, J=9.2 Hz, 1H), 8.29 (s, 1H), 8.21 (s, 1H), 7.87 (d, J=9.2 Hz, 1H), 7.49 (d, J=8.4 Hz, 1H), 7.40 (s, 1H), 7.06 (s, 1H), 7.03 (d, J=8.4 Hz, 1H), 6.77 (s, 1H), 5.04 (dd, J=13.2, 5.2 Hz, 1H), 4.31 (d, J=16.8 Hz, 1H), 4.19 (d, J=16.8 Hz, 1H), 3.76 (s, 3H), 3.20 (s, 3H), 3.05-2.98 (m, 6H), 2.96-2.81 (m, 2H), 2.75-2.71 (m, 4H), 2.62-2.55 (m, 4H), 2.46-2.34 (m, 4H), 2.01-1.82 (m, 6H), 1.77-1.39 (m, 10H), 1.26-1.23 (m, 3H), 0.84 (t, J=7.6 Hz, 3H).

| Compd # | Characterizations |
|---|---|
| 48 | LCMS: [M + H]⁺ = 1076.6. ¹H NMR (400 MHz, DMSO-d₆) δ 10.94 |
| | (s, 1H), 9.00 (d, J = 2.0 Hz, 1H), 8.93 (d, J = 2.0 Hz, 1H), 8.89 (s, |
| | 1H), 8.65 (d, J = 8.8 Hz, 1H), 8.29 (s, 1H), 8.21 (s, 1H), 7.87 (d, |
| | J = 9.6 Hz, 1H), 7.50 (d, J = 8.4 Hz, 1H), 7.40 (s, 1H), 7.07 (s, 1H), |
| | 7.05 (d, J = 8.5 Hz, 1H), 6.79 (s, 1H), 5.04 (dd, J = 13.2, 5.2 Hz, |
| | 1H), 4.31 (d, J = 17.2 Hz, 1H), 4.23 (d, J = 16.8 Hz, 1H), 3.77 (s, |
| | 3H), 3.25-3.20 (m, 5H), 3.05-2.95 (m, 6H), 2.94-2.85 (m, 2H), |
| | 2.76-2.65 (m, 4H), 2.63-2.56 (m, 3H), 2.49-2.41 (m, 3H), 2.36- |
| | 2.25 (m, 4H), 2.01-1.95 (m, 3H), 1.86-1.77 (m, 2H), 1.68-1.61 (m, |
| | 2H), 1.60-1.51 (m, 5H), 0.93-0.81 (m, 3H). |
| 49 | LCMS: [M + H]⁺ = 1037.90. ¹H NMR (400 MHz, DMSO-d₆) δ 10.94 |
| | (s, 1H), 9.00 (d, J = 2.0 Hz, 1H), 8.93 (d, J = 2.0 Hz, 1H), 8.86 (s, |
| | 1H), 8.65 (d, J = 9.2 Hz, 1H), 8.29 (s, 1H), 8.22 (s, 1H), 7.98-7.86 |
| | (m, 1H), 7.69 (dd, J = 7.6, 1.6 Hz, 0.5H), 7.53-7.40 (m, 2H), 7.22 |
| | (td, J = 7.2, 1.6 Hz, 0.6H), 7.08 (s, 1H), 6.78 (s, 1H), 5.05 (dd, J = |
| | 13.2, 5.2 Hz, 1H), 4.33 (d, J = 16.8 Hz, 1H), 4.21 (d, J = 16.8 Hz, |
| | 1H), 4.01-3.92 (m, 2H), 3.76 (s, 3H), 3.33-3.25 (m, 8H), 3.04-2.99 |
| | (m, 4H), 2.96-2.84 (m, 4H), 2.82-2.67 (m, 6H), 2.59-2.53 (m, 2H), |
| | 2.44-2.38 (m, 2H), 2.01-1.83 (m, 4H), 1.62-1.51 (m, 4H), 0.86- |
| | 0.76 (m, 3H). |
| 50 | LCMS: [M + H]⁺ = 1077.6. ¹H NMR (400 MHz, DMSO-d₆) δ 10.92 |
| | (s, 1H), 9.91 (br, 1H), 9.00 (d, J = 1.6 Hz, 1H), 8.93 (d, J = 1.6 Hz, |
| | 1H), 8.86 (s, 1H), 8.65 (d, J = 8.0 Hz, 1H), 8.29 (s, 1H), 8.22 (s, |
| | 1H), 7.88 (d, J = 8.8 Hz, 1H), 7.49 (d, J = 8.0 Hz, 1H), 7.41 (s, 1H), |
| | 6.78 (s, 1H), 6.51 (s, 1H), 6.48 (d, J = 8.0 Hz, 1H), 5.03 (dd, J = |
| | 13.2, 5.2 Hz, 1H), 4.30 (d, J = 16.8 Hz, 1H), 4.18 (d, J = 16.8 Hz, |
| | 1H), 3.77 (s, 3H), 3.64 (d, J = 24.8 Hz, 4H), 3.25-3.16 (m, 4H), |
| | 3.15-2.98 (m, 6H), 2.97-2.83 (m, 3H), 2.77-2.65 (m, 3H), 2.62- |
| | 2.55 (m, 2H), 2.48-2.31 (m, 5H), 2.08-1.88 (m, 7H), 1.59-1.51 (m, |
| | 2H), 1.29-1.21 (m, 3H), 0.89-0.81 (m, 3H). |

Compounds 48, 49, 50 were synthesized via a procedure similar to Compound 47.

### Example 21: Preparation of N-16-(5-bromo-2-((4-(4-(4-((1(2-(2,6-dioxopiperidin-3-yl)-1-oxoisoindolin-5-yl)piperidin-4-yl)methyl) piperazin-1-yl)piperidin-1-yl)-5-ethyl-2-methoxyphenyl)amino)pyrimidin-4-yl)amino)quinoxalin-5-yl)-N-methylmethanesulfonamide (Compound 93

### Step 1: Preparation of N-(6-((5-bromo-2-chloropyrimidin-4-yl)amino)quinoxalin-5-yl)-N-methylmethanesulfonamide

To a solution of N-(6-((5-bromo-2-chloropyrimidin-4-yl)amino)quinoxalin-5-yl) methanesulfonamide (1.000 g, 2.327 mmol, 1 eq) in MeCN (10.0 mL) were added K₂CO₃ (802.923 mg, 5.818 mmol, 2.5 eq) and KI (463.601 mg, 2.793 mmol, 1.2 eq). The resulting mixture was stirred at 50° C. for 13 h. LCMS indicated complete consumption of starting material and formation of product with the desired mass. The mixture was poured into water (30 mL) and extracted with EtOAc (20 mL×3). The combined organic layer was washed with brine (50 mL), dried over anhydrous Na₂SO₄ and concentrated under reduced pressure. The residue was purified by silica gel chromatography (EA/PE=⅓) to afford N-(6-((5-bromo-2-chloropyrimidin-4-yl)amino)quinoxalin-5-yl)-N-methylmethanesulfonamide (600.000 mg, yield 58.10%) as a white solid. LCMS: [M+H]⁺=443.4.

### Step 2: Preparation of benzyl 4-(1-(4-((5-bromo-4-((5-(N-methylmethylsulfonamido) quinoxalin-6-yl)amino)pyrimidin-2-yl)amino)-2-ethyl-5-methoxyphenyl)piperidin-4-yl)piperazine-1-carboxylate

To a mixture of N-(6-((5-bromo-2-chloropyrimidin-4-yl)amino)quinoxalin-5-yl)-N-methylmethanesulfonamide (500.000 mg, 1.127 mmol, 1 eq) and benzyl 4-(1-(4-amino-2-ethyl-5-methoxyphenyl)piperidin-4-yl)piperazine-1-carboxylate (510.018 mg, 1.127 mmol, 1 eq) in DMSO (10 mL) was added DIEA (436.096 mg, 3.381 mmol, 3 eq). The mixture was stirred at 100° C. for 12 h. LCMS indicated complete consumption of starting material and formation of product with the desired mass. The reaction mixture was quenched with water (15 mL) and extracted with EA (20 mL*3). The organic phase was washed with brine (10 mL) and the mixture was concentrated under reduced pressure. The crude residue was purified by flash column (EA/PE=5/1) to give benzyl 4-(1-(4-((5-bromo-4-((5-(N-methylmethylsulfonamido)quinoxalin-6-yl)amino)pyrimidin-2-yl)amino)-2-ethyl-5-methoxyphenyl)piperidin-4-yl)piperazine-1-carboxylate (180.000 mg, yield 18.58%) as a white solid. LCMS [M+H]⁺=859.3.

### Step 3: Preparation of N-(6-((5-bromo-2-((5-ethyl-2-methoxy-4-(4-(piperazin-1-yl) piperidin-1-yl)phenyl)amino)pyrimidin-4-yl)amino)quinoxalin-5-yl)-N-methylmethanesulfonamide

To a solution of benzyl 4-(1-(4-((5-bromo-4-((5-(N-methylmethylsulfonamido) quinoxalin-6-yl)amino)pyrimidin-2-yl)amino)-2-ethyl-5-methoxyphenyl)piperidin-4-yl)piperazine-1-carboxylate (180.000 mg, 0.209 mmol, 1 eq) in EtOH (6 mL) was added Pd/C (10%, wet). The resulting mixture was stirred under an atmosphere of H₂ balloon for 12 h. The mixture was filtered through a celite pad. The celite pad was washed with EtOAc and the filtrate was concentrated in vacuo to afford N-(6-((5-bromo-2-((5-ethyl-2-methoxy-4-(4-(piperazin-1-yl)piperidin-1-yl)phenyl)amino)pyrimidin-4-yl)amino)quinoxalin-5-yl)-N-methylmethanesulfonamide (140.000 mg, yield 92.15%). The residue was used next directly without further purification.

### Step 4: Preparation of N-(6-((5-bromo-2-((4-(4-(4-((1-(2-(2,6-dioxopiperidin-3-yl)-1-oxoisoindolin-5-yl)piperidin-4-yl)methyl) piperazin-1-yl)piperidin-1-yl)-5-ethyl-2-methoxyphenyl)amino)pyrimidin-4-yl)amino)quinoxalin-5-yl)-N-methylmethanesulfonamide

N-(6-((5-bromo-2-((5-ethyl-2-methoxy-4-(4-(piperazin-1-yl)piperidin-1-yl)phenyl)amino)pyrimidin-4-yl)amino)quinoxalin-5-yl)-N-methylmethanesulfonamide (130.000 mg, 0.179 mmol, 1 eq) and DIEA (23.108 mg, 0.179 mmol, 1 eq) in MeOH (6 mL) was stirred at 25° C. for 20 minutes. AcOH (21.496 mg, 0.358 mmol, 2 eq) and 4-(2-(2,6-dioxopiperidin-3-yl)-1-oxoisoindolin-5-yl)cyclohexane-1-carbaldehyde (95.228 mg, 0.269 mmol, 1.5 eq) were added. The mixture was stirred at 25° C. for 0.5 h, before NaBH₃CN (11.285 mg, 0.179 mmol, 1 eq) was added. The mixture was stirred at 25° C. for further 1 h. LCMS indicated complete consumption of starting material and formation of product with the desired mass. The reaction mixture was concentrated under reduced pressure to afford a residue. The residue was purified by Prep-HPLC to give N-(6-((5-bromo-2-((4-(4-(4-((1-(2-(2,6-dioxopiperidin-3-yl)-1-oxoisoindolin-5-yl)piperidin-4-yl)methyl) piperazin-1-yl)piperidin-1-yl)-5-ethyl-2-methoxyphenyl)amino)pyrimidin-4-yl)amino)quinoxalin-5-yl)-N-methylmethanesulfonamide (35.100 mg, yield 18.40%) as a white solid. LCMS: [M+H]⁺=1065.85.1H NMR (400 MHZ, DMSO-d₆) δ 10.92 (s, 1H), 8.97 (d. J=2.0 Hz, 1H), 8.92 (d, J=2.0 Hz, 1H), 8.80 (d, J=9.6 Hz, 1H), 8.57 (s, 1H), 8.36 (s, 1H), 8.30 (s, 1H), 7.91 (d. J=9.6 Hz, 1H), 7.51 (d, J=8.8 Hz, 1H), 7.37 (s, 1H), 706 (s, 1H), 7.04 (d, J=7.6 Hz, 1H), 6.80 (s, 1H), 5.03 (dd, J=13.2, 5.2 Hz, 1H), 4.32 (d, J=16.8 Hz, 1H), 4.20 (d, J=16.8 Hz, 1H), 3.87 (d, J=12.8 Hz, 2H), 3.76 (s, 3H), 3.23 (s, 3H), 3.06-3.01 (m, 3H), 2.98-2.93 (m, 1H), 2.92-2.84 (m, 3H), 2.76-2.60 (m, 7H), 2.59-2.56 (m, 1H), 2.49-2.41 (m, 5H), 2.38-2.55 (m, 2H), 2.25-2.18 (m, 2H), 1.99-1.89 (m, 3H), 1.79-1.21 (m, 4H), 1.65-1.53 (m, 2H), 1.25-1.15 (m, 2H), 0.93 (t, J=7.6 Hz, 3H).

### Example 22: Preparation of N-(6-((5-bromo-2-((4-(4-(4-(2-(1-(4-(2,6-dioxopiperidin-3-yl)-3,5-difluorophenyl)piperidin-4-yl)ethyl) piperazin-1-yl)piperidin-1-yl)-5-ethyl-2-methoxyphenyl)amino)pyrimidin-4-yl)amino)quinoxalin-5-yl) methanesulfonamide (Compound 90

### Step 1: Preparation of 2,6-bis(benzyloxy)-3-(4-bromo-2,6-difluorophenyl)pyridine

To a solution of 5-bromo-1,3-difluoro-2-iodobenzene (2.000 g, 6.272 mmol, 1 eq), 2,6-bis(benzyloxy)-3-(4,4,5,5-tetramethyl-1,3,2-dioxaborolan-2-yl)pyridine (2.617 g, 6.272 mmol, 1 eq) in dioxane (10 mL)/H₂O (2 mL) were added Pd (dppf) Cl₂ (458.480 mg, 0.627 mmol, 0.1 eq), K₂CO₃ (2.597 g, 18.816 mmol, 3 eq). The resulting solution was stirred at 110° C. under N₂ atmosphere for 3 h, then the reaction solution was washed with brine, extracted with EA (15 mL*3). The organic phase was dried over Na₂SO₄, concentrated under reduced pressure to afford crude product which was purified by flash column (EA/PE= 1/10) to obtain 2,6-bis(benzyloxy)-3-(4-bromo-2,6-difluorophenyl)pyridine (1.100 g, yield 59%) as a brown solid. LCMS: [M+H]⁺=482.2. Step 2: Preparation of ethyl 2-(1-(4-(2,6-bis(benzyloxy)pyridin-3-yl)-3,5-difluorophenyl)piperidin-4-yl)acetate

A mixture of 2,6-bis(benzyloxy)-3-(4-bromo-2,6-difluorophenyl)pyridine (1.8 g, 4.147 mmol, 1 eq), ethyl 2-(piperidin-4-yl)acetate (1.065 g, 6.220 mmol, 1.5 eq), Pd (dba)₂ (379.416 mg, 0.415 mmol, 0.1 eq), Davephos (326.373 mg, 0.829 mmol, 0.2 eq), Cs₂CO₃ (3.369 g, 10.367 mmol, 2.5 eq) in 2-methyl-THF (30 mL) and H₂O (3 mL) was stirred overnight at 100° C. under nitrogen atmosphere. The resulting mixture was extracted with EtOAc. The combined organic layers were washed with brine, dried over anhydrous Na₂SO₄. After filtration, the filtrate was concentrated under reduced pressure. The residue was purified by silica gel column chromatography, eluted with PE/EtOAc (1/1) to afford ethyl 2-(1-(4-(2,6-bis(benzyloxy)pyridin-3-yl)-3,5-difluorophenyl)piperidin-4-yl)acetate (1.800 g, yield 75.80%). LCMS: [M+H]⁺=573.1.

### Step 3: Preparation of 2-(1-(4-(2,6-bis(benzyloxy)pyridin-3-yl)-3,5-difluorophenyl)piperidin-4-yl) ethan-1-ol

To a stirred mixture of ethyl 2-(1-(4-(2,6-bis(benzyloxy)pyridin-3-yl)-3,5-difluorophenyl)piperidin-4-yl)acetate (1.500 g, 2.619 mmol, 1 eq) in THF (15 mL) was added LiAlH₄ (497.031 mg, 13.097 mmol, 5 eq) in portion at room temperature, then the mixture was stirred at room temperature overnight. The reaction was quenched with water (20 mL) at room temperature, extracted with EtOAc (3×30 mL). The combined organic layers were washed with brine, dried over anhydrous Na₂SO₄. After filtration, the filtrate was concentrated under reduced pressure to afford 2-(1-(4-(2,6-bis(benzyloxy)pyridin-3-yl)-3,5-difluorophenyl)piperidin-4-yl) ethan-1-ol (900.000 mg, yield 64.75%) as a light yellow solid. LCMS: [M+H]⁺=531.2.

### Step 4: Preparation of 3-(2,6-difluoro-4-(4-(2-hydroxyethyl) piperidin-1-yl)phenyl)piperidine-2,6-dione

To a stirred mixture of 2-(1-(4-(2,6-bis(benzyloxy)pyridin-3-yl)-3,5-difluorophenyl)piperidin-4-yl) ethan-1-ol (900.000 mg, 1.696 mmol, 1 eq) and Pd/C (217.109 mg, 3.392 mmol, 2 eq, 10%, wet) in EtOH (15 mL) and DCM (5 mL) was added AcOH (5 mL) at rt and the mixture was stirred at 40° C. under hydrogen atmosphere overnight. The resulting mixture was filtered and the filter cake was washed with MeOH (20 mL). The filtrate was concentrated under reduced pressure to afford 3-(2,6-difluoro-4-(4-(2-hydroxyethyl) piperidin-1-yl)phenyl)piperidine-2,6-dione (550.000 mg, yield 92.02%) as a yellow solid which was used directly in next step without further purification. LCMS: [M+H]⁺=353.3.

### Step 5: Preparation of 2-(1-(4-(2,6-dioxopiperidin-3-yl)-3,5-difluorophenyl)piperidin-4-yl) acetaldehyde

To a mixture of 3-(2,6-difluoro-4-(4-(2-hydroxyethyl) piperidin-1-yl)phenyl)piperidine-2,6-dione (260.000 mg, 0.738 mmol, 1 eq) in DCM (5 mL) at rt was added Dess-Martin periodinane (625.688 mg, 1.476 mmol, 2 eq). The mixture was stirred at rt for 2 h. Saturated NaHCO₃ solution was added, and the mixture was extracted with EA (10 mL×2). The combined organic layers were washed with water and brine, dried and concentrated to afford 2-(1-(4-(2,6-dioxopiperidin-3-yl)-3,5-difluorophenyl)piperidin-4-yl) acetaldehyde (200.000 mg, yield 77.37%, crude) which was used in next step without purification. LCMS: [M+H]⁺=351.2.

### Step 6: Preparation of N-(6-((5-bromo-2-((4-(4-(4-(2-(1-(4-(2,6-dioxopiperidin-3-yl)-3,5-difluorophenyl)piperidin-4-yl)ethyl) piperazin-1-yl)piperidin-1-yl)-5-ethyl-2-methoxyphenyl)amino)pyrimidin-4-yl)amino)quinoxalin-5-yl) methanesulfonamide

N-(6-((5-bromo-2-((5-ethyl-2-methoxy-4-(4-(piperazin-1-yl)piperidin-1-yl)phenyl)amino)pyrimidin-4-yl)amino)quinoxalin-5-yl) methanesulfonamide (101.564 mg, 0.143 mmol, 1 eq) and DIEA (18.410 mg, 0.143 mmol, 1 eq) in MeOH (2 mL) was stirred at 25° C. for 10 minutes, then AcOH (17.125 mg, 0.285 mmol, 2 eq) and 2-(1-(4-(2,6-dioxopiperidin-3-yl)-3,5-difluorophenyl)piperidin-4-yl) acetaldehyde (50.000 mg, 0.143 mmol, 1 eq) were added. The mixture was stirred at 25° C. for 0.5 h, then NaBH₃CN (8.991 mg, 0.143 mmol, 1 eq) was added. The mixture was stirred at 25° C. for further 1 h. LCMS indicated complete consumption of starting material and formation of product with desired mass. The reaction mixture was concentrated under reduced pressure to afford a residue. The residue was purified by Prep-HPLC (FA) to give N-(6-((5-bromo-2-((4-(4-(4-(2-(1-(4-(2,6-dioxopiperidin-3-yl)-3,5-difluorophenyl)piperidin-4-yl)ethyl) piperazin-1-yl)piperidin-1-yl)-5-ethyl-2-methoxyphenyl)amino)pyrimidin-4-yl)amino)quinoxalin-5-yl) methanesulfonamide (20.900 mg, yield 14.00%) as a white solid. LCMS: [M+H]⁺=1045.3.1H NMR (400 MHZ, DMSO-d₆) δ 10.86 (s, 1H), 9.00 (d, J=2.0 Hz, 1H), 8.93 (d, J=2.0 Hz, 1H), 8.86 (s, 1H), 8.64 (d, J=9.2 Hz, 1H), 8.29 (s, 1H), 8.21 (s, 1H), 7.87 (d, J=9.2 Hz, 1H), 7.39 (s, 1H), 6.77 (s, 1H), 6.61 (d. J=12.8 Hz, 2H), 4.04 (dd, J=8.4, 4.4 Hz, 1H), 3.78-3.71 (m, 6H), 3.03-2.99 (m, 6H), 2.82-2.52 (m, 14H), 2.44-2.34 (m, 3H), 2.15-2.08 (m, 1H), 2.00-1.97 (m, 1H), 1.89 (d. J=13.6 Hz, 2H), 1.72 (d, J=12.4 Hz, 2H), 1.62-1.53 (m, 2H), 1.46-1.41 (m, 2H), 1.24-1.21 (m, 2H), 1.19-1.16 (m, 1H), 0.84-0.80 (m, 3H).

### Example 23: Preparation of N-(6-((5-bromo-2-((4-(4-((9-(2-(2,6-dioxopiperidin-3-yl)-1-oxoisoindolin-5-yl)-3,9-diazaspiro[5.5]undecan-3-yl)methyl) piperidin-1-yl)-5-ethyl-2-methoxyphenyl)amino)pyrimidin-4-yl)amino)quinoxalin-5-yl) methanesulfonamide (Compound 54

### Step 1: Preparation of 3-(1-oxo-5-(3,9-diazaspiro[5.5]undecan-3-yl) isoindolin-2-yl) piperidine-2,6-dione hydrochloride

tert-Butyl 9-(2-(2,6-dioxopiperidin-3-yl)-1-oxoisoindolin-5-yl)-3,9-diazaspiro[5.5]undecane-3-carboxylate (200.000 mg, 0.402 mmol, 1 eq) was dissolved in HCl/EA (10 mL, 4 M). After stirring for 30 minutes, LCMS indicated complete consumption of starting material and formation of product with the desired mass. The reaction mixture was concentrated to afford 3-(1-oxo-5-(3,9-diazaspiro[5.5]undecan-3-yl) isoindolin-2-yl) piperidine-2,6-dione hydrochloride (156.000 mg, yield 97.69%) as a white solid which was directly used in the next step without purification. LCMS: [M+H]⁺=396.49.

### Step 2: Preparation of benzyl (4-(4-((9-(2-(2,6-dioxopiperidin-3-yl)-1-oxoisoindolin-5-yl)-3,9-diazaspiro[5.5]undecan-3-yl)methyl) piperidin-1-yl)-5-ethyl-2-methoxyphenyl) carbamate

3-(1-Oxo-5-(3,9-diazaspiro[5.5]undecan-3-yl) isoindolin-2-yl)piperidine-2,6-dione hydrochloride (240.237 mg, 0.555 mmol, 1.1 eq) and DIEA (97.798 mg, 0.757 mmol, 1.5 eq) were dissolved in MeOH (15 mL) at 15° C. benzyl (5-ethyl-4-(4-formylpiperidin-1-yl)-2-methoxyphenyl) carbamate (200.000 mg, 0.504 mmol, 1 eq) and AcOH (121.166 mg, 2.018 mmol, 4 eq) were added and the reaction mixture was stirred for 1 h. NaBH₃CN (63.307 mg, 1.009 mmol, 2 eq) was added and the reaction mixture was stirred for another 3 h. LCMS indicated complete consumption of starting material and formation of product with the desired mass. The mixture was concentrated and the residue was purified by column chromatography (DCM:MeOH=4:1) to afford benzyl (4-(4-((9-(2-(2,6-dioxopiperidin-3-yl)-1-oxoisoindolin-5-yl)-3,9-diazaspiro[5.5]undecan-3-yl)methyl) piperidin-1-yl)-5-ethyl-2-methoxyphenyl) carbamate (260.52 mg, yield 86.7%) as a yellow solid. LCMS: [M+H]⁺=776.98.

### Step 3: Preparation of 3-(5-(9-((1-(4-amino-2-ethyl-5-methoxyphenyl)piperidin-4-yl)methyl)-3,9-diazaspiro[5.5]undecan-3-yl)-1-oxoisoindolin-2-yl)piperidine-2,6-dione

Benzyl(4-(4-((9-(2-(2,6-dioxopiperidin-3-yl)-1-oxoisoindolin-5-yl)-3,9-diazaspiro[5.5]undecan-3-yl)methyl) piperidin-1-yl)-5-ethyl-2-methoxyphenyl) carbamate (300.000 mg, 0.386 mmol, 1 eq) was dissolved in EtOH (10 mL). Pd/C (41.090 mg, 10%, wet) was slowly added and the mixture was stirred at room temperature under H₂ atmosphere overnight. LCMS indicated complete consumption of starting material and formation of product with the desired mass. The reaction mixture was filtered and washed with EA, and the organic phase was collected and dried under reduced pressure to afford 3-(5-(9-((1-(4-amino-2-ethyl-5-methoxyphenyl)piperidin-4-yl)methyl)-3,9-diazaspiro[5.5]undecan-3-yl)-1-oxoisoindolin-2-yl)piperidine-2,6-dione (220.000 mg, yield 88.63%) as a white solid which was used directly in next step without further purification. LCMS: [M+H]⁺=642.85.

### Step 4: Preparation of N-(6-((5-bromo-2-((4-(4-((9-(2-(2,6-dioxopiperidin-3-yl)-1-oxoisoindolin-5-yl)-3,9-diazaspiro[5.5]undecan-3-yl)methyl) piperidin-1-yl)-5-ethyl-2-methoxyphenyl)amino)pyrimidin-4-yl)amino)quinoxalin-5-yl) methanesulfonamide

3-(5-(9-((1-(4-Amino-2-ethyl-5-methoxyphenyl)piperidin-4-yl)methyl)-3,9-diazaspiro[5.5]undecan-3-yl)-1-oxoisoindolin-2-yl)piperidine-2,6-dione (100.000 mg, 0.156 mmol, 1 eq), N-(6-((5-bromo-2-chloropyrimidin-4-yl)amino)quinoxalin-5-yl) methanesulfonamide (80.209 mg, 0.187 mmol, 1.2 eq) were dissolved in n-BuOH (10 ml), followed by addition of TsOH (53.575 mg, 0.311 mmol, 2 eq). The reaction mixture was stirred overnight at 80° C. LCMS indicated complete consumption of starting material and formation of product with the desired mass. The reaction mixture was concentrated under reduced pressure and the resulting crude residue was purified by Prep-HPLC (FA) to afford N-(6-((5-bromo-2-((4-(4-((9-(2-(2,6-dioxopiperidin-3-yl)-1-oxoisoindolin-5-yl)-3,9-diazaspiro[5.5]undecan-3-yl)methyl) piperidin-1-yl)-5-ethyl-2-methoxyphenyl)amino)pyrimidin-4-yl)amino)quinoxalin-5-yl) methanesulfonamide (50.000 mg, yield 31.02%) as a yellow solid. LCMS: [M+H]⁺=1036.06. ¹H NMR (400 MHZ, DMSO-d₆) § 10.92 (s, 1H), 8.98 (d, J=2.0 Hz, 1H), 8.92-8.84 (m, 2H), 8.65 (d, J=9.6 Hz, 1H), 8.28 (s, 1H), 8.21 (d, J=8.4 Hz, 1H), 7.84 (d, J=9.6 Hz, 1H), 7.50 (d. J=8.8 Hz, 1H), 7.38 (s, 1H), 7.06-7.01 (m, 2H), 6.78 (s, 1H), 5.04 (dd, J=13.2, 5.2 Hz, 1H), 4.32 (d, J=16.8 Hz, 1H), 4.20 (d, J=16.8 Hz, 1H), 3.76 (s, 3H), 3.02 (s, 3H), 2.98-2.83 (m, 4H), 2.71-2.55 (m, 4H), 2.45-2.37 (m, 6H), 2.23 (d, J=7.2 Hz, 2H), 2.02-1.91 (m, 2H), 1.80 (d, J=11.6 Hz, 2H), 1.58-1.48 (m, 8H), 1.26-1.24 (m, 5H), 0.87-0.82 (m, 3H).

| Compd # | Characterizations |
|---|---|
| 56 | LCMS: [M + H]⁺ = 1008.01. |
| | ¹H NMR (400 MHz, DMSO-d₆) δ 10.93 (s, 1H), 9.01 (d, J = 2.0 |
| | Hz, 1H), 8.94 (d, J = 2.0 Hz, 1H), 8.86 (s, 1H), 8.67 (s, 1H), 8.30 |
| | (s, 1H), 8.23 (s, 1H), 7.88 (d, J = 9.2 Hz, 1H), 7.49 (dd, J = 8.0, |
| | 5.6 Hz, 1H), 7.39 (s, 1H), 7.12 (d, J = 8.0 Hz, 1H), 6.79 (s, 1H), |
| | 6.59-6.43 (m, 2H), 5.04 (dd, J = 13.2, 5.2 Hz, 1H), 4.31 (d, J = |
| | 17.2 Hz, 1H), 4.19 (d, J = 16.8 Hz, 1H), 3.77 (s, 3H), 3.75-3.65 |
| | (m, 4H), 3.03 (s, 3H), 2.99-2.85 (m, 4H), 2.73-2.65 (m, 2H), |
| | 2.49-2.41 (m, 6H), 2.10-1.92 (m, 3H), 1.83-1.74 (m, 6H), 1.32- |
| | 1.22 (m, 4H), 0.89-0.81 (m, 3H). |
| 58 | LCMS: [M + H⁺] = 1009. |
| | ¹H NMR (400 MHz, DMSO-d₆) δ 10.95 (s, 1H), 9.01 (d, J = 2.0 |
| | Hz, 1H), 8.94 (d, J = 2.0 Hz, 1H), 8.88 (s, 1H), 8.65 (s, 1H), 8.30 |
| | (s, 1H), 8.23 (s, 1H), 7.88 (d, J = 9.2 Hz, 1H), 7.52 (d, J = 8.4 |
| | Hz, 1H), 7.40 (s, 1H), 7.13 (s, 1H), 7.06 (s, 1H), 6.80 (s, 1H), |
| | 6.67 (br, 1H), 5.05 (dd, J = 13.2, 5.2 Hz, 1H), 4.33 (d, J = 16.8 |
| | Hz, 1H), 4.21 (d, J = 16.8 Hz, 1H), 3.77 (s, 3H), 3.45-3.41 (m, |
| | 1H), 3.05-3.02 (m, 3H), 2.99-2.87 (m, 4H), 2.72-2.60 (m, 4H), |
| | 2.48-2.42 (m, 3H), 2.39-2.35 (m, 1H), 2.03-1.95 (m, 3H), 1.85- |
| | 1.75 (m, 7H), 1.38-1.27 (m, 5H), 0.91-0.82 (m, 4H). |

Compounds 56 and 58 were synthesized via a similar procedure to compound 54.

### Example 24: Preparation of N-(6-((5-bromo-2-((4-(4-(4-(2-(1-(2,6-dioxopiperidin-3-yl)-3-ethyl-2-oxo-2,3-dihydro-1H-benzo[d]imidazol-4-yl)ethyl) piperazin-1-yl) piperidin-1-yl)-2-methoxy-5-(1-methyl-1H-pyrazol-4-yl)phenyl)amino)pyrimidin-4-yl)amino)quinoxalin-5-yl) methanesulfonamide (Compound 28

### Step 1: Preparation of tert-butyl 4-(1-(2-bromo-5-methoxy-4-nitrophenyl)piperidin-4-yl) piperazine-1-carboxylate

To a solution of 1-bromo-2-fluoro-4-methoxy-5-nitrobenzene (3 g, 0.012 mol) in DMA (50 mL) was added tert-butyl 4-(piperidin-4-yl)piperazine-1-carboxylate (3.2 g, 0.012 mol) and K₂CO₃ (5 g, 0.036 mol). The reaction mixture was stirred at 80° C. for 2 hours. The mixture was diluted with water and extracted with EA. The combined organic layer was dried over Na₂SO₄ and concentrated in vacuum. The residue was purified by flash chromatography with EA in PE=0˜30% to give the title compound (4.75 g, 79.2% yield) as a yellow oil. LC/MS: 499.1 [M+H]⁺.

### Step 2: Preparation of tert-butyl 4-(1-(5-methoxy-2-(1-methyl-1H-pyrazol-4-yl)-4-nitrophenyl)piperidin-4-yl)piperazine-1-carboxylate

To a solution of tert-butyl 4-[1-(2-bromo-5-methoxy-4-nitrophenyl)piperidin-4-yl]piperazine-1-carboxylate (4 g, 0.008 mol) in dioxane (120 mL) and H₂O (20 mL) was added 1-methyl-4-(4,4,5,5-tetramethyl-1,3,2-dioxaborolan-2-yl)pyrazole (2.16 g, 0.010 mol), K₃PO₄ (3.4 g, 0.016 mol) and Pd (dppf) Cl₂-DCM (0.66 g, 0.008 mol). The reaction mixture was stirred at 100° C. for 17 hours under Ar. The mixture was diluted with water and extracted with EA. The combined organic layer was dried over Na₂SO₄ and concentrated in vacuum. The residue was purified by flash chromatography with MeOH in DCM=0˜10% to give the title compound (2.0 g, 51.2% yield) as a brown oil. LC/MS: 501.3 [M+H]⁺.

### Step 3: Preparation of tert-butyl 4-(1-(4-amino-5-methoxy-2-(1-methyl-1H-pyrazol-4-yl)phenyl)piperidin-4-yl)piperazine-1-carboxylate

A mixture of tert-butyl 4-{1-[5-methoxy-2-(1-methylpyrazol-4-yl)-4-nitrophenyl]piperidin-4-yl}piperazine-1-carboxylate (500 mg, 1.00 mmol) and iron powder (279 mg, 4.99 mmol) in EtOH (10 mL) and NH₄Cl solution (2 mL) was stirred at 80° C. for hour. The solid was filtered off and the filtrate was concentrated in vacuum. The residue was purified by flash chromatography with EA in PE=0˜30% to give the title compound (271 mg, 57.6% yield) as a brown solid. LC/MS: 417.3 [M+H]⁺.

### Step 4: Preparation of tert-butyl 4-(1-(4-((5-bromo-4-((5-(methylsulfonamido)quinoxalin-6-yl)amino)pyrimidin-2-yl)amino)-5-methoxy-2-(1-methyl-1H-pyrazol-4-yl)phenyl)piperidin-4-yl)piperazine-1-carboxylate

To a solution of tert-butyl 4-(1-(4-amino-5-methoxy-2-(1-methyl-1H-pyrazol-4-yl)phenyl)piperidin-4-yl)piperazine-1-carboxylate (1.62 g, 3.45 mmol) and N-(6-((5-bromo-2-chloropyrimidin-4-yl)amino)quinoxalin-5-yl) methanesulfonamide (1.0 g, 2.3 mmol) in n-BuOH (20 mL) was added TFA (1.18 g, 10.3 mmol). The reaction mixture was stirred at 80° C. for 16 hours. The reaction was quenched with water and extracted with EA. The organic phase was washed with brine and dried over Na₂SO₄. The solvent was removed in vacuum and the residue was purified by flash column chromatography with PE/EA=1/1 to give the title compound (500 mg, 26.0% yield) as a white solid. LC/MS: 862.9 [M+H]⁺.

### Step 5: Preparation of N-(6-((5-bromo-2-((2-methoxy-5-(1-methyl-1H-pyrazol-4-yl)-4-(4-(piperazin-1-yl)piperidin-1-yl)phenyl)amino)pyrimidin-4-yl)amino)quinoxalin-5-yl) methanesulfonamide

A solution of tert-butyl 4-(1-(4-((5-bromo-4-((5-(methylsulfonamido)quinoxalin-6-yl)amino)pyrimidin-2-yl)amino)-5-methoxy-2-(1-methyl-1H-pyrazol-4-yl)phenyl)piperidin-4-yl)piperazine-1-carboxylate (300 mg, 0.34 mmol) in HCl-dioxane (4 M, 5 mL) was stirred at 25° C. for 2 hours. The solution was concentrated in vacuum to give the desired compound (280 mg HCl salt, crude) as a white solid. LC/MS: 763.3 [M+H]⁺.

### Step 6: Preparation of N-(6-((5-bromo-2-((4-(4-(4-(2-(1-(2,6-dioxopiperidin-3-yl)-3-ethyl-2-oxo-2,3-dihydro-1H-benzo[d]imidazol-4-yl)ethyl) piperazin-1-yl)piperidin-1-yl)-2-methoxy-5-(1-methyl-1H-pyrazol-4-yl)phenyl)amino)pyrimidin-4-yl)amino)quinoxalin-5-yl) methanesulfonamide

To a solution of N-(6-((5-bromo-2-((2-methoxy-5-(1-methyl-1H-pyrazol-4-yl)-4-(4-(piperazin-1-yl)piperidin-1-yl)phenyl)amino)pyrimidin-4-yl)amino)quinoxalin-5-yl) methanesulfonamide (185 mg HCl salt, crude) and 2-(1-(2,6-dioxopiperidin-3-yl)-3-ethyl-2-oxo-2,3-dihydro-1H-benzo[d]imidazol-4-yl) acetaldehyde (62 mg, 0.19 mmol) in DMA (5 mL) was added DIEA (254 mg, 1.94 mmol). The mixture was stirred at 25° C. for hour. STAB (125 mg, 0.58 mmol) was added. The final mixture was stirred at 25° C. for hours. The reaction was quenched with water and extracted with EA. The organic phase was washed with brine and dried over Na₂SO₄. The solution was concentrated in vacuum to give a crude product. The crude product was purified by Prep-HPLC (Gemini-C18:150×21.2 mm, 5 um. ACN-H₂O (0.1% FA, 25-40%) to give compound 28 (18 mg, 8.9% yield) as a white solid. LC/MS: 1062.3 [M+H]⁺. ¹H NMR (400 MHZ, DMSO) δ 11.09 (s, 1H), 8.95 (d, J=1.6 Hz, 1H), 8.87 (d, J=1.8 Hz, 2H), 8.68 (brs, 1H), 8.38 (s, 1H), 8.28 (s, 1H), 8.24 (s, 1H), 7.98 (s, 1H), 7.76 (s, 1H), 7.59 (s, 1H), 7.44 (brs, 1H), 6.99 (d, J=4.9 Hz, 2H), 6.95-6.92 (m, 1H), 6.81 (s, 1H), 5.37 (dd, J=12.6, 5.3 Hz, 1H), 4.05-3.99 (m, 2H), 3.79 (s, 6H), 3.13 (d, J=10.9 Hz, 2H), 3.03-2.94 (m, 6H), 2.92-2.84 (m, 1H), 2.75-2.54 (m, 13H), 2.29-2.21 (m, 1H), 2.05-1.98 (m, 1H), 1.90-1.83 (m, 2H), 1.63-1.52 (m, 2H), 1.25 (t, J=6.9 Hz, 3H).

### Example 25: Preparation of N-(6-((5-bromo-2-((4-(4-(4-((1-(2-(2,6-dioxopiperidin-3-yl)-1-oxoisoindolin-5-yl)piperidin-4-yl)methyl) piperazin-1-yl)piperidin-1-yl)-2-methoxy-5-(1-methyl-1H-pyrazol-4-yl)phenyl)amino)pyrimidin-4-yl)amino)quinoxalin-5-yl) methanesulfonamide (Compound 32

### Step 1: Preparation of 3-(5-(4-(dimethoxymethyl) piperidin-1-yl)-1-oxoisoindolin-2-yl) piperidine-2,6-dione

To a solution of 3-(5-bromo-1-oxo-3H-isoindol-2-yl)piperidine-2,6-dione (1 g, 3.1 mmol) and 4-(dimethoxymethyl) piperidine (0.99 g, 6.2 mmol) in dioxane (10 mL) was added Pd-PEPPSI-IPentCl (0.15 g, 0.16 mmol) and Cs₂CO₃ (3.03 g, 9 mmol). The reaction was stirred under nitrogen at 100° C. for 2 hours. The mixture was concentrated in vacuum. The residue was purified by flash with MeOH in DCM=0˜ 10% to give the desired compound (1.1 g, 90% purity, 80.6% yield) as a white solid. LC/MS: 402.1 [M+H]⁺.

### Step 2: Preparation of 1-(2-(2,6-dioxopiperidin-3-yl)-1-oxoisoindolin-5-yl)piperidine-4-carbaldehyde

A solution of 3-{5-[4-(dimethoxymethyl) piperidin-1-yl]-1-oxo-3H-isoindol-2-yl}piperidine-2,6-dione (223 mg, 90% purity, 0.49 mmol) in DCM/TFA (6 mL, 3/1) was stirred at room temperature for 1 hour. The solution was concentrated in vacuum to give the desired compound (200 mg TFA salt, crude) as a yellow oil. LC/MS: 356.2 [M+H]⁺.

### Step 3: Preparation of N-(6-((5-bromo-2-((4-(4-(4-((1-(2-(2,6-dioxopiperidin-3-yl)-1-oxo isoindolin-5-yl)piperidin-4-yl)methyl) piperazin-1-yl)piperidin-1-yl)-2-methoxy-5-(1-methyl-1H-pyrazol-4-yl)phenyl)amino)pyrimidin-4-yl)amino)quinoxalin-5-yl) methanesulfonamide

To a solution of 1-[2-(2,6-dioxopiperidin-3-yl)-1-oxo-3H-isoindol-5-yl]piperidine-4-carbaldehyde (50 mg TFA salt, crude), N-{6-[(5-bromo-2-{[2-methoxy-5-(1-methyl pyrazol-4-yl)-4-[4-(piperazin-1-yl)piperidin-1-yl]phenyl]amino}pyrimidin-4-yl)amino]quinoxalin-5-yl}methanesulfonamide (86 mg, 0.11 mmol) in DMA/THF (2 mL, 1/1) was added DIEA (146 mg, 1.12 mmol). The mixture was stirred at room temperature for 0.5 hours. STAB (72 mg, 0.33 mmol) was added. The final mixture was stirred at room temperature for 16 hours. The reaction was quenched with water and extracted with EA. The organic phase was washed with brine and dried over Na₂SO₄. The solution was concentrated in vacuum and the residue was purified by Prep-HPLC (Gemini-C18 150×21.2 mm, 5 um; ACN-H₂O (0.1% FA)15-30%) to give the desired compound (38 mg, 29.0% yield) as a white solid. LC/MS: 1102.3 [M+H]⁺. ¹H NMR (400 MHZ, DMSO) δ 10.95 (s, 1H), 8.97 (d, J=1.7 Hz, 1H), 8.88 (d, J=1.7 Hz, 1H), 8.84 (s, 1H), 8.68 (brs, 1H), 8.39 (s, 1H), 8.29 (s, 1H), 8.20 (s, 1H), 7.98 (s, 1H), 7.76 (s, 1H), 7.60 (s, 1H), 7.55-7.24 (m, 2H), 7.07-7.02 (m, 2H), 6.81 (s, 1H), 5.05 (dd, J=13.3, 5.1 Hz, 1H), 4.32 (d, J=16.9 Hz, 1H), 4.20 (d. J=16.9 Hz, 1H), 3.91-3.84 (m, 2H), 3.81-3.77 (m, 6H), 3.16-3.09 (m, 2H), 3.02 (s, 3H), 2.98-2.72 (m, 4H), 2.69-2.54 (m, 7H), 2.45-2.23 (m, 6H), 2.17-2.11 (m, 2H), 2.00-1.93 (m, 1H), 1.90-1.74 (m, 5H), 1.63-1.52 (m, 2H), 1.24-1.14 (m, 2H).

### Example 26: Preparation of N-(6-((5-bromo-2-((4-(4-(4-(4-(2,6-dioxopiperidin-3-yl)-3,5-difluorophenethyl) piperazin-1-yl)piperidin-1-yl)-2-methoxy-5-(1-methyl-1H-pyrazol-4-yl)phenyl)amino)pyrimidin-4-yl)amino)quinoxalin-5-yl) methanesulfonamide (Compound 70

To a solution of N-(6-((5-bromo-2-((2-methoxy-5-(1-methyl-1H-pyrazol-4-yl)-4-(4-(piperazin-1-yl)piperidin-1-yl)phenyl)amino)pyrimidin-4-yl)amino)quinoxalin-5-yl) methanesulfonamide (185 mg HCl salt, crude) and 2-(4-(2,6-dioxopiperidin-3-yl)-3,5-difluorophenyl) acetaldehyde (52 mg, 0.19 mmol) in DMA (5 mL) was added DIEA (254 mg, 1.94 mmol). The mixture was stirred at 25° C. for 1 hour. STAB (125 mg, 0.58 mmol) was added. The final mixture was stirred at 25° C. for 16 hours. The reaction was quenched with water and extracted with EA. The organic phase was washed with brine and dried over Na₂SO₄. The solution was concentrated in vacuum to give a crude product. The crude product was purified by Prep-HPLC (Gemini-C18:150×21.2 mm, 5 um. ACN-H₂O (0.1% FA, 25-40%) to give the desired product (5.5 mg, 2.7% yield) as a white solid. LC/MS: 1014.3 [M+H]⁺. ¹H NMR (400 MHZ, DMSO) δ 10.94 (s, 1H), 8.95 (d, J=1.8 Hz, 1H), 8.88-8.83 (m, 2H), 8.67 (brs, 1H), 8.37 (s, 1H), 8.28 (s, 1H), 8.23 (s, 1H), 7.97 (s, 1H), 7.76 (s, 1H), 7.58 (s, 1H), 7.45 (brs, 1H), 7.03 (d. J=10.0 Hz, 2H), 6.80 (s, 1H), 4.23-4.15 (m, 1H), 3.80-3.76 (m, 6H), 3.14-3.07 (m, 2H), 3.01 (s, 3H), 2.84-2.75 (m, 4H), 2.64-2.55 (m, 6H), 2.83-2.06 (m, 4H), 2.05-1.96 (m, 2H), 1.89-1.81 (m, 2H), 1.63-1.52 (m, 2H), 1.31-1.18 (m, 4H).

### Example 27: Preparation of N-(6-((5-bromo-2-((4-(4-(4-(4-((2,6-dioxopiperidin-3-yl)amino) phenethyl) piperazin-1-yl)piperidin-1-yl)-2-methoxy-5-(1-methyl-1H-pyrazol-4-yl)phenyl)amino)pyrimidin-4-yl)amino)quinoxalin-5-yl) methanesulfonamide (Compound 78

### Step 1: Preparation of 4-(2-((tert-butyldimethylsilyl)oxy) ethyl)aniline

To a solution of 2-(4-aminophenyl) ethan-1-ol (5.5 g, 0.04 mol) in DCM (50 mL) was added imidazole (4.1 g, 0.06 mol) and TBSCl (6.35 g, 0.04 mol) at 0° C. The mixture was stirred at room temperature for 1 hour. The reaction was quenched with saturated NaHCO₃ solution and extracted with DCM. The organic phase was washed with brine, dried over Na₂SO₄, and concentrated in vacuum. The residue was purified by flash chromatography on silica gel eluting with PE:EA=3:1 to afford the desired compound (8.3 g, 90.6% yield) as a white solid. LC/MS: 251.9 [M+H]⁺.

### Step 2: Preparation of 3-((4-(2-((tert-butyldimethylsilyl)oxy) ethyl)phenyl)amino) piperidine-2,6-dione

To a solution of 4-(2-((tert-butyldimethylsilyl)oxy) ethyl)aniline (1 g, 0.004 mol) in ACN (20 mL) was added DIEA (1 g, 0.008 mol) and 3-bromopiperidine-2,6-dione (0.77 g, 0.004 mol). The reaction was stirred at 80° C. for 8 hours. The mixture was concentrated in vacuum. The residue was purified by flash chromatography on silica gel eluting with PE:EA=3:1 to afford the desired compound (0.4 g, 27.5% yield) as a yellow oil. LC/MS: 362.9 [M+H]⁺.

### Step 3: Preparation of 3-((4-(2-hydroxyethyl)phenyl)amino) piperidine-2,6-dione

A solution of 3-((4-(2-((tert-butyldimethylsilyl)oxy) ethyl)phenyl)amino) piperidine-2,6-dione (400 mg, 1.10 mmol) in HCl-dioxane (4M, 20 mL) was stirred at room temperature for 2 hours. The mixture was concentrated in vacuum. The residue was purified by Prep-TLC with DCM/MeOH=20/1 to give the title compound (150 mg, 47.7% yield) as a white solid. LC/MS: 249.2 [M+H]⁺.

### Step 4: Preparation of 4-((2,6-dioxopiperidin-3-yl)amino) phenethyl 4-methylbenzenesulfonate

To a solution of 3-((4-(2-hydroxyethyl)phenyl)amino) piperidine-2,6-dione (70 mg, 0.28 mmol) in DCM (10 mL) was added TsCl (107 mg, 0.56 mmol), DIEA (109 mg, 0.85 mmol) and DMAP (3 mg, 0.03 mmol). The reaction was stirred at room temperature for hours. The mixture was concentrated in vacuum. The residue was purified by Prep-TLC with DCM:MeOH=10:1 to afford the desired compound (32 mg, 28.2% yield) as a white solid. LC/MS: 403.1 [M+H]⁺.

### Step 5: Preparation of N-(6-((5-bromo-2-((4-(4-(4-(4-((2,6-dioxopiperidin-3-yl)amino) phenethyl) piperazin-1-yl)piperidin-1-yl)-2-methoxy-5-(1-methyl-1H-pyrazol-4-yl)phenyl)amino)pyrimidin-4-yl)amino)quinoxalin-5-yl) methanesulfonamide

To a solution of 4-((2,6-dioxopiperidin-3-yl)amino) phenethyl 4-methylbenzenesulfonate (15 mg, 0.037 mmol) in DMSO (1 mL) was added N-(6-((5-bromo-2-((2-methoxy-5-(1-methyl-1H-pyrazol-4-yl)-4-(4-(piperazin-1-yl)piperidin-1-yl)phenyl)amino)pyrimidin-4-yl)amino)quinoxalin-5-yl) methanesulfonamide (29 mg, 0.037 mmol), DIEA (10 mg, 0.075 mmol) and KI (12 mg, 0.075 mmol). The reaction mixture was stirred at 80° C. for 16 hours. The solution was concentrated in vacuum. The residue was purified by Prep-HPLC (Gemini-C18:150×21.2 mm, 5 um. ACN-H₂O (0.1% FA, 40-70%) to give the desired compound (3 mg, 7.7% yield) as a yellow solid. LC/MS: 993.3 [M+H]⁺. ¹H NMR (400 MHZ, DMSO) δ 10.77 (brs, 1H), 9.01-8.90 (m, 2H), 8.84 (d, J=1.6 Hz, 1H), 8.68 (brs, 1H), 8.35 (s, 3H), 8.27 (s, 1H), 7.98 (s, 1H), 7.77 (s, 1H), 7.59 (s, 1H), 7.46 (brs, 1H), 6.94 (d, J=8.4 Hz, 2H), 6.81 (s, 1H), 6.60 (d, J=8.4 Hz, 2H), 5.64 (d, J=7.4 Hz, 1H), 4.30-4.22 (m, 1H), 3.82-3.76 (m, 6H), 3.13 (d, J=10.9 Hz, 2H), 3.00 (s, 3H), 2.75-2.54 (m, 10H), 2.48-2.40 (m, 5H), 2.32-2.20 (m, 2H), 2.14-2.08 (m, 1H), 1.88-1.82 (m, 2H), 1.62-1.52 (m, 2H).

### Example 28: Preparation of N-(6-((5-bromo-2-((4-(4-(2-(4-(4-((2,6-dioxopiperidin-3-yl)amino)phenyl)piperidin-1-yl) acetyl) piperazin-1-yl)-2-methoxy-5-(1-methyl-1H-pyrazol-4-yl)phenyl)amino)pyrimidin-4-yl)amino)quinoxalin-5-yl) methanesulfonamide (Compound 79

### Step 1: Preparation of tert-butyl 4-(4-((2,6-dioxopiperidin-3-yl)amino)phenyl)piperidine-1-carboxylate

A mixture of tert-butyl 4-(4-aminophenyl)piperidine-1-carboxylate (3 g, 0.01 mol), 3-bromopiperidine-2,6-dione (2.09 g, 0.01 mol) and Na₂CO₃ (3.47 g, 0.03 mol) in DMF (30 mL) was stirred at 85° C. for 16 hours. The reaction mixture was concentrated in vacuum and washed with EA (3×100 mL). The combined organic layer was concentrated in vacuum. The residue was purified by flash chromatography with EA in PE=0˜30% to give the title compound (0.5 g, 11.8% yield) as a white oil. LC/MS: 331.9 [M−56]+.

### Step 2: Preparation of 3-((4-(piperidin-4-yl)phenyl)amino) piperidine-2,6-dione

A solution of tert-butyl 4-{4-[(2,6-dioxopiperidin-3-yl)amino]phenyl}piperidine-1-carboxylate (400 mg, 1.03 mmol) in HCl-dioxane (4 M, 10 mL) was stirred at room temperature for 1 hour. The solution was concentrated in vacuum to give the desired compound (400 mg HCl salt, crude) as a yellow solid. LC/MS: 288.3 [M+H]⁺.

### Step 3: Preparation of tert-butyl 2-(4-(4-((2,6-dioxopiperidin-3-yl)amino)phenyl)piperidin-1-yl)acetate

To a solution of 3-{[4-(piperidin-4-yl)phenyl]amino}piperidine-2,6-dione (450 mg, 1.56 mmol) and tert-butyl 2-bromoacetate (400 mg HCl salt, crude) in DMF (10 mL) was added DIEA (607 mg, 4.69 mmol). The reaction mixture was stirred at room temperature for 16 hours. The mixture was concentrated in vacuum and washed with EA (3×100 mL). The combined organic layer was concentrated in vacuum. The residue was purified by flash chromatography with EA in PE=0˜30% to give the title compound (400 mg, 53.0% yield) as a yellow solid. LC/MS: 402.3 [M+H]⁺.

### Step 4: Preparation of 2-(4-(4-((2,6-dioxopiperidin-3-yl)amino)phenyl)piperidin-1-yl) acetic acid

A solution of tert-butyl 2-(4-{4-[(2,6-dioxopiperidin-3-yl)amino]phenyl}piperidin-1-yl)acetate (150 mg, 0.37 mmol) in DCM/TFA (4 mL, 3/1) was stirred at room temperature for 1 hour. The solution was concentrated in vacuum to give the desired compound (200 mg TFA salt, crude) as a yellow oil. LC/MS: 346.0 [M+H]⁺.

### Step 5: Preparation of N-(6-((5-bromo-2-((4-(4-(2-(4-(4-((2,6-dioxopiperidin-3-yl)amino)phenyl)piperidin-1-yl) acetyl) piperazin-1-yl)-2-methoxy-5-(1-methyl-1H-pyrazol-4-yl)phenyl)amino)pyrimidin-4-yl)amino)quinoxalin-5-yl) methanesulfonamide

To a solution of (4-{4-[(2,6-dioxopiperidin-3-yl)amino]phenyl}piperidin-1-yl) acetic acid (50 mg TFA salt, crude) and N-{6-[(5-bromo-2-{[2-methoxy-5-(1-methylpyrazol-4-yl)-4-(piperazin-1-yl)phenyl]amino}pyrimidin-4-yl)amino]quinoxalin-5-yl}methanesulfon amide (59 mg, 0.086 mmol) in DMA (2 mL) was added DIEA (33 mg, 0.26 mmol) and HATU (49 mg, 0.13 mmol). The reaction was stirred at room temperature for 2 hours. The mixture was concentrated in vacuum and the residue was purified by Prep-HPLC (Gemini-C18:150×21.2 mm, 5 um. ACN-H₂O (0.1% FA)15-40%) to give the desired product (24 mg, 26.0% yield) as a white solid. LC/MS: 1007.3 [M+H]⁺. ¹H NMR (400 MHZ, DMSO) δ 10.77 (s, 1H), 8.94 (d, J=1.7 Hz, 1H), 8.83 (s, 2H), 8.69 (brs, 1H), 8.42 (s, 1H), 8.30 (s, 1H), 8.12-8.05 (m, 2H), 7.75 (s, 1H), 7.63 (s, 1H), 7.45 (brs, 1H), 6.92 (d, J=8.5 Hz, 2H), 6.80 (s, 1H), 6.56 (d, J=8.5 Hz, 2H), 5.64 (d, J=7.5 Hz, 1H), 4.28-4.22 (m, 1H), 3.83-3.73 (m, 8H), 3.69-3.61 (m, 2H), 3.23 (s, 2H), 3.01 (s, 3H), 2.98-2.86 (m, 2H), 2.91-2.86 (m, 2H), 2.85-2.79 (m, 2H), 2.76-2.69 (m, 1H), 2.63-2.56 (m, 1H), 2.36-2.28 (m, 1H), 2.17-2.05 (m, 3H), 1.89-1.80 (m, 1H), 1.74-1.66 (m, 2H), 1.65-1.54 (m, 2H).

### Example 29: Preparation of N-(6-((5-bromo-2-((4-(4-(2-(4-(4-((2,6-dioxopiperidin-3-yl)amino)phenyl)piperazin-1-yl) acetyl) piperazin-1-yl)-2-methoxy-5-(1-methyl-1H-pyrazol-4-yl)phenyl)amino)pyrimidin-4-yl)amino)quinoxalin-5-yl) methanesulfonamide (Compound 80

### Step 1: Preparation of tert-butyl 4-(4-((2,6-dioxopiperidin-3-yl)amino)phenyl)piperazine-1-carboxylate

A mixture of tert-butyl 4-(4-aminophenyl)piperazine-1-carboxylate (3 g, 0.01 mol), 3-bromopiperidine-2,6-dione (2.07 g, 0.01 mol) and Na₂CO₃ (3.43 g, 0.03 mol) in DMF (30 mL) was stirred at 85° C. for 16 hours. The reaction mixture was concentrated in vacuum and washed with EA (3×100 mL). The combined organic layer was concentrated under vacuum. The residue was purified by flash chromatography with EA in PE=0˜30% to give the title compound (1 g, 25.7% yield) as a white oil. LC/MS: 389.0 [M−56]+.

### Step 2: Preparation of 3-((4-(piperazin-1-yl)phenyl)amino) piperidine-2,6-dione

A solution of tert-butyl 4-{4-[(2,6-dioxopiperidin-3-yl)amino]phenyl}piperazine-1-carboxylate (900 mg, 2.31 mmol) in HCl-dioxane (4 M, 10 mL) was stirred at room temperature for 1 hour. The solution was concentrated in vacuum to give the desired compound (1.4 g HCl salt, crude) as a yellow solid. LC/MS: 289.3 [M+H]⁺.

### Step 3: Preparation of tert-butyl 2-(4-(4-((2,6-dioxopiperidin-3-yl)amino)phenyl)piperazin-1-yl)acetate

To a solution of 3-{[4-(piperazin-1-yl)phenyl]amino}piperidine-2,6-dione (1.4 g HCl salt, crude), tert-butyl 2-bromoacetate (405 mg, 2.08 mmol) and tert-butyl 2-bromo acetate (405 mg, 2.08 mmol) in DMF (10 mL) was added DIEA (607 mg, 4.69 mmol). The reaction was stirred at room temperature for 16 hours. The mixture was concentrated in vacuum and washed with EA (3×100 mL). The combined organic layer was concentrated in vacuum. The residue was purified by flash chromatography with EA in PE=0˜30% to give the title compound (350 mg, 41.8% yield) as a yellow solid. LC/MS: 403.3 [M+H]⁺.

### Step 4: Preparation of 2-(4-(4-((2,6-dioxopiperidin-3-yl)amino)phenyl)piperazin-1-yl) acetic acid

A solution of tert-butyl 2-(4-{4-[(2,6-dioxopiperidin-3-yl)amino]phenyl}piperazin-1-yl)acetate (150 mg, 0.37 mmol) in DCM/TFA (4 mL, 3/1) was stirred at room temperature for 1 hour. The solution was concentrated in vacuum to give the desired compound (200 mg TFA salt, crude) as a yellow oil. LC/MS: 347.2 [M+H]⁺.

### Step 5: Preparation of N-(6-((5-bromo-2-((4-(4-(2-(4-(4-((2,6-dioxopiperidin-3-yl)amino)phenyl)piperazin-1-yl) acetyl) piperazin-1-yl)-2-methoxy-5-(1-methyl-1H-pyrazol-4-yl)phenyl)amino)pyrimidin-4-yl)amino)quinoxalin-5-yl) methanesulfonamide

To a solution of (4-{4-[(2,6-dioxopiperidin-3-yl)amino]phenyl}piperazin-1-yl) acetic acid (50 mg TFA salt, crude) and N-{6-[(5-bromo-2-{[2-methoxy-5-(1-methylpyrazol-4-yl)-4-(piperazin-1-yl)phenyl]amino}pyrimidin-4-yl)amino]quinoxalin-5-yl}methanesulfonamide (58 mg, 0.08 mmol) in DMA (2 mL) was added DIEA (33 mg, 0.26 mmol) and HATU (49 mg, 0.13 mmol). The reaction mixture was stirred at room temperature for 2 hours. The mixture was concentrated in vacuum and the residue was purified by Prep-HPLC (Gemini-C18:150×21.2 mm, 5 um. ACN-H₂O (0.1% FA)15-40%) to give the desired product (5.5 mg, 6.0% yield) as a white solid. LC/MS: 1008.2 [M+H]⁺. ¹H NMR (400 MHZ, DMSO) δ 10.76 (s, 1H), 8.93 (d, J=1.5 Hz, 1H), 8.82 (d, J=3.2 Hz, 2H), 8.67 (brs, 1H), 8.42 (s, 1H), 8.29 (s, 1H), 8.18 (brs, 1H), 8.12 (s, 1H), 7.75 (s, 1H), 7.62 (s, 1H), 7.46 (brs, 1H), 6.79 (s, 1H), 6.72 (d, J=8.8 Hz, 2H), 6.58 (d, J=8.8 Hz, 2H), 5.43-5.33 (m, 1H), 4.22-4.15 (m, 1H), 3.83-3.74 (m, 8H), 3.65 (s, 2H), 3.24 (s, 2H), 3.00 (s, 3H), 2.98-2.91 (m, 4H), 2.88-2.81 (m, 4H), 2.75-2.68 (m, 1H), 2.60-2.55 (m, 4H), 2.38-2.26 (m, 1H), 2.13-2.07 (m, 1H), 1.88-1.81 (m, 1H).

### Example 30: Preparation of N-(6-((5-bromo-2-((4-(4-(6-(2-(1-(4-((2,6-dioxopiperidin-3-yl)amino)-2-fluorophenyl)-4-hydroxypiperidin-4-yl) acetyl)-2,6-diazaspiro[3.3]heptan-2-yl)piperidin-1-yl)-2-methoxy-5-(1-methyl-1H-pyrazol-4-yl)phenyl)amino)pyrimidin-4-yl)amino)quinoxalin-5-yl) methane sulfonamide (Compound 81

### Step 1: Preparation of tert-butyl 6-(1-(5-methoxy-2-(1-methyl-1H-pyrazol-4-yl)-4-nitrophenyl)piperidin-4-yl)-2,6-diazaspiro[3.3]heptane-2-carboxylate

To a solution of 4-(2-fluoro-4-methoxy-5-nitrophenyl)-1-methyl-1H-pyrazole (600 mg, 2.39 mmol) in DMSO (10 mL) was added K₂CO₃ (990 mg, 7.17 mmol) and tert-butyl 6-(piperidin-4-yl)-2,6-diazaspiro[3.3]heptane-2-carboxylate (739 mg, 2.63 mmol). The reaction mixture was stirred at 90° C. for 16 hours. The reaction was diluted with water (30 mL) and extracted with DCM (30 mL×3). The organic phase was washed with brine and dried over Na₂SO₄. The solution was concentrated in vacuum and the residue was purified by pre-TLC (DCM/MeOH=20/1) to give the desired compound (760 mg, 62.8% yield) as a light yellow solid. LC/MS: 513.3 [M+H]⁺.

Step 2: Preparation of tert-butyl 6-(1-(4-amino-5-methoxy-2-(1-methyl-1H-pyrazol-4-yl)phenyl)piperidin-4-yl)-2,6-diazaspiro[3.3]heptane-2-carboxylate

To a solution of tert-butyl 6-(1-(5-methoxy-2-(1-methyl-1H-pyrazol-4-yl)-4-nitrophenyl)piperidin-4-yl)-2,6-diazaspiro[3.3]heptane-2-carboxylate (740 mg, 1.44 mmol) in EtOH/H₂O (9 mL, 2/1) was added NH₄Cl (386 mg, 7.22 mmol) and iron powder (323 mg, 5.77 mmol). The reaction was stirred at 80° C. for 2 hours. The solid was filtered off. The filtrate was concentrated in vacuum. The residue was dissolved with DCM (150 mL) and washed with water (30 mL). The organic phase was dried over Na₂SO₄ and concentrated in vacuum to give the desired compound (500 mg, 71.7% yield) as a white solid. LC/MS: 483.4 [M+H]⁺.

### Step 3: Preparation of tert-butyl 6-(1-(4-((5-bromo-4-((5-(methylsulfonamido)quinoxalin-6-yl)amino)pyrimidin-2-yl)amino)-5-methoxy-2-(1-methyl-1H-pyrazol-4-yl)phenyl)piperidin-4-yl)-2,6-diazaspiro[3.3]heptane-2-carboxylate

To a solution of tert-butyl 6-(1-(4-amino-5-methoxy-2-(1-methyl-1H-pyrazol-4-yl)phenyl)piperidin-4-yl)-2,6-diazaspiro[3.3]heptane-2-carboxylate (292 mg, 0.61 mmol) in n-BuOH (5 mL) was added N-(6-((5-bromo-2-chloropyrimidin-4-yl)amino)quinoxalin-5-yl) methanesulfonamide (200 mg, 0.47 mmol) and TFA (212 mg, 1.86 mmol). The reaction mixture was stirred at 90° C. for 7 hours. The mixture was diluted with water (30 mL) and extracted with DCM (30 mL×3). The organic phase was washed with brine and dried over Na₂SO₄. The solution was concentrated in vacuum and the residue was purified by Prep-TLC (DCM/MeOH=20/1) to give the desired compound (280 mg, 64.7% yield) as a white solid. LC/MS: 875.3 [M+H]⁺.

### Step 4: Preparation of N-(6-((2-((4-(4-(2,6-diazaspiro[3.3]heptan-2-yl)piperidin-1-yl)-2-methoxy-5-(1-methyl-1H-pyrazol-4-yl)phenyl)amino)-5-bromopyrimidin-4-yl)amino)quinoxalin-5-yl) methanesulfonamide

A solution of tert-butyl 6-(1-(4-((5-bromo-4-((5-(methylsulfonamido)quinoxalin-6-yl)amino)pyrimidin-2-yl)amino)-5-methoxy-2-(1-methyl-1H-pyrazol-4-yl)phenyl)piperidin-4-yl)-2,6-diazaspiro[3.3]heptane-2-carboxylate (80 mg, 9.13 mmol) in HCl-dioxane (4 M, 2 mL) and DCM (2 mL) was stirred at room temperature for 1 hour. The mixture was concentrated in vacuum to give the desired compound (70 mg HCl salt, crude) as a yellow solid. LC/MS: 775.3 [M+H]⁺.

### Step 5: Preparation of N-(6-((5-bromo-2-((4-(4-(6-(2-(1-(4-((2,6-dioxopiperidin-3-yl)amino)-2-fluorophenyl)-4-hydroxypiperidin-4-yl) acetyl)-2,6-diazaspiro[3.3]heptan-2-yl) piperidin-1-yl)-2-methoxy-5-(1-methyl-1H-pyrazol-4-yl)phenyl)amino)pyrimidin-4-yl)amino)quinoxalin-5-yl) methanesulfonamide

To a solution of 2-(1-(4-((2,6-dioxopiperidin-3-yl)amino)-2-fluorophenyl)-4-hydroxypiperidin-4-yl) acetic acid (35 mg, 0.09 mmol) in DMF (2 mL) was added DIEA (36 mg, 0.28 mmol), N-(6-((2-((4-(4-(2,6-diazaspiro[3.3]heptan-2-yl)piperidin-1-yl)-2-methoxy-5-(1-methyl-1H-pyrazol-4-yl)phenyl)amino)-5-bromopyrimidin-4-yl)amino)quinoxalin-5-yl) methanesulfonamide (70 mg HCl salt, crude) and HATU (53 mg, 0.14 mmol). The reaction was stirred at room temperature for 1 hour. The mixture was diluted with water (10 mL) and extracted with DCM (10 mL×3). The organic phase was washed with brine and dried over Na₂SO₄. The solution was concentrated in vacuum and the residue was purified by Prep-HPLC (22% ACN in H₂O with 0.5% FA) to give the desired compound (60 mg, 57.1% yield) as a white solid. LC/MS: 1138.2 [M+H]⁺. ¹H NMR (400 MHZ, DMSO) δ 10.76 (s, 1H), 8.97 (d, J=1.8 Hz, 1H), 8.88 (d, J=1.8 Hz, 1H), 8.82 (s, 1H), 8.69 (brs, 1H), 8.38 (s, 1H), 8.29 (s, 1H), 8.13 (s, 1H), 7.95 (s, 1H), 7.77-7.72 (m, 2H), 7.68-7.65 (m, 1H), 7.59 (s, 1H), 6.87-6.82 (m, 1H), 6.79 (s, 1H), 6.54-6.46 (m, 2H), 6.41 (d, J=8.7 Hz, 1H), 5.76 (d, J=7.8 Hz, 1H), 4.31-4.27 (m, 2H), 4.01-3.97 (m, 2H), 3.82 (s, 3H), 3.79 (s, 3H), 3.76 (s, 2H), 3.67 (t. J=6.4 Hz, 1H), 3.01 (s, 3H), 2.91-2.82 (m, 4H), 2.68-2.66 (m, 1H), 2.63-2.57 (m, 4H), 2.20 (s, 2H), 2.11-2.06 (m, 2H), 2.02-1.97 (m, 2H), 1.84-1.78 (m, 2H), 1.74-1.70 (m, 2H), 1.64-1.60 (m, 2H), 1.53-1.48 (m, 2H), 1.47-1.41 (m, 2H).

### Example 31: Preparation of N-(6-((5-bromo-2-((4-(4-(4-(2-(2,6-dioxopiperidin-3-yl)-1,3-dioxoisoindolin-5-yl)piperazin-1-yl)piperidin-1-yl)-2-methoxy-5-(1-methyl-1H-pyrazol-4-yl)phenyl)amino)pyrimidin-4-yl)amino)quinoxalin-5-yl) methanesulfonamide (Compound 82

To a solution of N-(6-((5-bromo-2-((2-methoxy-5-(1-methyl-1H-pyrazol-4-yl)-4-(4-(piperazin-1-yl)piperidin-1-yl)phenyl)amino)pyrimidin-4-yl)amino)quinoxalin-5-yl) methanesulfonamide (20 mg, 0.026 mmol) in DMSO (1 mL) was added 2-(2,6-dioxopiperidin-3-yl)-5-fluoroisoindoline-1,3-dione (7 mg, 0.026 mmol) and K₂CO₃ (11 mg, 0.079 mmol). The reaction mixture was stirred at 100° C. for 2 hours. The mixture was concentrated in vacuum to give a crude product. The crude product was purified by Prep-HPLC (ACN-H₂O (0.1% TFA)) to give compound 82 (4.7 mg, 17.3% yield) as a yellow solid. LC/MS: 1019.2 [M+H]⁺. ¹H NMR (400 MHZ, DMSO) δ 11.07 (s, 1H), 9.88 (s, 1H), 9.80 (brs, 1H), 8.94 (d, J=1.9 Hz, 1H), 8.84 (d, J=1.8 Hz, 1H), 8.80 (s, 1H), 8.63 (brs, 1H), 8.41 (s, 1H), 8.27 (s, 1H), 7.92 (s, 1H), 7.80 (s, 1H), 7.75 (d, J=8.6 Hz, 1H), 7.57 (s, 1H), 7.49 (s, 1H), 7.37 (d, J=7.8 Hz, 1H), 6.76 (s, 1H), 5.11-5.04 (m, 1H), 4.36-4.17 (m, 2H), 3.80-3.75 (m, 6H), 3.70-3.65 (m, 2H), 3.30-3.15 (m, 8H), 2.98 (s, 3H), 2.66-2.57 (m, 3H), 2.19-2.10 (m, 2H), 2.01-1.93 (m, 2H), 1.95-1.78 (m, 2H).

### Example 32: Preparation of N-(6-((5-bromo-2-((4-(4-((4-(2-(2,6-dioxopiperidin-3-yl)-1,3-dioxoisoindolin-5-yl)piperazin-1-yl)methyl) piperidin-1-yl)-2-methoxy-5-(1-methyl-1H-pyrazol-4-yl)phenyl)amino)pyrimidin-4-yl)amino)quinoxalin-5-yl) methanesulfonamide (Compound 83

### Step 1: Preparation of 4-(2-fluoro-4-methoxy-5-nitrophenyl)-1-methyl-1H-pyrazole

To a solution of 1-bromo-2-fluoro-4-methoxy-5-nitrobenzene (4 g, 16 mmol) in dioxane/H₂O (21 mL, 2/1) was added 1-methyl-4-(4,4,5,5-tetramethyl-1,3,2-dioxaborolan-2-yl)pyrazole (4 g, 19.2 mmol), Na₂CO₃ (5.09 g, 48 mmol) and Pd (dppf) Cl₂ (1.16 g, 1.6 mmol). The reaction mixture was stirred at 80° C. for 16 hours under nitrogen. The reaction was quenched with water and extracted with EA. The organic phase was washed with brine and dried over Na₂SO₄. The solvent was removed in vacuum and the residue was purified by flash column chromatography with PE/EA=3/1 to give the title compound (3 g, 74.3% yield) as a yellow solid. LC/MS: 251.8 [M+H]⁺.

### Step 2: Preparation of (1-(5-methoxy-2-(1-methyl-1H-pyrazol-4-yl)-4-nitrophenyl)piperidin-4-yl) methanol

To a solution of 4-(2-fluoro-4-methoxy-5-nitrophenyl)-1-methyl-1H-pyrazole (1.2 g, 4.8 mmol) in DMSO (10 mL) was added piperidin-4-ylmethanol (550 mg, 4.8 mmol) and K₂CO₃ (2 g, 14.4 mmol). The reaction mixture was stirred at 100° C. for 16 hours. The mixture was diluted with water (50 mL) and extracted with DCM (3×50 mL). The combined organic layer was washed with brine, dried over anhydrous Na₂SO₄, and concentrated in vacuum. The residue was purified by flash chromatography on silica gel with DCM:MeOH=10:1 to afford the desired compound (1.3 g, 79.1% yield) as a yellow oil. LC/MS: 347.2 [M+H]⁺.

### Step 3: Preparation of (1-(4-amino-5-methoxy-2-(1-methyl-1H-pyrazol-4-yl)phenyl)piperidin-4-yl) methanol

To a solution of (1-(5-methoxy-2-(1-methyl-1H-pyrazol-4-yl)-4-nitrophenyl)piperidin-4-yl) methanol (1.3 g, 3.75 mmol) in EtOH (30 mL) and water (6 mL) was added iron powder (1.0 g, 18.77 mmol) and NH₄Cl (1.2 g, 22.52 mmol). The reaction mixture was stirred at 75° C. for 2 hours. The solid was filtered off. The filtrate was concentrated in vacuum. The residue was dissolved with DCM (150 mL) and washed with water (30 mL). The organic phase was dried over Na₂SO₄ and concentrated in vacuum to give a crude product. The crude product was purified by flash chromatography on silica gel eluting with DCM:MeOH=10:1 to afford the desired compound (600 mg, 50.5% yield) as a yellow solid. LC/MS: 317.0 [M+H]⁺.

### Step 4: Preparation of N-(6-((5-bromo-2-((4-(4-(hydroxymethyl) piperidin-1-yl)-2-methoxy-5-(1-methyl-1H-pyrazol-4-yl)phenyl)amino)pyrimidin-4-yl)amino)quinoxalin-5-yl) methanesulfonamide

To a solution of (1-(4-amino-5-methoxy-2-(1-methyl-1H-pyrazol-4-yl)phenyl)piperidin-4-yl) methanol (100 mg, 0.32 mmol) and N-{6-[(5-bromo-2-chloropyrimidin-4-yl)amino]quinoxalin-5-yl}methanesulfonamide (136 mg, 0.32 mmol) in n-Butanol (5 mL) was added TFA (144 mg, 1.26 mmol). The reaction mixture was stirred at 90° C. for 5 hours. The solution was concentrated in vacuum. The residue was purified by Prep-TLC with DCM:MeOH=10:1 to afford the desired compound (100 mg, 44.5% yield) as a white solid. LC/MS: 709.1 [M+H]⁺.

### Step 5: Preparation of N-(6-((5-bromo-2-((4-(4-formylpiperidin-1-yl)-2-methoxy-5-(1-methyl-1H-pyrazol-4-yl)phenyl)amino)pyrimidin-4-yl)amino)quinoxalin-5-yl) methanesulfonamide

To a solution of oxalyl chloride (230 mg, 0.42 mmol) in DCM (5 mL) was added dimethyl sulfoxide (164 mg, 0.49 mmol) dropwise at −70° C. The mixture was stirred at −70° C. for 30 minutes. A solution of N-(6-((5-bromo-2-((4-(4-(hydroxymethyl) piperidin-1-yl)-2-methoxy-5-(1-methyl-1H-pyrazol-4-yl)phenyl)amino)pyrimidin-4-yl)amino)quinoxalin-5-yl) methanesulfonamide (50 mg, 0.07 mmol) in DCM (5 mL) was added at −70° C. dropwise, followed by addition of TEA (177 mg, 0.42 mmol). The reaction mixture was stirred at −70° C. for 1 hour and then warmed to room temperature. The mixture was diluted with water (10 mL) and extracted with DCM (3×50 mL). The combined organic layer was washed with brine, dried over anhydrous Na₂SO₄, and concentrated in vacuum. The residue was purified by Prep-TLC with DCM:MeOH=10:1 to afford the desired compound (17 mg, 34.0% yield) as a white solid. LC/MS: 706.5 [M+H]⁺.

### Step 6: Preparation of N-(6-((5-bromo-2-((4-(4-((4-(2-(2,6-dioxopiperidin-3-yl)-1,3-dioxoisoindolin-5-yl)piperazin-1-yl)methyl) piperidin-1-yl)-2-methoxy-5-(1-methyl-1H-pyrazol-4-yl)phenyl)amino)pyrimidin-4-yl)amino)quinoxalin-5-yl) methanesulfonamide

To a solution of N-(6-((5-bromo-2-((4-(4-formylpiperidin-1-yl)-2-methoxy-5-(1-methyl-1H-pyrazol-4-yl)phenyl)amino)pyrimidin-4-yl)amino)quinoxalin-5-yl) methanesulfonamide (17 mg, 0.024 mmol) in DCM (2 mL) and DMA (1 mL) was added NaOAc (6 mg, 0.072 mmol), 2-(2,6-dioxopiperidin-3-yl)-5-(piperazin-1-yl) isoindole-1,3-dione hydrochloride (9 mg, 0.024 mmol) and NaBH₃CN (5 mg, 0.072 mmol). The reaction mixture was stirred at room temperature for 2 hours. The mixture was concentrated in vacuum. The residue was purified by Prep-HPLC (ACN-H₂O (0.1% TFA)) to give the desired compound (3.3 mg, 12.9% yield) as a yellow solid. LC/MS: 1033.1 [M+H]⁺, 1H NMR (400 MHZ, DMSO) δ 11.09 (brs, 1H), 9.21 (brs, 1H), 8.86 (s, 1H), 8.81-8.60 (m, 2H), 8.49-8.26 (m, 3H), 8.24 (s, 1H), 7.98 (s, 1H), 7.81 (s, 1H), 7.75-7.65 (m, 1H), 7.61 (s, 1H), 7.36 (s, 1H), 7.27 (d, J=7.3 Hz, 1H), 6.83 (s, 1H), 5.40-5.27 (m, 1H), 5.08 (dd, J=12.8, 4.9 Hz, 1H), 4.22 (t, J=6.2 Hz, 1H), 3.86-3.72 (m, 6H), 3.11 (d, J=10.1 Hz, 2H), 3.00 (s, 3H), 2.95-2.82 (m, 2H), 2.68-2.55 (m, 4H), 2.30 (d, J=6.3 Hz, 2H), 2.07-1.95 (m, 2H), 1.80 (d, J=11.3 Hz, 2H), 1.75-1.58 (m, 2H), 1.47-1.30 (m, 3H), 0.95-0.82 (m, 2H).

### Example 33: Preparation of N-(6-((5-bromo-2-((4-(4-((1-(2-(2,6-dioxopiperidin-3-yl)-1,3-dioxoisoindolin-5-yl)piperidin-4-yl)methyl) piperazin-1-yl)-2-methoxy-5-(1-methyl-1H-pyrazol-4-yl)phenyl)amino)pyrimidin-4-yl)amino)quinoxalin-5-yl) methanesulfonamide (Compound 84

### Step 1: Preparation of tert-butyl 4-(5-methoxy-2-(1-methyl-1H-pyrazol-4-yl)-4-nitrophenyl)piperazine-1-carboxylate

To a solution of 4-(2-fluoro-4-methoxy-5-nitrophenyl)-1-methyl-1H-pyrazole (3 g, 11.94 mmol) and tert-butyl piperazine-1-carboxylate (6.7 g, 35.82 mmol) in DMSO (50 mL) was added K₂CO₃ (4.9 g, 35.83 mmol). The reaction mixture was stirred under N₂ at 100° C. for 16 hours. The mixture was quenched with water and extracted with EA. The organic phase was washed with brine and dried over Na₂SO₄. The solution was concentrated in vacuum and the residue was purified by flash chromatography on silica gel with MeOH:DCM=1:10 to give the desired compound (2.5 g, 50.4% yield) as a white solid. LC/MS: 417.9 [M+H]⁺.

### Step 2: Preparation of tert-butyl 4-(4-amino-5-methoxy-2-(1-methyl-1H-pyrazol-4-yl)phenyl)piperazine-1-carboxylate

To a solution tert-butyl 4-(5-methoxy-2-(1-methyl-1H-pyrazol-4-yl)-4-nitrophenyl)piperazine-1-carboxylate (2.7 g, 6.45 mmol) in EtOH/H₂O (30 mL, 2/1) was added NH₄Cl (1.7 g, 32.26 mmol) and iron powder (1.8 g, 32.26 mmol). The reaction mixture was stirred at 80° C. for 3 hours. The mixture was filtered, and the filtrate was concentrated in vacuum. The residue was dissolved with DCM (150 mL) and washed with water (30 mL). The organic phase was washed with brine and dried over Na₂SO₄. The solution was concentrated in vacuum to give the desired compound (2.0 g, 79.7% yield) as a yellow solid. LC/MS: 388.0 [M+H]⁺.

### Step 3: Preparation of N-(6-((5-bromo-2-((2-methoxy-5-(1-methyl-1H-pyrazol-4-yl)-4-(piperazin-1-yl)phenyl)amino)pyrimidin-4-yl)amino)quinoxalin-5-yl) methanesulfonamide

To a solution of tert-butyl 4-(4-amino-5-methoxy-2-(1-methyl-1H-pyrazol-4-yl)phenyl)piperazine-1-carboxylate (2.03 g, 5.24 mmol) in n-BuOH (30 mL) was added N-(6-((5-bromo-2-chloropyrimidin-4-yl)amino)quinoxalin-5-yl) methanesulfonamide (1.50 g, 3.49 mmol) and TFA (1.59 g, 13.96 mmol). The reaction mixture was stirred at 90° C. for hours. The mixture was diluted with water (30 mL) and extracted with DCM (30 mL×3). The organic phase was washed with brine and dried over Na₂SO₄. The solution was concentrated in vacuum and the residue was purified by flash chromatography on silica gel with MeOH:DCM=1:10 to give the title compound (1 g, 42.1% yield) as a yellow solid. LC/MS: 679.6 [M+H]⁺.

### Step 4: Preparation of N-(6-((5-bromo-2-((4-(4-((1-(2-(2,6-dioxopiperidin-3-yl)-1,3-dioxoisoindolin-5-yl)piperidin-4-yl)methyl) piperazin-1-yl)-2-methoxy-5-(1-methyl-1H-pyrazol-4-yl)phenyl)amino)pyrimidin-4-yl)amino)quinoxalin-5-yl) methanesulfonamide

To a solution of N-(6-((5-bromo-2-((2-methoxy-5-(1-methyl-1H-pyrazol-4-yl)-4-(piperazin-1-yl)phenyl)amino)pyrimidin-4-yl)amino)quinoxalin-5-yl) methanesulfonamide (90 mg, 0.13 mmol), DIEA (102 mg, 0.79 mmol), and 1-(2-(2,6-dioxopiperidin-3-yl)-1,3-dioxoisoindolin-5-yl)piperidine-4-carbaldehyde (97 mg, 0.26 mmol) in DCM (2 mL) was added STAB (84 mg, 0.39 mmol). The reaction mixture was stirred at room temperature for 2 hours. The reaction was diluted with water (10 mL) and extracted with DCM (10 mL×3). The organic phase was washed with brine and dried over Na₂SO₄. The solution was concentrated in vacuum and the residue was purified by Prep-HPLC (40% ACN in H₂O with 0.5% FA) to give the desired compound (60 mg, 44% yield) as a white solid. LC/MS: 1033.3 [M+H]⁺. ¹H NMR (400 MHZ, DMSO) δ 11.06 (s, 1H), 9.89 (s, 1H), 8.96 (d, J=1.8 Hz, 1H), 8.87 (d, J=1.8 Hz, 1H), 8.81 (s, 1H), 8.67 (brs, 1H), 8.44-8.25 (m, 2H), 7.99 (s, 1H), 7.77 (s, 1H), 7.65 (d. J=8.5 Hz, 1H), 7.59 (s, 1H), 7.46 (brs, 1H), 7.33-7.28 (m, 1H), 7.26-7.19 (m, 1H), 6.84 (s, 1H), 5.75 (s, 1H), 5.06 (dd, J=12.7, 5.4 Hz, 1H), 4.06 (d. J=12.5 Hz, 2H), 3.78 (d. J=11.8 Hz, 6H), 3.03-2.95 (m, 5H), 2.92-2.81 (m, 5H), 2.62-2.53 (m, 4H), 2.29-2.20 (m, 2H), 2.04-1.98 (m, 1H), 1.90-1.78 (m, 3H), 1.29-1.09 (m, 3H).

### Example 34: Preparation of N-(6-((5-bromo-2-((4-(4-(4-((1-(2-(2,6-dioxopiperidin-3-yl)-1,3-dioxoisoindolin-5-yl)piperidin-4-yl)methyl) piperazin-1-yl)piperidin-1-yl)-2-methoxy-5-(1-methyl-1H-pyrazol-4-yl)phenyl)amino)pyrimidin-4-yl)amino)quinoxalin-5-yl) methanesulfonamide (Compound 85

To a solution of N-(6-((5-bromo-2-((2-methoxy-5-(1-methyl-1H-pyrazol-4-yl)-4-(4-(piperazin-1-yl)piperidin-1-yl)phenyl)amino)pyrimidin-4-yl)amino)quinoxalin-5-yl) methanesulfonamide (95 mg HCl salt, crude) and 1-(2-(2,6-dioxopiperidin-3-yl)-1,3-dioxoisoindolin-5-yl)piperidine-4-carbaldehyde (39 mg, 0.10 mmol) in DMA (5 mL) was added DIEA (135 mg, 1.04 mmol). The mixture was stirred at 25° C. for 1 hour. STAB (66 mg, 0.31 mmol) was added. The reaction mixture was stirred at 25° C. for 16 hours. The reaction was quenched with water and extracted with EA. The organic phase was washed with brine and dried over Na₂SO₄. The solution was concentrated in vacuum to give a crude product. The crude product was purified by Prep-HPLC (Gemini-C18:150×21.2 mm, 5 um. ACN-H₂O (0.1% FA, 25-40%) to give the desired product (10 mg, 8.5% yield) as a white solid. LC/MS: 1116.3 [M+H]⁺. ¹H NMR (400 MHz, DMSO) δ 11.09 (s, 1H), 8.97-8.92 (m, 1H), 8.86-8.84 (m, 1H), 8.68 (brs, 1H), 8.38 (s, 1H), 8.27 (s, 1H), 7.98 (s, 1H), 7.77 (s, 1H), 7.65 (d, J=8.6 Hz, 1H), 7.59 (s, 1H), 7.32-7.29 (m, 1H), 7.25-7.20 (m, 1H), 6.81 (s, 1H), 5.07 (dd, J=13.0, 5.3 Hz, 1H), 4.19-4.00 (m, 2H), 3.82-3.76 (m, 5H), 3.14-3.07 (m, 2H), 3.00 (s, 3H), 2.96-2.83 (m, 3H), 2.64-2.54 (m, 6H), 2.43-2.36 (m, 2H), 2.29-2.18 (m, 2H), 2.15-2.11 (m, 2H), 2.04-1.94 (m, 2H), 1.89-1.74 (m, 4H), 1.65-1.50 (m, 4H), 1.28-1.21 (m, 3H), 1.18-1.11 (m, 2H), 0.89-0.79 (m, 2H).

### Example 35: Preparation of N-(6-((5-bromo-2-((5-cyclopropyl-4-(4-(4-(2-(1-(2,6-dioxopiperidin-3-yl)-3-ethyl-2-oxo-2,3-dihydro-1H-benzo[d]imidazol-4-yl)ethyl) piperazin-1-yl)piperidin-1-yl)-2-methoxyphenyl)amino)pyrimidin-4-yl)amino)quinoxalin-5-yl) methanesulfonamide (Compound 86

### Step 1: Preparation of tert-butyl 4-(1-(2-cyclopropyl-5-methoxy-4-nitrophenyl)piperidin-4-yl) piperazine-1-carboxylate

To a solution of tert-butyl 4-[1-(2-bromo-5-methoxy-4-nitrophenyl)piperidin-4-yl]piperazine-1-carboxylate (500 mg, 1.00 mmol) and cyclopropylboronic acid (258 mg, 3.00 mmol) in DMA/water (18 mL, 8/1) was added K₃PO₄ (1.1 g, 5.00 mmol) and Pd (dppf) Cl₂ (73 mg, 0.10 mmol). The reaction mixture was stirred at 95° C. under nitrogen for 16 hours. The mixture was diluted with water and extracted with EA (3×100 mL). The combined organic layer was dried over Na₂SO₄ and concentrated in vacuum. The residue was purified by flash chromatography with EA in PE=0˜30% to give the title compound (207 mg, 44.8% yield) as a yellow solid. LC/MS: 461.3 [M+H]⁺.

### Step 2: Preparation of tert-butyl 4-(1-(4-amino-2-cyclopropyl-5-methoxyphenyl)piperidin-4-yl)piperazine-1-carboxylate

A mixture of tert-butyl 4-[1-(2-cyclopropyl-5-methoxy-4-nitrophenyl)piperidin-4-yl]piperazine-1-carboxylate (200 mg, 0.43 mmol) and iron powder (121 mg, 2.17 mmol) in NH₄Cl/MEOH (4 mL, 1/1) was stirred at room temperature for 1 hour. The solid was filtered off and the filtrate was concentrated in vacuum. The residue was purified by flash chromatography with EA in PE=0˜30% to give the title compound (86 mg, 46.0% yield) as a yellow solid. LC/MS: 430.9 [M+H]⁺.

### Step 3: Preparation of tert-butyl 4-(1-(4-((5-bromo-4-((5-(methylsulfonamido)quinoxalin-6-yl)amino)pyrimidin-2-yl)amino)-2-cyclopropyl-5-methoxyphenyl)piperidin-4-yl) piperazine-1-carboxylate

To a solution of tert-butyl 4-[1-(4-amino-2-cyclopropyl-5-methoxyphenyl)piperidin-4-yl]piperazine-1-carboxylate (90 mg, 0.20 mmol) and N-{6-[(5-bromo-2-chloropyrimidin-4-yl)amino]quinoxalin-5-yl}methanesulfonamide (89 mg, 0.20 mmol) in n-BuOH (10 mL) was added trifluoroacetic acid (95 mg, 0.83 mmol). The reaction mixture was stirred at 90° C. for 16 hours. The mixture was concentrated in vacuum, dissolved with EA (300 mL), and washed with water. The organic layer was dried over Na₂SO₄ and concentrated in vacuum. The residue was purified by Prep-TLC with DCM/MeOH=10/1 to give the title compound (95 mg, 55.1% yield) as a yellow solid. LC/MS: 823.2 [M+H]⁺.

### Step 4: Preparation of N-(6-((5-bromo-2-((5-cyclopropyl-2-methoxy-4-(4-(piperazin-1-yl) piperidin-1-yl)phenyl)amino)pyrimidin-4-yl)amino)quinoxalin-5-yl) methanesulfonamide

A solution of tert-butyl 4-{1-[4-({5-bromo-4-[(5-methanesulfonamidoquinoxalin-6-yl)amino]pyrimidin-2-yl}amino)-2-cyclopropyl-5-methoxyphenyl]piperidin-4-yl}piperazine-1-carboxylate (100 mg, 0.12 mmol) in DCM/TFA (4 mL, 3/1) was stirred at room temperature for 1 hour. The solution was concentrated in vacuum to give the desired compound (200 mg TFA salt, crude) as a yellow oil. LC/MS: 722.7 [M+H]⁺.

### Step 5: Preparation of N-(6-((5-bromo-2-((5-cyclopropyl-4-(4-(4-(2-(1-(2,6-dioxopiperidin-3-yl)-3-ethyl-2-oxo-2,3-dihydro-1H-benzo[d]imidazol-4-yl)ethyl) piperazin-1-yl)piperidin-1-yl)-2-methoxyphenyl)amino)pyrimidin-4-yl)amino)quinoxalin-5-yl) methanesulfonamide

To a solution of N-(6-{[5-bromo-2-({5-cyclopropyl-2-methoxy-4-[4-(piperazin-1-yl) piperidin-1-yl]phenyl}amino)pyrimidin-4-yl]amino}quinoxalin-5-yl) methanesulfonamide (200 mg TFA salt, crude) and 2-[1-(2,6-dioxopiperidin-3-yl)-3-ethyl-2-oxo-1,3-benzodiazol-4-yl]acetaldehyde (176 mg, 50% purity, 0.28 mmol) in DMA/THF (2 mL, 1/1) was added DIEA (121 mg, 0.93 mmol). The reaction mixture was stirred at 40° C. for 0.5 hours. STAB (59 mg, 0.28 mmol) was added, and the mixture was stirred at 40° C. for 16 hours. The reaction mixture was concentrated in vacuum and the residue was purified by Prep-HPLC (Gemini-C18:150×21.2 mm, 5 um. ACN-H₂O (0.1% FA)15-40%) to give the desired product (8.8 mg, 8.7% yield) as a yellow solid. LC/MS: 1022.3 [M+H]⁺, 1H NMR (400 MHZ, CDCl₃) δ 9.08 (s, 1H), 8.88 (d, J=1.9 Hz, 1H), 8.83 (d, J=1.8 Hz, 1H), 8.68 (d, J=9.4 Hz, 1H), 8.35-8.20 (m, 2H), 8.16 (s, 1H), 8.01 (d, J=9.5 Hz, 1H), 7.55-7.43 (m, 2H), 7.03-6.94 (m, 1H), 6.92-6.84 (m, 1H), 6.72-6.68 (m, 1H), 6.57 (s, 1H), 5.23-5.18 (m, 1H), 4.16-4.10 (m, 2H), 3.83 (s, 3H), 3.42-3.37 (m, 2H), 3.15-3.00 (m, 7H), 2.96-2.92 (m, 7H), 2.82-2.77 (m, 3H), 2.76-2.67 (m, 3H), 2.28-2.21 (m, 2H), 2.18-2.08 (m, 4H), 1.37 (t. J=7.1 Hz, 4H), 1.32-1.27 (m, 2H), 0.88-0.82 (m, 2H).

### Example 36: Preparation of N-(6-((5-bromo-2-((4-(4-(4-(2-(1-(2,6-dioxopiperidin-3-yl)-3-ethyl-2-oxo-2,3-dihydro-1H-benzo[d]imidazol-4-yl)ethyl) piperazin-1-yl) piperidin-1-yl)-5-isopropyl-2-methoxyphenyl)amino)pyrimidin-4-yl)amino)quinoxalin-5-yl) methanesulfonamide (Compound 87

### Step 1: Preparation of tert-butyl 4-(1-(5-methoxy-4-nitro-2-(prop-1-en-2-yl)phenyl)piperidin-4-yl)piperazine-1-carboxylate

To a solution of tert-butyl 4-[1-(2-bromo-5-methoxy-4-nitrophenyl)piperidin-4-yl]piperazine-1-carboxylate (500 mg, 1.00 mmol) and 4,4,5,5-tetramethyl-2-(prop-1-en-2-yl)-1,3,2-dioxaborolane (202 mg, 1.2 mmol) in dioxane/H₂O (6 mL, 5/1) was added K₃PO₄ (425 mg, 2.00 mmol) and Pd (dppf) Cl₂ (73 mg, 0.10 mmol). The reaction mixture was stirred under nitrogen at 110° C. for 16 hours. The mixture was diluted with water and extracted with EA (3×50 mL). The combined organic layer was dried over Na₂SO₄, filtered, and concentrated in vacuum. The residue was purified by flash chromatography with EA in PE=0˜30% to give the title compound (219 mg, 47.3% yield) as a yellow solid. LC/MS: 461.1 [M+H]⁺.

### Step 2: Preparation of tert-butyl 4-(1-(4-amino-2-isopropyl-5-methoxyphenyl)piperidin-4-yl) piperazine-1-carboxylate

To a solution of tert-butyl 4-{1-[5-methoxy-4-nitro-2-(prop-1-en-2-yl)phenyl]piperidin-4-yl}piperazine-1-carboxylate (230 mg, 0.50 mmol) in MeOH (4 mL) was added Pd/C (26 mg, 0.25 mmol). The reaction mixture was stirred at room temperature for 16 hours under H₂. The catalyst was filtered off. The filtrate was concentrated in vacuum. The residue was purified by Flash chromatography with EA in PE=0˜30% to give the title compound (85 mg, 39.3% yield) as a yellow solid. LC/MS: 433.3 [M+H]⁺.

### Step 3: Preparation of tert-butyl 4-(1-(4-((5-bromo-4-((5-(methylsulfonamido)quinoxalin-6-yl)amino)pyrimidin-2-yl)amino)-2-isopropyl-5-methoxyphenyl)piperidin-4-yl)piperazine-1-carboxylate

To a solution of tert-butyl 4-[1-(4-Amino-2-isopropyl-5-methoxyphenyl)piperidin-4-yl]piperazine-1-carboxylate (130 mg, 0.30 mmol) and N-{6-[(5-bromo-2-chloropyrimidin-4-yl)amino]quinoxalin-5-yl}methanesulfonamide (129 mg, 0.30 mmol) in n-BuOH (5 mL) was added trifluoroacetic acid (137 mg, 1.2 mmol). The reaction mixture was stirred at 90° C. for 16 hours. The mixture was concentrated in vacuum and washed with EA (300 mL). The organic layer was dried over Na₂SO₄ and concentrated in vacuum. The residue was purified by Prep-TLC with DCM/MeOH=10/1 to give the title compound (52 mg, 21.0% yield) as a yellow solid. LC/MS: 825.2 [M+H]⁺.

### Step 4: Preparation of N-(6-((5-bromo-2-((5-isopropyl-2-methoxy-4-(4-(piperazin-1-yl) piperidin-1-yl)phenyl)amino)pyrimidin-4-yl)amino)quinoxalin-5-yl) methanesulfonamide

A solution of tert-butyl 4-{1-[4-({5-bromo-4-[(5-methanesulfonamidoquinoxalin-6-yl)amino]pyrimidin-2-yl}amino)-2-isopropyl-5-methoxyphenyl]piperidin-4-yl}piperazine-1-carboxylate (50 mg, 0.06 mmol) in DCM/TFA (4 mL, 3/1) was stirred at room temperature for 1 hour. The solution was concentrated in vacuum to give the desired compound (120 mg TFA salt, crude) as a yellow oil. LC/MS: 725.3 [M+H]⁺.

### Step 5: Preparation of N-(6-((5-bromo-2-((5-isopropyl-4-(4-(4-(2-(1-(2,6-dioxopiperidin-3-yl)-3-ethyl-2-oxo-2,3-dihydro-1H-benzo[d]imidazol-4-yl)ethyl) piperazin-1-yl)piperidin-1-yl)-2-methoxyphenyl)amino)pyrimidin-4-yl)amino)quinoxalin-5-yl) methanesulfonamide

To a solution of N-(6-{[5-bromo-2-({5-isopropyl-2-methoxy-4-[4-(piperazin-1-yl) piperidin-1-yl]phenyl}amino)pyrimidin-4-yl]amino}quinoxalin-5-yl) methanesulfonamide (60 mg TFA salt, crude) in DMA/THF (2 mL, 1/1) was added 2-[1-(2,6-dioxopiperidin-3-yl)-3-ethyl-2-oxo-1,3-benzodiazol-4-yl]acetaldehyde (78 mg, 50% purity, 0.12 mmol). The mixture was stirred at 40° C. for 0.5 hours. STAB (52 mg, 0.24 mmol) was added. The reaction mixture was stirred at 40° C. for 16 hours. The mixture was concentrated in vacuum and the residue was purified by Prep-HPLC (Gemini-C18:150×21.2 mm, 5 um. ACN-H₂O (0.1% FA)15-40%) to give the desired product (5 mg, 4.0% yield) as a yellow solid. LC/MS: 1024.3 [M+H]⁺. ¹H NMR (400 MHZ,) δ 11.07 (s, 1H), 8.94 (d, J=1.7 Hz, 1H), 8.87 (d, J=1.7 Hz, 1H), 8.81 (s, 1H), 8.63 (brs, 1H), 8.32 (s, 1H), 8.24 (s, 1H), 8.14 (s, 1H), 7.77 (d, J=6.1 Hz, 1H), 7.27 (s, 1H), 6.97-6.87 (m, 4H), 6.80 (s, 1H), 5.34 (dd, J=12.7, 5.3 Hz, 1H), 4.00-3.95 (m, 2H), 3.70 (s, 4H), 3.00-2.94 (m, 6H), 2.75-2.66 (m, 3H), 2.57-2.49 (m, 7H), 2.35-2.27 (m, 3H), 1.99-1.95 (m, 1H), 1.88-1.79 (m, 3H), 1.62-1.48 (m, 3H), 1.24-1.16 (m, 5H), 0.93 (d, J=6.4 Hz, 6H).

### Example 37: Preparation of N-(6-((5-bromo-2-((5-(4,4-difluorocyclohexyl)-4-(4-(4-(2-(1-(2,6-dioxopiperidin-3-yl)-3-ethyl-2-oxo-2,3-dihydro-1H-benzo[d]imidazol-4-yl)ethyl) piperazin-1-yl)piperidin-1-yl)-2-methoxyphenyl)amino)pyrimidin-4-yl)amino)quinoxalin-5-yl) methanesulfonamide (Compound 88

### Step 1: Preparation of tert-butyl 4-(1-(4′,4′-difluoro-4-methoxy-5-nitro-2′,3′,4′,5′-tetrahydro-[1,1′-biphenyl]-2-yl)piperidin-4-yl)piperazine-1-carboxylate

To a solution of tert-butyl 4-(1-(2-bromo-5-methoxy-4-nitrophenyl)piperidin-4-yl) piperazine-1-carboxylate (500 mg, 1.0 mmol) in DMF/H₂O (12 mL, 5/1) was added 2-(4,4-difluorocyclohex-1-en-1-yl)-4,4,5,5-tetramethyl-1,3,2-dioxaborolane (293 mg, 1.2 mmol), tripotassium phosphate (637 mg, 3 mmol) and 1,1′-Bis(diphenylphosphino) ferrocenepalladiumdichloride (146 mg, 0.2 mmol). The reaction mixture was stirred at 90° C. for 2 hours under nitrogen. The reaction was quenched with water and extracted with EA. The organic phase was washed with brine and dried over Na₂SO₄. The solvent was removed in vacuum and the residue was purified by flash column chromatography with PE/EA=3/1 to give the title compound (400 mg, 74.1% yield) as a white solid. LC/MS: 537.4 [M+H]⁺.

### Step 2: Preparation of tert-butyl 4-(1-(4-amino-2-(4,4-difluorocyclohexyl)-5-methoxyphenyl)piperidin-4-yl)piperazine-1-carboxylate

To a solution of tert-butyl 4-(1-(4′,4′-difluoro-4-methoxy-5-nitro-2′,3′,4′,5′-tetrahydro-[1,1′-biphenyl]-2-yl)piperidin-4-yl)piperazine-1-carboxylate (320 mg, 0.59 mmol) in i-PrOH (10 mL) was added Pd/C (126 mg, 1.19 mmol). The reaction was stirred at 25° C. for 96 hours under H₂. The catalyst was filtered off. The filtrate was concentrated in vacuum to afford the desired compound (280 mg, 92.3% yield) as a white solid. LC/MS: 509.3 [M+H]⁺.

### Step 3: Preparation of tert-butyl 4-(1-(4-((5-bromo-4-((5-(methylsulfonamido)quinoxalin-6-yl)amino)pyrimidin-2-yl)amino)-2-(4,4-difluorocyclohexyl)-5-methoxyphenyl)piperidin-4-yl)piperazine-1-carboxylate

To a solution of tert-butyl 4-(1-(4-amino-2-(4,4-difluorocyclohexyl)-5-methoxyphenyl)piperidin-4-yl)piperazine-1-carboxylate (280 mg, 0.55 mmol) in n-BuOH (10 mL) was added N-{6-[(5-bromo-2-chloropyrimidin-4-yl)amino]quinoxalin-5-yl}methanesulfonamide (307 mg, 0.71 mmol) and TFA (251 mg, 2.2 mmol). The reaction mixture was stirred at 80° C. for 16 hours. The reaction was quenched with water and extracted with EA. The organic phase was washed with brine and dried over Na₂SO₄. The solvent was removed in vacuum and the residue was purified by flash with PE:EA=1:1 to afford the desired compound (220 mg, 44.3% yield) as a brown solid. LC/MS: 901.3 [M+H]⁺.

### Step 4: Preparation of N-(6-((5-bromo-2-((5-(4,4-difluorocyclohexyl)-2-methoxy-4-(4-(piperazin-1-yl)piperidin-1-yl)phenyl)amino)pyrimidin-4-yl)amino)quinoxalin-5-yl) methanesulfonamide

A solution of tert-butyl 4-(1-(4-((5-bromo-4-((5-(methylsulfonamido)quinoxalin-6-yl)amino)pyrimidin-2-yl)amino)-2-(4,4-difluorocyclohexyl)-5-methoxyphenyl)piperidin-4-yl) piperazine-1-carboxylate (200 mg, 0.22 mmol) in TFA/DCM (6 mL, 1:3) was stirred at 25° C. for 1 hour. The solvent was removed in vacuum to afford the desired compound (240 mg TFA salt, crude) as a brown solid. LC/MS: 801.3 [M+H]⁺.

### Step 5: Preparation of N-(6-((5-bromo-2-((5-(4,4-difluorocyclohexyl)-4-(4-(4-(2-(1-(2,6-dioxopiperidin-3-yl)-3-ethyl-2-oxo-2,3-dihydro-1H-benzo[d]imidazol-4-yl)ethyl) piperazin-1-yl)piperidin-1-yl)-2-methoxyphenyl)amino)pyrimidin-4-yl)amino)quinoxalin-5-yl) methanesulfonamide

To a solution of N-(6-((5-bromo-2-((5-(4,4-difluorocyclohexyl)-2-methoxy-4-(4-(piperazin-1-yl)piperidin-1-yl)phenyl)amino)pyrimidin-4-yl)amino)quinoxalin-5-yl) methanesulfonamide (160 mg TFA salt, crude) in DMA (5 mL) was added 2-[1-(2,6-dioxopiperidin-3-yl)-3-ethyl-2-oxo-1,3-benzodiazol-4-yl]acetaldehyde (140 mg, 50% purity, 0.22 mmol). The mixture was stirred at 25° C. for 1 hour. STAB (95 mg, 0.44 mmol) was added. The reaction mixture was stirred at 25° C. for 16 hours. The reaction was quenched with water and extracted with EA. The organic phase was washed with brine and dried over Na₂SO₄. The solution was concentrated in vacuum to give a crude product. The crude product was purified by Prep-HPLC (Gemini-C18:150×21.2 mm, 5 um. ACN-H₂O (0.1% FA, 25-40%) to give the desired product (31 mg, 17.9% yield) as a white solid. LC/MS: 1100.3 [M+H]⁺. ¹H NMR (400 MHZ, DMSO) δ 11.10 (s, 1H), 9.96 (brs, 1H), 8.99 (d, J=1.6 Hz, 1H), 8.91 (d, J=1.6 Hz, 1H), 8.84 (s, 1H), 8.67 (brs, 1H), 8.36 (s, 1H), 8.29 (s, 1H), 8.14 (s, 1H), 7.83 (d, J=8 Hz, 1H), 7.28 (brs, 1H), 7.02-6.93 (m, 3H), 6.88 (s, 1H), 5.38 (dd, J=12.5, 5.3 Hz, 1H), 4.05-3.98 (m, 2H), 3.75 (s, 3H), 3.05-2.87 (m, 10H), 2.84-2.62 (m, 13H), 2.04-1.87 (m, 6H), 1.85-1.60 (m, 6H), 1.56-1.46 (m, 2H), 1.25 (t, J=6.9 Hz, 3H).

### Example 38: Preparation of N-(6-((5-bromo-2-((5-cyclopentyl-4-(4-(4-(2-(1-(2,6-dioxopiperidin-3-yl)-3-ethyl-2-oxo-2,3-dihydro-1H-benzo[d]imidazol-4-yl)ethyl) piperazin-1-yl)piperidin-1-yl)-2-methoxyphenyl)amino)pyrimidin-4-yl)amino)quinoxalin-5-yl) methanesulfonamide (Compound 89

### Step 1: Preparation of tert-butyl 4-(1-(2-(cyclopent-1-en-1-yl)-5-methoxy-4-nitrophenyl)piperidin-4-yl)piperazine-1-carboxylate

To a solution of tert-butyl 4-[1-(2-bromo-5-methoxy-4-nitrophenyl)piperidin-4-yl]piperazine-1-carboxylate (500 mg, 1.00 mmol) and 2-(cyclopent-1-en-1-yl)-4,4,5,5-tetramethyl-1,3,2-dioxaborolane (291 mg, 1.50 mmol) in dioxane/H₂O (12 mL, 5/1) was added K₃PO₄ (637 mg, 3.00 mmol) and Pd (dppf) Cl₂-DCM (81 mg, 0.10 mmol). The reaction mixture was stirred under nitrogen at 90° C. for 2 hours. The mixture was diluted with water and extracted with EA (3×50 mL). The combined organic layer was dried over Na₂SO₄, filtered, and the filtrate was concentrated in vacuum. The residue was purified by flash chromatography with EA in PE=0˜30% to give the title compound (332 mg, 68.2% yield) as a yellow solid. LC/MS: 487.4 [M+H]⁺.

### Step 2: Preparation of tert-butyl 4-(1-(4-amino-2-cyclopentyl-5-methoxyphenyl)piperidin-4-yl)piperazine-1-carboxylate

To a solution of tert-butyl 4-{1-[2-(cyclopent-1-en-1-yl)-5-methoxy-4-nitrophenyl]piperidin-4-yl}piperazine-1-carboxylate (300 mg, 0.61 mmol) in i-PrOH (10 mL) was added Pd/C (32 mg, 0.30 mmol). The reaction mixture was stirred at room temperature for 48 hours under H₂. The catalyst was filtered off. The filtrate was concentrated in vacuum. The residue was purified by flash chromatography with EA in PE=0˜30% to give the title compound (200 mg, 70.5% yield) as a yellow oil. LC/MS: 459.1 [M+H]⁺.

### Step 3: Preparation of tert-butyl 4-(1-(4-((5-bromo-4-((5-(methylsulfonamido)quinoxalin-6-yl)amino)pyrimidin-2-yl)amino)-2-cyclopentyl-5-methoxyphenyl)piperidin-4-yl) piperazine-1-carboxylate

To a solution of tert-butyl 4-[1-(4-amino-2-cyclopentyl-5-methoxyphenyl)piperidin-4-yl]piperazine-1-carboxylate (210 mg, 0.45 mmol) and N-{6-[(5-bromo-2-chloropyrimidin-4-yl)amino]quinoxalin-5-yl}methanesulfonamide (196 mg, 0.45 mmol) in n-BuOH (10 mL) was added trifluoroacetic acid (208 mg, 1.83 mmol). The reaction was stirred at 90° C. for 16 hours. The mixture was concentrated in vacuum. The residue was dissolved with EA (300 mL) and washed with water. The organic layer was dried over Na₂SO₄ and concentrated in vacuum. The residue was purified by Prep-TLC with DCM/MeOH=10/1 to give the title compound (161 mg, 41.4% yield) as a yellow solid. LC/MS: 851.3 [M+H]⁺.

### Step 4: Preparation of N-(6-((5-bromo-2-((5-cyclopentyl-2-methoxy-4-(4-(piperazin-1-yl) piperidin-1-yl)phenyl)amino)pyrimidin-4-yl)amino)quinoxalin-5-yl) methanesulfonamide

A solution of tert-butyl 4-{1-[4-({5-bromo-4-[(5-methanesulfonamidoquinoxalin-6-yl)amino]pyrimidin-2-yl}amino)-2-cyclopentyl-5-methoxyphenyl]piperidin-4-yl}piperazine-1-carboxylate (170 mg, 0.19 mmol) in DCM/TFA (4 mL, 3/1) was stirred at room temperature for 1 hour. The solution was concentrated in vacuum to give the desired compound (250 mg TFA salt, crude) as a yellow oil. LC/MS: 751.3 [M+H]⁺.

### Step 5: Preparation of N-(6-((5-bromo-2-((5-cyclopentyl-4-(4-(4-(2-(1-(2,6-dioxopiperidin-3-yl)-3-ethyl-2-oxo-2,3-dihydro-1H-benzo[d]imidazol-4-yl)ethyl) piperazin-1-yl)piperidin-1-yl)-2-methoxyphenyl)amino)pyrimidin-4-yl)amino)quinoxalin-5-yl) methanesulfonamide

To a solution of N-(6-{[5-bromo-2-({5-cyclopentyl-2-methoxy-4-[4-(piperazin-1-yl) piperidin-1-yl]phenyl}amino)pyrimidin-4-yl]amino}quinoxalin-5-yl) methanesulfonamide (250 mg TFA salt, crude) in DMA/THF (2 mL, 1/1) was added 2-[1-(2,6-dioxopiperidin-3-yl)-3-ethyl-2-oxo-1,3-benzodiazol-4-yl]acetaldehyde (188 mg, 50% purity, 0.29 mmol). The mixture was stirred at 40° C. for 0.5 hours. STAB (126 mg, 0.59 mmol) was added. The reaction mixture was stirred at 40° C. for 16 hours. The mixture was concentrated in vacuum and the residue was purified by Prep-HPLC (Gemini-C18:150× 21.2 mm, 5 um. ACN-H₂O (0.1% FA)15-30%) to give the desired product (14 mg, 6.3% yield) as a yellow solid. LC/MS: 1050.3 [M+H]⁺. ¹H NMR (400 MHz, DMSO) δ 11.07 (s, 1H), 8.94 (d, J=1.7 Hz, 1H), 8.87 (d, J=1.7 Hz, 1H), 8.81 (s, 1H), 8.63 (brs, 1H), 8.32 (s, 1H), 8.24 (s, 1H), 8.14 (s, 1H), 7.77 (d, J=6.1 Hz, 1H), 7.27 (brs, 1H), 6.96-6.88 (m, 4H), 6.80 (s, 1H), 5.36-5.31 (m, 1H), 4.00-3.94 (m, 2H), 3.70 (s, 4H), 2.99-2.95 (m, 6H), 2.75-2.67 (m, 3H), 2.58-2.52 (m, 7H), 2.34-2.26 (m, 3H), 1.99-1.95 (m, 1H), 1.87-1.80 (m, 3H), 1.61-1.50 (m, 3H), 1.24-1.18 (m, 5H), 0.93 (d, J=6.4 Hz, 8H).

### EGFR Degradation Assay

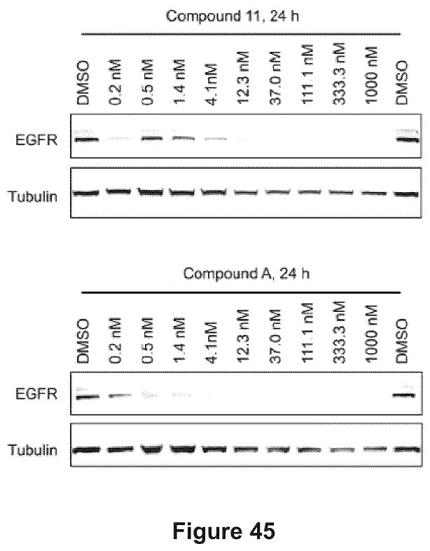

A₄₃₁ (EGFR_WT), NCl-H2073 (EGFR_WT), NCl-H292 (EGFR_WT), BaF3_YVAM (Her2_A775insYVAM), BaF3_SVD (EGFR_D770insSVD), BaF3_ASV (EGFR_A776inASV), or BaF3_DTC (EGFR_Exon19del/T790M/C797S) cells were seeded in a 24-well plate at a density of 4×10⁵ cells per well for EGFR WT cells and 8× cells per well for EGFR or Her2 mutant cells, and the plates were incubated overnight. The cells were treated with the desired compounds for 24 h. The culture medium was removed, and 100 μL of RIPA buffer (Beyotime, P0013B) containing 1% Protease Inhibitor Cocktail (Bimake, B14001), 1% Phosphatase Inhibitor Cocktail 2 (Sigma, P5726), and 1% Phosphatase Inhibitor Cocktail 3 (Sigma, P0044) were added to lyse the cells. Then, the cell lysates were transferred to 1.5 mL microcentrifuge tubes and centrifuged at 12,000 rpm and 4° C. for 30 min. The supernatants were transferred into new tubes, and the protein concentration was determined using the BCA protein quantification method. All samples were diluted to the same concentration using RIPA buffer, loading buffer was added, samples were denatured, and 15 μg of protein per lane was loaded onto a SurePAGE 4-20% Bis-Tris gel. Samples were separated via electrophoresis in MOPS running buffer at 180 V for 100 min. Then, proteins were transferred onto PVDF membranes using the MiniBlot™ Electrophoretic Transfer Cell Device (Beyotime, E6050). The membrane was blocked using block buffer (LI-COR 927-60001) to prevent non-specific binding. The membrane was incubated with primary antibodies (1:500 dilution, EGFR-CST4405, 1:2000 dilution, Tubulin-ABclonal #AC021) at 4° C. overnight. The membrane was washed three times with 1× TBST buffer for 15 min each. The membrane was incubated with secondary antibodies IRDye 680RD Goat anti-Rabbit IgG Secondary Antibody (1:2000 dilution, LI-COR 926-68071) and IRDye 800CW Goat anti-Mouse IgG Secondary Antibody (1:2000 dilution, LI-COR 926-32210). Finally, the membrane was washed again with 1× TBST buffer 3 times. Fluorescence was detected using the Odyssey CLx Imaging System, and the data were analyzed with Empiria Studio Software and GraphPad Prism 9. Table 4 and Table 5 list the degradation results, where the degradation potency is represented by DC₅₀, the concentration of compounds to cause the degradation of 50% of the corresponding proteins. FIGS. 45-49 show western blots illustrating the EGFR degradative activity of exemplary Compound of the present disclosure and a comparator Compound A in different cell lines 24 h after administration. The Compound referred herein as Compound A is disclosed in WO 2022/171123 A₁ (see, compound 133, page 46; published Aug. 18, 2022). WO 2022/171123 A₁ discloses phosphine oxide derived molecules with EGFR degradation activity. The structure of Compound A is:

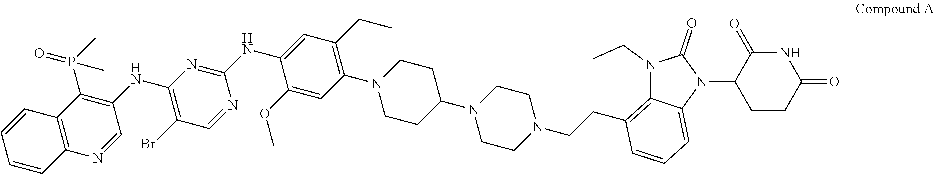 [SMILES: CCc1cc(Nc2ncc(Br)c(Nc3cnc4ccccc4c3P(C)(C)=O)n2)c(OC)cc1N1CCC(N2CCN(CCc3cccc4c3n(CC)c(=O)n4C3CCC(=O)NC3=O)CC2)CC1]

The structure of Compound 11 of the present disclosure is:

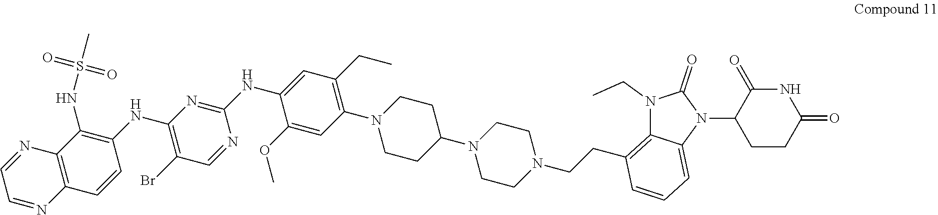 [SMILES: CCc1cc(Nc2ncc(Br)c(Nc3ccc4nccnc4c3NS(C)(=O)=O)n2)c(OC)cc1N1CCC(N2CCN(CCc3cccc4c3n(CC)c(=O)n4C3CCC(=O)NC3=O)CC2)CC1]

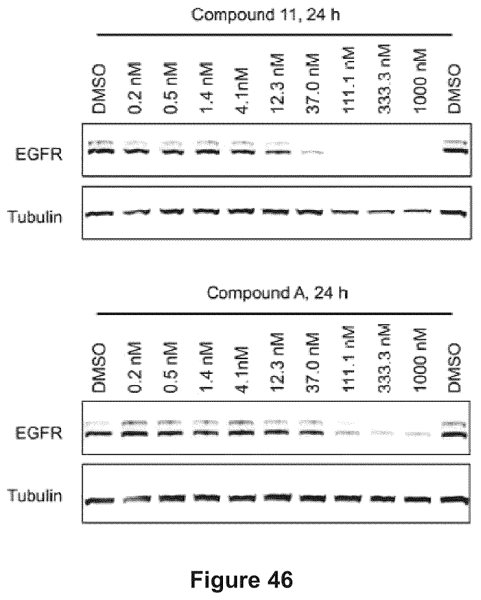

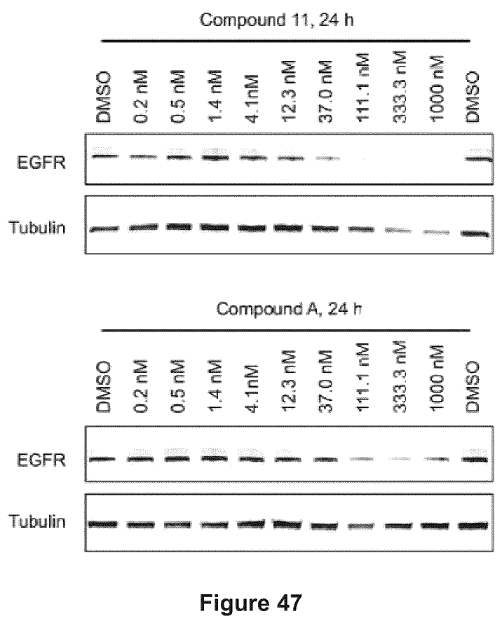

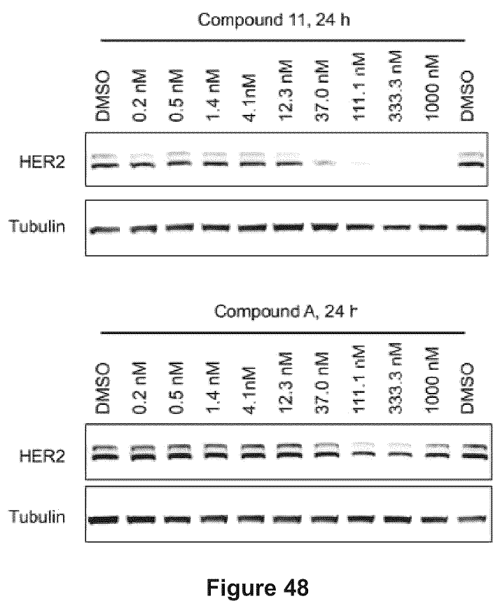

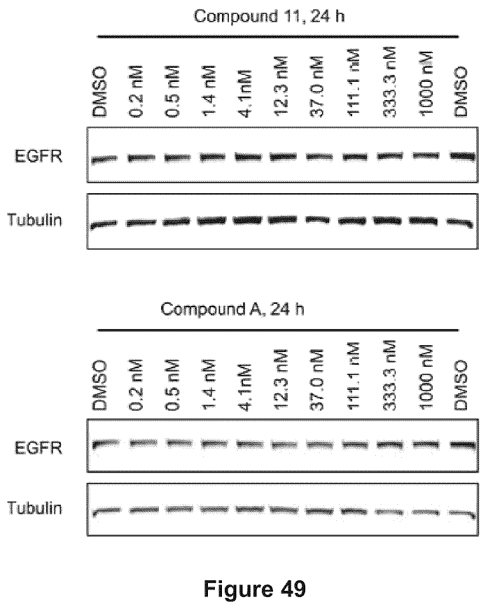

In particular, FIG. 45 illustrates the EGFR degradative activity of Compound 11 and the comparator Compound A in engineered BAF3_EGFR Del19/T790M/C797S cells hours after administration. FIG. 46 illustrates the EGFR degradative activity of Compound 11 and the comparator Compound A in engineered BAF3_EGFR ASV cells hours after administration. FIG. 47 illustrates the EGFR degradative activity of Compound 11 and the comparator Compound A in engineered BAF3_EGFR SVD cells hours after administration. FIG. 48 illustrates the EGFR degradative activity of Compound 11 and the comparator Compound A in engineered BAF3_HER2YVMA cells hours after administration. FIG. 49 illustrates the EGFR degradative activity of Compound 11 and a comparator Compound A in NCl-H2073 cells 24 hours after administration.

TABLE 4
Degradation of mutant and WT EGFR by
the quinoxaline derived sulfonamides

| | A431 | BaF3_DTC | BaF3_ASV |
|---|---|---|---|
| Compound # | DC₅₀ (nM) | DC₅₀ (nM) | DC₅₀ (nM) |
| 6 | >1000 | 5.937 | 288.7 |
| 7 | >1000 | 2.433 | 16.62 |
| 8 | >1000 | 23.34 | >1000 |
| 9 | >1000 | 8.048 | 250.8 |
| 10 | >1000 | 1.212 | 19.96 |
| 11 | >1000 | 1.196 | 22.42 |
| 28 | N/A | N/A | 93.64 |
| 32 | N/A | 3.097 | 25.96 |
| 35 | N/A | 66.04 | 86.45 |
| 36 | N/A | 1.272 | 102.1 |
| 37 | N/A | 8.598 | 101.6 |
| 38 | >1000 | 4.202 | 35.12 |
| 39 | N/A | 3.132 | N/A |
| 40 | N/A | 20.84 | 93.78 |
| 41 | >1000 | >100 | 335.4 |
| 42 | N/A | >100 | >1000 |
| 43 | N/A | 0.3232 | N/A |
| 44 | N/A | 4.11 | >1000 |
| 47 | N/A | 14.53 | N/A |
| 48 | >1000 | 4.265 | 23.7 |
| 49 | >1000 | 2.357 | 19.67 |
| 50 | N/A | 38.46 | 95.25 |
| 53 | N/A | 5.987 | >1000 |
| 54 | N/A | 13.28 | 33.49 |
| 55 | N/A | 41.76 | 55.26 |
| 56 | N/A | 9.324 | 18 |
| 57 | >1000 | 4.73 | 44.48 |
| 58 | N/A | 10.43 | 22.99 |
| 62 | N/A | 5.178 | 75.21 |
| 63 | N/A | 12.86 | 18.19 |
| 71 | >1000 | 3.512 | 55.1 |
| 77 | >1000 | 2.697 | >1000 |
| 90 | N/A | 38.62 | 134.8 |
| 91 | N/A | 8.04 | 53.7 |
| 92 | >1000 | 3.973 | 24.5 |

TABLE 5
Degradation of WT EGFR, mutant EGFR and mutant
Her2 by Compound 11 and a comparator Compound A

| | Compound 11 | Compound A |
|---|---|---|
| Cell lines | DC₅₀ (nM), 24 h | DC₅₀ (nM), 24 h |
| BaF3_Del19/T790M/C797S | 1.196 | 0.6224 |
| BaF3_SVD | 21.63 | 279.6 |
| BaF3_ASV | 22.42 | 108.9 |
| BaF3_YVMA | 33.26 | >1000 |
| NCI-H2073 | >1000 | >1000 |
| NCI-H292 | >1000 | >1000 |
| A431 | >1000 | >1000 |

As shown in Table 5, an exemplary compound of the present disclosure (i.e., Compound 11) showed significantly increased potency when compared with Compound A in degrading EGFR exon20 insertion mutant forms (BaF3_ASV, BaF3_SVD) and the Her2 mutant form (BaF3_YVMA).

### Cell Growth Inhibition Assay

HCC827_LTC and HCC827_DTC cell lines were generated by first transducing HCC827 cells (ATCC) with lentiviral particles expressing codon-optimized human EGFR L858R/T790M/C797S (LTC) mutant or Del19/T790M/C797S (DTC) mutant, and then selecting under 5 μg/ml puromycin treatment for a week. The endogenous EGFR alleles were subsequently abolished using the Alt-R™ CRISPR-Cas9 technology (Integrated DNA Technologies). Single clones of HCC827_LTC and HCC827_DTC were selected and validated for endogenous EGFR knockout using Sanger sequencing.

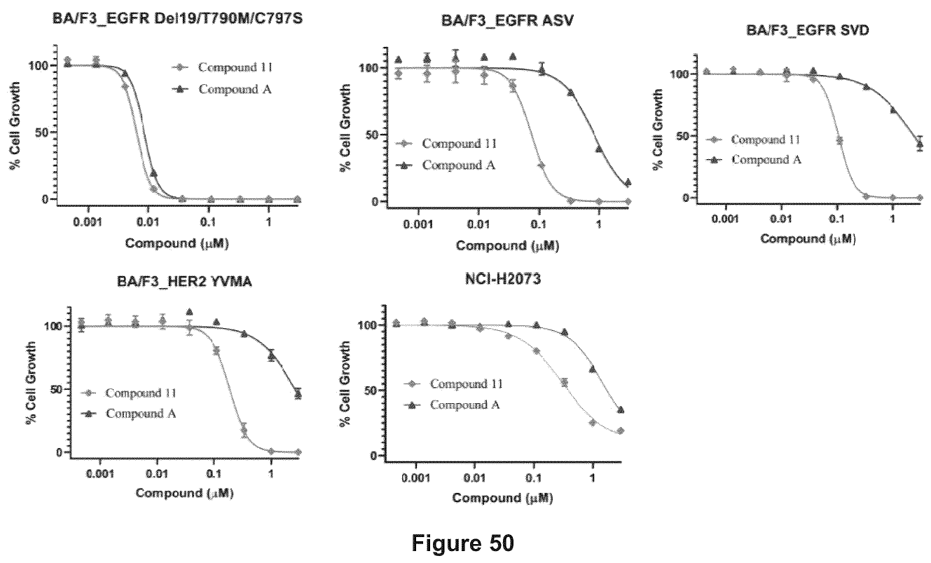

Cells were seeded in a 96-well plate (Corning 3610) with 90 μL of culture medium per well, and plates were incubated at 37° C. overnight. A 1,000× DMSO stock solution of the desired compound was prepared the following day. A compound dilution plate was prepared by diluting the stock solution 1:100 in the corresponding cell culture medium, 10 μL of the diluted compound solution was added to each 96-well plate well containing the seeded cells. Then, the cells were cultured at 37° C. for three days following compound treatment, 100 μL of CellTiter-Glo reagents (Promega G7573) was added to each well of the 96-well plate. The content of the wells was mixed by shaking continuously for 10 min, and luminescence was measured using Envision (PerkinElmer). The data were analyzed with GraphPad Prism 9 (see FIG. 50).

Table 6, Table 7, and Table 8 list the cell growth inhibition results, where Gls0 represents the compound concentration to cause 50% cell growth inhibition in comparison with DMSO. As shown in Table 7, an exemplary compound of the present disclosure (i.e., Compound 11) showed significantly increased potency when compared with Compound A in cell growth inhibition of engineered EGFR or HER2 exon20 insertion cells (BaF3_ASV, BaF3_SVD) and the Her2 mutant form (BaF3_YVMA).

TABLE 6
Inhibition of cell growth by quinoxaline derived sulfonamides

| | A431 | BaF3_DTC | BaF3_ASV |
|---|---|---|---|
| Compound # | GI₅₀ (nM) | GI₅₀ (nM) | GI₅₀ (nM) |
| 6 | >1000 | 11.22 | 282.3 |
| 7 | >1000 | 2.840 | 41.89 |
| 8 | >1000 | 24.68 | 857.9 |
| 9 | >1000 | 14.74 | 563.1 |
| 10 | >1000 | 9.603 | 72.66 |
| 11 | >1000 | 6.372 | 75.25 |
| 28 | 271 | 15.83 | 56.31 |
| 32 | 213.6 | 10.87 | 35.58 |
| 35 | >1000 | 13.65 | 125.9 |
| 36 | >1000 | 9.123 | 77.57 |
| 37 | 382.1 | 7.943 | 55.69 |
| 38 | >1000 | 9.14 | 90.27 |
| 39 | 440.8 | 11.06 | 73.41 |
| 40 | 399.3 | 7.992 | 60.19 |
| 41 | >1000 | 122.9 | 52.67 |
| 42 | 606 | 86.01 | 105.4 |
| 43 | >1000 | 14.99 | 119.2 |
| 44 | >1000 | 18.38 | 527.1 |
| 47 | >1000 | 12.09 | 47.49 |
| 48 | >1000 | 6.038 | 36.75 |
| 49 | 1000 | 4.429 | 32.67 |
| 50 | >1000 | 12.99 | 106 |
| 53 | >1000 | 22.69 | 330.1 |
| 54 | >1000 | 4.772 | 31.04 |
| 55 | >1000 | 7.409 | 127.7 |
| 56 | >1000 | 6.085 | 37.25 |
| 57 | 359.9 | 5.893 | 51.54 |
| 58 | >1000 | 11.25 | 48.97 |
| 62 | 383.1 | 19.34 | 130.2 |
| 63 | >1000 | 6.232 | 40.75 |
| 71 | >3000 | 5.411 | 131.2 |
| 77 | >3000 | 5.392 | 131 |
| 90 | >1000 | 19.08 | 89.43 |
| 91 | 447.6 | 9.257 | 48.62 |
| 92 | >1000 | 14.62 | 92.89 |

TABLE 7
Cell growth inhibition by compound 11 and reference compound
in WT EGFR, mutant EGFR and mutant Her2 cells

| | Compound 11 | Compound A |
|---|---|---|
| Cell Lines | GI₅₀ (nM), 24 h | GI₅₀ (nM), 24 h |
| BaF3_Del19/T790M/C797S | 6.372 | 8.575 |
| BaF3_SVD | 105.8 | 2345 |
| BaF3_ASV | 75.25 | 823.5 |
| BaF3_YVMA | 187.8 | 2634 |
| NCI-H2073 | 315.1 | 1511 |
| NCI-H292 | 435.9 | >3000 |
| A431 | 2505 | >3000 |

TABLE 8
Inhibition of cell growth by quinoxaline derived sulfonamides

| | HCC827 | HCC827_LTC | HCC827_DTC | NCI-H2073 | |
|---|---|---|---|---|---|
| Compound # | GI₅₀ (nM) | GI₅₀ (nM) | GI₅₀ (nM) | GI₅₀ (nM) | A431 GI₅₀ (nM) |
| 28 | 0.52 | 1.03 | 4.42 | 339.90 | 150.00 |
| 70 | 0.96 | 0.45 | 1.53 | 134.50 | 160.50 |
| 78 | 1.00 | 1.81 | 1.96 | 402.90 | 436.50 |
| 79 | 0.22 | 0.77 | 2.60 | 653.60 | 163.90 |
| 80 | 6.81 | 11.00 | 79.43 | 720.90 | 389.30 |
| 81 | 2.41 | 2.03 | 4.21 | >3000 | 745.50 |
| 82 | 2.82 | 9.38 | 30.80 | >3000 | >3000 |
| 83 | 5.51 | 8.58 | 19.37 | >3000 | >3000 |
| 84 | 1.18 | 3.06 | 10.70 | >3000 | >3000 |
| 85 | 2.72 | 3.57 | 7.70 | >3000 | >3000 |

FIG. 50 illustrates the cell growth inhibitory activity of Compound 11 and the comparator Compound A in engineered BA/F3 cells and NCl-H2073 cells three days after administration.

1. At least one entity chosen from a compound of Formula (I), tautomers, stereoisomers or a mixture of stereoisomers, pharmaceutically acceptable salts, hydrates, and deuterated derivatives thereof: 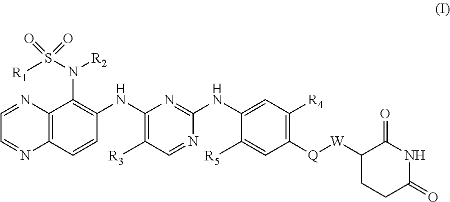
wherein:
R₁ is selected from hydroxy, amino, halogen, C₁-C₆ alkyl, C₃-C₆ cycloalkyl, C₁-C₆ haloalkyl, and C₁-C₆ alkoxy;
R₂ is selected from hydrogen, C₁-C₆ alkyl, C₃-C₆ cycloalkyl, and C₁-C₆ haloalkyl;
R₃ is selected from hydrogen, halogen, C₁-C₆ alkyl, C₃-C₆ cycloalkyl, C₁-C₆ haloalkyl, and C₁-C₆ alkoxy;
R₄ is selected from hydrogen, halogen, C₁-C₆ alkyl, C₃-C₆ cycloalkyl, C₁-C₄ alkenyl, C₁-C₅ alkoxy, C₃-C₆ cycloalkenyl, C₃-C₈ heterocyclyl, 5- to 10-membered heteroaryl, and 5- to 10-membered aryl; wherein each of the C₃-C₆ cycloalkyl, C₃-C₈ heterocyclyl, 5- to 10-membered heteroaryl, and 5- to 10-membered aryl is optionally substituted with 1-3 groups independently selected from halogen, hydroxy, amino, oxo, C₁-C₆ alkyl, C₃-C₆ cycloalkyl, and C₁-C₆ haloalkyl;
R₅ is selected from C₁-C₆ alkyl, deuterated C₁-C₆ alkyl, C₁-C₆ haloalkyl, C₃-C₆ cycloalkyl, deuterated C₁-C₆ alkoxy, C₁-C₆ alkoxy, and C₁-C₆ haloalkoxy;
Q is 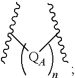
wherein each Q_{A} is independently selected from —C(H)(R_{Q})—, —O—, —N (R_{Q})—, —S(O)₂—, —C(O)—, —NH—, —C(O)N(R_{Q})—, C₃-C₆ cycloalkyl, 3- to 12-membered heterocycle, 6- to 12-membered bridged heterocycle, 6- to 12-membered spiro heterocycle, and 6- to 12-membered spiro cycloalkyl; wherein each of the C₃-C₆ cycloalkyl, 3- to 12-membered heterocycle, 6- to 12-membered bridged heterocycle, 6- to 12-membered spiro heterocycle, and 6- to 12-membered spiro cycloalkyl is optionally substituted with 1-3 R_{Q}, and wherein each R_{Q} is independently selected from hydrogen, hydroxyl, C₁-C₆ alkyl, and C₃-C₆ cycloalkyl;
n is 1, 2, 3, 4, 5, 6, 7, 8, 9, or 10; and
W is 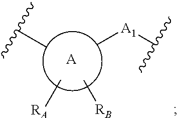
wherein Ring A is selected from 6- to 12-membered aryl and 5- to 12-membered heteroaryl; wherein A₁ is selected from a bond, —C(O) NH—, —NH—, —C(O)N(C₁-C₆ alkyl)-, and —C(O)O—; wherein R_{A} and R_{B}, which may be the same or different, are each independently selected from hydrogen, halogen, C₁-C₆ alkyl, C₃-C₆ cycloalkyl, and oxo; or R_{A} and R_{B} together with the carbon atom(s) to which they are attached form a 3- to 6-membered cycloalkyl or a 3- to 6-membered heterocycle.

2. The at least one entity according to claim 1, wherein R₁ is selected from C₁-C₆ alkyl.

3. The at least one entity according to claim 1, wherein R₁ is selected from methyl and ethyl.

4. The at least one entity according to claim 1, wherein R₂ is selected from hydrogen and C₁-C₆ alkyl.

5. The at least one entity according to claim 4, wherein R₂ is hydrogen or methyl.

6. The at least one entity according to claim 1, wherein R₃ is selected from hydrogen, halogen, C₁-C₆ alkyl, and C₁-C₆ haloalkyl.

7. The at least one entity according to claim 6, wherein R₃ is selected from Br, Cl, methyl, and trifluoromethyl.

8. The at least one entity according to claim 1, wherein R₄ is selected from hydrogen, halogen, C₁-C₆ alkyl, C₃-C₆ cycloalkyl, C₂-C₄ alkenyl, and 5- to 10-membered heteroaryl, wherein the 5- to 10-membered heteroaryl is optionally substituted with 1-2 groups independently selected from methyl, ethyl, propyl, butyl, and pentyl, and wherein the C₃-C₆ cycloalkyl is optionally substituted with 1-3 groups independently selected from halogen C₁-C₆ alkyl, and C₁-C₆ haloalkyl.

9. The at least one entity according to claim 1, wherein R₄ is selected from C₁-C₄ alkyl, C₂-C₄ alkenyl, C₃-C₆ cycloalkyl, and 5- to 10-membered heteroaryl, wherein the C₃-C₆ cycloalkyl is optionally substituted with 1-groups independently selected from halogen, and wherein the 5- to 10-membered heteroaryl is optionally substituted with one group selected from methyl, ethyl, propyl, butyl, and pentyl.

10. The at least one entity according to claim 1, wherein R₄ is selected from methyl, ethyl, C₂ alkenyl,

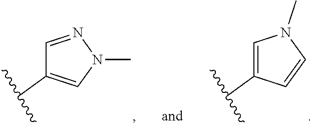

11. The at least one entity according to claim 1, wherein R₅ is C₁-C₆ alkoxy or deuterated C₁-C₆ alkoxy.

12. The at least one entity according to claim 11, wherein R₅ is —OCH₃ or —OCD₃.

13. The at least one entity according to claim 1, wherein each Q_{A} is independently selected from —C(H)(R_{Q})—, —O—, —N(R_{Q})—, —S(O)₂—, —C(O)—, —C(O)N(R_{Q})—, C₃-C₆ cycloalkyl, 3- to 12-membered heterocycle, 6- to 12-membered bridged heterocycle, 6- to 12-membered spiro heterocycle, and 6- to 12-membered spiro cycloalkyl; and wherein each R_{Q} is independently selected from hydrogen, C₁-C₆ alkyl, and C₃-C₆ cycloalkyl.

14. The at least one entity according to claim 1, wherein each Q_{A} is independently selected from —CH₂—, —O—, —C(O)—, —NH—, C₃-C₆ cycloalkyl, 4- to 8-membered heterocycle, 7- to 11-membered spiro heterocycle, and 7- to 11-membered spiro cycloalkyl.

15. The at least one entity according to claim 14, wherein each Q_{A} is independently selected from —CH₂—, —O—, —C(O)—, —NH—, cyclopropyl, piperidinyl, piperazinyl, pyrrolidinyl, azetidinyl, 2,7-diazaspiro[3.5]nonanyl, 3,9-diazaspiro[5.5]undecanyl, 2,6-diazaspiro[3.3]heptanyl, 7-azaspiro[3.5]nonanyl, 3-azaspiro[5.5]undecanyl, 2-azaspiro[3.5]nonanyl, and 2-azaspiro[3.3]heptanyl.

16. The at least one entity according to claim 1, wherein n is 2, 3, 4, 5, 6, 7, or 8.

17. The at least one entity according to claim 1, wherein Q is selected from

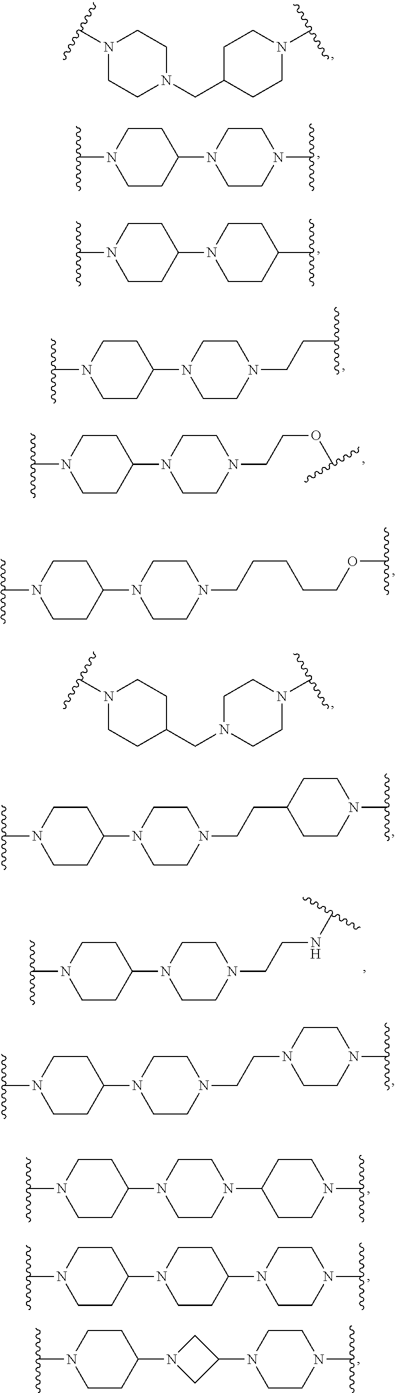

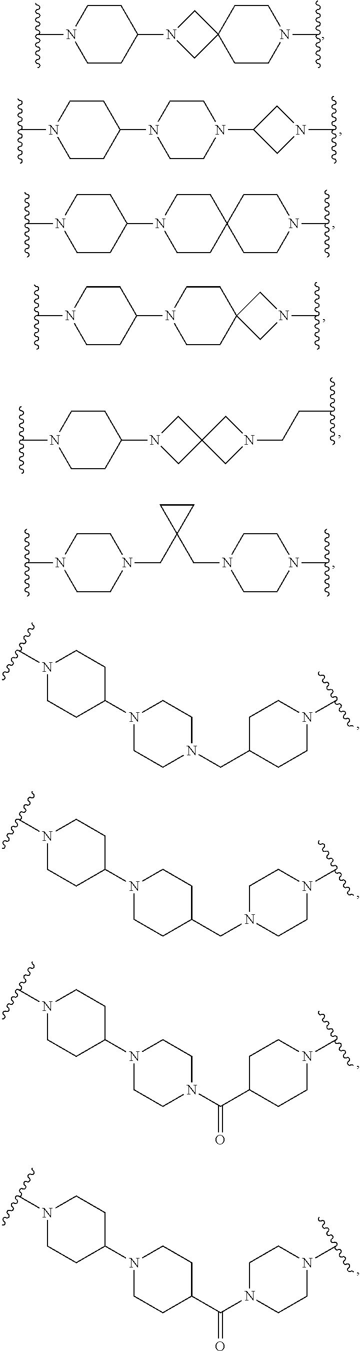

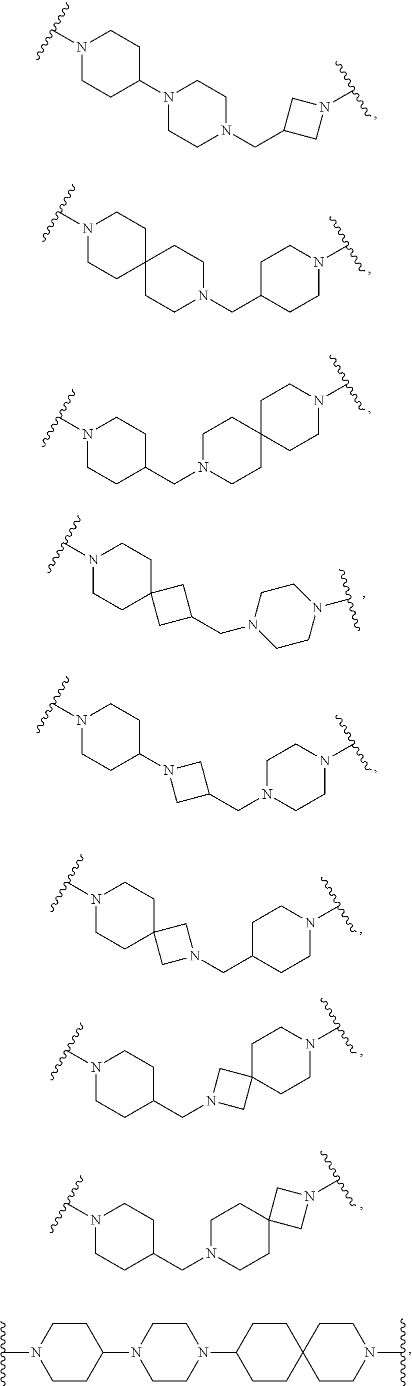

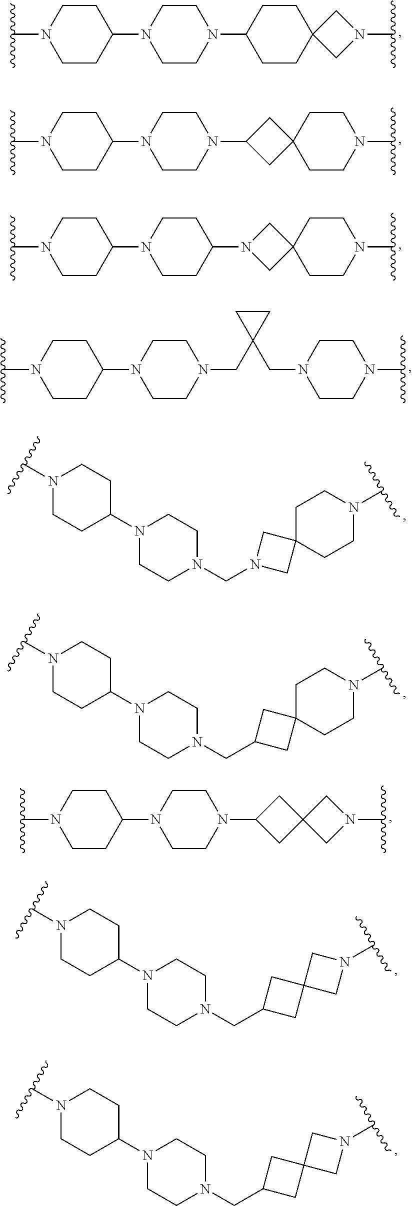

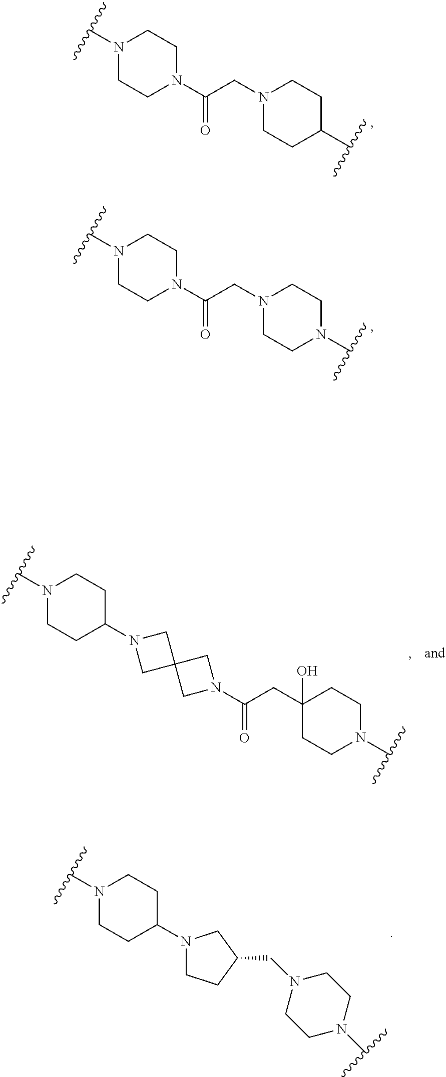 [SMILES: *C1CCN(CC(=O)N2CCN(*)CC2)CC1 ; *N1CCN(CC(=O)N2CCN(*)CC2)CC1 ; *N1CCC(N2CC3(CN(C(=O)CC4(O)CCN(*)CC4)C3)C2)CC1 ; *N1CCC(N2CC[C@@H](CN3CCN(*)CC3)C2)CC1]

18. The at least one entity according to claim 1, wherein W is 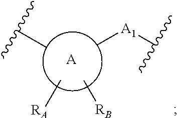
wherein Ring A is selected from 6- to 12-membered aryl and 9- to 12-membered heteroaryl; and wherein A₁ is a bond, —NH—, or —C(O)NH—.

19. The at least one entity according to claim 1, wherein Ring A is selected from

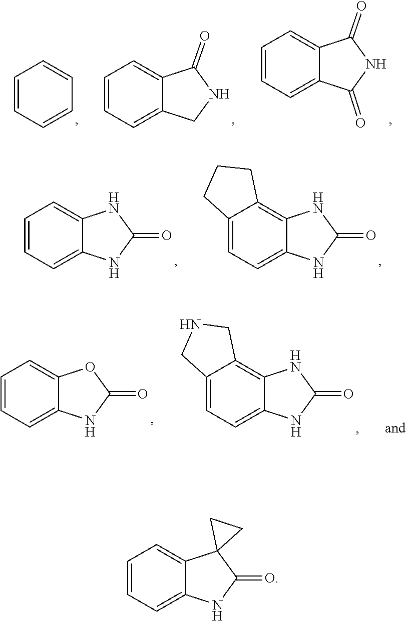

20. The at least one entity according to claim 1, wherein W is selected from

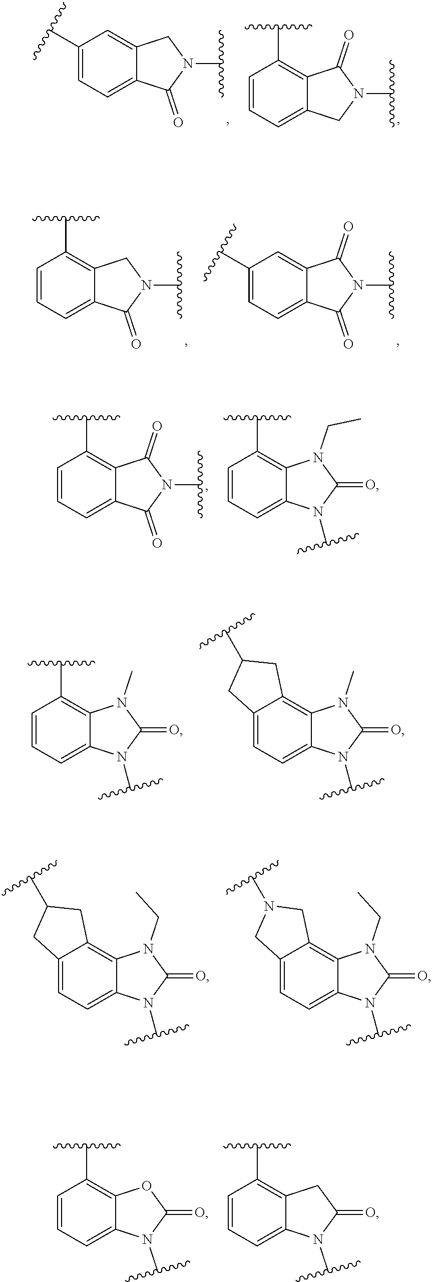

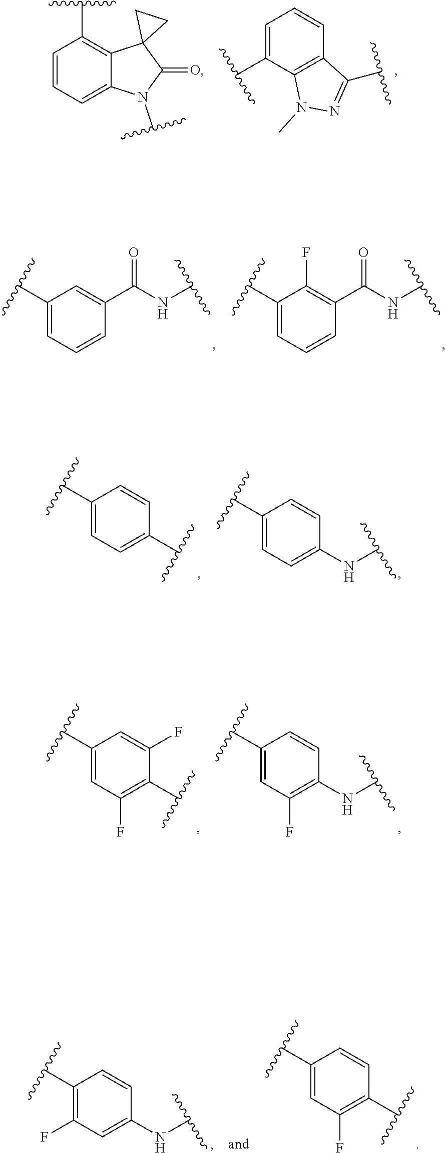

21. The at least one entity according to claim 1, wherein the at least one entity is selected from:
N-(6-((5-bromo-2-((4-(4-(4-((1-(2-(2,6-dioxopiperidin-3-yl)-1-oxoisoindolin-5-yl) piperidin-4-yl)methyl) piperazin-1-yl)piperidin-1-yl)-5-ethyl-2-methoxyphenyl)amino)pyrimidin-4-yl)amino)quinoxalin-5-yl) methanesulfonamide;
N-(6-((5-bromo-2-((4-(4-(4-(1-(2-(2,6-dioxopiperidin-3-yl)-1-oxoisoindolin-5-yl) piperidine-4-carbonyl) piperazin-1-yl)piperidin-1-yl)-5-ethyl-2-methoxyphenyl)amino)pyrimidin-4-yl)amino)quinoxalin-5-yl) methanesulfonamide;
N-(6-((5-bromo-2-((4-(4-(4-(2-(1-(2,6-dioxopiperidin-3-yl)-3-ethyl-2-oxo-2,3-dihydro-1H-benzo[d]imidazol-4-yl)ethyl) piperazin-1-yl)piperidin-1-yl)-5-ethyl-2-methoxyphenyl)amino)pyrimidin-4-yl)amino)quinoxalin-5-yl) methanesulfonamide;
N-(6-((5-bromo-2-((4-(4-(4-((1-(2-(2,6-dioxopiperidin-3-yl)-1-oxoisoindolin-5-yl) piperidin-4-yl)methyl) piperazin-1-yl)piperidin-1-yl)-2-methoxy-5-(1-methyl-1H-pyrazol-4-yl)phenyl)amino)pyrimidin-4-yl)amino)quinoxalin-5-yl) methanesulfonamide;
(S)-N-(6-((5-bromo-2-((4-(4-(4-(2-(2,6-dioxopiperidin-3-yl)-1-oxoisoindolin-5-yl) piperazin-1-yl)-[1,4′-bipiperidin]-1′-yl)-5-ethyl-2-methoxyphenyl)amino)pyrimidin-4-yl)amino)quinoxalin-5-yl) methanesulfonamide;
(S)-N-(6-((5-bromo-2-((4-(4-(4-(2-(2,6-dioxopiperidin-3-yl)-1-oxoisoindolin-5-yl) piperazin-1-yl)piperidin-1-yl)-5-ethyl-2-methoxyphenyl)amino)pyrimidin-4-yl)amino)quinoxalin-5-yl) methanesulfonamide;
(S)-N-(6-((5-bromo-2-((4-(4-(4-(2-(4-(2-(2,6-dioxopiperidin-3-yl)-1-oxoisoindolin-5-yl) piperazin-1-yl)ethyl) piperazin-1-yl)piperidin-1-yl)-5-ethyl-2-methoxyphenyl)amino)pyrimidin-4-yl)amino)quinoxalin-5-yl) methanesulfonamide;
N-(6-((5-bromo-2-((4-(4-(4-(7-(2-(2,6-dioxopiperidin-3-yl)-1-oxoisoindolin-5-yl)-7-azaspiro[3.5]nonan-2-yl)piperazin-1-yl)piperidin-1-yl)-5-ethyl-2-methoxyphenyl)amino)pyrimidin-4-yl)amino)quinoxalin-5-yl) methanesulfonamide;
N-(6-((5-bromo-2-((4-(4-(4-(1-(2-(2,6-dioxopiperidin-3-yl)-1-oxoisoindolin-5-yl) piperidin-4-yl)piperazin-1-yl)piperidin-1-yl)-5-ethyl-2-methoxyphenyl)amino)pyrimidin-4-yl)amino)quinoxalin-5-yl) methanesulfonamide;
N-(6-((5-bromo-2-((4-(4-((9-(2-(2,6-dioxopiperidin-3-yl)-1-oxoisoindolin-5-yl)-3,9-diazaspiro[5.5]undecan-3-yl)methyl) piperidin-1-yl)-5-ethyl-2-methoxyphenyl)amino)pyrimidin-4-yl)amino)quinoxalin-5-yl) methanesulfonamide;
N-(6-((5-bromo-2-((4-(4-((2-(2-(2,6-dioxopiperidin-3-yl)-1-oxoisoindolin-5-yl)-2,7-diazaspiro[3.5]nonan-7-yl)methyl) piperidin-1-yl)-5-ethyl-2-methoxyphenyl)amino)pyrimidin-4-yl)amino)quinoxalin-5-yl) methanesulfonamide;
N-(6-((5-bromo-2-((4-(4-((7-(2-(2,6-dioxopiperidin-3-yl)-1-oxoisoindolin-5-yl)-2,7-diazaspiro[3.5]nonan-2-yl)methyl) piperidin-1-yl)-5-ethyl-2-methoxyphenyl)amino)pyrimidin-4-yl)amino)quinoxalin-5-yl) methanesulfonamide;
(S)-N-(6-((5-bromo-2-((4-(4-(3-(4-(2-(2,6-dioxopiperidin-3-yl)-1-oxoisoindolin-5-yl) piperazin-1-yl) azetidin-1-yl)piperidin-1-yl)-5-ethyl-2-methoxyphenyl)amino)pyrimidin-4-yl)amino)quinoxalin-5-yl) methanesulfonamide;
N-(6-((5-bromo-2-((4-(4-((1-(2-(2,6-dioxopiperidin-3-yl)-1,3-dioxoisoindolin-5-yl) piperidin-4-yl)methyl) piperazin-1-yl)-2-methoxy-5-(1-methyl-1H-pyrazol-4-yl)phenyl)amino)pyrimidin-4-yl)amino)quinoxalin-5-yl) methanesulfonamide;
N-(6-((5-bromo-2-((4-(4-(4-((1-(2-(2,6-dioxopiperidin-3-yl)-1,3-dioxoisoindolin-5-yl) piperidin-4-yl)methyl) piperazin-1-yl)piperidin-1-yl)-2-methoxy-5-(1-methyl-1H-pyrazol-4-yl)phenyl)amino)pyrimidin-4-yl)amino)quinoxalin-5-yl) methanesulfonamide;
N-(6-((5-bromo-2-((4-(4-((S)-3-((4-(2-((S)-2,6-dioxopiperidin-3-yl)-1-oxoisoindolin-5-yl)piperazin-1-yl)methyl)pyrrolidin-1-yl)piperidin-1-yl)-5-ethyl-2-methoxyphenyl)amino)pyrimidin-4-yl)amino)quinoxalin-5-yl) methanesulfonamide;
and pharmaceutically acceptable salts thereof, deuterated derivatives thereof, and combinations thereof.

22. A pharmaceutical composition comprising the at least one entity according to claim 1 and at least one pharmaceutically acceptable carrier or excipient.

23. A method of decreasing EGFR activity by inhibition and/or degradation, comprising administering to a subject in need thereof the at least one entity according to claim 1 or the pharmaceutical composition according to claim 22.

24. A method of treating a disease or disorder associated with EGFR or HER2 in a subject, comprising administering to the subject the at least one entity according to claim 1 or the pharmaceutical composition according to claim 22.

25. The method according to claim 24, wherein the disease is cancer.

26. The method according to claim 25, wherein the cancer is associated with an EGFR or HER2 mutations, wherein the mutations comprise deletions, insertions, or point mutations in EGFR or Her2 exon 18-21.

27. The method according to claim 26, wherein the deletions, insertions or point mutations in EGFR comprise EGFR exon19 deletion, EGFR L858R, EGFR C797S, EGFR T790M, EGFR exon19 deletion/C797S, EGFR L858R/C797S, EGFR exon19 deletion/T790M/C797S, EGFR L858R/T790M/C797S, and/or EGFR or HER2 exon20 insertions.

28. The method according to claim 26, wherein the deletions, insertions or point mutations in Her2 exon 18-21 comprise HER2 exon20 insertions.

29. The method according to claim 25, wherein the cancer is selected from breast cancer, glioblastoma multiforme, urothelial cancer, head and neck cancer, and lung cancer.

30. The method according to claim 29, wherein the cancer is breast cancer.

31. The method according to claim 29, wherein the cancer is lung cancer.

32. The method according to claim 31, wherein the lung cancer is non-small cell lung cancer (NSCLC) or small cell lung cancer (SCLC).

33. The method according to claim 32, wherein the NSCLC is selected from adenocarcinoma, squamous cell carcinoma, and large cell carcinoma.

34. The method according to claim 32, wherein the lung cancer is SCLC.
